(19) United States
(12) Patent Application Publication
(10) Pub. No.: US 2025/0129016 A1
(43) Pub. Date: Apr. 24, 2025

(54) PHOTOREDOX METHODS FOR RADIOCYANATION OF ARENES AND USE THEREOF

(71) Applicant: THE UNIVERSITY OF NORTH CAROLINA AT CHAPEL HILL, Chapel Hill, NC (US)

(72) Inventors: Zibo Li, Chapel Hill, NC (US); David Nicewicz, Durham, NC (US); Xuedan Wu, Apex, NC (US)

(21) Appl. No.: 18/901,582

(22) Filed: Sep. 30, 2024

Related U.S. Application Data
(60) Provisional application No. 63/541,008, filed on Sep. 28, 2023.

(51) Int. Cl. C07C 253/30; B01J 31/02; B01J 31/16

(52) U.S. Cl. CPC C07C 253/30; B01J 31/0209; B01J 31/0244; B01J 31/0284; B01J 31/1625

(57) ABSTRACT

Carbon is one of the most common elements in bioactive organic compounds. Theoretically, nearly all carbon-based organic functional groups can be labeled with ¹¹C if an appropriate ¹¹C-synthon is developed and utilized. Although there are various reports on developing PET agents based on alkyl [¹¹C]nitriles, efficient and facile cyanation of arenes with radioisotope-containing reagents, particularly electron-rich arenes, still requires further improvements. The organic photoredox-catalyzed cyanation method reported herein introduces a [¹¹C]nitrile quickly with high radiochemical conversion (RCC) in a metal-free manner, which can also be further diversified to other functional groups such as [¹¹C]carboxylic acids, [¹¹C]amides, and [¹¹C]alkyl amines.

### RELATED APPLICATION DATA

The present application claims priority pursuant to 35 U.S.C. 119(e) to U.S. Provisional Patent Application Ser. No. 63/541,008 filed Sep. 28, 2023 which is incorporated herein by reference in its entirety.

### GOVERNMENTAL SUPPORT

This invention was made with government support under Grant No. EB029451 and CA233904 awarded by the National Institutes of Health (NIH). The government has certain rights in the invention.

### FIELD

The disclosure relates to organic photoredox-catalyzed cyanation methods to efficiently introduce a cyano functional group into an arene or heteroarene substrate. Furthermore, this method can be extended to include photoredox-catalyzed cyanation of arene and heteroarene substrates for introducing a radioisotope such as [¹¹C]nitrile in a metal-free manner, which can be further diversified to other functional groups such as [¹¹C]carboxylic acids, [¹¹C]amides, and [¹¹C]alkyl amines.

### BACKGROUND

Positron emission tomography (PET) is a commonly used functional imaging technique that employs radioactive probes to measure biological changes in a non-invasive manner. Advances in PET research often depend on the development of radiopharmaceuticals, the syntheses of which involve the introduction of radioisotopes into targeting ligands. As one of the most common elements in organic compounds, carbon has a PET isotope, ¹¹C, that is widely used for radiolabeling bioactive molecules. With a short half-life of ˜20 min, ¹¹C-labeling reactions generally need to be highly efficient and have rapid kinetics. Indeed, various efficient ¹¹C labeling methods have been developed over the past few decades, most of which rely on established synthons based on ¹¹CH₃I, ¹¹CH₃OTf, ¹¹CO₂, ¹¹CO, and [¹¹C]CN.

Among those labeled products, radiolabeled aryl [¹¹C]nitriles have attracted great attention. This is because aryl nitriles are ubiquitous in natural products, pharmaceuticals, pigments, and agrochemical compounds. Aryl nitriles have been discovered as inhibitors that interact and bind to various protein residues such as cysteine, leucine, arginine, and glutamine residues. Moreover, aryl nitriles can serve as intermediates that can be efficiently transformed into other ¹¹C-functional groups including amine, amide, ester, carboxylic acid, tetrazoles, aldehydes, and amidines³² within a half-life decay of ¹¹C. Clearly, easy access to aryl [¹¹C]nitriles and their derivatives could greatly benefit PET research by generating a spectrum of radiolabeled agents for evaluation.

Despite significant interest from a radiotracer development standpoint, there have been limited reports on efficient and convenient methods to generate aryl [¹¹C]nitriles, especially general methods for site-selective (radio)cyanation of arenes. As shown in Scheme 1a of FIG. 16, representative methods for ¹¹C labeling of arenes were mainly accomplished by employing transition metals such as palladium catalysis¹⁻⁴, Cu(I) catalysis^{5,6}, or stoichiometric amounts of Cu(II)^{7,8}, which typically required heating [¹¹C]cyanide with the pre-activated aryl precursors to high temperature. The precursors are mostly electron-neutral and deficient aryl halide, aryl-palladium^{1,4}, aryl boronic acids/esters⁷, and aryl stannanes⁸ that may involve multi-step synthesis and be complicated to prepare in certain situations.

Using significant amounts of metal reagents during the labeling reaction could also complicate purification procedures due to residual metal elemental analysis being required as part of the quality control of radiopharmaceutical synthesis. A more direct photoredox-catalyzed cyanation of arenes using radioactive cyanide sources under metal-free conditions would fill a gap in current PET reaction labeling technology and would benefit the generation of new PET probe agents for biomedical study and drug development.

### SUMMARY

The organic photoredox-catalyzed radiocyanation method reported herein introduces a [¹¹C]nitrile quickly with high radiochemical conversion (RCC) in a metal-free manner, which can also be further diversified to other functional groups such as [¹¹C]carboxylic acids, [¹¹C]amides, and [¹¹C]alkyl amines. This strategy provides easy access to new PET agents that were previously difficult to synthesize from readily available precursors.

As such, one aspect of the disclosure relates to a photoredox-catalyzed cyanation method comprising: a) obtaining a reaction mixture comprising an arene or heteroarene substrate, a photocatalyst, a base additive, and a solvent; b) contacting the reaction mixture with a cyanide source to afford a photocyanation reaction mixture; and c) exposing the photocyanation reaction mixture to blue-violet light to form a cyano arene or heteroarene product.

### BRIEF DESCRIPTION OF THE DRAWINGS

FIG. 1 shows the results of the cyanation reaction optimizations; The reactions were conducted in 0.1 mmol scale with photocatalyst (5.0 mol %), NaHCO₃ (5.0 equiv.), and the cyanide source (4.0 equiv.) in solvent (1 mL), irradiated with 36 W blue LEDs (450-460 nm); Note that footnote^{b} indicates that yields were determined by HPLC.

FIG. 2 shows additional results of the cyanation reaction optimizations. ^{a} The reactions were conducted on a 0.1 mmol scale with photocatalyst (x mol %), NaHCO₃ (b equiv.), and the cyanide source (c equiv.) in ethanol (1 mL), irradiated with two 36 W blue LED lights (450-460 nm); Note that footnotes indicate the following: ^{b} Yields were determined by HPLC on a Phenomenex, Kinetex® 5 μm EVO C18 column based on the UV integrations with the standard curves' correlation of compound 2a and 2b on the same HPLC; ^{c} in a dark, reaction vial covered with aluminum foil;

FIG. 3 shows studies of the Substrate Scope (simple molecules) for [¹²C]-cyanation by Catalyst S1; Isolated yields use standard reaction conditions (0.1 or 0.2 mmol) unless otherwise noted; Note that footnotes indicate the following: ^{b} 2.0 mmol scale reaction (followed general procedure 2 in Example 26);^{c} MeCN/EtOH (v/v=1/1, 0.1M) used as the solvent; ^{d} Acetone cyanohydrin (ACH) was used as the CN source; ^{e} Yields and product ratios were determined by the ¹H NMR analysis; ^{f} Yield was estimated by HPLC.

FIG. 4 shows examples of late-stage functionalization via [¹²C]-cyanation by catalyst S1; Isolated yields use standard reaction conditions (0.1 or 0.2 mmol) unless otherwise noted; Note that footnotes indicate the following: ^{b} MeCN/EtOH (v/v=1/1, 0.1M) used as the solvent; Reactions run at 22° C. for 60 h with 10 mol % S1 and 4 equiv. of TMSCN; ^{d} 0.05 mmol 38a with 5% mol S1, 4 equiv. of TMSCN at 22° C. for 22 hours; ^{e} S2 (10%) used as photocatalyst; HPLC estimated yield.¹²

FIG. 5 shows a summary of the Optimization for Arene Demethoxy ¹¹C-Radiocyanation; ^{a} the labeling reactions were conducted on a 0.03 mmol scale under 450 nm laser irradiation unless otherwise noted. The ¹¹C-labeled product mixture aliquots were injected and analyzed by an HPLC equipped with both UV and radio detectors. All RCCs were decay corrected.

FIG. 6 shows a summary for the Substrate Scope (simple molecules) for ¹¹C-Cyanation by Catalyst S1; Follows general labeling procedure 1 or 2; the radiochemical conversion (RCC) was reported as average±SD % of reactions run in triplicate unless otherwise noted; Note that footnotes indicate the following: ^{b} From 1,5-dimethoxy 2-isoproxy benzene (1a′); C Following the S_{N}2 labeling reaction procedure in Example 103; ^{d} Blue LED irradiation; ^{c} The labeling reaction was carried out with a flow device irradiated under a blue LED lamp; ^{d} 0.05 mmol precursor was used; ^{g} From 1,3,5 trimethoxy benzene (26a); ^{h} Catalyzed by S3 (4-CzIPN, 2.4 mg); ⁱ Catalyzed by S2 (Mes-Acr, 1.5 mg).

FIG. 7 shows a summary of the ¹¹C-Cyanation of Bioactive Molecules by Catalyst S1 and [¹¹C]Nitrile Hydrolysis; Follows general labeling procedure 1 or 2 in Example 26; the radiochemical conversion (RCC) was reported as average±SD % of reactions run in triplicate unless otherwise noted; Note that footnotes indicate the following: ^{b} Blue LED irradiation; C The labeling reaction was carried out with a flow device irradiated under a blue LED lamp; ^{d} Catalyzed by S3 (4-CzIPN, 2.4 mg); ^{e} Catalyzed by S2 (Mes-Acr, 1.5 mg); ^{f} From Trimethoprim; 9 Non-decay corrected isolated radiochemical yield (RCY) with 70-110 MBq nitrile product collected; ^{h} 11.1 GBq TBA⁺[¹¹C]CN⁻ was used. (General labeling procedure 3 in Example 26).

FIG. 8 shows a summary of the Substrate Scope for [11C]-Cyanation by Catalyst S2; The labeling reactions were conducted by using general labeling procedure 3 or 4 in Example 26; the RCC was reported as average±SD % of reactions run in triplicate unless otherwise noted; Note that footnotes indicate the following: ^{b} Blue LED irradiation; C The labeling reaction was carried out with a flow device with LED irradiation; ^{d} 1,3,5 trimethoxy benzene was used as the precursor; ^{e} S1 used as catalyst (2.0 mg).

FIG. 9 shows results of a small animal PET Imaging Study of [¹¹C]39b.

FIG. 10 shows HPLC data for UV calibration of compounds 1a and 1b with a Coefficient factor f1=Kb(slope, 1b)/Ka(slope, 1a)=1.338|Reaction yield y(1b)=y′/[(1−y′)1.338+y′) and compounds 2a and 2b with a Coefficient factor f2=Kb(slope, 2b)/Ka(slope, 2a)=0.967|Reaction yield of y(2b)=y′/[(1−y′)0.967+y′), wherein y′ is the peak area integration ratio Int[1b]/(Int[unreacted 1a]+Int[1b]) or Int[2b]/(Int[unreacted 2a]+Int[2b], usually auto-generated by the HPLC software) obtained on the HPLC chromatograph, considering almost no side product observed for the reaction condition screenings by using 1a and 2a.

FIG. 11A shows a schematic of a proposed mechanism for photoredox demethoxy (¹¹C-radio)cyanation.

FIG. 11B shows a schematic of an alternate proposed mechanism for photoredox demethoxy (¹¹C-radio)cyanation.

FIG. 12 shows an Irradiation profile for the Kessil A160WE TUNA BLUE lamp.

FIG. 13 shows the standard curve of 2,6-dimethylbenzonitrile (1b).

FIG. 14 shows PET/CT image that were acquired at 15 min (A1 and A2) and 45 min (B1 and B2) post intravenous tail vein injection of [¹¹C]39b. A1 and B1 show the liver coronal plane. A2 and B2 show the kidney coronal plane with the kidney circled. (C) Chemical structure of [¹¹C]39b.

FIG. 15 shows PET imaging results with [¹¹C]-39b in PC3-PSMA tumor-bearing mouse. ROI analysis of tumor and major organs of [¹¹C]39b at 15- and 45-minutes post-injection (N=1).

FIG. 16 shows synthetic approaches to ¹¹C-benzonitriles: (a) conventional transition metal-mediated ¹¹C-cyanation, (b) photoredox-mediated ¹⁸F-fluorination by acridinium catalysts, and (c) This work: photoredox-catalyzed ¹¹C-cyanation by RFTA catalyst or acridinium catalysts.

FIG. 17 shows the chemical structures of photocatalysts selected from the group consisting of Mes-Acr-Ph⁺, 4CzIPN, RFTA, EOSIN, Ir-ll, and a combination thereof.

FIG. 18 shows cyanation scale up and synthetic applications. ^{a} Reactions were carried out with a cooling fan at 22° C. for 60 h, b 72 h reaction with 3 equiv. of K¹³CN.

FIG. 19 shows ¹³C-cyanation of methoxyarenes. ^{a} Reactions were carried out with a cooling fan at 22° C. for 60 h. ^{b}72 h reaction with 3 equiv. of K¹³CN.

FIG. 20 shows HPLC estimated yield for S_{N}2 competitive reaction of Example 25.

FIG. 21 shows the general procedure for photocatalytic ¹¹C-cyanation—General procedure 1 by catalyst S1 (RFTA) with a laser or an LED lamp.

FIG. 22 shows the general procedure for photocatalytic ¹¹C-cyanation—General procedure 1 by catalyst S2 (RFTA) with a laser or an LED lamp.

FIG. 23 shows the general procedure for the photocatalytic ¹¹C-cyanation—General procedure 2 by catalyst S2 (Mes-Acr⁻BF4⁺) with a laser or an LED lamp.

FIG. 24 shows the general procedure for the photocatalytic ¹¹C-cyanation—General procedure 5 by catalyst S2 (Mes-Acr⁻BF4⁺) with a continuous flow device and an LED lamp.

### DETAILED DESCRIPTION

The presently disclosed subject matter will now be described more fully hereinafter. However, many modifications and other embodiments of the presently disclosed subject matter set forth herein will come to mind to one skilled in the art to which the presently disclosed subject matter pertains, having the benefit of the teachings presented in the foregoing descriptions. Therefore, it is to be understood that the presently disclosed subject matter is not to be limited to the specific embodiments disclosed, and that modifications and other embodiments are intended to be included within the scope of the appended claims. In other words, the subject matter described herein covers all alternatives, modifications, and equivalents. In the event that one or more of the incorporated literature, patents, and similar materials differs from or contradicts this application, including, but not limited to, defined terms, term usage, described techniques, or the like, this application controls. Unless otherwise defined, all technical and scientific terms used herein have the same meaning as commonly understood by one of ordinary skill in this field. All publications, patent applications, patents, and other references mentioned herein are incorporated by reference in their entirety.

As a non-invasive imaging technology, positron emission tomography (PET) plays a crucial role in personalized medicine, including early diagnosis, patient screening, and treatment monitoring. The advancement of PET research depends on the discovery of new PET agents, which requires the development of simple and efficient radiolabeling methods in many cases. As bioisosteres for halogen and carbonyl moieties, nitriles are important functional groups in pharmaceutical and agrochemical compounds. Herein, a mild organophotoredox-catalyzed method for efficient cyanation of a broad spectrum of arenes, including abundant and readily available veratroles and pyrogallol trimethyl ethers is described. Notably, the transformations not only are compatible with various affordable ¹²C and ¹³C-cyanide sources, but also can be applied to carbon-11 synthons to incorporate [¹¹C]nitriles into arenes. The aryl [¹¹C]nitriles can be further derivatized to [¹¹C]carboxylic acids, [¹¹C]amides, and [¹¹C]alkyl amines. The newly developed reaction can serve as a powerful tool for generating new PET agents.

A paper published in Cell Press entitled “¹¹C—, ¹²C—, and ¹³C-cyanation of electron-rich arenes via organic photoredox catalysis” was available online to the public online Jan. 2, 2023. The paper published in Vol. 9, Issue 2, on the Feb. 9, 2023 with the following authors: Xuedan Wu, Wei Chen, Natalie Holmberg-Douglas, Gerald Thomas Bida, Xianshuang Tu, Xinrui Ma, Zhanhong Wu, David A. Nicewicz, and Zibo Li. This publication originated from the inventors own work.

### I. Definitions

Listed below are definitions of various terms used to describe this invention. These definitions apply to the terms as they are used throughout this specification, unless otherwise limited in specific instances, either individually or as part of a larger group.

As used in the specification and the appended claims, the singular forms “a,” “an” and “the” include plural referents unless the context clearly dictates otherwise. Thus, for example, reference to “an alkyl group” or “a phenyl” includes mixtures of two or more such alkyl groups or phenyls.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. It is also understood that there are a number of values disclosed herein and that each value is also herein disclosed as “about” that particular value in addition to the value itself. For example, if the value “10” is disclosed, then “about 10” is also disclosed. It is also understood that each unit between two particular units is also disclosed. For example, if 10 and 15 are disclosed, then 11, 12, 13, and 14 are also disclosed. Further, unless specified by the term “integer,” the number specified includes fractions or numbers with decimals. For example, the range of “from about 1 to about 5” includes numbers such as 1, 1.1, 1.5, 2.0, 2.2, and so on. As used herein, the term “integer” refers to a number that is a whole number, and not a fraction.

References in the specification and concluding claims to parts by weight of a particular element or component in a composition denote the weight relationship between the element or component and any other elements or components in the composition or article for which a part by weight is expressed. Thus, in a compound containing 2 parts by weight of component X and 5 parts by weight component Y, X and Y are present at a weight ratio of 2:5, and are present in such ratio regardless of whether additional components are contained in the compositions.

Throughout this specification and the claims, the words “comprise,” “comprises,” and “comprising” are used in a non-exclusive sense, except where the context requires otherwise. It is understood that embodiments described herein include “consisting of” and/or “consisting essentially of” embodiments.

As used herein, the terms “increase,” “increases,” “increased,” “increasing”, “improve,” “enhance,” and similar terms indicate an elevation in the specified parameter of at least about 5%, 10%, 15%, 20%, 25%, 30%, 35%, 40%, 45%, 50%, 55%, 60%, 65%, 70%, 75%, 80%, 85%, 90%, 95%, 100%, 150%, 200%, 300%, 400%, 500%, or more.

As used herein, the terms “reduce,” “reduces,” “reduced,” “reduction,” “inhibit,” and similar terms refer to a decrease in the specified parameter of at least about 5%, 10%, 15%, 20%, 25%, 30%, 35%, 40%, 45%, 50%, 55%, 60%, 65%, 70%, 75%, 80%, 85%, 90%, 95%, 97%, or 100%.

As used herein, the “contacting” refers to reagents in close proximity so that a reaction may occur.

As used herein, the term “stereoisomer” refers to compounds which have identical chemical constitution, but differ with regards to the arrangement of the atoms or groups in space. These “stereoisomers” have a “stereogenic center” which may be a chiral center.

As used herein, the term “chiral” refers to molecules which have the property of non-superimposability of the mirror image partner, while the term “achiral” refers to molecules which are superimposable on their mirror image partner.

As used herein, the term “diastereomers” refers to a stereoisomer with two or more centers of chirality and whose molecules are not mirror images of one another. Diastereomers have different physical properties, e.g., melting points, boiling points, spectral properties, and reactivity. Mixtures of diastereomers may separate under high-resolution analytical procedures such as electrophoresis and chromatography.

As used herein, the term “enantiomers” refers to two stereoisomers of a compound which are non-superimposable mirror images of one another. Stereochemical definitions and conventions used herein generally follow S. P. Parker, Ed., McGraw-Hill Dictionary of Chemical Terms (1984) McGraw-Hill Book Company, New York; and Eliel, E. and Wiley, S., “Stereochemistry of Organic Compounds”, John Wiley & Sons, Inc., New York, 1994. The compounds of the invention may contain asymmetric or chiral centers, and therefore exist in different stereoisomeric forms. It is intended that all stereoisomeric forms of the compounds of the invention, including, but not limited to, diastereomers, enantiomers and atropisomers, as well as mixtures thereof such as racemic mixtures, form part of the present invention.

As used herein, the term “arene” refers to an aromatic hydrocarbon with an alternating double and single bond between carbon atoms forming rings, e.g., benzene, a phenyl group, etc.

As used herein, the term “cyano arene” refers to arene, which is substituted with one nitrile group (—CN). For example, benzonitrile. However, the cyano arene can further contain additional substituents. For example, methoxybenzyl cyanide.

As used herein, the term “heteroarene” refers to a heterocyclic compound formally derived from an arene by replacement of one or more methine by trivalent or divalent heteroatoms (e. g., —N, —O, —S) respectively in such a way as to retain its aromaticity. For example, pyridine.

As used herein, the term “cyano heteroarene” refers to a heteroarene, which is substituted with one nitrile group (—CN). For example, nicotinonitrile. However, the cyano heteroarene can further contain additional substituents. For example, 3-cyano, 4-methoxypyridine.

As used herein, the term “substituted” refers to a moiety (such as heteroaryl, aryl, cycloalkyl, alkyl, and/or alkenyl) wherein the moiety is bonded to one or more additional organic or inorganic substituent radicals. In some embodiments, the substituted moiety comprises 1, 2, 3, 4, or 5 additional substituent groups or radicals. Suitable organic and inorganic substituent radicals include, but are not limited to, halogen, hydroxyl, cycloalkyl, aryl, substituted aryl, heteroaryl, heterocyclic ring, substituted heterocyclic ring, amino, mono-substituted amino, di-substituted amino, acyloxy, nitro, cyano, carboxy, carboalkoxy, alkyl carboxamide, substituted alkyl carboxamide, dialkyl carboxamide, substituted dialkyl carboxamide, alkylsulfonyl, alkylsulfinyl, thioalkyl, alkoxy, substituted alkoxy or haloalkoxy radicals, wherein the terms are defined herein. Unless otherwise indicated herein, the organic substituents can comprise from 1 to 4 or from 5 to 8 carbon atoms. When a substituted moiety is bonded thereon with more than one substituent radical, then the substituent radicals may be the same or different.

As used herein, the term “unsubstituted” refers to a moiety (such as heteroaryl, aryl, alkenyl, and/or alkyl) that is not bonded to one or more additional organic or inorganic substituent radicals as described above, meaning that such a moiety is only substituted with hydrogens.

As used herein, the term “alkyl” refers to a straight or branched chain hydrocarbon containing from 1 to 10 carbon atoms. Representative examples of alkyl include, but are not limited to: methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, n-decyl, and the like. These groups may be substituted with groups selected from halo (e.g., haloalkyl), alkyl, haloalkyl, alkenyl, alkynyl, cycloalkyl, cycloalkylalkyl, aryl, arylalkyl, heterocyclo, heterocycloalkyl, hydroxyl, alkoxy (thereby creating a polyalkoxy such as polyethylene glycol), alkenyloxy, alkynyloxy, haloalkoxy, cycloalkoxy, cycloalkylalkyloxy, aryloxy, arylalkyloxy, heterocyclooxy, heterocyclolalkyloxy, mercapto carboxy, alkylamino, alkenylamino, alkynylamino, haloalkylamino, cycloalkylamino, cycloalkylalkylamino, arylamino, arylalkylamino, heterocycloamino, heterocycloalkylamino, disubstituted-amino, ester, amide, nitro, or cyano.

The term “cycloalkyl” refers to a hydrocarbon 3-8 membered monocyclic or 7-14 membered bicyclic ring system having at least one saturated ring or having at least one non-aromatic ring, wherein the non-aromatic ring may have some degree of unsaturation. Cycloalkyl groups may be optionally substituted with one or more substituents. In one embodiment, 0, 1, 2, 3, or 4 atoms of each ring of a cycloalkyl group may be substituted by a substituent. Representative examples of cycloalkyl group include: cyclopropyl, cyclopentyl, cyclohexyl, cyclobutyl, cycloheptyl, cyclopentenyl, cyclopentadienyl, cyclohexenyl, cyclohexadienyl, and the like.

As used herein, the term “heterocycloalkyl” refers to a nonaromatic 3-8 membered monocyclic, 7-12 membered bicyclic, or 10-14 membered tricyclic ring system comprising 1-3 heteroatoms if monocyclic, 1-6 heteroatoms if bicyclic, or 1-9 heteroatoms if tricyclic, said heteroatoms selected from O, N, S, B, P or Si, wherein the nonaromatic ring system is completely saturated. Heterocycloalkyl groups may be optionally substituted with one or more substituents. In one embodiment, 0, 1, 2, 3, or 4 atoms of each ring of a heterocycloalkyl group may be substituted by a substituent. Representative heterocycloalkyl groups include piperidinyl, piperazinyl, tetrahydropyranyl, morpholinyl, thiomorpholinyl, 1,3-dioxolanyl, tetrahydrofuryl, tetrahydrothienyl, thienyl, and the like.

As used herein, the terms “alkenyl” and “alkene” refer to unsaturated aliphatic groups analogous in length and possible substitution to the alkyls described above, but that contain at least one double bond. For example, the term “alkenyl” includes straight-chain alkenyl groups (e.g., ethylenyl, propenyl, butenyl, pentenyl, hexenyl, heptenyl, octenyl, nonenyl or decenyl), branched-chain alkenyl groups and cycloalkenyl (alicyclic) groups (cyclopropenyl, cyclopentenyl, cyclohexenyl, cycloheptenyl or cyclooctenyl) groups. The term alkenyl further includes alkenyl groups that include oxygen, nitrogen, sulfur or phosphorous atoms replacing one or more carbons of the hydrocarbon backbone. In certain embodiments, a straight chain or branched chain alkenyl group with 10 or fewer carbon atoms in its backbone (e.g., C2-C10 for straight chain, C3-C10 for branched chain) is used. Likewise, cycloalkenyl groups may have from 3-8 carbon atoms in their ring structure, and more preferably have 5 or 6 carbons in the ring structure. The term C2-C10 includes alkenyl groups containing 2 to 10 carbon atoms. Certain alkene compounds of the invention may exist as a mixture of E and Z isomers, predominantly as E isomers, or predominantly Z isomers. In certain embodiments, compounds of the invention may be enriched in either the E or Z isomer. For example, a compound of the invention may have greater than 50%, 60%, 70%, 80%, 90%, or 95% or more of the E or Z isomer.

As used herein, the term “heteroaryl” or “heteroaromatic” refers to a monovalent aromatic radical of 5- or 6-membered rings, and includes fused ring systems (at least one of which is aromatic) of 5-20 atoms, containing one or more heteroatoms independently selected from nitrogen, oxygen, and sulfur. Examples of heteroaryl groups are pyridinyl (including, for example, 2-hydroxypyridinyl), imidazolyl, imidazopyridinyl, pyrimidinyl (including, for example, 4-hydroxypyrimidinyl), pyrazolyl, triazolyl (including, for example, 3-amino-1,2-4-triazole or 3-mercapto-1,2,4-triazole), pyrazinyl (including, for example, aminopyrazine), tetrazolyl, furyl, thienyl, isoxazolyl, thiazolyl, oxadiazolyl, oxazolyl, isothiazolyl, pyrrolyl, quinolinyl, isoquinolinyl, tetrahydroisoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, thiadiazolyl, furazanyl, benzofurazanyl, benzothiophenyl, benzothiazolyl, benzoxazolyl, quinazolinyl, quinoxalinyl, naphthyridinyl, oxazol-2(3H)-onyl, and furopyridinyl. The heteroaryl groups are thus, in some embodiments, monocyclic or bicyclic. Heteroaryl groups are optionally substituted independently with one or more substituents described herein.

As used herein, the term “aryl” refers to a hydrocarbon monocyclic, bicyclic or tricyclic aromatic ring system. Aryl groups may be optionally substituted with one or more substituents. In one embodiment, 0, 1, 2, 3, 4, 5 or 6 atoms of each ring of an aryl group may be substituted by a substituent. Examples of aryl groups include phenyl, naphthyl, anthracenyl, fluorenyl, indenyl, azulenyl, and the like.

As used herein, the term “unsubstituted” refers to a moiety (such as an alkyl group) that is not bonded to one or more additional organic or inorganic substituent radical as described above, meaning that such a moiety is only substituted with hydrogens.

As used herein, the term “alkoxy”, used alone or as part of another group, means the radical —OR, where R is an alkyl group as defined herein.

As used herein, the terms “halo,” “halogen,” and “halide” refer to any suitable halogen, including —F, —Cl, —Br, and —I.

As used herein, the term “mercapto” refers to an —SH group.

As used herein, the term “cyano” refers to a —CN group.

As used herein, the term “carboxylic acid” refers to a —C(O)OH group.

As used herein, the term “hydroxyl” refers to an —OH group.

As used herein, the term “nitro” refers to an —NO₂ group.

As used herein, the term “sulfonyl” refers to the SO₂⁻ group. The “sulfonyl” may refer to a sulfonyl group, which is, for example, an alkylsulfonyloxy group such as a methylsulfonyloxy or ethylsulfonyloxy group and an aromatic sulfonyloxy group such as a benzenesulfonyloxy or tosyloxy group.

As used herein, the terms “ether” and “alkylether” are represented by the formula Rₐ—O—R_{b}, where Rₐ and R_{b} can be, independently, an alkyl, cycloalkyl, alkenyl, cycloalkenyl, alkynyl, cycloalkynyl, aryl, or heteroaryl group as described herein. The term “polyether” as used herein is represented by the formula —(Rₐ—O—R_{b})ₓ—, where Rₐ and R_{b} can be, independently, an alkyl, cycloalkyl, alkenyl, cycloalkenyl, alkynyl, cycloalkynyl, aryl, or heteroaryl group described herein and “x” is from about 1 to about 500. Examples of polyether groups include polyethylene oxide, polypropylene oxide, and polybutylene oxide.

As used herein, the term “acyl”, used alone or as part of another group, refers to a —C(O)R radical, where R is any suitable substituent such as aryl, alkyl, alkenyl, alkynyl, cycloalkyl or other suitable substituent as described herein.

As used herein, the term “amino” means the radical —NH₂.

As used herein, the term “alkylamino” or “mono-substituted amino”, used alone or as part of another group, means the radical —NHR, where R is an alkyl group.

As used herein, the term “disubstituted amino”, used alone or as part of another group, means the radical —NRₐR_{b}, where Rₐ and R_{b} are independently selected from the groups alkyl, haloalkyl, alkenyl, alkynyl, cycloalkyl, cycloalkylalkyl, aryl, arylalkyl, heterocyclo, and heterocycloalkyl.

As used herein, the term “ester”, used alone or as part of another group, refers to a —C(O)OR radical, where R is any suitable substituent such as alkyl, cycloalkyl, alkenyl, alkynyl or aryl.

As used herein, the term “amide”, used alone or as part of another group, refers to a —C(O)NRₐR_{b} radical, where Rₐ and R_{b} are any suitable substituent such as alkyl, cycloalkyl, alkenyl, alkynyl or aryl.

It will be understood that the structures provided herein and any recitation of “substitution” or “substituted with” includes the implicit proviso that such structures and substitution are in accordance with permitted valence of the substituted atom and the substituent, and that the substitution results in a stable compound, e.g., which does not spontaneously undergo transformation such as by rearrangement, cyclization, elimination, etc.

The phrase “pharmaceutically acceptable” indicates that the substance or composition is compatible chemically and/or toxicologically with the other ingredients comprising a formulation, and/or the subject being treated therewith.

The phrase “pharmaceutically acceptable salt” as used herein, refers to pharmaceutically acceptable organic or inorganic salts of a compound disclosed herein. Exemplary salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucuronate, saccharate, formate, benzoate, glutamate, methanesulfonate “mesylate”, ethanesulfonate, benzenesulfonate, p-toluenesulfonate, pamoate (i.e., 1,1′-methylene-bis-(2-hydroxy-3-naphthoate)) salts, alkali metal (e.g., sodium and potassium) salts, alkaline earth metal (e.g., magnesium) salts, and ammonium salts. A pharmaceutically acceptable salt may involve the inclusion of another molecule such as an acetate ion, a succinate ion or other counter ion. The counter ion may be any organic or inorganic moiety that stabilizes the charge on the parent compound. Furthermore, a pharmaceutically acceptable salt may have more than one charged atom in its structure. In instances where multiple charged atoms are part of the pharmaceutically acceptable salt, the salt can have multiple counter ions. Hence, a pharmaceutically acceptable salt can have one or more charged atoms and/or one or more counter ions.

“Carriers” as used herein include pharmaceutically acceptable carriers, excipients, or stabilizers that are nontoxic to the cell or mammal being exposed thereto at the dosages and concentrations employed. Often the physiologically acceptable carrier is an aqueous pH buffered solution. Non-limiting examples of physiologically acceptable carriers include buffers such as phosphate, citrate, and other organic acids; antioxidants including ascorbic acid; low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™, polyethylene glycol (PEG), and PLURONICS™. In certain embodiments, the pharmaceutically acceptable carrier is a non-naturally occurring pharmaceutically acceptable carrier.

As used herein, the term “pharmaceutical composition” refers to the active agent in combination with a pharmaceutically acceptable carrier, e.g., a carrier commonly used in the pharmaceutical industry. The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. In some embodiments of any of the aspects, a pharmaceutically acceptable carrier can be a carrier other than water. In some embodiments of any of the aspects, a pharmaceutically acceptable carrier can be an emulsion, liposome, nanoparticle, and/or solution. In some embodiments of any of the aspects, a pharmaceutically acceptable carrier can be an artificial or engineered carrier, e.g., a carrier that the active ingredient would not be found to occur in nature.

The term “administration” or “administering” includes routes of introducing the compound(s) to a subject to perform their intended function. Examples of routes of administration which can be used include injection (including, but not limited to, subcutaneous, intravenous, parenterally, intraperitoneally, intrathecal), topical, oral, inhalation, rectal and transdermal.

The term “effective amount” includes an amount effective, at dosages and for periods of time necessary, to achieve the desired result. An effective amount of compound may vary according to factors such as the disease state, age, and weight of the subject, and the ability of the compound to elicit a desired response in the subject (i.e., being able to conduct imaging studies).

The term “subject” refers to animals such as mammals, including, but not limited to, primates (e.g., humans), cows, sheep, goats, horses, dogs, cats, rabbits, rats, mice and the like. In certain embodiments, the subject is a human.

As used herein, a subject is “in need of” a treatment if such subject would benefit biologically, medically or in quality of life from such treatment.

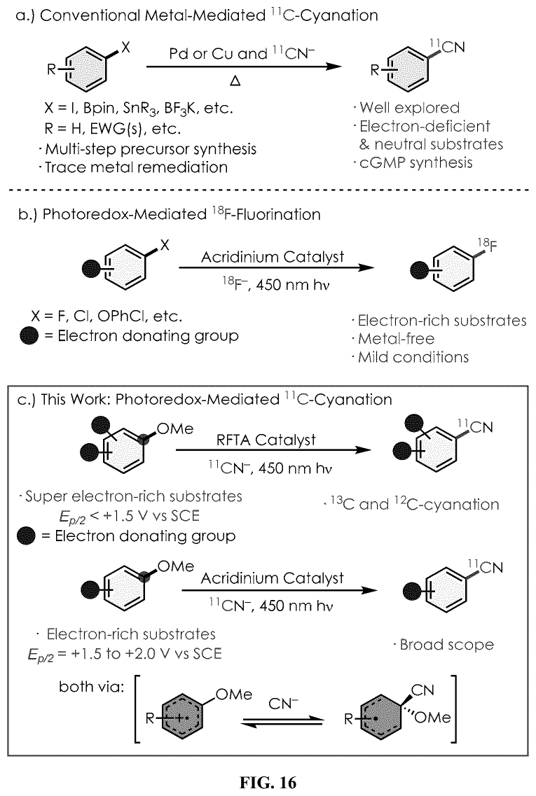

II. Photoredox System In light of the abundance of arenes and aryl nitriles in therapeutics and given the recent progress in developing photoredox-mediated ¹⁸F-fluorination of arenes⁹-1¹ (Scheme 1b of FIG. 16), the current disclosure is directed towards organic photoredox catalysis mediating ¹¹C-cyanation on arenes, at the C—O bonds in a site-selective manner via arene cation radical intermediates (Scheme 1c of FIG. 16).¹² Recently, it was found that organic photoredox-catalyzed cation radical-accelerated nucleophilic aromatic substitution (CRA-S_{N}Ar) reactions are possible with readily available aryl ether substrates, where the alkoxy group serves as the nucleofuge.

Unfortunately, the substrate scope and catalytic efficiency were somewhat limited due to catalyst decomposition by adventitious cyanide. The current disclosure, however, developed a photoredox system that not only greatly improves the catalytic efficiency (up to 99% yield), but also expands the reaction substrate scope to heteroarene and to more electron-rich arenes (E₂<+1.50 V vs SCE) such as veratrole and pyrogallol trimethyl ether, derived from catechol, guaiacol, pyrogallol and syringol, which are important moieties and functional groups in natural products, synthetic drug candidates, and organic material structures. This photoredox-catalyzed transformation could be adapted to ¹¹C- and ¹³C-cyanation for efficient late-stage (radio) labeling on a variety of readily available complex precursors with pyrogallol or veratrole cores.

Thus, one aspect of the current disclosure is a photoredox system comprising a photocatalyst, a cyanide source, a base additive, a blue-violet light, and a solvent. Such a photoredox system can then be used to introduce cyanide functional groups into a variety of arene and heteroarene substrates. As already mentioned above there is an abundance of therapeutics and natural products that contain aromatic nitriles.

Another aspect of the current disclosure is to employ a radioactive cyanide source in the disclosed photoredox system. Cyanation of arenes/heteroarenes with radioactive cyanide sources (which is also referred to as radiocyanation) render radioactive cyano arenes/heteroarenes, which can be used in a variety of application, e.g., PET and MRI imaging modalities.

Therefore, each component of the disclosed photoredox system as well as the arene/heteroarene substrate is described in more detail below. It should be noted that any mention of a concentration (in M), catalyst loading amount (in mol %), or relative amounts of certain components of the photoredox system provided as equivalents (equiv.) are all based on the amount of arene/heteroarene substrate employed in methods using the disclosed photoredox system (and its components). A skilled artisan would be very familiar with this kind of expression of these units in this particular manner.

### A. Photocatalyst

The photoredox system as disclosed herein requires a photocatalyst. As used herein, the term “photocatalyst” can be any material that exhibits photocatalytic properties, i.e., the ability to foster and accelerate specific chemical reactions upon stimulation by light of suitable wavelengths.

The photocatalyst can be any material as along as it exhibits a suitable excited state reduction potential. As used herein, the term “excited state reduction potential” refers to the likelihood of a chemical element to be reduced, wherein the chemical element temporarily occupies an energy state that is greater than the ground state. In some embodiments, the excited state reduction potential is up to about +2.5V vs. SCE, about +2.2V vs. SCE, about +2.0V vs. SCE, about +1.8V vs. SCE, about +1.6V vs. SCE, about +1.4V vs. SCE, about +1.2V vs. SCE, about +1.0V vs. SCE, about +0.8V vs. SCE, about +0.6V vs. SCE, about +0.4V vs. SCE, or about +0.2V vs. SCE (saturated calomel electrode) in acetonitrile.

In some embodiments, the excited state reduction potential ranges from about +2.5V vs. SCE to about 0.1V vs. SCE, from about 2.2V vs. SCE to about 0.2V vs. SCE, from about 2.0V vs. SCE to about 0.5V vs. SCE, from about 0.75V vs. SCE to about 1.75V vs. SCE, or from about 1V vs. SCE to about 1.5V vs. SCE (saturated calomel electrode) in acetonitrile.

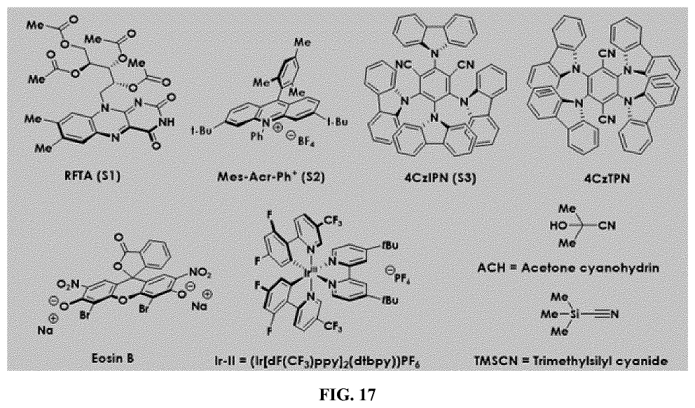

In some embodiments, the photocatalyst is selected from the group consisting of Mes-Acr-Ph⁺, 4CzIPN, RFTA, EOSIN, Ir-ll, and a combination thereof. The chemical structures of these photocatalysts are shown in FIG. 17.

In some embodiments, the photocatalyst is Mes-Acr-Ph⁺ or RFTA. In some embodiments, the photocatalyst does not contain a metal, i.e., the photocatalyst is metal-free.

The amount of photocatalyst loading present in the photoredox system can vary. In some embodiments, the photocatalyst is present in an amount of from about 0.1 mol % to about 10 mol %, from about 1 mol % to about 8 mol %, from about 1 mol % to about 5 mol %, from about 1.5 mol % to about 5 mol %, from about 1.75 mol % to about 4.5 mol %, from about 2 mol % to about 4 mol %, from about 2 mol % to about 3.5 mol %, or from about 2 mol % to about 3 mol %.

In some embodiments, the photocatalyst can be present in an amount of at least about 0.1 mol %, about 0.5 mol %, about 1 mol %, about 1.25 mol %, about 1.75 mol %, about 2.0 mol %, about 2.25 mol %, about 2.50 mol %, about 2.75 mol %, about 3.0 mol %, about 3.25 mol %, about 3.5 mol %, about 3.75 mol %, about 4.0 mol %, about 4.25 mol %, about 4.5 mol %, about 4.75 mol %, about 5 mol %, about 5.5 mol %, about 6 mol %, about 6.5 mol %, about 7 mol %, or at least about 7.5 mol %. In addition, or in the alternative, the photocatalyst can be present in an amount of less than about 10 mol %, about 9.5 mol %, about 9.0 mol %, about 8.5 mol %, about 8.0 mol %, about 7.75 mol %, about 7 mol %, about 6.5 mol %, about 6.0 mol %, about 5.5 mol %, about 5 mol %, about 4.5 mol %, about 4 mol %, about 3.5 mol %, about 3.0 mol %, or less than about 2.5 mol %.

In alternate embodiments, the photoredox system contains radioactivity. In such embodiments, the photocatalyst is present in an amount of from about 0.1 mg to about 5 mg, from about 0.5 mg to about 4.5 mg, from about 0.75 mg to about 4 mg, from about 0.8 mg to about 3.5 mg, from about 1 mg to about 3 mg, from about 1.25 mg to about 2.75 mg, from about 1.5 mg to about 2.5 mg or from about 2 mg to about 2.5 mg. In some embodiments, the photocatalyst is present in an amount of at least about 0.1 mg, at least about 0.5 mg, at least about 0.75 mg, at least about 1 mg, at least about 1.25 mg, at least about 1.50 mg, at least about 1.75 mg, at least about 2.0 mg, at least about 2.25 mg at least about 2.50 mg, or at least about 2.75 mg. In addition, or in the alternative, the photocatalyst is present in an amount of less than about 3.5 mg, less than about 3.25 mg, less than about 3.0 mg, less than about 2.75 mg, less than about 2.5 mg, less than about 2.25 mg, less than about 2.0 mg, less than about 1.75 mg, less than about 1.5 mg, or less than about 1.25 mg.

### B. Cyanide Source

The photoredox system disclosed herein requires a cyanide source. Exemplary cyanide sources include, but are not limited to, acetone cyanhydrin (ACH), tetrabuylammonium cyanide (TBACN or NBu₄CN), trimethylsilyl cyanide (TMSCN), sodium cyanide (NaCN), potassium cyanide (KCN), and combinations thereof. In such embodiments, the cyanide source contains carbon isotopes ¹²C or ¹³C and/or nitrogen isotopes ¹⁴N or ¹⁵N. In some embodiments, the cyanide source is [¹³C]TMSCN or [¹³C]KCN. In some embodiments, the cyanide source is [¹⁵N]TMSCN or [¹⁵N]KCN.

The amount of (non-radioactive) cyanide source present in the photoredox system can vary. In some embodiments, the cyanide source does not contain a radioisotope and is present in the photoredox system in an amount that ranges from about 1 equiv. to about 10 equiv., from about 1 equiv. to about 8 equiv., from about 1 equiv. to about 6 equiv., from about 1 equiv. to about 5 equiv., from about 1.5 equiv. to about 4 equiv., from about 1.5 equiv. to about 3 equiv., or from about 2 equiv. to about 3 equiv. In some embodiments, the base additive is present in the photoredox system in an amount that ranges from about 0.1 equiv. to about 5 equiv. from about 0.5 equiv. to about 4.5 equiv., from about 0.75 equiv. to about 4 equiv., from about 0.8 equiv. to about 3 equiv., from about 1.0 equiv. to about 3.0 equiv., from about 1.5 equiv. to about 2.5 equiv., from about 1.75 equiv. to about 2.5 equiv., or from about 2.0 equiv. to about 2.5 equiv. In some embodiments, the base additive is present in the photoredox system in an amount of less than about 7 equiv., less than about 5 equiv., less than about 4 equiv., less than about 3.5 equiv., less than about 3.0 equiv., less than about 2.5 equiv., less than about 2.0 equiv., less than about 1.5 equiv., less than about 1.0 equiv., less than about 0.75 equiv., less than about 0.5 equiv., less than about 0.25 equiv., less than about 0.1 equiv., or less than about 0.01 equiv. In addition, or in the alternative, the base additive is present in the photoredox system in an amount of at least about 0.0001 equiv., at least about 0.001 equiv., at least about 0.01 equiv., at least about 0.1 equiv., at least about 1 equiv., at least about 1.5 equiv., at least about 1.75 equiv., or at least about 2 equiv.

In some embodiments, the cyanide source may contain a radioisotope. Exemplary radioisotopes include, but are not limited to, ¹¹C and ¹³N. Exemplary cyanide sources containing a radioisotope include, but are not limited to [¹¹C]TMSCN or [¹¹C]TBACN. In some embodiments, the cyanide source containing the radioisotope (also referred to as radioligand) is dissolved in a solvent to form a solution (referred to herein as radioactive cyanide source) to promote ease of handling of the radioligand. The solvent used for such solution can vary but are selected to ensure that (a) the radioligand is fully soluble in the chosen solvent; and/or (b) that the radioligand is stable in the chosen solvent. In some embodiments, the solvent is a polar protic solvent or an aprotic polar solvent as is described in more detail below. In some embodiments, the solvent is ethanol.

The amount of cyanide source containing a radioisotope present in a photoredox system can vary. In some embodiments, the amount of the radioactive cyanide source used in the disclosed photoredox system is significantly smaller compared to the amount of non-radioactive cyanide source employed in the disclosed photoredox system. In such embodiments, the amount of radioactive cyanide source is provided as a function of radioactivity measured in becquerels (Bq).

Thus, in some embodiments, the radioactivity of the cyanide sources ranges from about 0.001 GBq to about 2 GBq, from about 0.001 GBq to about 1.5 GBq, from about 0.01 GBq to about 1.5 GBq, from about 0.1 GBq to about 1.25 GBq, from about 0.1 GBq to about 1.20 GBq, from about 0.1 GBq to about 1.15 GBq, from about 0.11 GBq to about 1.10 GBq, from about 0.11 GBq to about 0.8 GBq, from about 0.11 GBq to about 0.6 GBq, from about 0.11 GBq to about 0.4 GBq, or from about 0.11 GBq to about 0.2 GBq. In some embodiments, the radioactivity of the cyanide source is at least about 0.001 GBq, about 0.005 GBq, about 0.008 GBq, about 0.01 GBq, about 0.08 GBq, about 0.06 GBq, about 0.04 GBq, about 0.02 GBq, about 0.1 GBq, about 0.12 GBq, about 0.14 GBq, about 0.16 GBq, about 0.18 GBq, about 0.2 GBq, about 0.25 GBq, about 0.3 GBq. In addition, or in the alternative, the radioactivity of the cyanide source is less than about 2.5 GBq, about 2.25 GBq, about 2.0 GBq, about 1.75 GBq, about 1.50 GBq, about 1.25 GBq, about 1.15 GBq, or less than about 1 GBq.

In some embodiments, the amount of radioactive cyanide source is provided as a function of radioactivity measured in curies (Ci). Thus, in some embodiments, the radioactivity of the cyanide source ranges from about 1 mCi to about 500 mCi, from about 2 mCi to about 400 mCi, from about 3 mCi to 300 mCi, from about 4 mCi to about 200 mCi, from about 5 mCi to about 100 mCi, or from about 10 mCi to about 50 mCi.

### C. Base Additive

The photoredox system as disclosed herein requires a base additive. In some embodiments, the base additive is an organic base selected from the group consisting of ammonia, methylamine, tetrabutylammonium hydroxide (TBAOH), trimethylamine, ethylamine, di-isopropylethylamine (DIPEA), phenylamine, pyridine, imidazole, histidine, guanidine, benzimidazole, phosphazene, and a combination thereof. In some embodiments, the base additive is DIPEA or TBAOH.

In some embodiments, the base additive is an inorganic base selected from the group consisting of alkali carbonates, alkali bicarbonates, alkali acetates, and a combination thereof. In some embodiments, the base additive is selected from the group consisting of Na₂CO₃, NaOAc, KHCO₃, NaHCO₃, KOAc, and a combination thereof. In some embodiments, the base additive is NaHCO₃.

In some embodiments, the base additive is selected from the group consisting of Na₂CO₃, NaOAc, KHCO₃, NaHCO₃, KOAc, DIPEA, TBAOH, and a combination thereof.

In some embodiments, the base additive is a buffer solution. Exemplary buffer solutions include, but are not limited to, a borate buffer, a phosphate buffer, and a combination thereof.

The pH of the base additive can vary. In some embodiments, the base additive is an organic base or inorganic with a pH neat or in solution ranging from about 7.5 to about 10.5, from about 7.5 to about 10.0, from about 7.8 to about 9.5, from about 7.8 to about 9.0, or from about 8 to about 9. In some embodiments, the pH of organic or inorganic base near or in solution is at least about 7.5, about 7.6, about 7.7, about 7.8, about 7.9 about 8.0, about 8.1, about 8.2, about 8.3, about 8.4, about 8.5, about 8.6, about 8.7, about 8.8, or at least about 8.9. In addition, or in the alternative, the pH of the organic or inorganic base neat or in solution is less than about 9.2, less than about 9.1, less than about 9.0, less than about 8.9, less than about 8.8, less than about 8.7, less than about 8.6, less than about 8.5, less than about 8.4, less than about 8.3, less than about 8.2, or less than about 8.1.

In some embodiments, the base additive is in a solid form. In some embodiments, the base additive is in a liquid form. Exemplary liquid forms include, but are not limited to, aqueous solutions and/or saturated solution (e.g., in water). In some embodiments, the base additive is a NaHCO₃ in solid form. In some embodiments, the base additive is NaHCO₃ in the form of an aqueous saturated solution.

The amount of base additive present in the photoredox system can vary. In some embodiments, the base additive is present in an amount that ranges from about 1 equiv. to about 10 equiv., from about 1 equiv. to about 8 equiv., from about 1 equiv. to about 6 equiv., from about 1 equiv. to about 5 equiv., from about 1 equiv. to about 4 equiv., from about 1 equiv. to about 3 equiv., or from about 1 equiv. to about 2 equiv. In some embodiments, the base additive is present in the photoredox system in an amount that ranges from about 0.1 equiv. to about 5 equiv. from about 0.2 equiv. to about 4.5 equiv., from about 0.5 equiv. to about 4 equiv., from about 0.5 equiv. to about 3 equiv., from about 0.5 equiv. to about 2.5 equiv., from about 0.5 equiv. to about 2.0 equiv., from about 0.5 equiv. to about 1.5 equiv., or from about 0.75 equiv. to about 1.25 equiv. In some embodiments, the base additive is present in the photoredox system in an amount of less than about 7 equiv., less than about 5 equiv., less than about 4 equiv., less than about 3.5 equiv., less than about 3.0 equiv., less than about 2.5 equiv., less than about 2.0 equiv., less than about 1.5 equiv., less than about 1.0 equiv., less than about 0.75 equiv., less than about 0.5 equiv., less than about 0.25 equiv., less than about 0.1 equiv., less than about 0.01 equiv., less than about 0.001 equiv., less than about 0.0001 equiv., or less than about 0.00001 equiv. In addition, or in the alternative, the base additive is present in the photoredox system in an amount of at least about 0.0001 equiv., at least about 0.001 equiv., at least about 0.01 equiv., at least about 0.1 equiv., at least about 1 equiv., at least about 2 equiv., or at least about 3 equiv.

### D. Solvent

The photoredox system as disclosed herein requires a solvent. The solvent can be any solvent as long as it promotes a CRA-S_{N}AR reaction mechanism as already describes above (see Scheme 1c of FIG. 16). In some embodiments, the solvent is selected from the group consisting of a polar protic solvent, a poler aprotic solvent, and a combination thereof.

In some embodiments, the solvent is a polar protic solvent. Exemplary polar protic solvents include, but are not limited to, water, ammonia, acetic acid, dimethylacetamide (DMA), n-propanol (n-PrOH), t-butanol, methanol (MeOH), ethanol (EtOH), and isopropyl alcohol (iPr-OH). In some embodiments, the solvent is ethanol.

In some embodiments, the solvent is a polar aprotic solvent. Exemplary aprotic solvents include, but are not limited to, chloroform, dichloromethane (DCM), dichloroethanol (DCE), trifluoroethanol (TFE), tetrahydrofuran (THF), ethyl acetate, dimethyl formamide (DMF), N-methyl-2-pyrrolidone (NMP), dimethyl sulfoxide (DMSO), acetone, diethyl ether, and hexamethylphosphoric triamide (HMPT). In some embodiments, the polar aprotic solvent is dichloroethanol and/or trifluoroethanol. In some embodiments, the polar aprotic solvent is acetonitrile.

In some embodiments, the solvent is selected from the group consisting of ethanol, water, acetonitrile, trifluoroethanol, and a combination thereof.

The amount of solvent present in the photoredox system can vary. In some embodiments, the solvent is present in the photoredox system at a concentration of about 0.01M to about 0.5M, from about 0.01M to about 0.3M, from about 0.05M to about 0.25M, from about 0.05M to about 0.20M, from about 0.08M to about 0.18M, from about 0.08M to about 0.15M, from about 0.08M to about 0.12M, or about 0.1M. In some embodiments, the solvent present in the photoredox system is at least about 0.01M at least about 0.02M, at least about 0.03M, at least about 0.04M, at least about 0.05M, at least about 0.06M, at least about 0.07M, at least about 0.08M, at least about 0.09M, at least about 0.1M. In addition, or in the alternative, the solvent present in the photoredox system is less than about 0.5M, less than about 0.45M, less than about 0.40M, less than about 0.35M, less than about 0.30M, less than about 0.25M, less than about 0.20M, less than about 0.18M, less than about 0.16M, less than about 0.14M, less than about 0.12M, less than about 0.1M, less than about 0.8M, less than about 0.6M, less than about 0.4M, less than about 0.2M, less than about 0.08M, less than about 0.06M, less than about 0.04M, less than about 0.02M.

In some embodiments, the solvent is a combination of a first solvent and a second solvent. In some embodiments, such first and second solvents can individually be selected from any of the above mentioned polar protic solvents and polar aprotic solvents. In some embodiments, the solvent is a combination of ethanol as a first solvent and acetonitrile as a second solvent. In some embodiments, the solvent is a combination of ethanol as a first solvent and water as a second solvent. In some embodiments, the solvent is a combination of trifluoroethanol as a first solvent and difluoroethanol as a second solvent.

The amount of each first and second solvent in the above solvent combinations can vary. In some embodiments, the first solvent and second solvent are present as a weight ratio ranging from about 1:20 to about 20:1, from about 1:15 to about 15:1, from about 1:12 to about 12:1, from about 1:10 to about 10:1, from about 1:8 to about 8:1, from about 1:6 to about 6:1, from about 1:5 to about 5:1, from about 1:4 to about 4:1, from about 1:3 to about 3:1, from about 1:2 to about 2:1, or about 1:2, first solvent to second solvent. In some embodiments, the first solvent and second solvent are present as a weight ratio ranging from about 1:12, about 1:11, about 1:10, about 1:9, about 1:8, about 1:7, about 1:8, about 1:6, about 1:5, about 1:4, about 1:3, or about 1:2, first solvent to second solvent.

In some embodiments, the solvent can also be a combination of more than two solvents, e.g., a first solvent, a second solvent, and a third solvent or more.

In alternate embodiments, the photoredox system contains radioactivity. In such embodiments, the solvent is present in an amount of from about 100 μl to about 1000 μl, from about 100 μl to about 800 μl, from about 150 μl to about 750 μl, from about 200 μl to about 700 μl, from about 250 μl to about 650 μl, from about 300 μl to about 600 μl, from about 350 μl to about 575 μl, from about 400 μl to about 550 μl, from about 450 μl to about 550 μl, or from about 475 μl to about 525 μl. In some embodiments, the solvent is present in an amount of at least about 125 μl, about 225 μl, about 325 μl, about 425 μl, about 450 μl, about 475 μl, or at least about 500 μl. In addition, or in the alternative, the solvent is present in an amount of less than about 725 μl, about 700, μl about 675 μl, about 650 μl, about 625 μl, less about 600 μl, about 575 μl, about 550 μl, about 525 μl, or less than about 500 μl. In some embodiments, the solvent is present in an amount of about 500 μl.

In some embodiments, the solvent of a photoredox system containing radioactivity is selected from the group consisting of water, ethanol, and a combination thereof.

### E. Blue-Violet Light

The photoredox system as disclosed herein requires a blue-violet light. Blue-violet light refers to blue light rays with the shortest wavelengths (and highest energy), wherein the wavelengths range from about 415 nm to about 460 nm. In some embodiments, the blue-violet light has a wavelength ranging from about 420 nm to about 460 nm, from about 430 nm to about 460 nm, from about 440 nm to about 460 nm, or from about 450 nm to about 460 nm. Not to be bound by theory, but it is believed that blue-violet light is required in the disclosed photoredox system to promote the formation an aromatic cation radical species, which can then subsequently react with the cyanide source as disclosed herein to generate the desired cyanated product (see Scheme 1c of FIG. 16).

Several sources of blue-violet light are available, such as sunlight, and various artificial sources such as, but not limited to, fluorescent light, LED light, CFL (compact fluorescent light), and/or lasers (e.g., neodymium-doped yttrium aluminum garnet (Nd-YAG) and/or krypton fluoride (KrF), Xenon monochloride (XeCl)). In some embodiments, the disclosed photoredox system comprises an LED light as a blue-violet light source. In some embodiments, the disclosed photoredox system comprises a laser as a blue-violet light source.

### III. Arene or Heteroarene Substrate

The photoredox system disclosed herein can be used to introduce a cyano functional group into arene and heteroarene substrates. In some embodiments, the arene or heteroarene substrate contains at least one alkoxy group, —OR, wherein R is selected from the group consisting of a (C₁-C₆) alkyl group, an aryl group, and a heteroaryl group, which is either substituted or unsubstituted. In some embodiments, the alkoxy group —OR is a substituted or unsubstituted —O(C₁-C₆) alkyl group. Exemplary unsubstituted —O(C₁-C₆) alkyl groups include, but are not limited to, —OCH₃, —OCH₂CH₃, —OCH(CH₃)₂ and —OC(CH₃)₃. In some embodiments, the arene or heteroarene is substituted with at least one —OCH₃. In some embodiments, the arene or heteroarene is substituted with at least two —OCH₃ (e.g., veratrole). In some embodiments, the arene or heteroarene is substituted with at least three —OCH₃ (e.g., pyrogallol trimethyl ether).

In some embodiments, the arene and/or heteroarene substrate comprises a monocyclic aromatic ring or a bicyclic aromatic ring, which can be substituted or unsubstituted. In some embodiments, the arene substrate comprises a 6-membered aryl ring (e.g., phenyl), which can be substituted or unsubstituted. In some embodiments, the heteroarene substrate comprises a 6-membered heteroaryl ring (e.g., pyridinyl), which can be substituted or unsubstituted.

In some embodiments, the arene substrate comprises a bicyclic aromatic ring, which can be substituted or unsubstituted, such as naphthalene. In some embodiments, the heteroarene substrate comprises a bicyclic aromatic ring, which can be substituted or unsubstituted, such as quinoline.

In some embodiments, the arene substrate or heteroarene substrate can be a biologically active molecule. Such biologically active molecules can be found in nature (e.g., a natural product) or can be present in the body of a mammal (e.g., a neurotransmitter). In some embodiments, the arene or heteroarene substrate is not derived from nature but is prepared synthetically. In such embodiments, the arene or heteroarene substrate can be a pharmaceutical agent or a pharmacological agent.

The biologically active molecules represented by the arene substrate or heteroarene substrate disclosed herein comprise a monocyclic or bicyclic aromatic ring. In such instances, the monocyclic or bicyclic aromatic ring represents a portion of the biologically active compound. For example, the monocyclic or bicyclic aromatic ring can be attached with at least one covalent bond to the remaining chemical structure of the biologically active molecule acting as the arene or heteroarene substrate. As already mentioned above these monocyclic or bicyclic aromatic rings comprise at least one alkoxy group (—OR) as described above. Examples of such biologically active molecules selected as arene or heteroarene substrates include, but are not limited to, podophyllotoxin, colchicine, troxipide, benzyl guanidine derivatives, trimethoprim, letrozole, cinepazide, trimebultine.

Thus, in some embodiments, the arene substrate is a biologically active molecule comprising a monocyclic aromatic ring according to Formula (I):

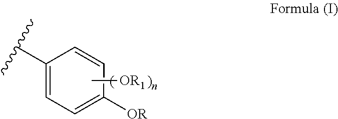

wherein R and each R₁ are independently selected from the group consisting of a (C₁-C₆) alkyl group, an aryl group, and a heteroaryl group, which in each case can be either substituted or unsubstituted; and
n is an integer selected from the group consisting of 0, 1, 2, 3 and 4.

In some embodiments, R is —CH₃ or —CH₂CH₃.

In some embodiments, R₁ is —CH₃.

In some embodiments, and n is 0, 1 or 2.

In some embodiments, the arene substrate comprises a monocyclic aromatic ring such as:

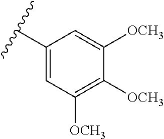 [SMILES: *c1cc(OC)c(OC)c(OC)c1]

Another aspect of the current disclosure relates to arene and heteroarene substrates comprising a monocyclic aromatic ring according to a compound of Formula (II):

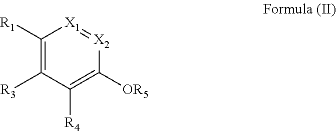

wherein X₁ and X₂ are each independently selected from the group consisting of —N and —CR₂;
R₁ and R₂ are each independently selected from the group consisting of —H, substituted or unsubstituted —(C₁-C₆) alkyl, substituted or unsubstituted —O(C₁-C₆) alkyl, substituted or unsubstituted —O(benzyl), substituted or unsubstituted —CH₂CO(C₁-C₆) alkyl, substituted or unsubstituted —(C₁-C₆) alkyl-NH(R₆), substituted or unsubstituted —N(C₁-C₆ alkyl)(R₆), —CH═CHCOOH, substituted or unsubstituted —CO(C₁-C₆) alkyl, substituted or unsubstituted —COO(C₁-C₆) alkyl, substituted or unsubstituted heteroaryl, and —COOH;
R₃ is selected from the group consisting of —H, substituted or unsubstituted —O(C₁-C₆) alkyl, substituted or unsubstituted —(C₁-C₆) alkyl, substituted or unsubstituted —CH₂CO(C₁-C₆) alkyl and substituted or unsubstituted —(C₁-C₆) alkyl-NH(R₆);
R₄ is selected from the group consisting of —H, substituted or unsubstituted —O(C₁-C₆) alkyl, substituted or unsubstituted —(C₁-C₆) alkyl, —Cl and —F;
R₅ is selected from the group consisting of substituted or unsubstituted (C₁-C₆) alkyl group, substituted or unsubstituted aryl group, and substituted or unsubstituted heteroaryl group;
R₆ is —H or an amine protecting group (PG); and
a pharmaceutically acceptable salt form thereof.

In some embodiments, R₅ is a substituted or unsubstituted (C₁-C₆) alkyl group. In some embodiments, R₅ is —CH₃. In some embodiments, R₅ is —CH₂CH₃.

In some embodiments, X₁ and X₂ both are —CR₂. In some embodiments, X₁ is —N. In some embodiments, X₂—N.

In some embodiments, R₁ is selected from the group consisting of —H, substituted or unsubstituted —(C₁-C₆) alkyl. In some embodiments, R₁ is selected from the group consisting of —H, —OCH₂CH₃, —OCH₃, and —OCH(CH₃)₂. In some embodiments, R₁ is selected from the group consisting of —H and substituted or unsubstituted —O(C₁-C₆) alkyl. In some embodiments, each R₁ is independently selected from the group consisting of —H, —OCH₂CH₃, —OCH₃, and —OCH(CH₃)₂.

In some embodiments, R₁ is selected from the group consisting of —H, substituted or unsubstituted —O(benzyl), and substituted or unsubstituted —CH₂CO(C₁-C₆) alkyl. In some embodiments, R₁ is selected from the group consisting of —H, —CH₂COCH₃, and —CH₂COCH₂CH₃.

In some embodiments, R₁ is selected from the group consisting of —H, substituted or unsubstituted —(C₁-C₆) alkyl-NH(R₆), and substituted or unsubstituted —N(C₁-C₆ alkyl)(R₆), wherein R₆ is —H or an amine protecting group (PG). In some embodiments, R₁ is selected from the group consisting of —H, —CH₂CH₂NH(BOC), —N(CH₃)(BOC), and —CH₂NH(BOC).

In some embodiments, R₁ is selected from the group consisting of —H, —CH═CHCOOH, substituted or unsubstituted —CO(C₁-C₆) alkyl, substituted or unsubstituted —COO(C₁-C₆) alkyl, substituted or unsubstituted heteroaryl, and —COOH. In some embodiments, R₁ is selected from the group consisting of —H, —CH═CHCOOH, —COCH₃, —COCH₂CH₃ substituted or unsubstituted —COOCH₃, -pyridinyl, and —COOH.

In some embodiments, R₁ is selected from the group consisting of —CH₃, —OCH₃, —H, —Cl, -Ph, —C(CH₃)₃, —CH₂COCH₃, —CH₂CH₂NH(BOC), —N(CH₃)(BOC), —Br, —CN, —CH═CHCOOH; -pyridin-2-yl, —CH₂C₁, —CH₂CN, —CH₂OH, —CH₂OCH₂CH₃, —CH₂NH(BOC), —CH₂N₃, —COCH₂CH₂COOCH₃, —COH, —COCH₃, —COOCH₃, and —COOH.

In some embodiments, each R₂ is independently selected from the group consisting of —H, substituted or unsubstituted —O(C₁-C₆) alkyl, and substituted or unsubstituted —(C₁-C₆) alkyl. In some embodiments, each R₂ is independently selected from the group consisting of —H, substituted or unsubstituted —O(C₁-C₆) alkyl. In some embodiments, each R₂ is independently selected from the group consisting of —H, —OCH₂CH₃, —OCH₃, and —OCH(CH₃)₂. In some embodiments, each R₂ is independently selected from the group consisting of —H and substituted or unsubstituted —(C₁-C₆) alkyl. In some embodiments, each R₂ is independently selected from the group consisting of —H and —CH₃. In some embodiments, each R₂ is independently selected from the group consisting of —H, —CH₃ and —OCH₃.

In some embodiments, R₃ is selected from the group consisting of —H, substituted or unsubstituted —O(C₁-C₆) alkyl. In some embodiments R₃ is selected from the group consisting of —H and —OCH₂CH₃, —OCH₃.

In some embodiments, R₃ is selected from the group consisting of —H and substituted or unsubstituted —CH₂CO(C₁-C₆) alkyl. In some embodiments, R₃ is selected from the group consisting of —H, —CH₂COCH₃—CH₂COCH₂CH₃, and —CH₂COCH(CH₃)₂.

In some embodiments, R₃ is selected from the group consisting of —H and —(C₁-C₆) alkyl-NH(R₆), wherein R₆ is —H or an amine protecting group (PG). In some embodiments, R₃ is selected from the group consisting of —H, —CH₂NH(R₆) and —CH₂CH₂NH(R₆), wherein R₆ is —H or a BOC group (—COOC(CH₃)₃). In some embodiments, R₃ is selected from the group consisting of —H and —CH₂CH₂NH(R₆), wherein R₆ is —H. In some embodiments, R₃ is selected from the group consisting of —H and —CH₂CH₂NH(R₆), wherein R₆ is a BOC group (—COOC(CH₃)₃).

In some embodiments, R₄ is selected from the group consisting of —H, substituted or unsubstituted —O(C₁-C₆) alkyl, —Cl and —F. In some embodiments, R₄ is selected from the group consisting of —H, —OCH₃, —OCH₂CH₃, —OCH(CH₃)₂, —Cl and —F.

In some embodiments, R₄ is selected from the group consisting of —H, substituted or unsubstituted —(C₁-C₆) alkyl, —Cl and —F. In some embodiments, R₄ is selected from the group consisting of —H, —CH₃, —CH₂CH₃, —CH(CH₃)₂, —Cl and —F.

In some embodiments, R₄ is selected from the group consisting of —CH₃, —OCH₃, —OCH₂CH₃, —OCH(CH₃)₂, and —F.

In some embodiments, R₅ is a substituted or unsubstituted (C₁-C₆) alkyl group. In some embodiments, R₅ is selected from the group consisting of —OCH₃, —OCH₂CH₃, and —OCH(CH₃)₂.

In some embodiments, R₅ is a substituted or unsubstituted aryl or heteroaryl group. In some embodiments, R₅ is phenyl or pyridinyl.

In some embodiments, R₄ is —OCH₃ or —CH₃, R₅ is —OCH₃ or —OCH₂CH₃, and X₂ is —CR₂, wherein R₂ is —H, —CH₃ or —OCH₃.

In some embodiments, R₄ is —OCH₃, R₅ is —OCH₃ or —OCH₂CH₃, and X₂ is —CR₂, wherein R₂ is —H or —OCH₃.

In some embodiments, the arene and heteroarene substrate disclosed herein comprises a bicyclic ring according to a compound of Formula (III):

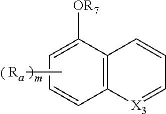

wherein X₃ is selected from the group consisting of —N and —CR₉;
R₇ is selected from the group consisting of substituted or unsubstituted —O(C₁-C₆) alkyl, substituted or unsubstituted —O(aryl), and substituted or unsubstituted —O(heteroaryl);
R₈ and R₉ are each independently selected from the group consisting of —H, substituted or unsubstituted —(C₁-C₆) alkyl, substituted or unsubstituted —O(C₁-C₆) alkyl, substituted or unsubstituted —O(benzyl), substituted or unsubstituted —CH₂CO(C₁-C₆) alkyl, substituted or unsubstituted —(C₁-C₆) alkyl-NH(R₁₀), substituted or unsubstituted —N(C₁-C₆ alkyl)(R₁₀), —CH═CHCOOH, substituted or unsubstituted —CO(C₁-C₆) alkyl, substituted or unsubstituted —COO(C₁-C₆) alkyl, substituted or unsubstituted heteroaryl, and —COOH;
R₁₀ is —H or an amine protecting group (PG);
m is an integer selected from the group consisting of 0, 1, 2, 3, and 4; and
a pharmaceutically acceptable salt form thereof.

In some embodiments, X₃ is selected from the group consisting of —N and —CR₉, wherein R₉ is —H.

In some embodiments, R₇ is —O(C₁-C₆) alkyl. In some embodiments, R₇ is selected from the group consisting of —OCH₃, —OCH₂CH₃, and —OCH(CH₃)₂.

In some embodiments, m is 0. In some embodiments, m is 0 and R₇ is —OCH₃.

In some embodiments, m is 1. In some embodiments, m is 1 and R₇ and R₈ are each independently selected from the group consisting of —OCH₃, —OCH₂CH₃, and —OCH(CH₃)₂.

In some embodiments, m is 2. In some embodiments, m is 2 and R₇, in each instance, and R₈ are each independently selected from the group consisting of —OCH₃, —OCH₂CH₃, and —OCH(CH₃)₂.

In some embodiments, the arene or heteroarene substrates are a biologically active molecule and the monocyclic aromatic ring of Formula (II) or the bicyclic aromatic ring of Formula (III) have at least one of their respective R-substituents selected from the group consisting of R₁, R₂, R₃, R₄, R₆, R₈, R₉, and R₁₀ form a covalent bond with the remaining chemical structure of the biologically active molecule.

The arene and heteroarene substrates described herein may in some cases exist as in stereoisomeric forms and/or pharmaceutically acceptable salt as will be described in more detail below.

### IV. Methods of Using the Photoredox System

The photoredox system described herein can be used in the cyanation of arene and heteroarene substrates. As already described above, the photoredox system contains the following components: a photocatalyst, a base additive, a cyanide source, a solvent, and blue-violet light. Disclosed herein is a photoredox-catalyzed cyanation method using the disclosed photoredox system to introduce a cyanide (—CN) functional group into an arene or heteroarene substrate, the photoredox-catalyzed cyanation method comprising: a) obtaining a reaction mixture comprising an arene or heteroarene substrate, a photocatalyst, a base additive, and a solvent; b) contacting the reaction mixture with a cyanide source to afford a photocyanation reaction mixture; and c) exposing the photocyanation reaction mixture to blue-violet light to form a cyano arene or heteroarene product.

As already described above, in some instances the cyanide source is not radioactive and the disclosed cyanation method can provide non-radioactive arenes and heteroarene products. However, in some instances, the cyanide source can be radioactive (meaning it contains a radioisotope) and then the disclosed cyanation method is able to provide radioactive arenes and heteroarene products. Since the photoredox system and its components are already described in detail above, the method steps will be described in more detail below.

In some embodiments, the reaction mixture in step a) can be obtained by adding the arene or heteroarene substrate, photocatalyst, base additive and solvent (also referred to as “components”) into a container. The order of addition of these agents can vary and it not particularly relevant. For example, in some embodiments, the arene or heteroarene substrate, photocatalyst, and base additive were added first into the container prior to the addition of the solvent. In some embodiments, all of the agents in the container further undergo mixing, which can be carried out via stirring, rotating, shaking, or sonicating to obtain the reaction mixture in step a).

In some embodiments, the container used in this cyanation method can be any suitable container that can hold all of the reagents, such as a flask, a vial, an Eppendorf vial, etc. In some embodiments, the container belongs to a reaction set-up used for execution of the cyanation method. For example, the reaction set-up can be located in a hot cell when the cyanide source is radioactive or the container belongs to a continuous flow chemistry set-up.

In some embodiments, the contacting step b) is carried out by addition of the cyanide source to the reaction mixture inside the container. In such embodiments, the cyanide source is in a liquid form (i.e., either neat or dissolved in a solvent). In some embodiments, the cyanide source is added rapidly to the container holding the reaction mixture (i.e., all at once) or added slowly over a certain time period. In some embodiments, this time period (also referred to as “addition time period”) ranges from one or more seconds to several minutes. In some embodiments, the addition time period ranges from about 1 minute to about 120 minutes, from about 2 minutes to about 100 minutes, from about 5 minutes to about 90 minutes, from about 8 minutes to about 80 minutes, from about 10 minutes to about 70 minutes, from about 15 minutes to about 60 minutes, from about 20 minutes to about 50 minutes, from about 25 minutes to about 40 minutes, or from about 30 minutes to about 40 minutes. In some embodiments, the addition period is less than about 60 seconds, less than about 55 seconds, less than about 50 seconds, less than about 45 seconds, less than about 40 seconds, less than about 35 seconds, less than about 30 seconds, less than about 25 seconds, less than about 20 seconds, less than about 15 seconds, less than about 10 seconds, less than about 5 seconds, or less than about 1 second.

In some embodiments, the reaction mixture continues mixing during the contacting step to ensure that the photocyanation reaction mixture is homogenous.

In some embodiments, the exposing step c) comprises irradiating or shining the photocyanation reaction mixture with blue-violet light for a certain time period (referred to as “irradiation time”). During this irradiation time period blue-violet light is shining/irradiating onto the photocyanation reaction mixture located inside the container. In some embodiments, this irradiation time period ranges from several minutes to several hours. In some embodiment, the photocyanation reaction mixture is exposed to blue-violet light for an irradiation time period of at least about 1 minute, at least about 2 minutes, at least about 3 minutes, at least about 4 minutes, at least about 5 minutes, at least about 6 minutes, at least about 7 minutes at least about 8 minutes, at least about 9 minutes, or at least about 10 minutes. In addition, or in the alternative, the photocyanation reaction mixture is exposed to blue-violet light for an irradiation time period of less than about 20 minutes, less than about 18 minutes, less than about 16 minutes, less than about 14 minutes, less than about 12 minutes, less than about 10 minutes, less than about 9 minutes, less than about 8 minutes, less than about 7 minutes, less than about 6 minutes, less than about 5 minutes, less than about 4 minutes, less than about 3 minutes, less than about 2 minute, or less than about 1 minute. In some embodiments, the photocyanation reaction mixture is exposed to blue-violet light for an irradiation time period of at least about 1 hour, at least about 5 hours, at least about 10 hours, at least about 15 hours, at least about 18 hours, at least about 20 hours, at least about 25 hours, at least about 30 hours, at least about 32 hours, at least about 34 hours, at least about 40 hours, at least about 45 hours, or at least about 50 hours. In addition, or in the alternative, the photocyanation reaction mixture is exposed to blue-violet light for an irradiation time period of less than about 50 hours, less than about 45 hours, less than about 40 hours, less than about 38 hours, less than about 36 hours, less than about 30 hours, less than about 25 hours, less than about 20 hours, less than about 18 hours, less than about 15 hours, less than about 10 hours, or less than about 5 hours.

In some embodiments, the temperature at which each step of the disclosed cyanation method is carried out can vary. In some embodiments, the temperature of at least one step of the disclosed cyanation method is carried out at room temperature (i.e., about 20° C.). In some embodiments, the temperature of at least one step of the disclosed cyanation method is carried out at a temperature above room temperature (i.e., about 20° C.). In some embodiments, the temperature of at least one step of the disclosed cyanation method is carried out at a temperature of from about 22° C. to about 120° C., from about 25° C. to about 100° C., from about 28° C. to about 80° C., from about 30° C. to about 60° C., from about 30° C. to about 40° C., or from about 32° C. to about 38° C. In some embodiments, the temperature of at least one step of the disclosed cyanation method is carried out at a temperature of at least about 20° C., about 22° C., about 25° C., about 27° C., about 30° C., about 32° C., about 34° C., about 35° C., about 36° C., about 37° C., about 38° C., about 40° C., about 45° C., about 50° C., about 55° C., about 60° C., about 65° C., about 70° C., about 75° C., about 80° C., about 85° C. or at least about 90° C. In addition to, or in the alternative, the temperature of at least one step of the disclosed cyanation method is carried out at a temperature of less than about 100° C., about 95° C., about 90° C., about 85° C., about 80° C., about 75° C., about 70° C., about 65° C., about 60° C., about 55° C., about 50° C., about 45° C., about 40° C., less than about 38° C., less than about 35° C., less than about 30° C.

In some embodiments, the temperature of at least one step of the disclosed cyanation method is carried out at a temperature where the reaction mixture and/or the photocyanation mixture refluxes.

In some embodiments, the at least one step of the disclosed cyanation is step a) and/or b) and/or c).

In some embodiments, the cyano arene or heteroarene product obtained from the method disclosed herein can be further modified. Thus, the method disclosed herein can further comprise at least one additional step, which modifies one or more functional groups present in the cyano arene or heteroarene product. Exemplary functional groups that can be modified include, but are not limited to, cyano (—CN) groups, hydroxyl (—OH) groups, amines (—NH₂), carboxylic acids (—COOH), esters (—COCH₃) and the like.

In some embodiments, the one or more functional groups being modified by at least one additional step further comprised in the disclosed method is a cyano (—CN) functional group. A skilled artisan would generally be aware of the possible modifications that can be made to such a functional group. For example, in some embodiments, the cyano (—CN) functional group present in the cyano arene or heteroarene product can be modified to afford a carboxylic acid functional group, an amide group functionality, or an amine group functionality. A skilled artisan would generally be aware what reagents would be required to transform a cyano (—CN) functional group into any one of these.

For example, in some embodiments, the cyanation method disclosed herein further comprises a contacting step d), wherein the cyano arene or heteroarene product obtained in step c) is exposed to an acid, e.g., sulfuric acid, followed by a base, e.g., sodium hydroxide, to obtain an amide-containing arene or heteroarene product. In some embodiments, contacting step d) can occur at elevated temperature above room temperature (e.g., at about 130° C.).

In additional embodiments, the obtained amide-containing arene or heteroarene product from the contacting step d) can be further modified with a reducing agent to afford an alkyl amine-containing arene or heteroarene product. Suitable reducing agents include, but are not limited to, aluminum-containing reducing agents (e.g., LiAlH₄) or boron-containing reducing agents (e.g., NaBH₄).

In another example, the method disclosed herein further comprises contacting the cyano arene or heteroarene product obtained in step c) with water to hydrolyze the cyano (—CN) functional group into a carboxylic acid group (—COOH).

These are just examples of reagents and reaction conditions to modify the cyano (—CN) functional group present in the cyantated arene or hetereoarene product and the disclosed methods are meant to be exemplary and not limiting thereto.

In an alternate embodiment, the method further comprises at least one additional step, which modifies one or more functional groups present in the cyano arene or heteroarene product, which are not cyano groups.

For example, in some embodiments, the method disclosed herein further comprises contacting the cyano arene or heteroarene product obtained in step c) with suitable reagents which are able to remove protecting groups (PG) from amine (e.g., BOC) and/or hydroxy groups. A skilled artisan would generally be aware as to what such suitable reagents are and no further elaboration is required. See, Peter G. M. Wuts, Theodora W. Greene “Greene's Protective Groups in Organic Synthesis” 10 Apr. 2006, Copyright © 2007 John Wiley & Sons, Inc., which is hereby incorporated by reference in its entirety.

In some embodiments, the photoredox-catalyzed cyanation method further comprises isolation and/or purification of the cyano arene or heteroarene product of the disclosed method. In some embodiments, the cyano arene or heteroarene product is isolated using chromatography techniques (e.g., HPLC, gravity), filtration, or distillation techniques. In some embodiments, the cyano arene or heteroarene product is isolated by simply removing any solvent and/or other volatile components present.

The yield by which the cyano arene or heteroarene can be obtained can vary. In some embodiments, the cyano arene or heteroarene product is not radioactive and can be obtained with a yield of at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 98%. In addition, or in the alternative, the non-radioactive cyano arene or heteroarene product can be obtained with a yield of less than about 99%, less than about 95%, less than about 90%, less than about 85%, less than about 80%, less than about 75%, less than about 70%, less than about 65%, less than about 50%, less than about 45, or less than about 40%. In some embodiments, the non-radioactive cyano arene or heteroarene product is not radioactive and can be obtained with a yield of about 50% to about 99%, from about 60% to about 99%, from about 70% to about 99%, from about 75% to about 99%, from about 80% to about 99%, from about 85% to about 99%, or from about 90% to about 99%. In some embodiments, the yield can be an isolated yield (yield taken post purification and/or isolation of desired arene or heteroarene product) or a crude yield (yield taken of arene or heteroarene product without any purification and/or isolation thereof).

In some embodiments, the cyano arene or heteroarene product is radioactive and can be obtained with a radiochemical yield (RCY) of at least about 20%, at least about 25%, at least about 30%, at least about 35%, at least about 40%, at least about 50%, at least about 55%, at least about 60%, at least about 65%, at least about 70%, at least about 75%, at least about 80%, at least about 85%, or at least about 90%. In addition, or in the alternative, the cyano arene or heteroarene product can be obtained with a radiochemical yield (RCY) of less than about 99%, less than about 95%, less than about 90%, less than about 80%, less than about 75%, less than about 70%, less than about 60%, less than about 50%, less than about 40%, less than about 30%, or less than about 20%. In some embodiments, the cyano arene or heteroarene product can be obtained with a radiochemical yield (RCY) of from about 10% to about 80% from about 15% to about 70%, from about 18% to about 60%, from about 20% to about 50%, from about 20% to about 40%, or from about 25% to about 35%. In some embodiments, the RCY is decay corrected.

In some embodiments, the cyano arene or heteroarene product is radioactive and can be obtained with a radiochemical conversion (RCC) of at least about 25%, at least about 35%, at least about 45%, at least about 55%, at least about 65%, at least about 75%, at least about 85% or at least about 95%. In addition, or in the alternative, the cyano arene or heteroarene product can be obtained with a radiochemical conversion (RCC) of less than about 99%, less than about 95%, less than about 90%, less than about 80%, less than about 70%, less than about 60%, less than about 50%, or less than about 40%. In some embodiments, the cyano arene or heteroarene product can be obtained with a radiochemical conversion (RCC) of from about 30% to about 100%, from about 40% to about 100%, from about 45% to about 98%, from about 50% to about 95%, from about 55% to about 95%, from about 60% to about 95%, from about 65% to about 93%, from about 70% to about 93%, from about 75%, to about 93%, from about 80% to about 93%, or from about 85% to about 93%. In some embodiments, the RCC is decay corrected.

Additional studies that further describe various aspects of the photoredox system and its use in methods for preparing cyano arene and heteroarene products are discussed in more detail below.

### V. Optimization of Photoredox-Catalyzed Cyanation Methods

An abundance of electron-rich arenes and aryl nitriles can be found in various therapeutics and, given recent progress in developing photoredox-mediated ¹⁸F-fluorination of electron-rich arenes⁹⁻¹¹ (Scheme 1b of FIG. 16), the current disclosure explored whether organic photoredox catalysis could mediate ¹¹C-cyanation on electron-rich arenes at C-O bonds in a site-selective manner via arene cation radical intermediates (Scheme 1c of FIG. 16).¹² Recently, it was found that organic photoredox-catalyzed cation radical-accelerated nucleophilic aromatic substitution (CRA-S_{N}Ar) reactions are possible with readily available aryl ether substrates, where the alkoxy group serves as the nucleofuge. Despite the success of these methods, the substrate scope and catalytic efficiency were somewhat limited due to catalyst decomposition by adventitious cyanide. In the current disclosure, a photoredox system is developed that not only greatly improves the catalytic efficiency (up to 99% yield), but also expands the reaction substrate scope to more electron-rich arenes (E_{1/2}<+1.50 V vs SCE) such as veratrole and pyrogallol trimethyl ether, derived from catechol, guaiacol, pyrogallol and syringol, which are important moieties and functional groups in natural products, synthetic drug candidates, and organic material structures. This photoredox-catalyzed transformation can be adapted to ¹¹C- and ¹³C-cyanation for efficient late-stage (radio)labeling on a variety of readily available complex precursors with pyrogallol or veratrole cores.

### A. Photoredox-Catalyzed ¹²C-Cyanation by Catalyst S1

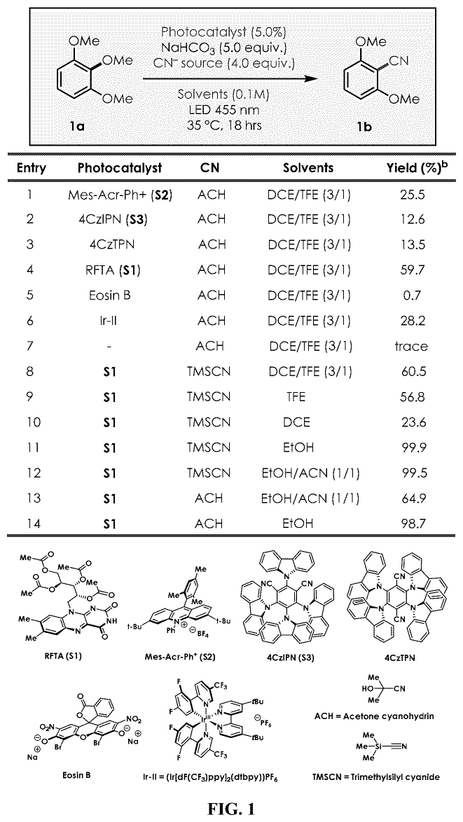

In a recent disclosure,¹² substrate 1a (pyrogallol trimethyl ether) had low reactivity (18% isolated yield) in the CRA-S_{N}Ar cyanation reaction catalyzed by Mes-Acr-Ph⁺ catalyst (S2, E_{1/2}=+2.11 V vs SCE) due to the electron-rich structure of the trimethoxy functionality. In developing efficient new cyanation methods that are compatible with electron rich arenes, compound 1a was used as a model substrate to evaluate the catalytic efficiency. First, different photocatalysts were screened using CRA-S_{N}Ar cyanation conditions. In brief, acetone cyanohydrin (4 equiv.), substrate 1a (0.1 mmol), and an excess amount of NaHCO₃ (5 equiv.) were mixed with a catalytic amount (5 mol %) of photocatalysts and the mixtures were irradiated with 455 nm LEDs for 18 hours. The resulting C—O cyanation product 1b (2,6-dimethoxy benzonitrile) was then visualized by UV-HPLC equipped with a C18 column. As shown in FIG. 1 (entries 1-7), riboflavin tetraacetate (RFTA, S1, E_{1/2}=+1.67 V vs SCE)¹³ resulted in the highest yield (59.7%, FIG. 1, Entry 4) among the catalysts tested.

Next, other organic cyanide and inorganic cyanide sources were screened and it was found that the reaction yields of 2b were not greatly improved (see Examples 19 and 21). However, compared with acetone cyanohydrin (ACH), trimethylsilyl cyanide (TMSCN) was found to give fewer byproducts based on HPLC analysis for both substrates 1a and 2a. Thus, TMSCN was then used for further optimization thereafter. It is noteworthy that acetone cyanohydrin remains a viable cyanide alternative as comparable reaction yields were obtained (FIG. 1, Entries 13 & 14; Example 19, Entries 1 & 2). Using TMSCN as the cyanide source, the impact of solvents was then investigated for both 1a (FIG. 1, Entries 8-12) and 2a (Example 22). We were pleased to find that the use of protic ethanol as the solvent afforded the highest reactivity, while acetonitrile could be employed as a non-protic co-solvent with little deleterious effect, which could aid in the solubility of certain substrates (FIG. 1, Entry 12).

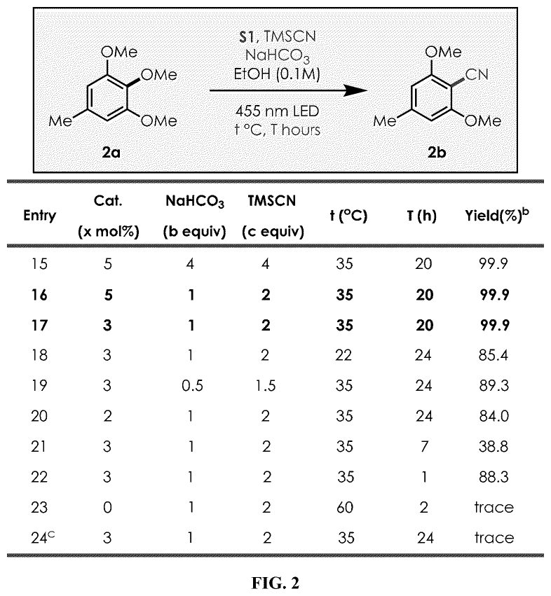

Next, commonly used organic or inorganic basic or acidic additives as well as aqueous buffer solutions were screened and it was identified that solid or aqueous sodium bicarbonate was an optimal additive for the generation of free cyanide in situ, likely by improving the cyanide anion reactivity via its weak basicity and preventing the oxidation of the cyanide anion by the photoredox catalyst (Example 24). Similarly, buffer solutions with weak basicity were also tested using substrate 2a. High yields were observed for the reactions with slightly basic (pH=8.0˜9.0) buffers (20 μL) in the reaction solutions. No reaction was observed in the absence of the basic additives (Example 24). Heating a catalyst-free mixture of 2a, NaHCO₃ and TMSCN in ethanol overnight generated almost none of the desired product (FIG. 2, Entry 23). For the most reactive substrate 2a, the catalyst loading can be further reduced to 3.0 mol % without impacting the yield (FIG. 2, Entry 17).

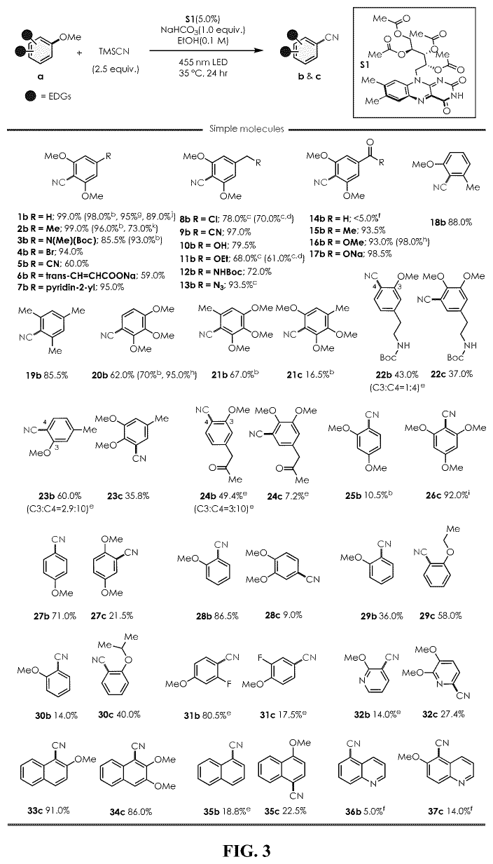

Having identified the optimal reaction conditions, the substrate scope and functional group tolerance were further examined. As shown in FIG. 3, various 5-position functionalized 1,2,3-trimethoxy benzenes were converted into the 2,6-dimethoxy aryl nitriles with minimal side reactions and remarkably high isolated yields. A series of functional groups such as tertiary amines (3a), bromides (4a), nitriles (5a), ketones (14a), esters (15a), benzoates (16a), and even alkenes (6a), were tolerated. Notably, a number of reactive benzylic functional groups such as benzyl chlorides (8a), nitriles (9a), alcohols (10a), ethers (11a), Boc protected amines (12a), and azides (13a), were also tolerated in the reaction regardless of potential competing S_{N}2 and oxidization reactions, and provided the desired demethoxycyanation products with good to excellent isolated yields (61.0%-97.0%). The broad substrate scope made the 2,6-dimethoxy aryl nitrile moiety a good building block to analogs of a number of synthetic bioactive complex compounds.

Single-site-activated substrates such as 18a, 19a, and 20a also produced the desired cyanation products with high yields site selectively. Notably, although unconverted 20a was observed when 2.5 equivalents of TMSCN was used in the standard conditions, a larger scale reaction with additional TMSCN (3.0-4.0 equiv.) or ACH (4.0 equiv.) and extended reaction times (96 h) increased yields to 95.0%. Penta-substituted substrate 21a favored the formation of 21b to 21c (21b:21c=4:1).

The standard conditions were also successfully applied to 3,4-dimethoxy-N-boc-tyramine (22a), leading to 43% of the demethoxycyanation products and 37% of the C—H cyanation product at the C5 position. Additional examples (23a, 24a and 28a) involving the veratrole motif or 1,4-dimethoxy benzene (27a) also demonstrated the preferential formation of demethoxycyanation adducts over the C—H cyanation products. Ethoxy (29a) and isopropoxy (30a) leaving groups were unsurprisingly found to be less reactive than methoxy substrates. The electronic influence of the fluorine atom surprisingly resulted in a dramatic difference on the demethoxycyanation reactivity. The 2-fluorine-4-methoxy benzonitrile product (31b) was generated as the major cyanation product in over 80.0% yield. Methoxy naphthalenes (33a & 34a) afforded C—H cyanation products, which matched with previous reports on photoredox-catalyzed C—H cyanation.¹⁴ A small amount of demethoxycyanation products were observed for hetero arenes such as 2,3-dimethoxy pyridine (32a), methoxy naphthalene (35a), and 5-methoxy quinoline (36a), while only moderate C—H cyanation product formation was observed for 6-methoxy quinoline (37a).

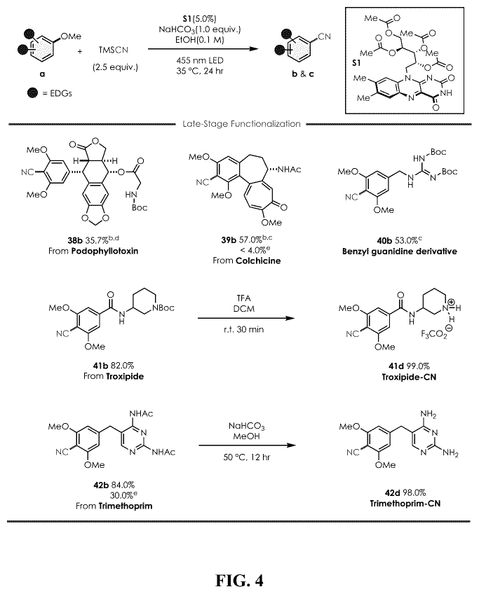

With these results in hand, site- and chemo-selective cyanation for late-stage functionalization (FIG. 4) of existing natural product molecules bearing the electron-rich pyrogallol cores was subsequently performed next. Most of these natural products have relatively narrow therapeutic indices and increased antibiotic resistance. Efforts have been made to modify these molecules to reduce their toxicity and drug resistance. Selectively tuning one of the three methoxy groups may refine their structure-activity relationship (SAR), which has been relatively unexplored thus far. Among the tested molecules, N-Boc-glycine-podophyllotoxin (38a), was recently disclosed to possess potent anti-cancer activity. Our photoredox-catalyzed reaction introduced the nitrile group to 38a site-selectively, leading to 38b in 35.7% isolated yield.

Colchicine (39a) is a natural product agent used to induce polyploidy in plant cells and an alternative agent for treating gout and inflammation in a variety of diseases. Alkoxyarene 39a was directly converted to the desired aryl nitrile analog (39b) in good yield (57.0%) by controlling the reaction at room temperature, which would greatly reduce side reactions such as photoisomerization and possible thermal decomposition.

Guanidine compounds have been found to be useful cardiac PET imaging agents. Under standard reaction conditions, a nitrile group was introduced onto the guanidine precursor (40b) in good yields (53.0%), suggesting the reaction may be used to produce ¹¹C labeled benzyl guanidine analogs efficiently. Troxipide is a marketed drug for gastroesophageal reflux disease. Recent studies have found that troxipide's analogs have anti-tumor properties. N-Boc-protected troxipide (41a) was functionalized with complete site selectivity to afford the corresponding aryl nitrile (41b) in 82.0% yield. Further Boc deprotection was also carried out without any impact on the nitrile group. Trimethoprim is a synthetic diaminopyrimidine antibiotic and a folate synthesis inhibitor used to treat infection and may also be used in combination with other drugs to treat pneumocystis pneumonia in HIV-infected patients. Site-selective cyanation of the trimethoprim diacetate 42b on the aromatic ring led to the nitrile product 42b with an excellent yield of 84.0%. The aryl nitrile analog of trimethoprim was successfully obtained by subsequent acetate deprotection in high yield.

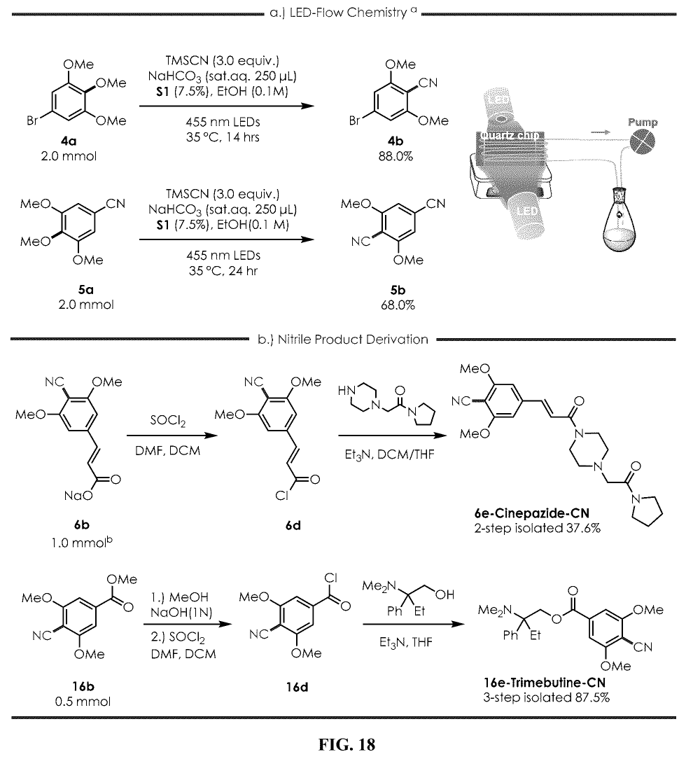

To demonstrate the cyanation reaction can be used in preparative scale, larger scale reactions (200 mg-1.0 g) were conducted in round-bottom flasks or flow devices (quartz chip). Scale-up reactions for 1a, 2a, 3a, 6a, 16a, 20a, 21a, 25a were performed using blue LED-irradiated round-bottom flasks (FIG. 3). All substrates maintained similar yields after scale-up. The yields were further increased by adding an additional cyanide source and prolonging the irradiation time, except for 25a. Substrates 4a and 5a (2.0 mmol) were converted into the corresponding aryl nitriles in 88% (14 h) and 68% (24 h) isolation yield, respectively, in the flow devices (quartz chip, Scheme 2 of FIG. 18) with good catalytic efficiency. As a proof-of-principle example, acyl chloride 6d was generated in high yield from the crude photocyanation reaction mixture containing 6b, which was then converted to 6e, a nitrile analog of a cardio-cerebral vascular drug cinepazide in an overall 37.6% isolated yield. Similarly, acyl chloride 16d was be easily obtained by hydrolysis of ester 16b in basic methanol solution, which was then converted into 16e as a cyanide analog of trimebutine in 87.5% yield.

### B. Photoredox-Catalyzed ¹³C-Demethoxycyanation by Catalyst S1

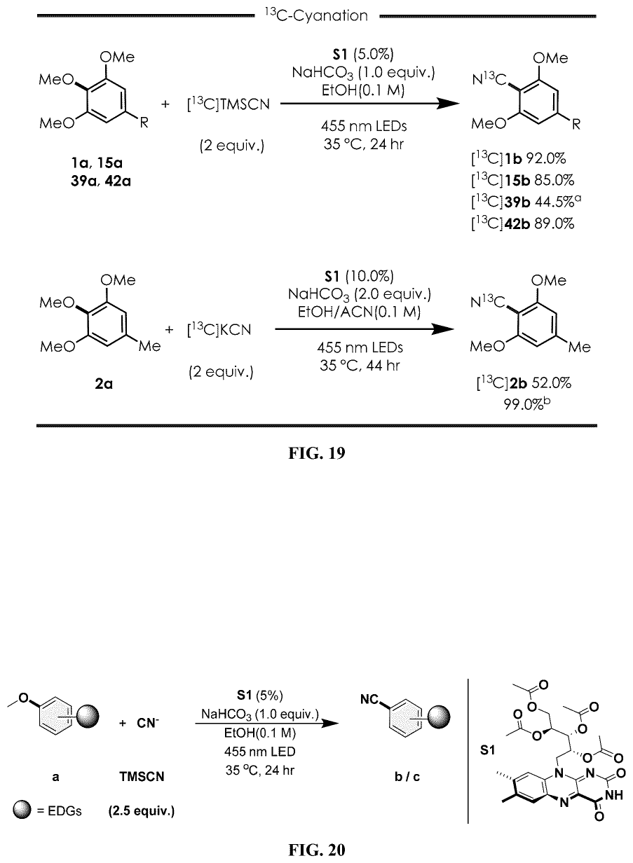

¹³C labeling is one of the stable isotope labeling techniques that can be utilized with mass spectrometry to help better understand cellular metabolism in living organisms. ¹³C-enriched reagents have been used in hyperpolarized carbon-13 magnetic resonance imaging (MRI). These isotopologs provide chemical and spatial information, offering a probe for specific metabolic pathways. Despite progress, there remains a need to develop new and efficient ¹³C labeling methods for highly functionalized molecules. The use of the photoredox method disclosed herein to synthesize aryl [¹³C]nitriles was evaluated. Compared with recently reported transition metal-catalyzed cyanide/[¹³C]cyanide exchange reactions, the method disclosed herein is metal-free, higher-yielding, and affords 100% [¹³C]CN enriched products. For example, [¹³C]2b was isolated in a good yield (52%) when 10.0 mol % of S1 and 2.0 equiv. of [¹³C]KCN were used. The yield could also be increased to up to 99% when 3.0 equivalents of [¹³C]KCN were added and irradiation time extended to 3 days. When [¹³C]TMSCN was used as the [¹³C]cyanide source, the corresponding carbon-13 enriched adducts [¹³C]1b, [¹³C]15b, [¹³C]39b, and [¹³C]42b were all forged in high isolated yields (Scheme 3 of FIG. 19).

The ability to perform photoredox-catalyzed ¹¹C-cyanation would be valuable since it could lead to new PET agents for various applications. Despite success in the aforementioned ^{12/13}C-demethoxycyanation reactions, it could be challenging to directly perform ¹¹C-radiocyanation by adopting these conditions because 1) only trace amounts of [¹¹C]CN⁻ can be used, compared with an excess of CN⁻ in the ^{12/13}C-demethoxycyanation; 2) the reaction requires fast kinetics (generally within 10 min) due to the short half-life of ¹¹C, and 3) the best cyanide source, [¹¹C]TMSCN, is difficult to generate rapidly in situ. Since TBA⁺[¹¹C]CN⁻ can be prepared by trapping [¹¹C]HCN in weak basic solutions, ¹¹C-demethoxycyanation conditions using TBA⁺[¹¹C]CN were first examined.

Indeed, TBA⁺[¹¹C]CN-reacted with 1a (0.03 mmol, 0.06 M) in the presence of S1 (2.0 mg, 12.2 mol %) to afford the expected product in 38% decay-corrected radiochemical conversion (RCC) in 4 min (FIG. 5, Entry 2). After an additional two min of LED irradiation, the resulting radioproduct increased to 50%. Previous studies demonstrated that laser irradiation (450 nm) improved RCCs in photoredox-catalyzed ¹⁸F-arene deoxyfluorination reactions⁹ and direct ¹⁸F-fluorination reactions of arenes.¹¹ When the same reaction were carried out with 450 nm laser irradiation for 6 min, the RCC increased to 91.6±1.8% (FIG. 5, Entry 1, N=3). For model substrate 1a, the laser irradiation time did not significantly affect the RCC (cf. 4 min, Entry 4; and 8 min, Entry 5). Replacing the N₂ atmosphere with O₂ did not change the RCC but led to more UV impurities (FIG. 5, Entry 6). No reaction was observed in the absence of photocatalyst S1, confirming that the photocatalyst was necessary ¹¹C-demethoxycyanation (FIG. 5, Entry 10).

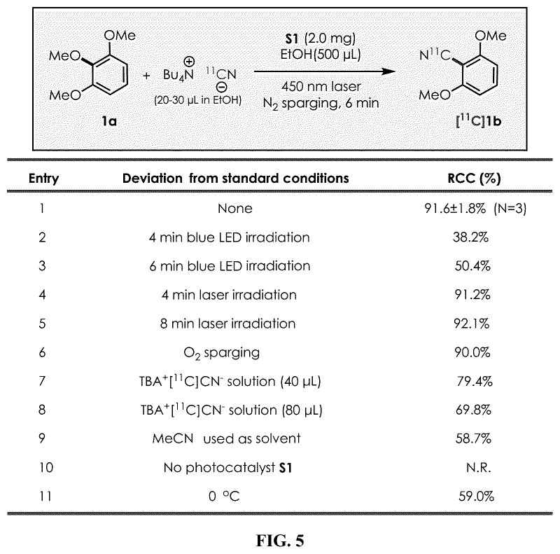

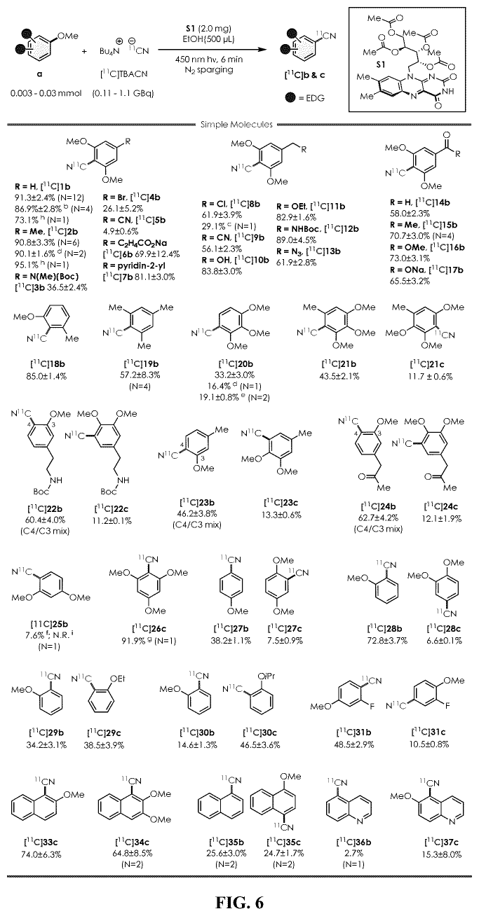

The substrate scope was assessed (FIG. 6) using optimized labeling conditions in FIG. 5, Entry 1. For most substrates, the RCCs of ¹¹C-radiocyanation were high, correlating well with the photoredox-catalyzed ¹²C-cyanation. Although 5b was isolated in over 60% yield in the corresponding ¹²C-cyanation reaction, minimal formation of [¹¹C]5b was observed in ¹¹C labeling reaction. Similarly, compared with 94% yield of 4b, substrate 4a (0.03 mmol) was not reactive under standard ¹¹C-labeling conditions. Interestingly, reducing the substrate loading from 0.03 mmol to 0.01 mmol led to greatly improved radiolabeling yields (26.1±5.2%, N=3). Aniline and mono-N-Boc protected aniline were unreactive substrates in this transformation, likely due to the high acidity of their respective cation radical species. In contrast, the doubly protected 3,4,5-trimethoxy aniline (3a, 0.03 mmol) was converted into [¹¹C]3b with a moderate RCC (36.5±2.4%, N=3). Decreasing 3a loading to 0.01 mmol resulted in an increase in RCC to 49.1% (N=1). Benzyl amines are compatible with the photoredox radiocyanation reactions, as N-Boc protected 3,4,5-trimethoxyl benzyl amine 12a (0.01 mmol) was converted into [¹¹C]12b with an excellent RCC (89.0±4.5%, N=3).

Chemoselectivity between a typical S_{N}2 radiolabeling reaction and the photoredox-catalyzed demethoxy radiocyanation using benzylic chloride substrate 8a were examined. Thermal S_{N}2 labeling conditions led to trimethoxybenzyl [¹¹C]nitrile [¹¹C]9a in a moderate RCC of 29.1% (N=1), and the photoredox-catalyzed radiocyanation conditions resulted in [¹¹C]8b in a RCC of 61.9±3.9% (N=3), with no benzylnitrile product [¹¹C]9a. Highly reactive ¹¹C-labeled products such as benzyl chloride ([¹¹C]8b), benzyl alcohol ([¹¹C]10b), and benzyl azide ([¹¹C]13b) could also serve as ¹¹C-intermediates that can be conjugated with bioactive molecules with corresponding reactive counterparts, such as alcohols, carboxylic acids, dibenzylcyclooctyne, and strained alkenes, to synthesize additional radiolabeled bioactive agents.

Compared with the previous ¹²C-cyanation reactions, a great increased preference of demethoxy ¹¹C-radiocyanation (leading to [¹¹C]22b, [¹¹C]23b, [¹¹C]24b, [¹¹C]27b, and [¹¹C]28b) over the C—H ¹¹C-radiocyanation reactions (leading to [¹¹C]22c, [¹¹C]23c, [¹¹C]24c, [¹¹C]27c, and [¹¹C]28c) was observed. The use of methoxynaphthylene and methoxy quinoline substrates mainly led to C—H ¹¹C-cyanation adducts ([¹¹C]33c-[¹¹C]37c) similar to that observed in the ¹²C-chemistry. Because LED light sources are more affordable than lasers, LED-promoted labeling under the standard conditions were also tested. For the most reactive substrate 2a, the RCC of [¹¹C]2b was >90%, which was comparable to laser irradiation. However, laser irradiation was more efficient when less reactive substrate 20a was tested.

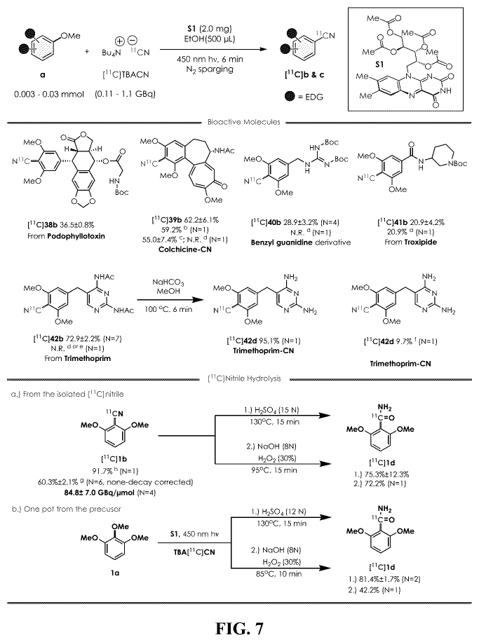

With these results, late-stage ¹¹C-radiocyanation of bioactive substrates was investigated next. As shown in FIG. 7, [¹¹C]39b and [¹¹C]42b were obtained in 62.2±6.1% (N=3) and 72.9±2.2% (N=7) RCCs, respectively. The protecting groups in [¹¹C]42b were easily removed in 95.2% yield, leading to a close analog of a bioactive agent, trimethoprim. In addition to [¹¹C]39b and [¹¹C]42b, other ¹¹C-labeled product, [¹¹C]38b, [¹¹C]40b, and [¹¹C]41b were also obtained with moderate RCCs. The aryl [¹¹C]-nitriles can also be further derivatized to other functional groups. For example, [¹¹C]-amide [¹¹C]1d could be obtained either from the isolated [¹¹C]1b (76.3±14.0% RCC, N=3), or through a one-pot method (81.4±1.7% RCC, N=2) that greatly shortens the synthesis from 1a. And the Am of [¹¹C]1b was also determined to be 84.8±7.0 GBq/μmol (N=4).

Overall, these results demonstrate the potential of using site-selective late-stage ¹¹C-radiolabeling strategy for PET probe development based on existing natural products and drug candidates bearing electron-rich arene moieties. The low precursor loading in these reactions also simplified the purification process. Moreover, the corresponding carbon-12 authentic standards could be prepared using the same photoredox-catalyzed demethoxy ¹²C-cyanation reaction with moderate to very high yields. Automation of this method disclosed herein would greatly facilitate its adoption at different PET centers. Because TBA⁺[¹¹C]CN⁻ can be easily obtained from cyclotron/procab produced [¹¹C]CN⁻, automation of the photoredox reaction would be key for this process.

### C. Photoredox-Catalyzed ¹¹C-Cyanation by Catalysts S2 and S3

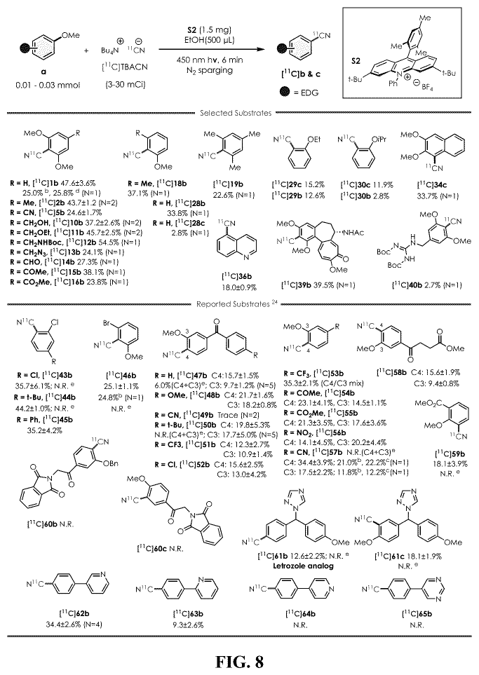

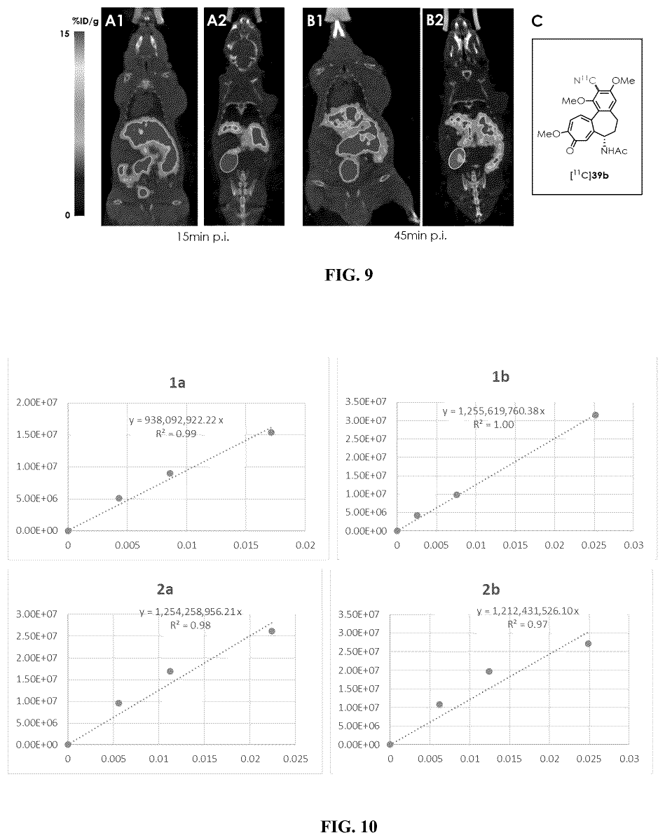

In addition to S1, the use of other catalysts for ¹¹C-radiocyanation was also examined. It was found that S3 (4-CzIPN) could also carry out the demethoxy ¹¹C-radiocyanation of 1a (0.006 mmol, 0.012 M), 2a (0.01 mmol, 0.02 M), and 41a (0.02 mmol, 0.04 M) in 73.1%, 95.1%, and 20.9% RCC, respectively (FIG. 6). However, no desired ¹¹C-radioproducts were detected when other bioactive molecules such as colchicine 39a and trimethoprim diacetate 42a were tested (FIG. 7). Recently, it was found that acridinium photocatalyst S2 could promote demethoxy ¹¹C-cyanation of over 20 arenes with an array of functional groups. Even though the yields may be lower, photocatalyst S2 may have a broader substrate scope as it is more oxidizing than S1. To validate this hypothesis, 19 substrates in FIG. 6 and FIG. 7 were randomly selected along with new substrates 43a-65a for demethoxy ¹¹C-radiocyanation reactions. As shown in FIG. 8, although the RCCs were relatively lower for the highly electron-rich arenes, S2 has a broader substrate scope than S1. For nitrile 5a, [¹¹C]5b was the major radio product when using catalyst S2, while it was a minor product when catalyst S1 was used. Electron-rich substrates (43a, 44a, 46a, 47a, 50a, 57a, and 59a) were successfully labeled using S2, but failed to yield any significant product using S1 as the catalyst. For S2-catalyzed ¹¹C-labeling reactions, the regioselectivity was identical to those obtained in ¹²C-cyanation conditions. Among other reactions tested, no C—H ¹¹C-radiocyanation product was observed except for substrate 61a, a letrozole analog, which led to 18.1% RCC of C—H cyanation product ([¹¹C]61c) and 12.6% RCC of the demethoxy ¹¹C-cyanation product ([¹¹C]61b). Substrates 62a and 63a could also be labeled with ¹¹C albeit in reduced RCCs, while substrate 49a, 60a, 64a, and 65a were all carefully examined and found to afford almost no desired ¹¹C-radiolabeling products though they all demonstrated good reactivity when treated with TMSCN.²⁵

### VI. Cyano Arene or Heteroarene Products

The cyano arene and heteroarene products prepared from the photoredox-catalyzed cyanation method disclosed herein, comprise a monocyclic aromatic ring of Formula (IV):

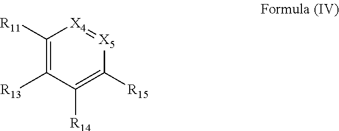

wherein X₄ and X₅ are each independently selected from the group consisting of —N and —CR₁₂;
R₁₁ and R₁₂ are each independently selected from the group consisting of —H, substituted or unsubstituted —(C₁-C₆) alkyl, substituted or unsubstituted —O(C₁-C₆) alkyl, substituted or unsubstituted —O(benzyl), substituted or unsubstituted —CH₂CO(C₁-C₆) alkyl, substituted or unsubstituted —(C₁-C₆) alkyl-NH(R₆), substituted or unsubstituted —N(C₁-C₆ alkyl)(R₆), —CH═CHCOOH, substituted or unsubstituted —CO(C₁-C₆) alkyl, substituted or unsubstituted —COO(C₁-C₆) alkyl, substituted or unsubstituted heteroaryl, and —COOH;
R₁₃ is selected from the group consisting of —H, substituted or unsubstituted —O(C₁-C₆) alkyl, substituted or unsubstituted —(C₁-C₆) alkyl, substituted or unsubstituted —CH₂CO(C₁-C₆) alkyl and substituted or unsubstituted —(C₁-C₆) alkyl-NH(R₁6);
R₁₄ is selected from the group consisting of —H, substituted or unsubstituted —O(C₁-C₆) alkyl, substituted or unsubstituted —(C₁-C₆) alkyl, —Cl and —F;
R₁₅ is CN or Im, wherein CN is selected from the group consisting of [¹²C]CN, [¹³C]CN, [¹⁴N]CN, and [¹⁵N]CN and Im is an imaging moiety selected from the group consisting of [¹¹C]CN and [¹³N]CN;
R₁₆ is —H or an amine protecting group (PG); and
a pharmaceutically acceptable salt form thereof.

In some embodiments, R₁₅ is CN selected from the group consisting of [¹²C]CN, [¹³C]CN and [¹⁴N]CN.

In some embodiments, R₁₅ is [¹¹C]CN.

In some embodiments, X₄ and X₅ both are —CR₁₂. In some embodiments, X₄ is —N. In some embodiments, X₅ is —N.

In some embodiments, R₁₁ is selected from the group consisting of —H, substituted or unsubstituted —(C₁-C₆) alkyl. In some embodiments, R₁₁ is selected from the group consisting —H, —OCH₂CH₃, —OCH₃, and —OCH(CH₃)₂. In some embodiments, each R₁₁ is selected from the group consisting of —H and substituted or unsubstituted —O(C₁-C₆) alkyl. In some embodiments, each R₁₁ is selected from the group consisting of —H, —OCH₂CH₃, —OCH₃, and —OCH(CH₃)₂.

In some embodiments, R₁₁ is selected from the group consisting of —H, substituted or unsubstituted —O(benzyl), and substituted or unsubstituted —CH₂CO(C₁-C₆) alkyl. In some embodiments, R₁₁ is selected from the group consisting of —H, —CH₂COCH₃, and —CH₂COCH₂CH₃.

In some embodiments, R₁₁ is selected from the group consisting of —H, substituted or unsubstituted —(C₁-C₆) alkyl-NH(R₁₆), and substituted or unsubstituted —N(C₁-C₆ alkyl)(R₁₆), wherein R₁₆ is —H or an amine protecting group (PG). In some embodiments, R₁₁ is selected from the group consisting of —H, —CH₂CH₂NH(BOC), —N(CH₃)(BOC), and —CH₂NH(BOC).

In some embodiments, R₁₁ is selected from the group consisting of —H, —CH═CHCOOH, substituted or unsubstituted —CO(C₁-C₆) alkyl, substituted or unsubstituted —COO(C₁-C₆) alkyl, substituted or unsubstituted heteroaryl, and —COOH. In some embodiments, R₁₁ is selected from the group consisting of —H, —CH═CHCOOH, —COCH₃, —COCH₂CH₃, substituted or unsubstituted —COOCH₃, -pyridinyl, and —COOH.

In some embodiments, R₁₁ is selected from the group consisting of —CH₃, —OCH₃, —H, —Cl, -Ph, —C(CH₃)₃, —CH₂COCH₃, —CH₂CH₂NH(BOC), —N(CH₃)(BOC), —Br, —CN, —CH═CHCOOH; -pyridin-2-yl, —CH₂C₁, —CH₂CN, —CH₂OH, —CH₂OCH₂CH₃, —CH₂NH(BOC), —CH₂N₃, —COCH₂CH₂COOCH₃, —COH, —COCH₃, —COOCH₃, and —COOH.

In some embodiments, each R₁₂ is independently selected from the group consisting of —H, substituted or unsubstituted —O(C₁-C₆) alkyl, and substituted or unsubstituted —(C₁-C₆) alkyl. In some embodiments, each R₁₂ is independently selected from the group consisting of —H, substituted or unsubstituted —O(C₁-C₆) alkyl. In some embodiments, each R₁₂ is independently selected from the group consisting of —H, —OCH₂CH₃, —OCH₃, and —OCH(CH₃)₂. In some embodiments, each R₁₂ is independently selected from the group consisting of —H and substituted or unsubstituted —(C₁-C₆) alkyl. In some embodiments, each R₁₂ is independently selected from the group consisting of —H and —CH₃. In some embodiments, each R₁₂ is independently selected from the group consisting of —H, —CH₃ and —OCH₃.

In some embodiments, R₁₃ is selected from the group consisting of —H, substituted or unsubstituted —O(C₁-C₆) alkyl. In some embodiments R₁₃ is selected from the group consisting of —H and —OCH₂CH₃, —OCH₃.

In some embodiments, R₁₃ is selected from the group consisting of —H and substituted or unsubstituted —CH₂CO(C₁-C₆) alkyl. In some embodiments, R₁₃ is selected from the group consisting of —H, —CH₂COCH₃—CH₂COCH₂CH₃, and —CH₂COCH(CH₃)₂.

In some embodiments, R₁₃ is selected from the group consisting of —H and —(C₁-C₆) alkyl-NH(R₆), wherein R₁₆ is —H or an amine protecting group (PG). In some embodiments, R₁₃ is selected from the group consisting of —H, —CH₂NH(R₁₆) and —CH₂CH₂NH(R₁₆), wherein R₁₆ is —H or a BOC group (—COOC(CH₃)₃). In some embodiments, R₁₃ is selected from the group consisting of —H and —CH₂CH₂NH(R₁₆), wherein R₁₆ is —H. In some embodiments, R₁₃ is selected from the group consisting of —H and —CH₂CH₂NH(R₁₆), wherein R₁₆ is a BOC group (—COOC(CH₃)₃).

In some embodiments, R₁₄ is selected from the group consisting of —H, substituted or unsubstituted —O(C₁-C₆) alkyl, —Cl and —F. In some embodiments, R₁₄ is selected from the group consisting of —H, —OCH₃, —OCH₂CH₃, —OCH(CH₃)₂, —Cl and —F.

In some embodiments, R₁₄ is selected from the group consisting of —H, substituted or unsubstituted —(C₁-C₆) alkyl, —Cl and —F. In some embodiments, R₁₄ is selected from the group consisting of —H, —CH₃, —CH₂CH₃, —CH(CH₃)₂, —Cl and —F.

In some embodiments, R₁₄ is selected from the group consisting of —CH₃, —OCH₃, —OCH₂CH₃, —OCH(CH₃)₂, and —F.

In some embodiments, R₁₅ is a substituted or unsubstituted (C₁-C₆) alkyl group. In some embodiments, R₁₅ is selected from the group consisting of —OCH₃, —OCH₂CH₃, and —OCH(CH₃)₂.

In some embodiments, R₁₅ is a substituted or unsubstituted aryl or heteroaryl group. In some embodiments, R₁₅ is phenyl or pyridinyl.

In some embodiments, R₁₄ is —OCH₃ or —CH₃, R₅ is —OCH₃ or —OCH₂CH₃, and X₅ is —CR₁₂, wherein R₁₂ is —H, —CH₃ or —OCH₃.

In some embodiments, R₁₄ is —OCH₃, R₁₅ is —OCH₃ or —OCH₂CH₃, and X₂ is —CR₁₂, wherein R₁₂ is —H or —OCH₃.

In another aspect of the current disclosure, the cyano arene and heteroarene products prepared from the photoredox-catalyzed cyanation method disclosed herein, comprise a bicyclic aromatic ring according to Formula (V):

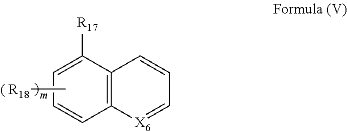

wherein X₆ is selected from the group consisting of —N and —CR₁₉;
R₁₇ is CN or Im, wherein CN is selected from the group consisting of [¹²C]CN, [¹³C]CN, [¹⁴N]CN, and [¹⁵N]CN and Im is an imaging moiety selected from the group consisting of [¹¹C]CN and [¹³N]CN;
R₁₈ and R₁₉ are each independently selected from the group consisting of —H, substituted or unsubstituted —(C₁-C₆) alkyl, substituted or unsubstituted —O(C₁-C₆) alkyl, substituted or unsubstituted —O(benzyl), substituted or unsubstituted —CH₂CO(C₁-C₆) alkyl, substituted or unsubstituted —(C₁-C₆) alkyl-NH(R₂₀), substituted or unsubstituted —N(C₁-C₆ alkyl)(R₂₀), —CH═CHCOOH, substituted or unsubstituted —CO(C₁-C₆) alkyl, substituted or unsubstituted —COO(C₁-C₆) alkyl, substituted or unsubstituted heteroaryl, and —COOH;
R₂₀ is —H or an amine protecting group (PG);
m is an integer selected from the group consisting of 0, 1, 2, 3, and 4; and
a pharmaceutically acceptable salt form thereof.

In some embodiments, X₆ is selected from the group consisting of —N and —CR₁₉, wherein R₁₉ is —H.

In some embodiments, R₁₇ is CN selected from the group consisting of [¹²C]CN, [¹³C]CN and [¹⁴N]CN.

In some embodiments, R₁₇ is [¹¹C]CN.

In some embodiments, m is 0.

In some embodiments, m is 1. In some embodiments, m is 1 and R₁₈ are each independently selected from the group consisting of —OCH₃, —OCH₂CH₃, and —OCH(CH₃)₂.

In some embodiments, m is 2. In some embodiments, m is 2 and R₁₈ is selected from the group consisting of —OCH₃, —OCH₂CH₃, and —OCH(CH₃)₂.

In some embodiments, the cyano arene or heteroarene products are biologically active molecules and the monocyclic aromatic ring of Formula (IV) or the bicyclic aromatic ring of Formula (V) have at least one of their respective R-substituents selected from the group consisting of R₁, R₁₂, R₁₃, R₁₄, R₁₆, R₁₈, R₁₉, and R₂₀ form a covalent bond with the remaining chemical structure of the biologically active molecule, as already previously described above for the arene and heteroarene substrates.

The cyano arene and heteroarene products described herein may in some cases exist as diastereomers, enantiomers, or other stereoisomeric forms. The arene and heteroarene substrates presented herein include all diastereomeric, enantiomeric, and epimeric forms as well as the appropriate mixtures thereof. Separation of stereoisomers may be performed by chromatography and/or recrystallization or by the forming diastereomers and separation thereof (Jean Jacques, Andre Collet, Samuel H. Wilen, “Enantiomers, Racemates and Resolutions”, John Wiley And Sons, Inc., 1981). Stereoisomers may also be obtained by stereoselective synthesis using synthetic methods known in the art. In some embodiments, the cyano arene and heteroarene products disclosed herein are enantiomers having an enantiomeric excess (% ee) of at least about 50%, about 60%, about 70%, about 80%, about 90%, about 95%, about 98%, or about 99.5%. In some embodiments, the cyano arene and heteroarene products disclosed herein are diastereomers having a diastereomeric excess (% de) of at least about 50%, about 60%, about 70%, about 80%, about 90%, about 95%, about 98%, or about 99.5%. In some embodiments, the arene and heteroarene substrates disclosed herein are present as enantiomeric or diastereomeric mixtures.

The methods and compositions described herein include the use of amorphous forms as well as crystalline forms (also known as polymorphs). The cyano arene and heteroarene products described herein may be in the form of pharmaceutically acceptable salts.

In some embodiments, the cyano arene and heteroarene products described herein may be formed as, and/or used as, pharmaceutically acceptable salts. The type of pharmaceutical acceptable salts, include, but are not limited to: (1) acid addition salts, formed by reacting the free base form of the arene and heteroarene substrates with a pharmaceutically acceptable: inorganic acid, such as, for example, hydrochloric acid, hydrobromic acid, sulfuric acid, phosphoric acid, metaphosphoric acid, and the like; or with an organic acid, such as, for example, acetic acid, propionic acid, hexanoic acid, cyclopentanepropionic acid, glycolic acid, pyruvic acid, lactic acid, malonic acid, succinic acid, malic acid, maleic acid, fumaric acid, trifluoroacetic acid, tartaric acid, citric acid, benzoic acid, 3-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, toluenesulfonic acid, 2-naphthalenesulfonic acid, 4-methylbicyclo-[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, butyric acid, phenylacetic acid, phenylbutyric acid, valproic acid, and the like; (2) salts formed when an acidic proton present in the parent compound is replaced by a metal ion, e.g., an alkali metal ion (e.g., lithium, sodium, potassium), an alkaline earth ion (e.g., magnesium, or calcium), or an aluminum ion. In some cases, arene and heteroarene substrates described herein may coordinate with an organic base, such as, but not limited to, ethanolamine, diethanolamine, triethanolamine, tromethamine, N-methylglucamine, dicyclohexylamine, tris(hydroxymethyl)methylamine. In other cases, cyano arene and heteroarene products described herein may form salts with amino acids such as, but not limited to, arginine, lysine, and the like. Acceptable inorganic bases used to form salts with cyano arene and heteroarene products that include an acidic proton, include, but are not limited to, aluminum hydroxide, calcium hydroxide, potassium hydroxide, sodium carbonate, sodium hydroxide, and the like.

Exemplary cyano arene or heteroarene products are shown in FIGS. 3, 6, 7 and 8.

### VII. Method of Using Cyano Arene or Heteroarene Products

The disclosed cyano arene or heteroarene products containing a radioisotope can be used in imaging modalities such as PET and MRI technologies. Typically, imaging modalities are employed to screen for and/or diagnose various disease states and/or follow treatment of various disease states in subjects.

Thus, one aspect of the current disclosure is to employ radioactive cyano arene or heteroarene products as disclosed herein in methods of imaging a subject for diagnosing a disease or assessing efficacy of treatment of a disease by a) administering to a subject in need thereof a radioactive cyano arene or heteroarene product as disclosed herein in an effective amount; and b) acquiring at least one image of at least a portion of the subject.

In some embodiments, the radioactive cyano arene or heteroarene product is formulated into a pharmaceutical composition comprising at least one pharmaceutically acceptable excipient and/or carriers. As will be apparent to those skilled in the art, that one or more pharmaceutically acceptable excipients or carriers will vary depending on the mode of administration of the cyano arene or heteroarene products to a subject in need thereof. In some embodiments, the pharmaceutical composition is in the form of a saline-based solution, a suspension, an emulsion, liposome-based preparation, microsphere-based preparation or any other pharmaceutical formulations in liquid form suitable for injection.

The effective amount of the radioactive cyano arene or heteroarene products can vary and depends on the mode of administration; the patient's age, weight, and health; as well as the area to be imaged. A skilled artisan would know how to best determine effective amounts of the disclosed radioactive cyano arene or heteroarene product.

In some embodiments, the imaging methods disclosed herein are employed for diagnosing a disease or assessing efficacy of treatment of a disease of the integumentary system, the skeletal system, the muscular system, the nervous system, the endocrine system, the cardiovascular system, the lymphatic system, the respiratory system, the digestive system, the urinary system, and the reproductive system.

In some embodiments, the imaging methods disclosed herein are employed to identify ulcers, infections, ischemia, irritable bowel syndrome, heart failure, cirrhosis, inflammation, and cancer, but should not be limited thereto.

### EXAMPLES

### General Information

Methods and Materials: Commercially available chemicals reagents were purchased from Sigma-Aldrich, Alfa Aesar, TCI, Acros, Combi-Blocks, Matrix Scientific, Oakwood Chemical, and Fisher Scientific etc. and used as received. Anhydrous acetonitrile (MeCN), dichloromethane (DCM), tetrahydrofuran (THF) and dimethylformamide (DMF) were dried by an inert solvent purification system (PS-MD-5). Nuclear magnetic resonance spectra were obtained using a Varian 400 MR spectrometer and a Varian 500 MHz spectrometer. ¹H NMR and ¹³C NMR spectra are referenced to Chloroform-d (¹H NMR: 7.26 ppm and ¹³C NMR: 77.16 ppm), Dimethyl sulfoxide-d6 (¹H NMR: 2.50 ppm and ¹³C NMR: 39.51 ppm), Acetone-d6(¹H NMR: 2.05 ppm and ¹³C NMR: 20.84 ppm), D₂0 (¹H NMR: 4.79 ppm). All spectra are reported as parts per million. 1H, 13C, and 19F NMR data are reported as follows: chemical shift (ppm), multiplicity (s=singlet, d=doublet, t=triplet, q=quartet, quint=quintet, dd=doublet of doublets, td=triplet of doublets, ddd=double of doublet of doublets, m=multiplet, app=apparent), coupling constants (Hz), and integration. High Resolution Mass Spectra (HRMS) were analyzed on either Thermo Fisher GC Exactive with an Electron Ionization (EI) source or Thermo Fisher Q Exactive HF-X (Thermo Fisher, Bremen, Germany) mass spectrometer with positive mode electrospray ionization (ESI). Reverse-phase flash liquid chromatography was performed using a Biotage® Isolera One instrument with a Biotage® SNAP Ultra C18 cartridge. High-performance liquid chromatography (HPLC) was accomplished on a SHIMADZU chromatography system (Model CBM-20A) with either a UV detector or a PDA detector and analyzed using LabSolutions software.

### Example 1: Preparation of the Photocatalysts and Methoxy Arene Substrates

Compounds 1a, 2a, 4a, 5a, 8a, 10a, 14a, 15a, 16a, 18a, 19a, 21a, 23a-29a, 31a-37a, 39a, and other synthetic starting materials were purchased and used directly. 43a-65a, 43b-65b were obtained and recently characterized by the inventors³².

Photocatalysts S1, S2, and S3 were synthesized following the procedures disclosed, and NMR data were well-matched with those in the references. Other photocatalysts were purchased and used directly.

### Major Photocatalysts

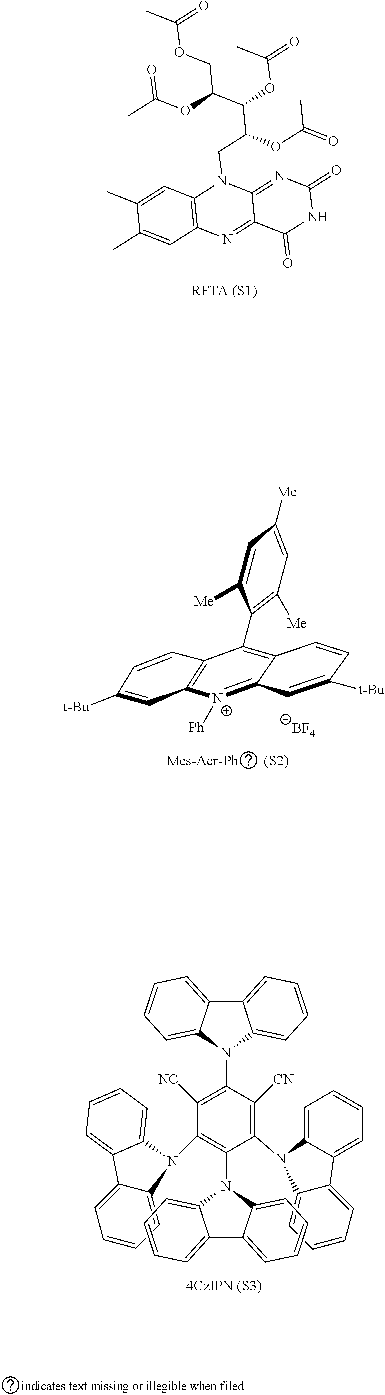

Riboflavin tetraacetate (RFTA, S1) was prepared according to a reported procedure¹⁶. ¹H NMR (400 MHz, Chloroform-d) δ 8.51 (s, 1H), 8.02 (d, J=1.1 Hz, 1H), 7.56 (s, 1H), 5.70-5.62 (m, 1H), 5.45 (s, 1H), 5.41 (ddd, J=6.5, 5.7, 2.9 Hz, 1H), 4.89 (s, 1H), 4.43 (dd, J=12.4, 2.9 Hz, 1H), 4.24 (dd, J=12.3, 5.7 Hz, 1H), 2.56 (s, 3H), 2.44 (d, J=0.9 Hz, 3H), 2.28 (s, 3H), 2.21 (s, 3H), 2.07 (s, 3H), 1.76 (s, 3H). ¹³C NMR (101 MHz, Chloroform-d) δ 170.76, 170.43, 170.02, 159.43, 154.50, 150.87, 148.29, 137.15, 136.20, 134.78, 133.14, 131.37, 115.67, 70.63, 69.60, 69.16, 62.03, 45.18, 21.61, 21.19, 20.94, 20.84, 20.47, 19.60.

3,6-Di-tert-butyl-9-mesityl-10-phenylacridin-10-ium, tetrafluoroborate (Mes-Acr-Ph-BF₄, S2) was prepared according to reported procedures.^{10 1}H NMR (400 MHz, Chloroform-d) δ 7.96 (t, J=7.5 Hz, 2H), 7.92-7.86 (m, 1H), 7.82-7.74 (m, 4H), 7.74-7.67 (m, 2H), 7.41 (s, 2H), 7.16 (s, 2H), 2.48 (s, 3H), 1.85 (s, 6H), 1.29 (s, 18H). ¹³C NMR (101 MHz, chloroform-d) δ 163.77, 162.49, 142.23, 140.33, 136.93, 136.23, 131.99, 131.76, 129.38, 129.08, 128.42, 128.10, 127.61, 124.16, 115.17, 36.80, 30.33, 21.41, 20.35. ¹⁹F NMR (376 MHz, Chloroform-d) 6-154.58 (d, J=19.6 Hz, 4F).

1,2,3,5-Tetrakis(carbazol-9-yl)-4,6-dicyanobenzene (4CzIPN, S3) was prepared according to reported procedures¹⁷. ¹H NMR (400 MHz, Chloroform-d) δ 8.23 (dt, J=7.8, 1.0 Hz, 1H), 7.76-7.65 (m, 4H), 7.49 (ddd, J=8.0, 6.6, 1.6 Hz, 1H), 7.33 (dt, J=7.6, 1.0 Hz, 1H), 7.28-7.17 (m, 2H), 7.14-7.03 (m, 3H), 6.87-6.78 (m, 2H), 6.63 (ddd, J=8.4, 7.3, 1.2 Hz, 1H). ¹³C NMR (101 MHz, Chloroform-d) δ 145.37, 144.77, 140.14, 138.34, 137.12, 134.91, 127.11, 125.92, 125.12, 124.89, 124.69, 124.00, 122.55, 122.08, 121.55, 121.13, 120.57, 119.79, 116.53, 111.76, 110.10, 109.62, 109.57.

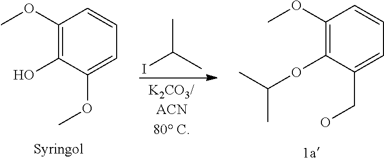

### Example 2: Synthesis of 2-isopropoxy-1,3-dimethoxybenzene (1a′)

A mixture of syringol (4.0 mmol), 2-iodopropane (6.0 mmol) and K₂CO₃ (8.0 mmol) in acetonitrile (30 mL) was refluxed in a 100 mL round-bottom flask equipped with a condenser for 24 hours and the reaction progress was monitored by TLC. After cooling down the reaction mixture, the salts were filtered and rinsed with acetone. The filtrate was then concentrated and H₂O (30 mL) and CH₂Cl₂ (20 mL) was added into the residue before extracting the product with CH₂Cl₂ (2×20 mL). The product solution was dried over Na₂SO₄ before rotary evaporation. A short silica column was used to remove possible iodide residue to offer a colorless liquid product 1a′ in a yield of 91.0%. 1H NMR (400 MHz, Chloroform-d) δ 6.97 (t, J=8.4 Hz, 1H), 6.57 (d, J=8.4 Hz, 2H), 4.39-4.31 (m, 1H), 3.83 (s, 6H), 1.30 (d, J=6.2 Hz, 6H). ¹³C NMR (101 MHz, Chloroform-d) δ 154.25, 136.45, 123.34, 105.44, 75.35, 56.18, 22.62. HRMS (ESI) m/z, calculated for [M+H]⁺: 197.1178; found: 197.11172; [M+Na]+: 219.0997; found: 219.0991.

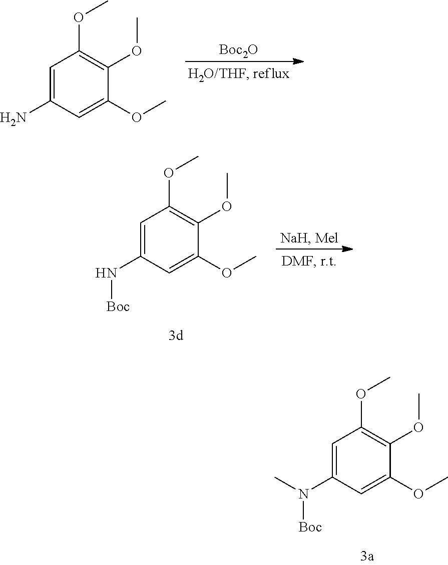

### Example 3: Synthesis of Methoxy Arene Substrates 3a and 3d

Tert-Butyl (3,4,5-trimethoxyphenyl)carbamate (3d). In a 100 mL round-bottom flask equipped with a condenser, a water (5 mL) and THF (10 mL) solution was added to 3,4,5-trimethoxyaniline (6.0 mmol), di-tert-butyl dicarbonate (9 mmol), and DMAP (0.6 mmol) before stirring the reaction mixture at 80° C. for 2 hours. Then it was cooled to room temperature before concentrating the reaction mixture in vacuo. The crude product was purified by flash column chromatography on silica gel (hexane/EtOAc=5:1-2:1) to afford an off-white solid product 3d in 77.0% yield. ¹H NMR (500 MHz, Chloroform-d) δ 6.65 (s, 2H), 6.45 (s, 1H), 3.83 (s, 6H), 3.79 (s, 3H), 1.51 (s, 9H). ¹³C NMR (126 MHz, Chloroform-d) δ 153.53, 152.86, 134.68, 133.85, 96.24, 80.65, 61.11, 56.20, 28.49.

Tert-Butyl methyl(3,4,5-trimethoxyphenyl)carbamate (3a). The title compound was prepared from reported literature.¹⁸ The product was purified by flash column chromatography on silica gel (hexane/EtOAc=6:1-3:1) to afford a white solid product in a yield of 92.0%. 1H NMR (500 MHz, Chloroform-d) δ 6.45 (s, 2H), 3.84 (s, 6H), 3.82 (s, 3H), 1.46 (s, 9H). ¹³C NMR (126 MHz, Chloroform-d) δ 154.87, 152.94, 139.75, 135.75, 103.35, 80.40, 60.98, 56.14, 37.82, 28.48. HRMS (ESI) m/z, calculated for [M+H]⁺: 298.1654; found: 298.1648.

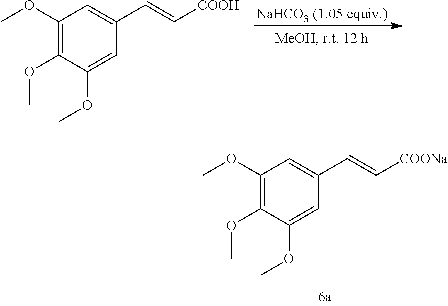

### Example 4: Synthesis of Sodium (E)-3-(3,4,5-trimethoxyphenyl)acrylate (6a)

(E)-3,4,5-Trimethoxycinnamic acid (2.0 mmol) was added into a 20 mL screw-capped scintillation vial along with NaHCO₃ (1.05 equiv.), 5 mL MeOH and a stir bar. It was stirred for 12 hours before retrieving the stir bar and evaporating the methanol to afford sodium form product 6a as a white solid powder with 100.2% yield. It could be directly used without further purification. 1H NMR (400 MHz, DMSO-d6) δ 7.03 (d, J=15.8 Hz, 1H), 6.78 (s, 2H), 6.35 (d, J=15.8 Hz, 1H), 3.79 (s, 6H), 3.65 (s, 3H), 3.34 (s, 4H), 2.50 (dd, J=3.6, 1.8 Hz, 4H). ¹³C NMR (101 MHz, DMSO-d6) δ 170.54, 152.90, 137.36, 135.25, 132.54, 129.97, 104.14, 59.99, 55.79.

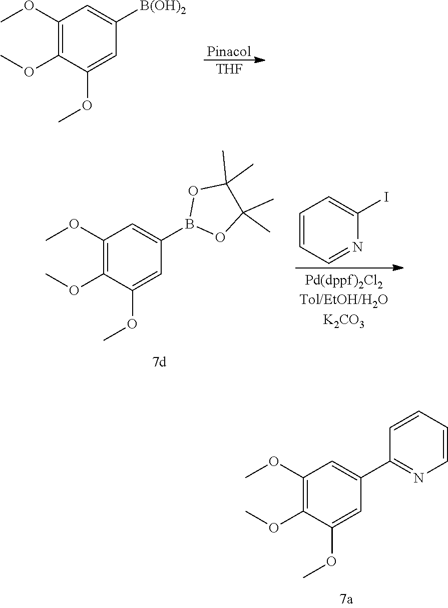

### Example 5: Synthesis of Methoxy Arene Substrates 7a and 7d

4,4,5,5-Tetramethyl-2-(3,4,5-trimethoxyphenyl)-1,3,2-dioxaborolane (7d). The title compound was prepared according to the literature report.¹⁹ The product was purified by flash column chromatography on silica gel (hexane/EtOAc=9:1-3:1) to afford a white solid product in a yield of 98.0% (1.0 mmol, 288 mg). ¹H NMR (400 MHz, Chloroform-d)¹H NMR (400 MHz, Chloroform-d) δ 7.03 (s, 2H), 3.90 (s, 6H), 3.87 (s, 3H), 1.34 (s, 12H)¹³C NMR (101 MHz, Chloroform-d) δ 153.05, 141.00, 111.44, 84.03, 77.48, 77.16, 76.84, 60.94, 56.31, 24.99.

2-(3,4,5-Trimethoxyphenyl)pyridine (7a). Pd(dppf)₂Cl₂ (10 mol %) was added to a suspension of 7d (0.5 mmol, 1 equiv.), 2-iodo-pyridine (1.5 equiv.), K₂CO₃ (1.5 equiv.), and mixed solvents of toluene (2.0 mL), ethanol (1.5 mL), and water (0.8 mL) in a 16 mL crimp-cap vial at r.t. followed by degassing for 10 minutes with nitrogen. The mixture was stirred at reflux for 6 hours then cooled to r.t. H₂O (10 mL) was added, and the mixture was extracted with EtOAc (10 mL×2). The organic layers were combined, washed with brine, dried (Na₂SO₄), filtered, and concentrated in vacuo. The residue was purified by flash column chromatography (silica gel, hexane/EtOAc from 9/1 to 3/1) to afford the desired product 7a as a colorless sticky gel (120 mg) in 49.0% yield. ¹H NMR (400 MHz, Chloroform-d) δ 8.67 (ddd, J=4.8, 1.8, 1.0 Hz, 1H), 7.74 (ddd, J=8.0, 7.3, 1.8 Hz, 1H), 7.68 (dt, J=8.0, 1.2 Hz, 1H), 7.24 (s, 2H), 7.22 (ddd, J=7.3, 4.8, 1.3 Hz, 1H), 3.97 (s, 6H), 3.90 (s, 3H). ¹³C NMR (101 MHz, Chloroform-d) δ 157.25, 153.66, 149.68, 139.22, 136.88, 135.20, 122.15, 120.50, 104.31, 61.10, 56.41.

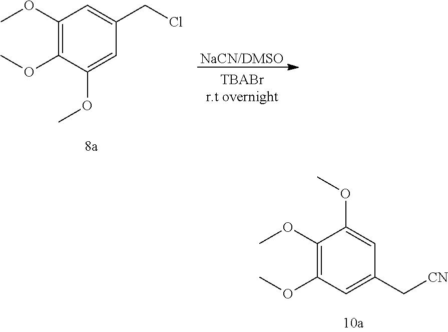

### Example 6: Synthesis of 2-(3,4,5-Trimethoxyphenyl)acetonitrile (10a)

To a 100 mL round-bottom flask, 3,4,5-trimethoxybenzyl chloride (8a, 5.0 mmol), sodium cyanide (7.5 mmol), and TBABr (1.0 mmol) were added before DMSO (20 ml) and a stir bar was added at room temperature. Then it was stirred at room temperature for 24 hours while monitoring the reaction mixture by TLC. The reaction mixture was poured into water (30 ml) in a separation funnel. The reaction mixture was extracted with EtOAc (3×20 ml). The combined organic phase was washed with water (2×50 ml) and brine (1×50 ml). Then it was dried over anhydrous Na₂SO₄ before evaporating the solvent under reduced pressure. Lastly, the crude product was passed through silica gel column using hexane/EtOAc (6/1 to 4/1) as eluent. The final product was obtained as an off-white solid with a yield of 89.0%. ¹H NMR (400 MHz, Chloroform-d) δ 6.51 (s, 2H), 3.86 (s, 6H), 3.82 (s, 3H), 3.69 (s, 2H). ¹³C NMR (101 MHz, Chloroform-d) δ 153.78, 137.82, 125.46, 117.95, 105.14, 60.98, 56.31, 23.91.

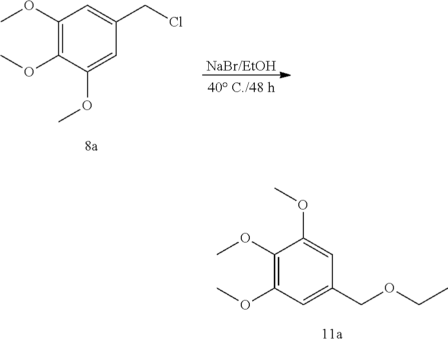 [SMILES: COc1cc(CCl)cc(OC)c1OC ; "NaBr/EtOH" ; CCOCc1cc(OC)c(OC)c(OC)c1]

### Example 7: Synthesis of 5-(Ethoxymethyl)-1,2,3-trimethoxybenzene (11a)

To a 100 mL round-bottom flask, 3,4,5-trimethoxybenzyl chloride (8a, 4.0 mmol), sodium bromide (1.0 mmol), and 30 mL ethanol was added. It was firstly stirred at r.t. for 4 hours while monitored by TLC before very slow conversion began happening. Then it was heated over a heat plate at 40° C. for another 2 days while TLC monitored full consumption of the chloride. The reaction mixture was then redissolved in DCM (20 mL) and water (20 mL) after evaporating the solvent under reduced pressure. The mixture was then transferred into a separation funnel followed by extraction with DCM (20 mL×2). The combined organic phase was washed with water (20 ml) and brine (20 ml). Then it was dried over anhydrous Na₂SO₄ before evaporating the solvent under reduced pressure. Lastly, the crude product was filtered and rinsed with DCM (10 mL). The product was obtained as a colorless liquid by removing all the solvent in vacuo with a yield of 97.0%. ¹H NMR (400 MHz, Chloroform-d) δ 6.57 (s, 2H), 4.43 (s, 2H), 3.86 (s, 6H), 3.82 (s, 3H), 3.55 (q, J=7.0 Hz, 2H), 1.25 (t, J=7.0 Hz, 1H). ¹³C NMR (101 MHz, Chloroform-d) δ 153.38, 137.46, 134.39, 104.75, 77.48, 77.16, 76.84, 73.05, 65.94, 60.94, 56.20, 15.37. HRMS (ESI) m/z, calculated for [M+Na]+: 249.1103; found: 249.1096.

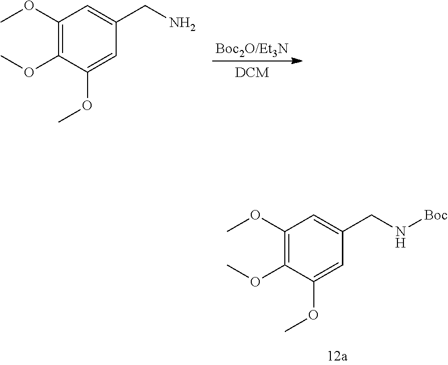 [SMILES: COc1cc(CN)cc(OC)c1OC ; "Boc2O/Et3N" ; COc1cc(CNC(=O)OC(C)(C)C)cc(OC)c1OC]

### Example 8: Synthesis of tert-Butyl (3,4,5-trimethoxybenzyl)carbamate (12a)

The title compound was prepared from reported literature²⁰. The product was purified by flash column chromatography on silica gel (hexane/EtOAc=9:1-3:1) to afford a white solid product in a yield of 90.0% (5.0 mmol). ¹H NMR (400 MHz, Chloroform-d) δ 6.50 (s, 2H), 4.85 (s, 1H), 4.23 (d, J=5.8 Hz, 2H), 3.84 (s, 6H), 3.82 (s 3H), 1.46 (s, 9H). ¹³C NMR (101 MHz, Chloroform-d) δ 156.05, 153.49, 137.30, 134.86, 104.54, 79.72, 60.98, 56.23, 45.11, 28.54.

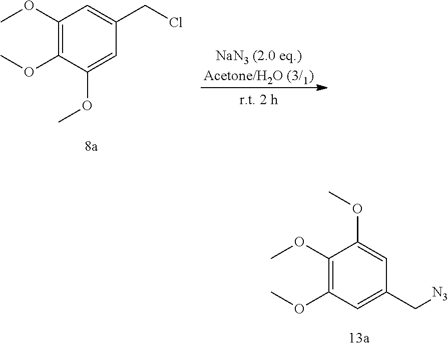 [SMILES: COc1cc(CCl)cc(OC)c1OC ; "NaN3 (2.0 eq.)Acetone/H2O (3/1)" ; COc1cc(CN=[N+]=[N-])cc(OC)c1OC]

### Example 9: Synthesis of 5-(Azidomethyl)-1,2,3-trimethoxybenzene (13a)

3,4,5-trimethoxybenzyl chloride (8a, 5.0 mmol) was added into 100 mL round-bottomed flask before adding 75% aqueous acetone (20 mL) and sodium azide (2.0 equiv.), the reaction mixture was stirred at 22° C. for 2 hours before a full consumption of the chloride was observed by TLC. Then, most of the acetone was gently removed by evaporation at 20° C. followed by extracting with dichloromethane 3 times (20 mL). Then, the combined extract was dried over anhydrous Na₂SO₄, and the solvent was evaporated under reduced pressure to obtain crude product mixture, which was then purified by a short silica gel column chromatography using hexanes/EtOAc (9/1 to 4/1) as the eluent, resulting in the pure product as yellow oil that could be stored at 2-8° C. ¹H NMR (400 MHz, Chloroform-d) δ 6.53 (s, 2H), 4.28 (s, 2H), 3.87 (s, 6H), 3.85 (s, 3H). ¹³C NMR (101 MHz, Chloroform-d) δ 153.62, 138.07, 131.12, 105.29, 60.98, 56.29, 55.27. HRMS (ESI) m/z, calculated for [M+H]⁺: 224.1035; found: 224.1029.

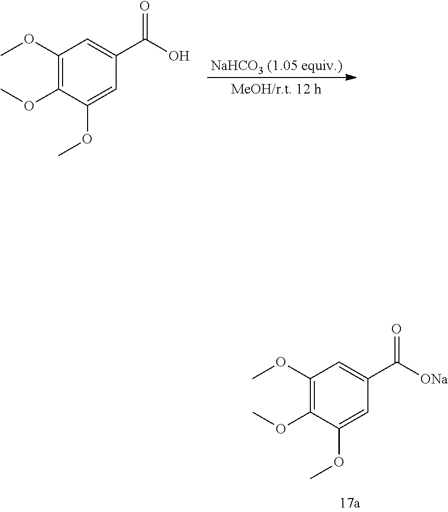

### Example 10: Synthesis of Sodium 3,4,5-trimethoxybenzoate (17a)

The title compound was synthesized following the same synthetic procedure as 6a. The white solid powder product was obtained in a yield of 99.0%. ¹H NMR (500 MHz, DMSO-d6) δ 7.21 (s, 2H), 3.76 (s, 6H), 3.65 (s, 3H).¹³C NMR (101 MHz, DMSO-d6) δ 169.26, 151.69, 138.15, 136.15, 106.24, 59.91, 55.58.

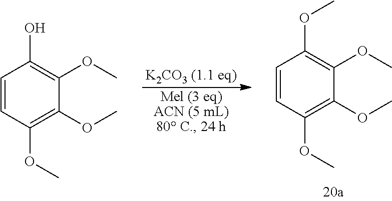

### Example 11: Synthesis of 1,2,3,4-Tetramethoxybenzene (20a)

The title compound was synthesized following a similar synthetic procedure as 1a′. The obtained crude product mixture was then purified by a short silica gel column chromatography using hexanes/EtOAc (8/1 to 3/1) as the eluent. A white solid powder 20a was obtained in a yield of 86.0%. ¹H NMR (500 MHz, Chloroform-d) δ 6.57 (s, 2H), 3.89 (s, 6H), 3.81 (s, 6H). ¹³C NMR (126 MHz, Chloroform-d) δ 147.87, 143.48, 106.49, 61.30, 56.48. HRMS (ESI) m/z, calculated for [M+H]⁺: 199.0970; found: 199.0964.

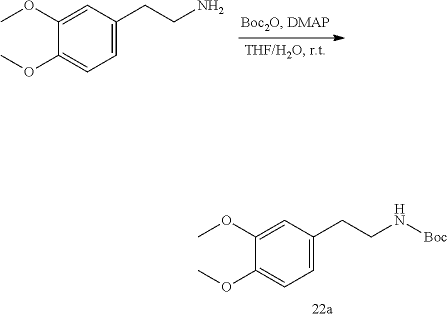 [SMILES: COc1ccc(CCN)cc1OC ; COc1ccc(CCNC(=O)OC(C)(C)C)cc1OC]

### Example 12: Synthesis of tert-Butyl (3,4-dimethoxyphenethyl)carbamate (22a)

The title compound was synthesized following a similar synthetic procedure with 3a′ while reacted at r.t. for overnight (18 hours) and purified by silica gel column chromatography using hexanes/EtOAc (8/1 to 3/1) as the eluent. An off-white solid product 22a was obtained in a yield of 83.0% (5.0 mmol). ¹H NMR (500 MHz, Chloroform-d) δ 6.80 (d, J=8.0 Hz, 1H), 6.74-6.68 (m, 2H), 4.57 (s, 1H), 3.86 (s, 3H), 3.85 (s, 3H), 3.34 (dd, J=13.2, 6.7 Hz, 2H), 2.73 (t, J=7.0 Hz, 2H), 1.42 (s, 9H). ¹³C NMR (126 MHz, Chloroform-d) δ 155.98, 148.90, 147.56, 131.52, 120.71, 111.86, 111.21, 79.32, 55.97, 55.88, 41.99, 35.82, 28.51. HRMS (ESI) m/z, calculated for [M+H]⁺: 282.1705; found: 282.1698.

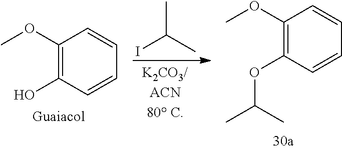

### Example 13: Synthesis of 1-Isopropoxy-2-methoxybenzene (30a)

The title compound was synthesized following a similar synthetic procedure as 1a′ starting with 10.0 mmol guaiacol. The product was obtained as a colorless liquid in a yield of 74.0%. ¹H NMR (400 MHz, Chloroform-d) δ 6.95-6.84 (m, 3H), 4.52 (hept, J=6.1 Hz, 1H), 3.85 (s, 3H), 1.37 (d, J=6.1 Hz, 6H). ¹³C NMR (101 MHz, Chloroform-d) δ 150.64, 147.48, 121.43, 120.91, 116.26, 112.28, 71.55, 56.05, 22.26.

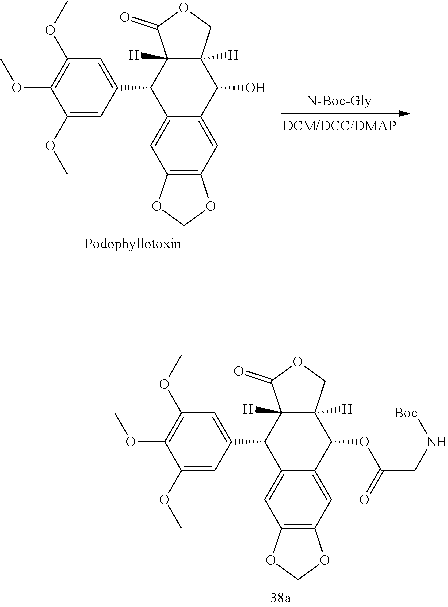 [SMILES: COc1cc([C@H]2c3cc4c(cc3[C@@H](O)[C@@H]3COC(=O)[C@@H]23)OCO4)cc(OC)c1OC ; COc1cc([C@H]2c3cc4c(cc3[C@@H](OC(=O)CNC(=O)OC(C)(C)C)[C@@H]3COC(=O)[C@@H]23)OCO4)cc(OC)c1OC]

### Example 14: Synthesis of (5S,5aS,8aS,9S)-8-oxo-9-(3,4,5-trimethoxyphenyl)-5,5a,6,8,8a,9-hexahydrofuro [3′,4′: 6,7]naphtho[2,3-d][1,3]dioxol-5-yl (tert-butoxycarbonyl)glycinate (38a)

The title compound was synthesized following a modified synthetic procedure from the literature starting with 0.5 mmol podophyllotoxin.¹⁴ N-Boc-Gly (1.2 eq.), DCC (2.0 eq.), and DMAP (10.0 mol %) in anhydrous DCM (15 mL) were used for the reaction. After monitoring a full consumption of the starting material, the solvent was removed and redissolved in ethyl acetate (10 mL) before filtration. The filtrate was then redissolved in acetonitrile (5 mL) before filtration. The filtrate was then evaporated and dried over vacuum. Then the crude product was purified by silica gel column chromatography using DCM/acetone (50/1 to 20/1) as the eluent. The desired white powder product 38a (255 mg, 0.5 mmol) was obtained in a yield of 89.0% after rotary evaporation. ¹H NMR (500 MHz, Chloroform-d) δ 6.78 (s, 1H), 6.54 (s, 1H), 6.38 (s, 2H), 6.01-5.93 (m, 3H), 5.02 (s, 1H), 4.61 (d, J=4.4 Hz, 1H), 4.40 (t, J=8.1 Hz, 1H), 4.19 (t, J=9.8 Hz, 1H), 4.03 (dd, J=18.2, 5.9 Hz, 1H), 3.95 (dd, J=18.2, 5.9 Hz, 1H), 3.81 (d, J=0.7 Hz, 3H), 3.76 (d, J=0.7 Hz, 6H), 2.97-2.80 (m, 2H), 1.45 (s, 9H). ¹³C NMR (101 MHz, Chloroform-d) δ 173.67, 171.14, 152.81, 148.44, 147.83, 137.35, 134.83, 132.57, 127.84, 109.89, 108.21, 107.21, 101.79, 80.60, 74.77, 71.36, 60.90, 56.30, 45.71, 43.85, 42.86, 38.75, 28.46, 28.42. HRMS (ESI) m/z, calculated for [M+H]⁺: 572.2132; found: 572.2134; [M+Na]⁺: 594.1951; found: 594.1956.

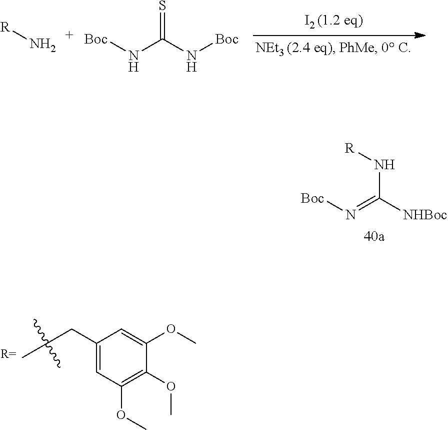

### Example 15: Synthesis of Compound 40a

The title compound was prepared as a white solid in 66.0% isolated yield via reported procedures from literature starting with 2.0 mmol benzyl amine.^{21 1}H NMR (400 MHz, Chloroform-d) δ 11.51 (s, 1H), 8.56 (s, 1H), 6.57 (s, 2H), 4.53 (d, J=5.2 Hz, 2H), 3.85 (s, 6H), 3.83 (s, 3H), 1.50 (d, J=14.0 Hz, 18H). ¹³C NMR (101 MHz, Chloroform-d) δ 163.71, 156.16, 153.52, 153.37, 137.60, 133.11, 105.43, 83.39, 79.53, 60.98, 56.27, 45.48, 28.46, 28.25, 28.20. HRMS (ESI) m/z, calculated for [M+H]⁺: 440.2397; found: 440.2389.

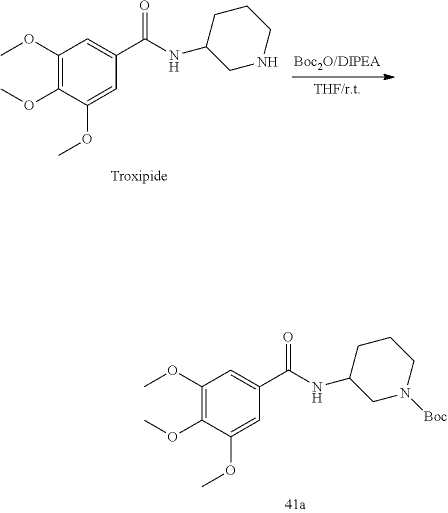 [SMILES: COc1cc(C(=O)NC2CCCNC2)cc(OC)c1OC ; COc1cc(C(=O)NC2CCCN(C(=O)OC(C)(C)C)C2)cc(OC)c1OC]

### Example 16: Synthesis of Compound 41a

The title compound was prepared as a white solid in 91.0% isolated yield via modifying reported procedures from literature²² starting with 2.0 mmol troxipide, to which 0.2 equiv. DIPEA was added. ¹H NMR (500 MHz, Chloroform-d, 55° C.) δ 7.00 (s, 2H), 6.31 (s, 1H), 4.12 (dq, J=10.1, 5.1 Hz, 1H), 3.89 (s, 6H), 3.87 (s, 3H), 3.64-3.48 (m, 3H), 3.32-3.24 (m, 1H), 1.93-1.82 (m, 2H), 1.68 (tdd, J=13.6, 9.0, 4.3 Hz, 1H), 1.58 (ddd, J=14.7, 9.9, 6.0 Hz, 1H), 1.46 (s, 9H). ¹³C NMR (126 MHz, Chloroform-d, 55° C.) δ 166.74, 155.80, 153.54, 141.84, 130.23, 105.30, 105.21, 80.18, 61.06, 60.92, 56.79, 56.64, 48.86, 46.49, 46.38, 44.89, 29.65, 28.69, 28.63, 28.56, 28.51, 22.50. HRMS (ESI) m/z, calculated for [M+H]⁺: 395.2182; found: 395.2174.

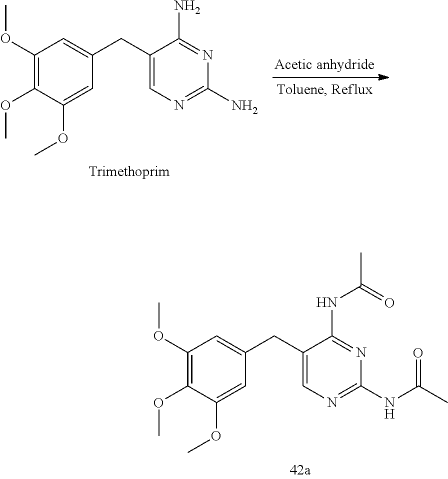 [SMILES: COc1cc(Cc2cnc(N)nc2N)cc(OC)c1OC ; COc1cc(Cc2cnc(NC(C)=O)nc2NC(C)=O)cc(OC)c1OC]

### Example 17: Synthesis of Compound 42a

The title compound was prepared as white powder in 61.0% isolated yield via reported procedures from literature starting with 5.0 mmol trimethoprim.^{23 1}Ha NMR (500 MHz, Chloroform-d) δ 10.02 (s, 1H), 9.58 (s, 1H), 8.26 (s, 1H), 6.37 (s, 2H), 3.83 (s, 2H), 3.83 (s, 3H), 3.80 (s, 6H), 2.56 (s, 3H), 2.24 (s, 3H). ¹³C NMR (126 MHz, Chloroform-d) δ 169.84, 160.04, 157.73, 155.75, 153.73, 137.13, 133.34, 106.18, 61.01, 56.31, 35.51, 25.50, 24.28. HRMS (ESI) m/z, calculated for [M+H]⁺: 375.1668; found: 375.1661.

### Example 18: Photocatalytic Arene Demethoxy Cyanation Methods

### I. Reagents and Equipment Information

All chemicals are ACS reagent grade and other solvents are either HPLC grade or analytical grade which are used without further purification and drying. Anhydrous ethanol was the Koptec's Pure Ethanol 200 Proof purchased from Fisher Scientific. Acetonitrile (HPLC grade) was used directly without further drying. The blue LED lamps (Wolezek 36W LED, 450-460 nm) were purchased from Amazon.com. The major irradiation peak centered around 450-460 nm.

### II. General HPLC Conditions for Crude Reaction Analysis

Column: Phenomenex, Kinetex® 5 m EVO C18 100 Å, 250×4.6 mm LC Column.

Condition A: Solvent A: 0.1% TFA water; Solvent B: 0.1% TFA acetonitrile. Isocratic elution at x % solvent B. Flow rate: 1 mL/minute. Injection volume: 1 mL (with 5% acetic acid solution).

Condition B: Solvent A: 10 mM COONH₄ water; Solvent B: acetonitrile. Isocratic elution at x % solvent B. Flow rate: 1 mL/minute. Injection volume: 1 mL (with water).

Condition C: Column: Phenomenex, Kinetex® 5 m F5 100 Å, 250×4.6 mm LC Column. Solvent A: 0.1% TFA water; Solvent B: 0.1% TFA acetonitrile; Isocratic elution at x % solvent B. Flow rate: 1 mL/minute. Injection volume: 1 mL (with 5% acetic acid solution).

### III. Reaction Optimization

Optimization procedure 1. To a dry 4.32-dram (16 mL) screw capped vial that contained a mini-Teflon-coated magnetic stir bar was added 0.00a mmol of the photocatalyst (e.g., S1, a mol %), base additive (e.g., NaHCO₃, 0.b mmol, b.0 equiv.) and arene (0.1 mmol, 1.0 equiv.). (Solid cyanide source, 0.c mmol, c equiv., was also added before solvent.) The reagent mixture was then attempted to be dissolved in the solvent (e.g., EtOH, 1.0 ml, 0.1 M). The vial was sealed with a PTFE-lined septum screw cap and stirred rapidly for approximately 5 minutes. (Then degas would be performed if needed.) Liquid cyanide source (e.g., TMSCN, 0.c mmol, c equiv.) was then subsequently added by a micro-syringe. The vial was positioned on a stir plate approximately 5 cm from two blue LED lamps (Wolezek 36W LED, 450-460 nm) before stirred and irradiated for a designated time (18-24 hours). Then 0.3-0.4 μL was taken from the crude reaction mixture followed by diluting with 40 μL EtOH and 700 μL 5% acetic acid for HPLC analysis.

Optimization procedure 2. To a dry 1.0-dram (3.7 ml) screw capped vial that contained a mini-Teflon-coated magnetic stir bar was added 0.003 mmol of S1, (3.0 mol %, taken from a stock solution of S1 in the corresponding reaction solvent), base additive (NaHCO₃, 0.b mmol, b.0 equiv.) and arene (0.1 mmol, 1.0 equiv.). (Solid cyanide source, 0.c mmol, c equiv., was also added before solvent.) The reagent mixture was then attempted to be dissolved in the solvent (e.g., EtOH, 1.0 ml, 0.1 M). The vial was sealed with a plastic screw cap and stirred rapidly for approximately 5 minutes. Liquid cyanide source (e.g., TMSCN, 0.c mmol, c equiv.) was then subsequently added. The vial was then capped and positioned on a stir plate approximately 5 cm from two blue LED lamps (Wolezek 36W LED, 450-460 nm) before stirred and irradiated for 23 hour. Then 0.3-0.4 μL was taken from the crude reaction mixture followed by diluting with 40 μL EtOH and 700 μL 5% acetic acid for HPLC analysis.

### Substrates and Products UV Calibration on HPLC

Standard curves (A[peak area integration]=k*n[amount of the substance]) of the substrate 1a and corresponding arene nitrile 1b, 2a and 2b were obtained on the same HPLC with the general HPLC condition A, isocratic 35%. See FIG. 10.

### Example 19: Photocatalyst Screening

The reactions were performed by following the optimization procedure 1.

| 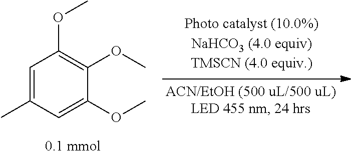 | | |
|---|---|---|
| 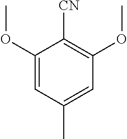 | | |
| Entry | Cat. (10 mol %) | HPLC Yield (%) |
| 1 | Mes-Acr-BF₄ (S2) | 48.8 |
| 2 | Rose Bengal | 0 |
| 3 | Eosin B | 0 |
| 4 | 4CzIPN (S3) | 99.9 |
| 5 | 4CzTPN | 22.6 |
| 6 | Eosin Y | 0 |
| 7 | Methylene Blue | 0 |
| 8 | Rhodamine B | 0 |
| 9 | RFTA (S1) | 99.9 |

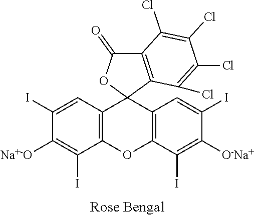

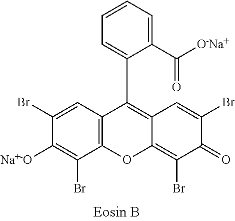

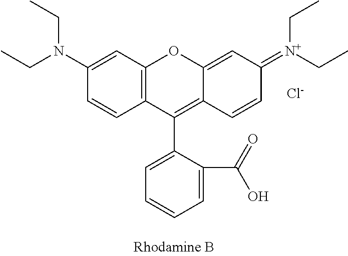 [SMILES: CCN(CC)c1ccc2c(-c3ccccc3C(=O)O)c3ccc(=[N+](CC)CC)cc-3oc2c1 ; [Cl-]]

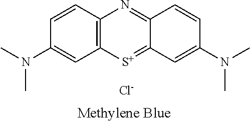

### Example 20: Screening of Additional Oxidants and/or De-Oxidants

The reactions were performed by following the optimization procedure 1.

| 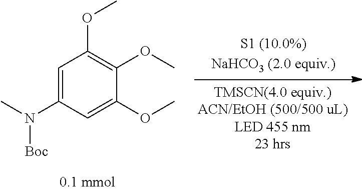 | | |
|---|---|---|
| 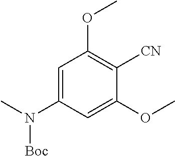 | | |
| 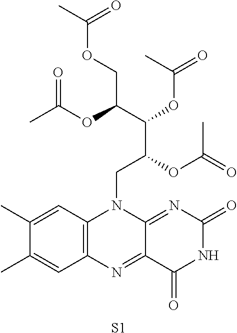 [SMILES: CC(=O)OC[C@H](OC(C)=O)[C@H](OC(C)=O)[C@@H](Cn1c2nc(=O)[nH]c(=O)c-2nc2cc(C)c(C)cc21)OC(C)=O ; "S1"] | | |
| 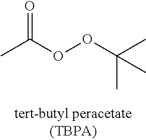 | | |
| Entry | Condition variations | HPLC Estimated Yield (%) |
| 1 | None | 97.0 |
| 2 | Degassed for 10 min | 18.0 |
| | N₂ protected | |
| 3 | Degassed for 10 min | 25.0 |
| | N₂ protected | |
| | TBPA (1.0 equiv.) | |
| 4 | Degassed for 10 min | 34.0 |
| | N₂ protected | |
| | TBPA (5.0 equiv.) | |
| 5 | TBPA (1.0 equiv.) | 90.0 |

### Example 21: Screening of Cyanide Sources

The reactions were performed by following the optimization procedure 1.

| 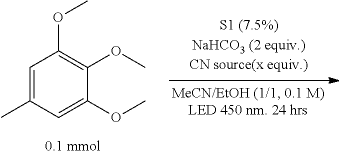 | | |
|---|---|---|
| 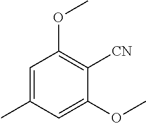 | | |
| 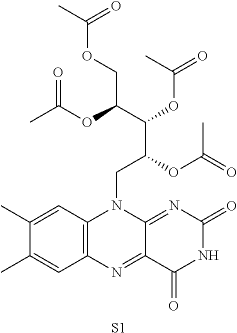 [SMILES: CC(=O)OC[C@H](OC(C)=O)[C@H](OC(C)=O)[C@@H](Cn1c2nc(=O)[nH]c(=O)c-2nc2cc(C)c(C)cc21)OC(C)=O ; "S1"] | | |
| Entry | CN source (x eq.) | HPLC Yield (%) |
| 1 | ACH (acetone cyanohydrin, 2 eq.) | 98.2 |
| 2 | ACH (4 eq.) | 85.8 |
| 3 | TBACN (4 eq.) | 21.0 |
| 4 | TMSCN (4 eq.) | >99.0 |
| 5 | TMSCN (3 eq.) | >99.0 |
| 6 | TMSCN (2 eq.) | >99.0 |
| 7 | NaCN (4 eq.) | 42.6 |
| 8 | p-Tol-SO₂-CN (4 eq.) | <1.0 |

### Example 22: Screening of Solvents

The reactions were performed by following the optimization procedure 2.

| 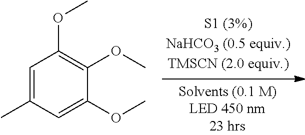 | | |
|---|---|---|
| 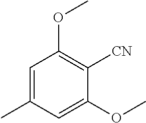 | | |
| 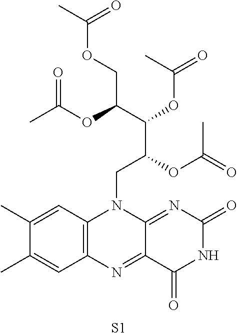 [SMILES: CC(=O)OC[C@H](OC(C)=O)[C@H](OC(C)=O)[C@@H](Cn1c2nc(=O)[nH]c(=O)c-2nc2cc(C)c(C)cc21)OC(C)=O ; "S1"] | | |
| Entry | Solvents (1 mL, 0.1 M) | HPLC Yield (%) |
| 1 | MeCN/EtOH (1:1) | 79.0 |
| 2 | EtOH | >99.9 |
| 3 | MeCN | 44.5 |
| 4 | MeOH | 49.8 |
| 5 | DCE | 27.3 |
| 6 | Acetone | 8.7 |
| 7 | THF | 2.3 |
| 8 | DMF | 45.9 |
| 9 | DMSO | 18.7 |
| 10 | tBuOH | 36.7 |
| 11 | H2O | 10.3 |
| 12 | NMP | 7.2 |
| 13 | DMA | 22.6 |
| 14 | iPrOH | 48.8 |

### Example 23: Additional Screening of Cyanide Sources

The reactions were performed by following the optimization procedure 2.

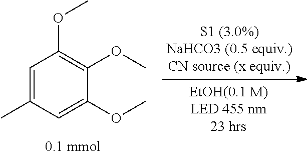

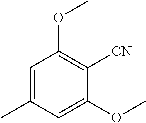

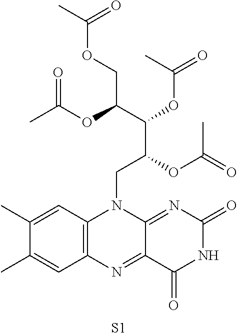 [SMILES: CC(=O)OC[C@H](OC(C)=O)[C@H](OC(C)=O)[C@@H](Cn1c2nc(=O)[nH]c(=O)c-2nc2cc(C)c(C)cc21)OC(C)=O ; "S1"]

| Entry | CN source (x eq.) | HPLC Yield (%) |
|---|---|---|
| 1 | TMSCN (2 eq.) | >99.5 |
| 2 | NaCN (4 eq.) | 24.7 |
| 3 | MeCN (50 uL) | <1.0 |
| 4 | ACH (4 eq.) | 55.0 |
| 5 | ACH (2 eq.) | 49.5 |
| 6 | p-Tol-SO₂-CN (4 eq.) | 0 |
| 7 | TBACN (4 eq.) | 26.0 |
| 8 | CuCN (4 eq.) | <1.0 |
| 9 | CH₂(CN)₂ (Malononitrile, 4 eq.) | <1.0 |
| 10 | AIBN (4 eq.) | <2.0 |

### Example 24: Screening of Base Additive

The reactions were performed by following the optimization procedure 2.

| 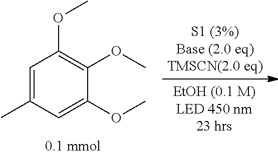 | | |
|---|---|---|
| 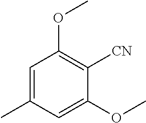 | | |
| 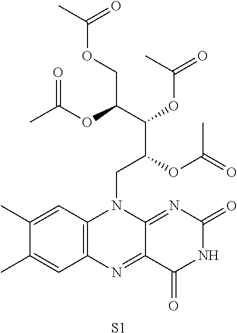 [SMILES: CC(=O)OC[C@H](OC(C)=O)[C@H](OC(C)=O)[C@@H](Cn1c2nc(=O)[nH]c(=O)c-2nc2cc(C)c(C)cc21)OC(C)=O ; "S1"] | | |
| Entry | Base additives | HPLC Yield (%) |
| 1 | Nₐ₂CO₃ | 63.8 |
| 2 | Na₂HPO₄ | 46.8 |
| 3 | NaOAc | 77.6 |
| 4 | KHCO₃ | 53.8 |
| 5 | NaH₂PO₄ | 35.8 |
| 6 | NaHCO₃ | >99.9 |
| 7 | — | N.R. |
| 8 | NaHCO₃ | 99.0 |
| | (sat. aq. 2.5 μL) | |
| 9 | NaHCO₃ | 95.2 |
| | (sat. aq. 5.0 μL) | |
| 10 | NaHCO₃ (0.5 eq) | 99.0 |
| | +H₂O (20 μL) | |
| 11 | NaHCO₃ (0.5 eq) | >99.9 |
| 12 | DIPEA | 62.6 |
| 13 | KOAc | 56.0 |
| 14 | Borate buffer | 89.1 |
| | (pH = 8.5, 20 μL) | |
| 15 | PBS buffer | 44.5 |
| | (pH = 7.4, 20 μL) | |
| 16 | 1N HCl | 6.9 |
| | (20 μL) | |
| 17 | Phosphate buffer | 72.1 |
| | (pH = 9.0, 20 μL) | |

### Example 25: Investigation of S_{N}2 Competitive Reaction

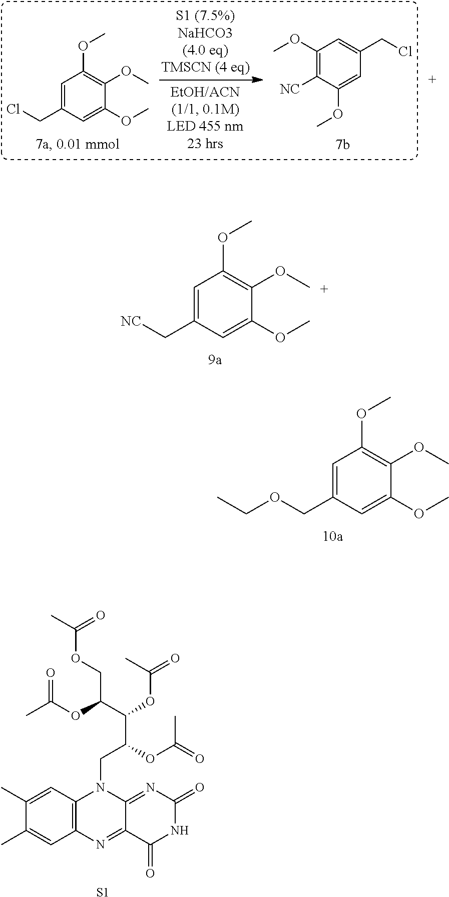

The reactions performed by following the optimization procedure 1.

| Entry | Condition variations | 7b (%) | 9a (%) | 10a (%) |
|---|---|---|---|---|
| 1 | None | 84.9 | 4.0 | 7.5 |
| 2 | Acetone cyanohydrin | 82.3 | 5.2 | 10.3 |
| | (4.0 equiv.) | | | |
| 3 | NaHCO₃ (0.5 equiv.) | 9.6 | 40.0 | 46.0 |
| | S1 (10.0%), EtOH | | | |
| 4 | NaHCO₃ (2.0 equiv.) | 41.0 | 15.0 | 37.5 |
| | EtOH | | | |
| 5 | MeCN | <1.0 | 90.0 | 5.0 |
| 6 | MeCN/EtOH (9/1) | <1.0 | 4.6 | 86.0 |
| 7 | S1 (5.0%) | 78.0 | 5.0 | 12.2 |

HPLC estimated yield is shown in FIG. 20.

### Example 26: Procedure and Data for Cyanation Product Synthesis

General procedure 1 for photocatalyzed cyanation. To a 4.32-dram (16 ml) vial that contained a mini-Teflon-coated magnetic stir bar was added 0.005-0.01 mmol of S1 (5 mol %), NaHCO₃ (0.1-0.2 mmol, 1.0 equiv.) and arene (0.1-0.2 mmol, 1.0 equiv.). The reagent mixture was then attempted to be dissolved in EtOH (1.0-2.0 ml, 0.1 M). The vial was sealed with a PTFE-lined septum screw cap and stirred rapidly for approximately 5 minutes. TMSCN (0.25-0.5 mmol, 2.5 equiv.) was then subsequently added. The vial was positioned on a stir plate approximately 5 cm from either one or two blue LED lamps (Wolezek 36W LED, 450-460 nm) before stirred and irradiated for a designated time. The crude reaction mixture was then concentrated in vacuo and purified by flash chromatography or reverse-phase flash liquid chromatography. Isolation yields reported in the substrate scope section were mostly obtained on a 0.10 or 0.20 mmol scale.

General procedure 2 for photocatalyzed cyanation. 2.0 mmol scale reaction. A round-bottom flask (100 mL) with a rubber septum stopper was used instead of the screw capped vial in general procedure 1. A normal sized Teflon-coated magnetic stir bar, 0.1 mmol of S1 (5 mol %), NaHCO₃ (2.0 mmol, 1.0 equiv.) and arene (2.0 mmol, 1.0 equiv.) was added into the flask. The reagent mixture was then attempted to be dissolved in EtOH (20 ml, 0.1 M). The flask was sealed with a rubber septum and stirred rapidly for approximately 5 minutes. TMSCN (6.0 mmol, 3.0 equiv.) was then subsequently added. The flask was positioned on a stir plate approximately 2.5 cm from two blue LED lamps (Wolezek 36W LED, 450-460 nm) before stirred and irradiated for a designated time. The reaction was also monitored by TLC or HPLC. Lastly, the crude reaction mixture was concentrated in vacuo and purified by aqueous work-up (brine [50 mL] rinse in DCM [50 mL]) and flash chromatography.

General procedure 3 for photocatalyzed cyanation. 5.0 mmol scale reaction. A round-bottom flask (250 mL) with a glass valve connector stopcock stopper was used. A normal sized Teflon-coated magnetic stir bar, 0.15 mmol of S1 (3 mol %), NaHCO₃ (5.0 mmol, 1.0 equiv.) and arene (5.0 mmol, 1.0 equiv.) was added into the flask. The reagent mixture was then attempted to be dissolved in EtOH (50 ml, 0.1 M). The flask was stirred rapidly for approximately 5 minutes. TMSCN (6.0 mmol, 3.0 equiv.) was then subsequently added. The flask was sealed with glass valve connector stopcock stopper installed to an air balloon and positioned on a stir plate approximately 2.5 cm from two blue LED lamps (Wolezek 36W LED, 450-460 nm) before stirred and irradiated for a designated time (33 h). The reaction was also monitored by TLC or HPLC. Lastly, the crude reaction mixture was concentrated in vacuo and purified by aqueous work-up (brine [1100 mL×2] rinse in DCM [1100 mL]) and flash chromatography.

General procedure 4 for photocatalyzed cyanation. 4.0 mmol scale reaction. A round-bottom flask (250 mL) with a glass valve connector stopcock stopper was used. A normal sized Teflon-coated magnetic stir bar, 0.2 mmol of S 1 (5 mol %), NaHCO₃ (8.0 mmol, 2.0 equiv.) and arene (4.0 mmol, 1.0 equiv.) was added into the flask. The reagent mixture was then attempted to be dissolved in EtOH/MeCN (35 ml/5 mL, 0.1 M). The flask was stirred rapidly for approximately 5 minutes. Acetone cyanohydrin (16.0 mmol, 4.0 equiv.) was then subsequently added. The flask was sealed with glass valve connector stopcock stopper installed to an air balloon and positioned on a stir plate approximately 2.5 cm from two blue LED lamps (Wolezek 36W LED, 450-460 nm) before stirred and irradiated for a designated time (96 h). The reaction was also monitored by TLC or HPLC. Lastly, the crude reaction mixture was concentrated in vacuo and purified by aqueous work-up (brine [80 mL×2] rinse in DCM [80 mL]) and flash chromatography.

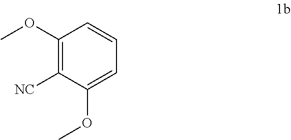

### Example 27: Synthesis of 2,6-dimethoxybenzonitrile (1b)

A colorless crystal solid was obtained for 1b after solvent removal and silica column chromatograph (Hexanes/EA, 9/1-4/1). It was also obtained with 18.0% isolated yield in the previous research, the NMR data were well matched with each other.¹¹ Starting from 1a, the following product amount and yield was obtained in various reaction scales. 0.1 mmol: 16.1 mg, 99.0% yield. 2.0 mmol: 320 mg, 98.0% yield. 5.0 mmol: 775 mg, 95.0% yield (¹H NMR yield was also determined to be ≥95.0%). Starting from 1a′, 0.2 mmol: 29.0 mg product was obtained in 89.0% yield. ¹H NMR (500 MHz, Chloroform-d) δ 7.46-7.39 (m, 1H), 6.55 (d, J=8.7 Hz, 2H), 3.90 (s, 6H). ¹³C NMR (126 MHz, Chloroform-d) δ 162.81, 134.81, 114.16, 103.56, 91.52, 56.33, 56.32. HRMS (ESI) m/z, calculated for [M+H]+: 164.0712; found: 164.0705.

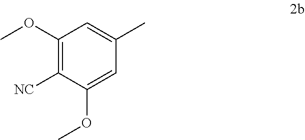

### Example 28: Synthesis of 2,6-dimethoxy-4-methylbenzonitrile (2b)

A white solid of 2b was obtained after solvent removal and silica column chromatograph (Hexanes/EA, 9/1-4/1). Starting from 2a, the following product amount and yield was obtained in various reaction scales. 0.1 mmol: 17.5 mg product obtained in 99.0% yield. 2.0 mmol: 340 mg product obtained in 96.0% yield. ¹H NMR (500 MHz, Chloroform-d) δ 6.38-6.33 (m, 2H), 3.88 (s, 6H), 2.38 (s, 3H). ¹³C NMR (126 MHz, Chloroform-d) δ 162.60, 146.33, 114.53, 104.54, 88.81, 56.22, 56.20, 23.01. HRMS (ESI) m/z, calculated for [M+H]+: 178.0868; found: 178.0861.

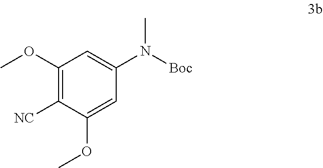 [SMILES: COc1cc(N(C)C(=O)OC(C)(C)C)cc(OC)c1C#N]

### Example 29: Synthesis of tert-butyl (4-cyano-3,5-dimethoxyphenyl)(methyl)carbamate (3b)

An off-white solid of 3b was obtained after solvent removal and silica column chromatograph (Hexanes/EA, 9/1-3/1). Starting from 3a, the following product amount and yield was obtained in various reaction scales. 0.1 mmol: 25.0 mg, 85.5% yield. 2.0 mmol: 543 mg, 93.0% yield. ¹H NMR (400 MHz, Chloroform-d) δ 6.50 (s, 2H), 3.89 (s, 6H), 3.28 (s, 3H), 1.49 (s, 9H). ¹³C NMR (101 MHz, Chloroform-d) δ 162.54, 153.89, 149.92, 114.16, 100.62, 87.98, 81.68, 56.32, 37.20, 28.45. HRMS (ESI) m/z, calculated for [M+H]⁺: 293.1501; found: 293.1493.

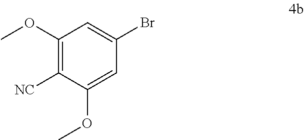

### Example 30: Synthesis of 4-bromo-2,6-dimethoxybenzonitrile (4b)

A white solid of 4b (45.3 mg, 94.0% yield in 0.2 mmol scale) was obtained after solvent removal and silica column chromatograph (Hexanes/EA, 9/1-4/1). ¹H NMR (500 MHz, Chloroform-d) δ 6.73 (s, 2H), 3.91 (s, 6H). ¹³C NMR (126 MHz, Chloroform-d) δ 162.83, 129.27, 113.44, 107.87, 90.95, 56.71. HRMS (ESI) m/z, calculated for [M+H]+: 241.9817; found: 241.9812.

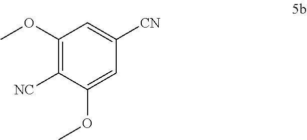

### Example 31: Synthesis of 2,6-dimethoxyterephthalonitrile (5b)

A gray solid of 5b (22.6 mg, 60.0% yield in 0.2 mmol scale) was obtained after solvent removal and silica column chromatograph (Hexanes/EA, 9/1-3/1). ¹H NMR (500 MHz, Chloroform-d) δ 6.83 (s, 2H), 3.96 (s, 6H). ¹³C NMR (126 MHz, Chloroform-d) δ 162.78, 117.84, 117.54, 112.30, 107.31, 96.27, 56.96. HRMS (ESI) m/z, calculated for [M+H]⁺: 189.0664; found: 189.0656.

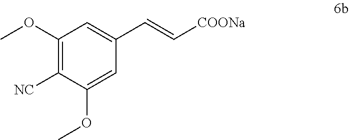

### Example 32: Synthesis of sodium (E)-3-(4-cyano-3,5-dimethoxyphenyl)acrylate (6b)

A white powder of 6b (30.1 mg, 59.0% yield in 0.2 mmol scale) was obtained after solvent removal and a reversed phase C18 column on the flash LC chromatograph (Water/MeCN, both containing 0.1% TFA, 19/1-4/1). ¹H NMR (400 MHz, DMSO-d6) δ 7.59 (d, J=16.0 Hz, 1H), 7.16 (s, 2H), 6.83 (d, J=16.0 Hz, 1H), 3.93 (s, 6H). ¹³C NMR (101 MHz, DMSO-d6) δ 167.25, 162.15, 142.60, 141.28, 123.30, 113.83, 104.04, 90.56, 56.74, 40.20, 40.15, 39.94, 39.73, 39.52, 39.31, 39.10, 38.89.

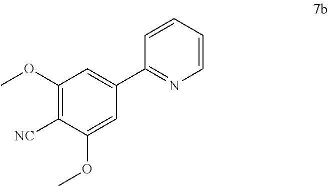 [SMILES: COc1cc(-c2ccccn2)cc(OC)c1C#N]

### Example 33: Synthesis of 2,6-dimethoxy-4-(pyridin-2-yl)benzonitrile (7b)

A yellow solid of 7b (45.6 mg, 95.0% yield in 0.2 mmol scale) was obtained after solvent removal and silica column chromatograph (Hexanes/EA, 9/1-2/1). ¹H NMR (400 MHz, Chloroform-d) δ 8.72 (ddd, J=4.8, 1.8, 0.9 Hz, 1H), 7.81 (dd, J=7.5, 1.8 Hz, 1H), 7.76-7.72 (m, 1H), 7.33 (ddd, J=7.4, 4.8, 1.2 Hz, 1H), 7.20 (s, 2H), 4.02 (s, 6H). ¹³C NMR (101 MHz, Chloroform-d) δ 163.00, 155.82, 150.01, 145.96, 137.21, 123.65, 121.25, 114.22, 102.33, 91.87, 56.50. HRMS (ESI) m/z, calculated for [M+H]⁺: 241.0977; found: 241.0973.

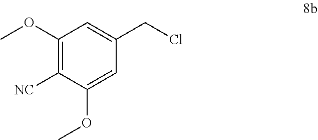

### Example 34: Synthesis of 4-(chloromethyl)-2,6-dimethoxybenzonitrile (8b)

A white solid of 8b was obtained after solvent removal and silica column chromatograph (Hexanes/EA, 9/1-4/1). With TMSCN, 0.2 mmol scale, 32.9 mg, 78.0% yield; with acetone cyanohydrin, 0.2 mmol scale, 29.5 mg, 70.0% yield. ¹H NMR (500 MHz, Chloroform-d) δ 6.58 (s, 2H), 4.54 (s, 2H), 3.92 (s, 6H). ¹³C NMR (101 MHz, Chloroform-d) δ 162.85, 144.72, 113.76, 103.74, 91.45, 77.48, 77.16, 76.84, 56.46, 45.87. HRMS (ESI) m/z, calculated for [M+H]⁺: 212.0478; found: 212.0475.

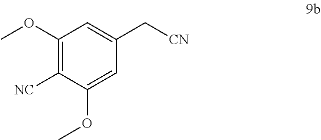

### Example 35: Synthesis of 4-(cyanomethyl)-2,6-dimethoxybenzonitrile (9b)

A pale yellow solid of 9b (39.2 mg, 97.0% yield in 0.2 mmol scale) was obtained after solvent removal and silica column chromatograph (Hexanes/EA, 9/1-3/1). ¹H NMR (400 MHz, Chloroform-d) δ 6.52 (s, 2H), 3.93 (s, 6H), 3.78 (s, 2H). ¹³C NMR (101 MHz, Chloroform-d) δ 163.11, 137.45, 116.72, 113.46, 103.42, 91.43, 56.58, 24.61. HRMS (ESI) m/z, calculated for [M+H]⁺: 203.0821; found: 203.0815; [M+Na]⁺: 225.0640; found: 225.0634.

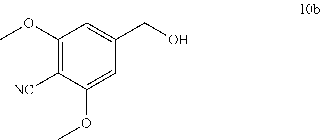

### Example 36: Synthesis of 4-(hydroxymethyl)-2,6-dimethoxybenzonitrile (10b)

An off-white solid of 10b (30.7 mg, 79.5% yield in 0.2 mmol scale) was obtained after solvent removal and silica column chromatograph (Hexanes/EA, 9/1-3/1). ¹H NMR (500 MHz, Chloroform-d) δ 6.57 (s, 2H), 4.72 (s, 2H), 3.91 (s, 6H). ¹³C NMR (126 MHz, Chloroform-d) δ 162.90, 149.14, 114.29, 101.38, 101.30, 90.20, 77.41, 77.16, 76.91, 64.85, 56.38, 56.31. HRMS (ESI) m/z, calculated for [M+H]⁺: 194.0817; found: 194.0816.

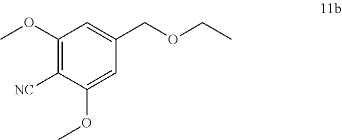 [SMILES: CCOCc1cc(OC)c(C#N)c(OC)c1]

### Example 37: Synthesis of 4-(ethoxymethyl)-2,6-dimethoxybenzonitrile (11b)

A white crystal off 11b (30.1 mg, 68.0% yield in 0.2 mmol scale) was obtained after solvent removal and silica column chromatograph (Hexanes/EA, 9/1-3/1). With acetone cyanohydrin, 26.9 mg product, 61.0% yield also obtained in 0.2 mmol scale reaction. ¹H NMR (400 MHz, Chloroform-d) δ 6.54 (s, 2H), 4.49 (s, 2H), 3.91 (s, 6H), 3.57 (q, J=7.0 Hz, 2H), 1.27 (t, J=7.0 Hz, 3H). ¹³C NMR (101 MHz, Chloroform-d) δ 162.82, 147.01, 114.30, 102.07, 90.31, 77.48, 77.16, 76.84, 72.34, 66.52, 56.32, 15.31. HRMS (ESI) m/z, calculated for [M+H]⁺: 222.1130; found: 222.1126; [M+Na]⁺: 244.0950; found: 244.0945.

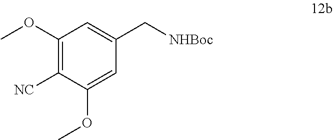 [SMILES: COc1cc(CNC(=O)OC(C)(C)C)cc(OC)c1C#N]

### Example 38: Synthesis of tert-butyl (4-cyano-3,5-dimethoxybenzyl)carbamate (12b)

An off-white solid of 12b (42.0 mg, 72.0% yield in 0.2 mmol scale) was obtained after solvent removal and silica column chromatograph (Hexanes/EA, 9/1-2/1). ¹H NMR (500 MHz, Chloroform-d) δ 6.47 (s, 2H), 4.95 (s, 1H), 4.30 (d, J=6.1 Hz, 2H), 3.89 (s, 6H), 1.47 (s, 9H). ¹³C NMR (101 MHz, Chloroform-d) δ 162.84, 156.10, 147.48, 114.16, 102.32, 90.17, 80.16, 56.28, 44.99, 28.44. HRMS (ESI) m/z, calculated for [M+H]⁺: 293.1501; found: 293.1498; [M+Na]⁺: 315.1321; found: 315.1318.

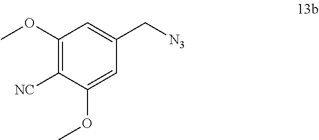 [SMILES: COc1cc(CN=[N+]=[N-])cc(OC)c1C#N]

### Example 39: Synthesis of 4-(azidomethyl)-2,6-dimethoxybenzonitrile (13b)

An off-white solid of 13b (20.4 mg, 93.5% yield in 0.1 mmol scale) was obtained after solvent removal and silica column chromatograph (Hexanes/EA, 9/1-3/1). ¹H NMR (400 MHz, Chloroform-d) δ 6.49 (s, 2H), 4.38 (s, 2H), 3.91 (s, 6H). ¹³C NMR (101 MHz, Chloroform-d) δ 162.95, 143.30, 113.80, 102.89, 91.12, 56.44, 54.77. HRMS (ESI) m/z, calculated for [M+H]⁺: 219.0882; found: 219.0877.

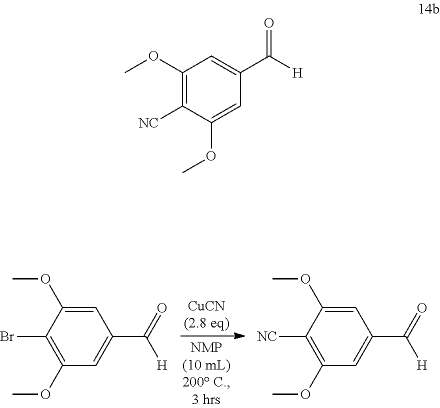

### Example 40: Synthesis of 4-formyl-2,6-dimethoxybenzonitrile (14b)

Less than 5% HPLC yield was observed and failed to isolate the product via the photoredox cyanation by S1. Therefore, the title compound (as the authentic standard product) was synthesized as a light brown solid via the Rosenmund-von Braun reaction²³ with an isolated yield of 61.0%. 2.0 mmol, 233 mg product obtained. ¹H NMR (500 MHz, Chloroform-d) δ 9.98 (s, H), 7.06 (s, 2H), 4.00 (s, 6H). ¹³C NMR (126 MHz, Chloroform-d) δ 190.77, 163.25, 140.88, 113.10, 104.24, 96.73, 56.77. HRMS (ESI) m/z, calculated for [M+H]⁺: 192.0661; found: 192.0655.

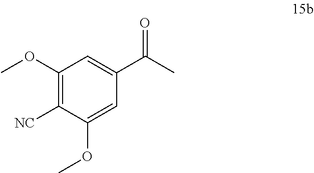

### Example 41: Synthesis of 4-acetyl-2,6-dimethoxybenzonitrile (15b)

An off-white solid of 15b (38.3 mg, 93.5% yield in 0.2 mmol scale) was obtained after solvent removal and silica column chromatograph (Hexanes/EA, 9/1-3/1). ¹H NMR (500 MHz, Chloroform-d) δ 7.08 (s, 2H), 3.97 (s, 6H), 2.62 (s, 3H). ¹³C NMR (126 MHz, Chloroform-d) δ 196.69, 162.80, 142.14, 113.33, 103.17, 95.44, 56.60, 26.99. HRMS (ESI) m/z, calculated for [M+H]⁺: 206.0817; found: 206.0810.

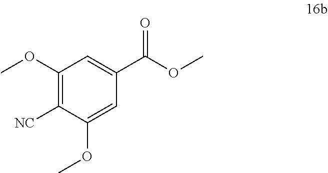

### Example 42: Synthesis of Methyl 4-cyano-3,5-dimethoxybenzoate (16b)

An off white solid of 16b (41.1 mg, 93.0% yield in 0.2 mmol scale) was obtained after solvent removal and silica column chromatograph (Hexanes/EA, 9/1-3/1). ¹H NMR (500 MHz, Chloroform-d) δ 7.22 (s, 2H), 3.97 (s, 6H), 3.95 (s, 3H). ¹³C NMR (126 MHz, Chloroform-d) δ 165.74, 162.59, 135.89, 113.35, 104.59, 95.44, 77.41, 77.16, 76.91, 56.66, 52.98, 52.97. HRMS (ESI) m/z, calculated for [M+H]⁺: 222.0766; found: 222.0760.

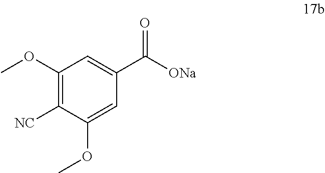

### Example 43: Synthesis of Sodium 4-cyano-3,5-dimethoxybenzoate (17b)

A white powder of 17b (45.1 mg, 98.5% yield in 0.2 mmol scale) was obtained after work-up. 4 mL ethanol was first added to dissolve the product. Then, the remaining base was filtered before the solvent was removed from the filtrate. Diethyl ether (10 mL) was used to rinse the product mixture before the soluble catalyst byproduct was filtered and the remaining solid was dried over vacuum to afford the white product. ¹H NMR (400 MHz, dmso-d6) δ 7.21 (s, 2H), 3.87 (s, 6H). ¹³C NMR (101 MHz, dmso-d6) δ 166.74, 161.39, 149.01, 114.47, 104.20, 89.24, 56.05.

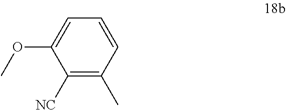

### Example 44: Synthesis of 2-methoxy-6-methylbenzonitrile (18b)

A colorless liquid of 18b (25.9 mg, 88.0% yield in 0.2 mmol scale) was obtained after solvent removal and silica column chromatograph (Hexanes/EA, 9/1-4/1). ¹H NMR (500 MHz, Chloroform-d) δ 7.39 (t, J=8.1 Hz, 1H), 6.86 (d, J=7.9 Hz, 1H), 6.77 (d, J=8.6 Hz, 1H), 3.90 (s, 3H), 2.49 (s, 3H).¹³C NMR (126 MHz, Chloroform-d) δ 161.73, 143.96, 133.61, 122.16, 115.80, 108.31, 102.46, 56.11, 20.57. HRMS (ESI) m/z, calculated for [M+H]⁺: 148.0762; found: 148.0756.

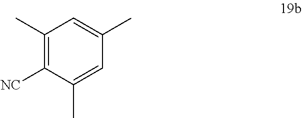

### Example 45: Synthesis of 2,4,6-trimethylbenzonitrile (19b)

A white crystal solid of 19b (24.8 mg, 85.5% yield in 0.2 mmol scale) was obtained after solvent removal and silica column chromatograph (Hexanes/EA, 9/1-4/1). ¹H NMR (500 MHz, Chloroform-d) δ 7.26, 6.93, 2.48, 2.32. ¹³C NMR (101 MHz, Chloroform-d) δ 142.93, 142.12, 128.33, 117.78, 110.47, 21.71, 20.77. HRMS (ESI) m/z, calculated for [M+H]⁺: 146.0970; found: 146.0964.

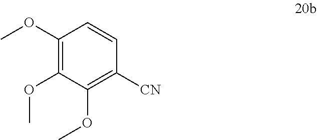

### Example 46: Synthesis of 2,3,4-trimethoxybenzonitrile (20b)

A white crystal solid of 20b (23.9 mg, 62.0% yield in 0.2 mmol scale) was obtained after solvent removal and silica column chromatograph (Hexanes/EA, 9/1-4/1). 2.0 mmol: 270.1 mg product obtained in 70.0% yield. 4.0 mmol: 749.0 mg product obtained in 97.0% yield. 1H NMR (400 MHz, Chloroform-d) δ 7.28 (d, J=8.7 Hz, 1H), 6.68 (d, J=8.7 Hz, 1H), 4.06 (s, 3H), 3.91 (s, 3H), 3.86 (s, 3H). ¹³C NMR (101 MHz, Chloroform-d) δ 158.14, 156.06, 142.04, 128.94, 116.71, 107.64, 99.35, 61.93, 61.29, 56.43. HRMS (ESI) m/z, calculated for [M+H]⁺: 194.0817; found: 194.0812.

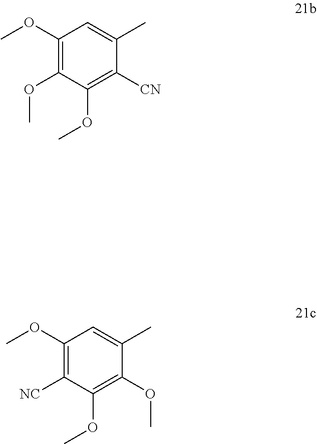

### Example 47: Synthesis of Compound 21b and 21c

2,3,4-trimethoxy-6-methylbenzonitrile (21b):2,3,6-trimethoxy-4-methyl benzonitrile (21c)=4:1, colorless gel-like solid (34.6 mg, 83.5 yield in 0.2 mmol scale) was obtained after solvent removal and silica column chromatograph (Hexanes/EA, 9/1-3/1). The two regioisomers were inseparable by column chromatograph while the amount ratio could be calculated by the NMR spectra. 2,3,4-trimethoxy-6-methylbenzonitrile (21b): ¹H NMR (500 MHz, Chloroform-d) δ 6.52 (s, 1H), 4.01 (s, 3H), 3.88 (s, 3H), 3.81 (s, 3H), 2.44 (s, 3H).

¹³C NMR (126 MHz, Chloroform-d) δ 157.29, 155.79, 139.60, 139.01, 115.91, 108.79, 99.84, 61.78, 61.20, 56.25, 20.69. 2,3,6-trimethoxy-4-methylbenzonitrile (21c): ¹H NMR (500 MHz, Chloroform-d) δ 6.45 (s, 1H), 4.01 (s, 3H), 3.84 (s, 3H), 3.74 (s, 3H), 2.29 (s, 3H). ¹³C NMR (126 MHz, Chloroform-d) δ 157.46, 155.92, 144.62, 139.43, 113.99, 107.90, 94.53, 61.46, 60.63, 56.31, 16.99.

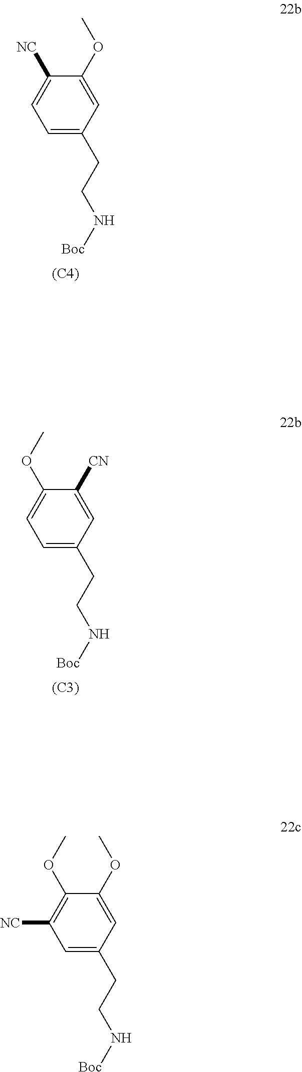

### Example 48: Synthesis of Compounds 22b (C4) and 22b (C3)

tert-butyl (4-cyano-3-methoxyphenethyl)carbamate [22b(C4)]: tert-butyl (3-cyano-4-methoxyphenethyl)carbamate [22b(C3)]=4:1, white crystal solid (23.8 mg, 43.0% yield in 0.2 mmol scale) was obtained after solvent removal and silica column chromatograph (Hexanes/EA, 9/1-2/1). The two regioisomers were inseparable by column chromatograph while the amount ratio could be calculated by the NMR spectra. 22b(C4): ¹H NMR (400 MHz, Chloroform-d) δ 7.47 (d, J=7.8 Hz, 1H), 6.83 (d, J=7.9 Hz, 1H), 6.79 (s, 1H), 4.58 (s, 1H), 3.91 (s, 3H), 3.38 (s, 2H), 2.84 (t, J=7.0 Hz, 2H), 1.42 (s, 9H). 22b(C3): ¹H NMR (400 MHz, Chloroform-d) δ 7.36 (d, J=2.1 Hz, 2H), 6.92-6.89 (m, 1H), 4.58 (s, 1H), 3.90 (s, 3H), 3.33 (s, 2H), 2.74 (t, J=7.0 Hz, 2H), 1.42 (s, 9H). ¹³C NMR (101 MHz, Chloroform-d) δ 161.50, 160.08, 155.90, 146.81, 134.94, 133.85, 133.82, 131.80, 128.34, 125.53, 121.41, 116.68, 116.56, 111.94, 111.65, 101.87, 99.98, 79.71, 56.21, 56.11, 41.76, 41.39, 37.01, 35.14, 28.50. tert-butyl (3-cyano-4,5-dimethoxyphenethyl)carbamate (22c), white crystal solid (22.7 mg, 37.0% yield in 0.2 mmol scale) was obtained after solvent removal and silica column chromatograph (Hexanes/EA, 9/1-2/1). ¹H NMR (400 MHz, Chloroform-d) δ 7.02 (s, 1H), 6.80 (s, 1H), 4.66 (s, 1H), 3.92 (s, 3H), 3.87 (s, 3H), 3.45-3.32 (m, 2H), 2.97 (t, J=7.0 Hz, 2H), 1.42 (s, 9H). ¹³C NMR (101 MHz, Chloroform-d) δ 155.96, 152.91, 147.91, 137.52, 118.51, 114.31, 112.60, 103.74, 79.60, 56.35, 56.26, 41.22, 34.89, 28.52.

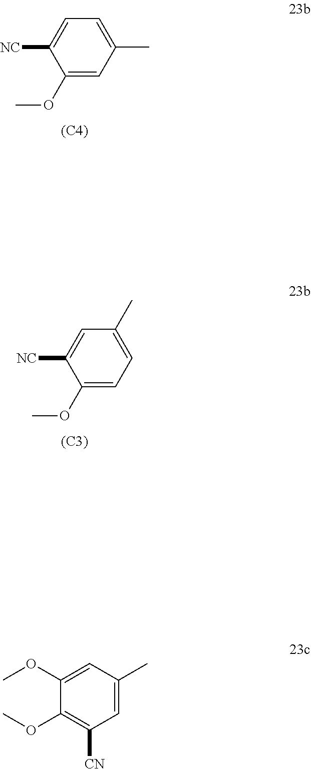

### Example 49: Synthesis of Compounds 23b (C4) and 23b (C3)

2-methoxy-4-methylbenzonitrile [23b(C4)]: 2-methoxy-5-methylbenzonitrile [23b(C3)]=10:2.9, white crystal solid (17.7 mg, 60.0% yield in 0.2 mmol scale) was obtained after solvent removal and silica column chromatograph (Hexanes/EA, 9/1-3/1). The two regioisomers were inseparable by column chromatograph while the amount ratio could be calculated by the NMR spectra. ¹H NMR (500 MHz, Chloroform-d) δ 7.43 (d, J=7.8 Hz, 1H), 7.34 (s, OH), 7.32 (d, J=8.9 Hz, OH), 6.86 (d, J=8.5 Hz, OH), 6.81 (d, J=7.8 Hz, 1H), 6.77 (s, 1H), 3.91 (s, 3H), 3.89 (s, 1H), 2.41 (s, 3H), 2.30 (s, 1H). ¹³C NMR (126 MHz, Chloroform-d) δ 161.35, 159.43, 145.83, 135.11, 133.91, 133.56, 130.45, 121.79, 116.96, 116.81, 112.17, 111.33, 101.56, 98.98, 56.16, 56.02, 22.44, 20.24. 2,3-dimethoxy-5-methylbenzonitrile (23c), white crystal solid (12.7 mg, 35.8% yield in 0.2 mmol scale) was obtained after solvent removal and silica column chromatograph (Hexanes/EA, 9/1-3/1). ¹H NMR (500 MHz, Chloroform-d) δ 6.99 (s, 1H), 6.73 (s, 1H), 3.90 (s, 3H), 3.86 (s, 3H), 2.48 (s, 3H). ¹³C NMR (126 MHz, Chloroform-d) δ 152.65, 147.28, 136.51, 118.73, 114.07, 112.80, 103.70, 77.41, 77.16, 76.91, 56.29, 56.14, 20.27.

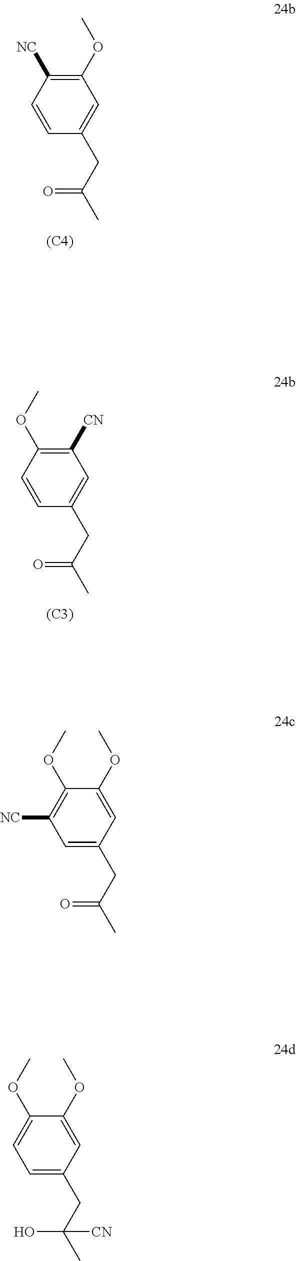

### Example 50: Synthesis of Compounds 24b (C4) and 24b (C3)

2-methoxy-4-(2-oxopropyl)benzonitrile [24b(C4)]: 2-methoxy-5-(2-oxopropyl)benzonitrile [24b(C3)]: 2,3-dimethoxy-5-(2-oxopropyl)benzonitrile (24c)=10:3:2.2, white crystal solid (21.9 mg, 49.4% (24b)+7.2% (24c) yield in 0.2 mmol scale) was obtained after solvent removal and silica column chromatograph (Hexanes/EA, 9/1-3/1). The two regioisomers 24b and the C—H cyanation product 24c were inseparable by column chromatograph while the amount ratio could be calculated by the NMR spectra. 24b(C4): ¹H NMR (400 MHz, Chloroform-d) δ 7.51 (d, J=7.8 Hz, 1H), 6.83 (d, J=7.8 Hz, 1H), 6.79 (s, 1H), 3.92 (s, 3H), 3.75 (s, 2H), 2.21 (s, 3H) 24b(C3): ¹H NMR (400 MHz, Chloroform-d) δ 7.35 (dt, J=8.7, 2.2 Hz, 2H), 6.94 (d, J=8.5 Hz, 1H), 3.90-3.88 (m, 5H), 2.19 (s, 3H). 24c: ¹H NMR (400 MHz, Chloroform-d) δ 7.05 (s, 1H), 6.74 (s, 1H), 3.90-3.88 (m, 5H), 3.67 (s, 3H), 2.27 (s, 1H). 3-(3,4-dimethoxyphenyl)-2-hydroxy-2-methylpropanenitrile (24d) white solid (4.4 mg, 9.9% yield in 0.2 mmol scale) was obtained after solvent removal and silica column chromatograph (Hexanes/EA, 9/1-4/1). ¹H NMR (500 MHz, Chloroform-d) δ 6.88 (s, 1H), 6.87 (d, J=1.7 Hz, 2H), 3.90 (s, 3H), 3.89 (s, 3H), 3.08 (d, J=13.9 Hz, 1H), 2.88 (d, J=13.9 Hz, 1H), 1.67 (s, 3H).

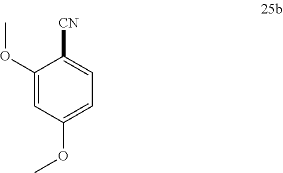

### Example 51: Synthesis of 2,4-dimethoxybenzonitrile (25b)

A white solid of 25b (34.2 mg, 10.5% yield in 2.0 mmol scale) was obtained after solvent removal and silica column chromatograph (Hexanes/EA, 9/1-3/1). ¹H NMR (400 MHz, Chloroform-d) δ 7.47 (d, J=8.6 Hz, 1H), 6.51 (dd, J=8.6, 2.2 Hz, 1H), 6.46 (d, J=2.2 Hz, 1H), 3.90 (s, 3H), 3.85 (s, 3H). ¹³C NMR (101 MHz, Chloroform-d) δ 164.76, 162.99, 135.08, 117.07, 105.86, 98.65, 94.21, 56.11, 55.84. HRMS (ESI) m/z, calculated for [M+H]⁺: 164.0712; found: 164.0705.

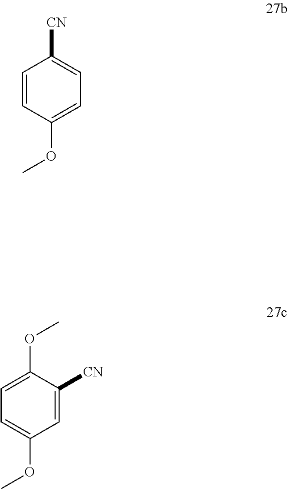

### Example 52: Synthesis of Compounds 27b and 27c

4-methoxybenzonitrile (27b): 2,5-dimethoxybenzonitrile (27c)=10:3, white solid (26.0 mg, 71.0% (27b)+21.5% (27c) yield in 0.2 mmol scale) was obtained after solvent removal and silica column chromatograph (Hexanes/EA, 9/1-3/1). The two products were inseparable by column chromatograph while the amount ratio could be calculated by the NMR spectra. ¹H NMR (500 MHz, Chloroform-d) δ 7.61-7.55 (m, 2H), 6.97-6.92 (m, 2H), 3.86 (s, 3H); δ 7.09 (dd, J=9.1, 3.1 Hz, 1H), 7.05 (d, J=3.1 Hz, 1H), 6.90 (d, J=9.1 Hz, 1H), 3.88 (s, 3H), 3.78 (s, 3H). ¹³C NMR (126 MHz, Chloroform-d) δ 162.98, 155.89, 153.30, 134.12, 120.97, 119.36, 117.74, 116.51, 114.88, 114.79, 112.75, 104.12, 101.96, 77.41, 77.16, 76.91, 56.55, 56.09, 55.68, 29.83.

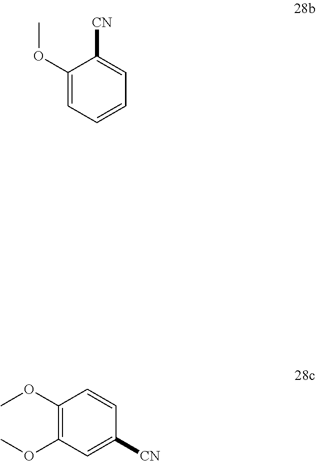

### Example 53: Synthesis of Compounds 28b and 28c

2-methoxybenzonitrile (28b): 3,4-dimethoxybenzonitrile (28c)=10:1, colorless oil (26.0 mg, 86.5% (28b)+9.0% (28c) yield in 0.2 mmol scale) was obtained after solvent removal and silica column chromatograph (Flash LC, UV=212 nm, Hexanes/EA, 9/1-3/1). The two products were inseparable by column chromatograph while the amount ratio could be calculated by the NMR spectra. 28b: ¹H NMR (500 MHz, Chloroform-d) δ 7.57-7.51 (m, J=10.0, 8.3, 4.6, 1.2 Hz, 2H), 7.00 (td, J=7.6, 0.9 Hz, 1H), 6.97 (d, J=8.4 Hz, 1H), 3.92 (s, 3H). 28c: ¹H NMR (500 MHz, Chloroform-d) 7.28 (dd, J=8.3, 1.9 Hz, 1H), 7.07 (d, J=1.9 Hz, 1H), 6.90 (d, J=8.4 Hz, 1H), 3.89 (s, 3H). 28b:28c=10:1. ¹³C NMR (126 MHz, Chloroform-d) δ 161.34, 152.99, 149.30, 134.49, 133.85, 126.58, 120.86, 119.34, 116.60, 114.04, 111.40, 111.37, 103.98, 101.90, 56.25, 56.21, 56.10.

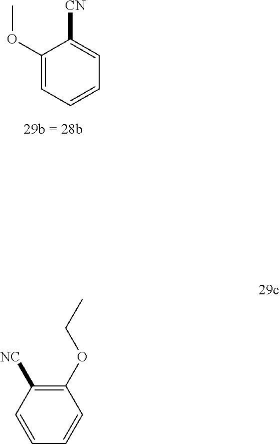

### Example 54: Synthesis of Compounds 29b and 29c

2-methoxybenzonitrile (29b), colorless oil (9.6 mg, 36.0% yield in 0.2 mmol scale) was obtained after solvent removal and silica column chromatograph (Flash LC, UV=212 nm, Hexanes/EA, 9/1-3/1). ¹H NMR (500 MHz, Chloroform-d) δ 7.54 (dd, J=12.8, 4.6 Hz, 2H), 7.03-6.94 (m, 2H), 3.92 (s, 3H). 2-ethoxybenzonitrile (29c), colorless oil (17.1 mg, 58.0% yield in 0.2 mmol scale) was obtained after solvent removal and silica column chromatograph (Flash LC, UV=212 nm, Hexanes/EA, 9/1-3/1). ¹H NMR (500 MHz, Chloroform-d) δ 7.59-7.46 (m, 2H), 7.03-6.89 (m, 2H), 4.14 (q, J=7.0 Hz, 2H), 1.48 (t, J=7.0 Hz, 3H). HRMS (ESI) m/z, calculated for [M+H]⁺: 148.0762; found: 148.0757

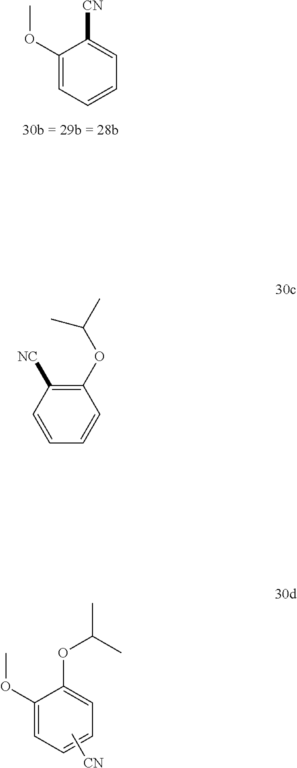

### Example 55: Synthesis of Compounds 30b, 30c, and 30d

2-methoxybenzonitrile (30b), colorless oil (3.7 mg, 14.0% yield in 0.2 mmol scale) was obtained after solvent removal and silica column chromatograph (Flash LC, UV=212 nm, Hexanes/EA, 9/1-3/1). ¹H NMR (400 MHz, Chloroform-d) δ 7.60-7.49 (m, 2H), 7.05-6.94 (m, 2H), 3.94 (s, 3H). ¹³C NMR (101 MHz, Chloroform-d) δ 161.39, 134.49, 133.92, 120.90, 116.62, 111.42, 102.02, 56.13. 2-isopropoxybenzonitrile (30c), colorless oil (12.9 mg, 40.0% yield in 0.2 mmol scale) was obtained after solvent removal and silica column chromatograph (Flash LC, UV=212 nm, Hexanes/EA, 9/1-3/1). ¹H NMR (400 MHz, Chloroform-d) δ 7.58-7.44 (m, 2H), 7.01-6.91 (m, 2H), 4.65 (dt, J=12.2, 6.1 Hz, 1H), 1.40 (d, J=6.1 Hz, 6H). ¹³C NMR (101 MHz, Chloroform-d) δ 160.09, 134.21, 134.08, 120.60, 116.86, 113.85, 103.22, 71.98, 22.00. C—H cyanation products mixture (30d), colorless solid (5.0 mg, in 0.2 mmol scale) was obtained after solvent removal and silica column chromatograph (Flash LC, UV=212 nm, Hexanes/EA, 9/1-3/1). 1H NMR (400 MHz, Chloroform-d) δ 7.27 (d, J=1.9 Hz, OH), 7.26-7.24 (m, 1H), 7.23 (d, J=1.9 Hz, 1H), 7.10 (d, J=1.9 Hz, 1H), 7.08 (d, J=1.9 Hz, 1H), 6.90 (s, 1H), 6.88 (s, 1H), 4.67-4.59 (m, 1H), 4.57-4.49 (m, 1H), 3.90 (s, 3H), 3.87 (s, 5H), 1.39 (dd, J=7.2, 6.1 Hz, 16H).

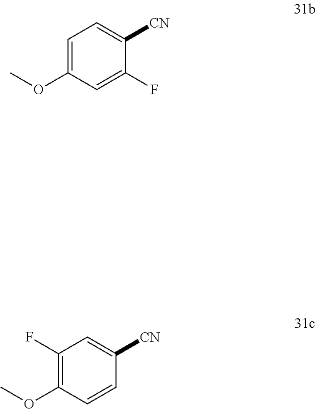

### Example 56: Synthesis of Compounds 31b and 31c

2-fluoro-4-methoxybenzonitrile (31b): 3-fluoro-4-methoxybenzonitrile (31c)=80.5:17.5, colorless solid (29.9 mg, 80.5% (31b)+17.5% (31c) yield in 0.2 mmol scale) was obtained after solvent removal and silica column chromatograph (Flash LC, UV=212 nm, Hexanes/EA, 9/1-3/1). 2-fluoro-4-methoxybenzonitrile (31b): 1H NMR (400 MHz, Chloroform-d) δ 7.51 (dd, J=8.6, 7.6 Hz, 1H), 6.76 (dd, J=8.7, 2.3 Hz, 1H), 6.71 (dd, J=10.9, 2.4 Hz, 1H), 3.86 (s, 3H). 3-fluoro-4-methoxybenzonitrile (31c): ¹H NMR (400 MHz, Chloroform-d) 67.45-7.41 (m, 1H), 7.37-7.33 (m, 1H), 7.04-6.99 (m, 1H), 3.95 (s, 3H). Mixture: ¹³C NMR (101 MHz, Chloroform-d) δ 166.04, 164.92 (d, J=11.1 Hz), 163.47, 153.12, 151.96 (d, J=10.3 Hz), 150.63, 134.33 (d, J=2.3 Hz), 129.83 (d, J=4.0 Hz), 119.66 (d, J=21.2 Hz), 118.12 (d, J=2.5 Hz), 114.51, 113.69 (d, J=2.5 Hz), 111.42 (d, J=2.8 Hz), 104.08 (d, J=8.3 Hz), 102.55, 102.32, 93.20 (d, J=15.8 Hz), 56.50, 56.17. ⁹F NMR (376 MHz, Chloroform-d) δ −104.23 (dd, J=10.9, 7.5 Hz, 81F, 31b), −120.39 (dd, J=12.2, 8.9 Hz, 2F, 31a), −131.92-−132.08 (m, 18F, 31c). 2.0% 31a, starting material left in the isolated products mixture, which indicated the three compounds were actually inseparable with the silica column condition above, and only the catalyst and inorganic additive were removed. All the NMR spectra of 31c matched with the reference data.¹

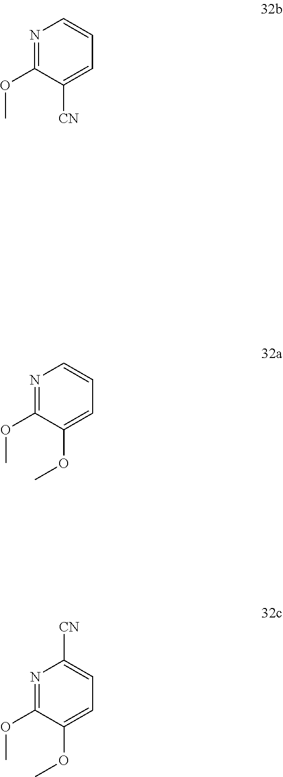

### Example 57: Synthesis of Compounds 32b, 32a, and 32c

2-methoxynicotinonitrile (32b) couldn't be separated from the starting material 32a while the ¹H NMR spectrum can be assigned in the oil mixture of 32b and 32a (3:10, 16.7 mg, 14.0% (32b) yield in 0.2 mmol scale), which was obtained after solvent removal and silica column chromatograph (Flash LC, UV=212 nm, Hexanes/EA, 9/1-2/1). 32b: ¹H NMR (400 MHz, Chloroform-d) δ 8.36 (s, 1H), 7.89-7.84 (m, 1H), 6.97 (dd, J=7.9, 4.2 Hz, 1H), 4.06 (s, J=3.7 Hz, 3H). 5,6-dimethoxypicolinonitrile (32c), white solid (9.0 mg, 27.4% yield in 0.2 mmol scale) was obtained after solvent removal and silica column chromatograph (Flash LC, UV=212 nm, Hexanes/EA, 9/1-2/1) ¹H NMR (400 MHz, Chloroform-d) δ 7.33 (d, J=8.0 Hz, 1H), 7.03 (d, J=8.0 Hz, 1H), 4.04 (s, 3H), 3.93 (d, J=4.2 Hz, 3H). ¹³C NMR (101 MHz, Chloroform-d) δ 154.96, 147.86, 123.95, 120.30, 117.84, 116.22, 56.23, 54.67.

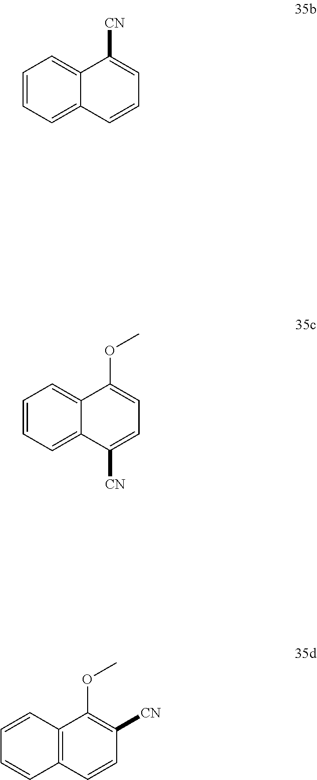

### Example 58: Synthesis of Compounds 35b and 35c

1-naphthonitrile (35b): 1-methoxy-2-naphthonitrile(35d)=3:1 couldn't be separated from a C—H cyanation product, possibly 35d while the ¹H NMR spectrum can be assigned in the oil mixture of 35b and 35d (3:1, 8.1 mg, 18.8% (35b) yield in 0.2 mmol scale), which was obtained after solvent removal and silica column chromatograph (Flash LC, UV=212 nm, Hexanes/DCM, 50/1-8/1). ¹H NMR (400 MHz, Chloroform-d) δ 8.25 (dd, J=8.4, 3.5 Hz, 1H), 8.09 (d, J=8.3 Hz, 1H), 7.96-7.89 (m, 2H), 7.86-7.83 (m), 7.71 (ddd, J=8.4, 6.9, 1.4 Hz, 1H), 7.66-7.50 (m, 3H), 7.47 (dd, J=8.5, 1.5 Hz), 4.31 (d, J=0.9 Hz, 1H). 4-methoxy-1-naphthonitrile (35c), white solid (8.2 mg, 22.5% yield in 0.2 mmol scale) was obtained after solvent removal and silica column chromatograph (Flash LC, UV=212 nm, Hexanes/DCM, 50/1-8/1). ¹H NMR (400 MHz, Chloroform-d) δ 8.32 (ddd, J=8.4, 1.3, 0.7 Hz, 1H), 8.17 (dt, J=8.3, 0.9 Hz, 1H), 7.86 (d, J=8.1 Hz, 1H), 7.70 (ddd, J=8.3, 6.9, 1.3 Hz, 1H), 7.59 (ddd, J=8.3, 6.9, 1.3 Hz, 1H), 6.84 (d, J=8.1 Hz, 1H). ¹³C NMR (101 MHz, Chloroform-d) δ 159.56, 134.20, 133.64, 129.10, 126.87, 125.39, 125.09, 122.92, 118.59, 103.48, 102.09, 56.14.

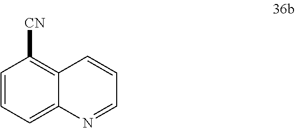

Reaction yield was estimated by HPLC. 36a and the product standard 36b were commercially available. Photoredox cyanation of 36a by S1, isocratic 30%, HPLC condition B. HPLC estimated yield of 36b by peak area integrations (data not shown): ˜14.0%, not a quantified yield.

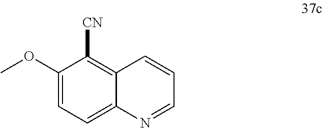

Reaction yield was estimated by HPLC. 37a and the product standard 37c were commercially available. Photoredox cyanation of 37a by S1, isocratic 30%, HPLC condition B. HPLC estimated yield of 37c by peak area integrations (data not shown): ˜5.0%, not a quantified yield.

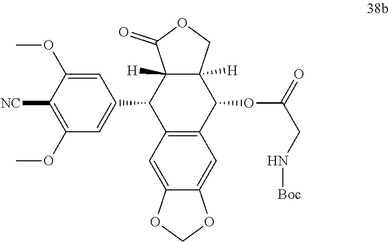

### Example 59: Synthesis of (5S,5aS,8aS,9S)-9-(4-cyano-3,5-dimethoxyphenyl)-8-oxo-5,5a,6,8,8a,9 hexahydrofuro[3′,4′: 6,7]naphtho[2,3-d][1,3]dioxol-5-yl (tert-butoxycarbonyl)glycinate (38b)

A white solid of 38b (10.1 mg, 35.7% yield in 0.05 mmol scale) was obtained after solvent removal and silica column chromatograph (DCM/Acetone, 50/1-10/1). ¹H NMR (400 MHz, Chloroform-d) δ 6.80 (s, 1H), 6.50 (s, 1H), 6.38 (s, 2H), 6.00 (q, J=1.3 Hz, 2H), 5.93 (d, J=9.3 Hz, 1H), 5.04 (s, 1H), 4.63 (d, J=4.8 Hz, 1H), 4.46-4.37 (m, 1H), 4.25-4.15 (m, 1H), 3.98 (qd, J=18.1, 5.8 Hz, 2H), 3.79 (s, 6H), 2.98 (dd, J=14.7, 4.7 Hz, 1H), 2.83-2.68 (m, 2H), 1.44 (s, 9H). ¹³C NMR (101 MHz, Chloroform-d) δ 173.27, 171.07, 162.22, 155.94, 148.65, 148.23, 147.03, 131.08, 128.07, 122.72, 113.94, 109.63, 107.46, 106.35, 101.97, 90.55, 80.69, 74.52, 71.40, 65.27, 56.34, 45.45, 44.38, 42.87, 38.94, 29.83, 29.46, 28.40. HRMS (ESI) m/z, calculated for [M+Na]+: 589.1798; found: 589.1798; [M+MeOH+Na]⁺: 621.2060; found: 621.2060.

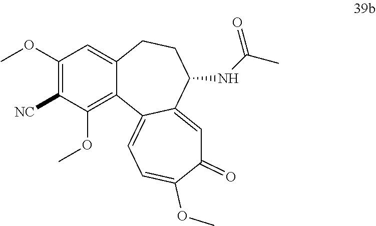

### Example 60: Synthesis of (S)—N-(2-cyano-1,3,10-trimethoxy-9-oxo-5,6,7,9-tetrahydrobenzo[a]heptalen-7-yl)acetamide (39b)

A yellow foam-like solid of 39b (22.5 mg, 57.0% yield in 0.1 mmol scale) was obtained solvent removal and a reversed phase C18 column on the flash LC chromatograph (Water/MeCN, both containing 0.1% TFA, 19/1-3/1). ¹H NMR (500 MHz, Acetone-d₆) δ 7.99 (d, J=7.0 Hz, 1H), 7.29 (s, 1H), 7.20 (d, J=10.5 Hz, 1H), 7.03 (d, J=10.7 Hz, 1H), 6.97 (s, 1H), 4.48 (dt, J=12.9, 6.7 Hz, 1H), 4.00 (s, 3H), 3.96 (s, 3H), 3.66 (s, 3H), 2.79 (dd, J=13.3, 6.3 Hz, 1H), 2.48 (td, J=13.0, 7.1 Hz, 1H), 2.25-2.15 (m, 1H), 2.03-1.94 (m, 1H), 1.93 (s, 3H). ¹³C NMR (126 MHz, Acetone-d₆) δ 169.91, 165.42, 162.88, 161.07, 151.20, 150.96, 148.02, 135.41, 134.80, 131.54, 126.29, 114.14, 112.70, 108.00, 96.18, 62.30, 57.03, 56.65, 52.72, 35.87, 31.21, 30.40, 22.64. HRMS (ESI) m/z, calculated for [M+H]⁺: 395.1607; found: 395.1593.

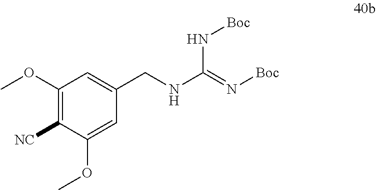

### Example 61: Synthesis of Compound 40b

A white solid of 40b (23.0 mg, 53.0% yield in 0.1 mmol scale) was obtained after solvent removal and silica column chromatograph (Hexanes/EA, 19/1-2/1). ¹H NMR (400 MHz, Chloroform-d) δ 11.49 (s, 1H), 8.69 (d, J=5.7 Hz, 1H), 6.55 (s, 2H), 4.59 (d, J=5.6 Hz, 2H), 3.89 (s, 6H), 1.49 (d, J=3.2 Hz, 18H). ¹³C NMR (101 MHz, Chloroform-d) δ 163.51, 162.88, 156.41, 153.41, 145.56, 114.03, 103.30, 90.65, 83.73, 79.71, 56.34, 45.10, 28.39, 28.16. HRMS (ESI) m/z, calculated for [M+H]⁺: 435.2244; found: 435.2242.

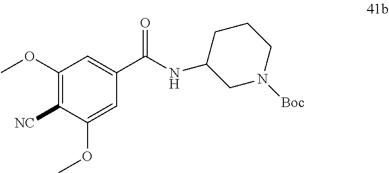

### Example 62: Synthesis of tert-butyl 3-(4-cyano-3,5-dimethoxybenzamido)piperidine-1-carboxylate (41b)

A white solid of 41b (31.9 mg, 82.0% yield in 0.1 mmol scale) was obtained after solvent removal and silica column chromatograph (Hexanes/EA, 9/1-2/1). ¹H NMR (500 MHz, Chloroform-d) δ 6.93 (s, 2H), 6.59 (s, 1H), 4.11 (dd, J=10.5, 4.2 Hz, 1H), 3.95 (s, 6H), 3.57 (d, J=23.8 Hz, 2H), 3.31 (s, 1H), 1.89 (d, J=3.1 Hz, 2H), 1.72-1.63 (m, 2H), 1.62-1.52 (m, 1H), 1.45 (s, 9H). ¹³C NMR (126 MHz, Chloroform-d) δ 165.77, 162.94, 141.16, 113.36, 102.66, 102.59, 94.34, 80.33, 56.91, 56.76, 56.61, 56.47, 48.62, 47.05, 46.92, 29.42, 28.60, 28.53, 22.57. HRMS (ESI) m/z, calculated for [M+H]⁺: 390.2029; found: 390.2021.

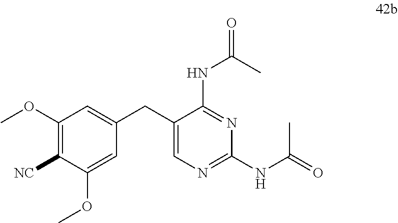

### Example 63: Synthesis of N,N′-(5-(4-cyano-3,5-dimethoxybenzyl)pyrimidine-2,4-diyl)diacetamide (42b)

A white solid of 42b (31.0 mg, 84.0% yield in 0.1 mmol scale) was obtained after work-up and purifications. Firstly, after solvent removal by vacuum drying, DCM (5 mL) was added in and followed by a filtration to remove the insoluble base additive. Then after removing the DCM, acetone (5 mL) was used to dissolve and remove the catalyst as well as byproducts after centrifuge to afford the white solid product. ¹H NMR (500 MHz, DMSO-d6) δ 10.44 (s, 1H), 10.17 (s, 1H), 8.40 (s, 1H), 6.62 (s, 2H), 3.95 (s, 2H), 3.83 (s, 6H), 2.16 (s, 3H), 2.14 (s, 3H).¹³C NMR (101 MHz, DMSO-d6) δ 170.14, 169.03, 161.93, 159.28, 157.84, 156.04, 148.18, 119.63, 113.98, 104.67, 87.74, 56.32, 34.48, 24.54, 23.99. HRMS (ESI) m/z, calculated for [M+H]+: 370.1515; found: 370.1507.

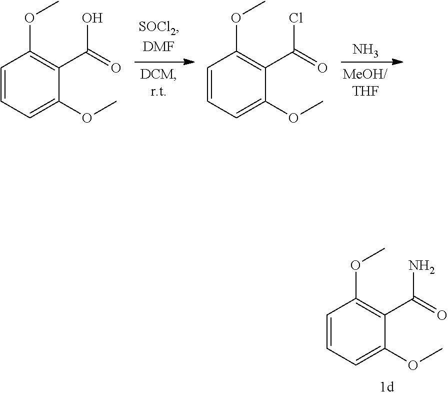

### Example 64: Synthesis of Compound 1d

The title compound was prepared as white powder in 94.0% (170 mg) isolated yield via reported procedures from literature in 1.0 mmol scale.²⁶ 2,6-dimethoxybenzamide (1d) 1H NMR (400 MHz, Chloroform-d) δ 7.31-7.26 (m, 1H), 6.57 (d, J=8.4 Hz, 2H), 5.81 (d, J=25.1 Hz, 2H), 3.84 (s, 6H). ¹³C NMR (101 MHz, Chloroform-d) δ 167.97, 157.55, 131.07, 114.98, 104.20, 56.18.

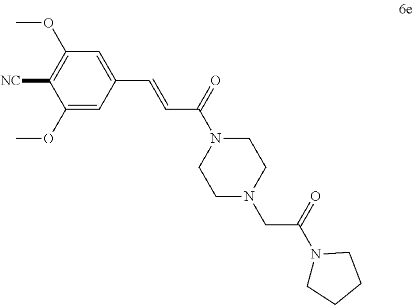

### Example 65: Synthesis of (E)-2,6-dimethoxy-4-(3-oxo-3-(4-(2-oxo-2-(pyrrolidin-1-yl)ethyl)piperazin-1-yl)prop-1-en-1-yl)benzonitrile (6e)

An off-white solid of 6e (155.1 mg, 37.6% total isolated in 1.0 mmol scale) was prepared by reported reactions and methods in reference.^{27 1}H NMR (400 MHz, Chloroform-d) δ 7.55 (d, J=15.4 Hz, 1H), 6.88 (d, J=15.4 Hz, 1H), 6.64 (s, 2H), 3.94 (s, 6H), 3.74 (d, J=39.9 Hz, 4H), 3.52-3.42 (m, 4H), 3.17 (s, 2H), 2.65 (s, 4H), 1.97 (p, J=6.5 Hz, 2H), 1.86 (p, J=6.8 Hz, 2H). ¹³C NMR (101 MHz, Chloroform-d) δ 167.60, 164.64, 162.81, 141.83, 141.49, 120.80, 113.89, 102.83, 77.48, 77.16, 76.84, 60.73, 56.47, 53.63, 53.26, 46.31, 46.00, 42.34, 26.35, 24.23. HRMS (ESI) m/z, calculated for [M+H]⁺: 413.2189; found: 413.2182.

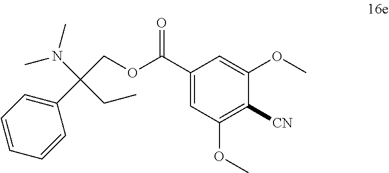

### Example 66: Synthesis of 2-(dimethylamino)-2-phenylbutyl 4-cyano-3,5-dimethoxy benzoate (16e)

A white powder of 16e (167.3 mg, 87.5% total yield in 0.5 mmol scale) was prepared by reported reactions and methods in reference.^{28 1}H NMR (500 MHz, Chloroform-d) δ 7.50-7.44 (m, 2H), 7.34 (t, J=7.7 Hz, 2H), 7.26-7.22 (m, 1H), 7.08 (s, 2H), 4.87 (d, J=11.9 Hz, 1H), 4.76 (d, J=11.9 Hz, 1H), 3.86 (s, 6H), 2.37 (s, 6H), 1.88 (ddt, J=36.5, 14.1, 7.2 Hz, 2H), 0.74 (t, J=7.4 Hz, 3H). ¹³C NMR (101 MHz, Chloroform-d) δ 165.08, 162.55, 142.05, 135.94, 128.04, 127.61, 126.75, 113.27, 104.54, 95.40, 77.48, 77.16, 76.84, 65.57, 64.99, 56.50, 39.75, 29.47, 8.68. HRMS (ESI) m/z, calculated for [M+H]⁺: 383.1971; found: 383.1964.

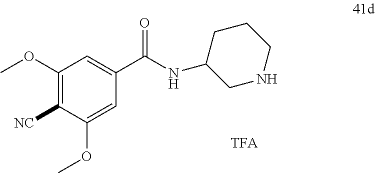

### Example 67: Synthesis of 4-cyano-3,5-dimethoxy-N-(piperidin-3-yl)benzamide (41d)

A yellow solid of 41d (29.5 mg, 99.0% yield with 30 mg 41b as the starting material) after blowing away the TFA (1 mL) and DCM (0.2 mL) residue with N₂ flow and vacuum drying. ¹H NMR (400 MHz, DMSO-d6) δ 9.01 (s, 1H), 8.80 (d, J=8.8 Hz, 1H), 8.74 (d, J=7.4 Hz, 1H), 7.19 (s, 2H), 4.15 (ddd, J=19.4, 11.8, 7.8 Hz, 1H), 3.95 (s, 6H), 3.35 (d, J=11.3 Hz, 1H), 3.24 (d, J=12.5 Hz, 1H), 2.86 (d, J=9.8 Hz, 2H), 1.94 (dd, J=25.9, 14.3 Hz, 3H), 1.76-1.55 (m, 3H). ¹³C NMR (101 MHz, DMSO-d6) δ 164.69, 161.91, 158.56, 158.23, 140.53, 113.43, 103.19, 91.98, 56.70, 46.06, 44.03, 42.98, 27.88, 20.85. ¹⁹F NMR (376 MHz, dmso) 6-73.99. HRMS (ESI) m/z, calculated for [M+H]⁺: 290.1505; found: 290.1498.

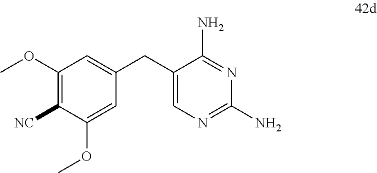

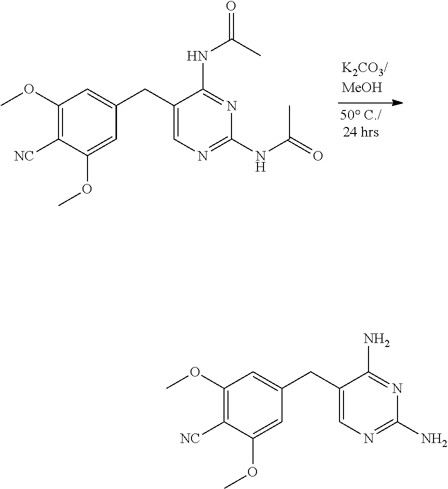 [SMILES: COc1cc(Cc2cnc(NC(C)=O)nc2NC(C)=O)cc(OC)c1C#N ; COc1cc(Cc2cnc(N)nc2N)cc(OC)c1C#N]

### Example 68: Synthesis of 4-((2,4-diaminopyrimidin-5-yl)methyl)-2,6-dimethoxy benzonitrile (42d)

A white solid of 42d (14.0 mg, 98.0% yield in 0.05 mmol scale) was obtained by solvent removal and filtration in acetonitrile (5 mL). The filtrate was finally dried over vacuum to afford the desired product. ¹H NMR (500 MHz, DMSO-d6) δ 7.58 (s, 1H), 6.70 (s, 2H), 6.16 (s, 2H), 5.74 (s, 2H), 3.83 (s, 6H), 3.63 (s, 2H).¹³C NMR (101 MHz, DMSO-d6) δ 162.39, 162.12, 161.86, 156.20, 156.08, 149.56, 114.11, 104.49, 104.40, 87.40, 56.33, 56.22, 33.66. HRMS (ESI) m/z, calculated for [M+H]+: 286.1304; found: 286.1296.

### Example 69: Scale Photocatalyzed Cyanation Reactions

The reactions performed by following the general procedure 3 for photocatalyzed cyanation.

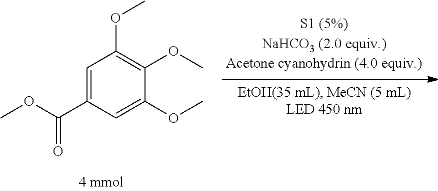

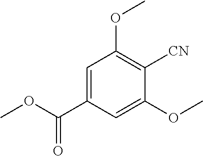

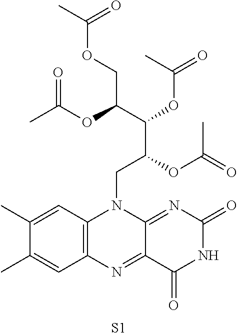 [SMILES: CC(=O)OC[C@H](OC(C)=O)[C@H](OC(C)=O)[C@@H](Cn1c2nc(=O)[nH]c(=O)c-2nc2cc(C)c(C)cc21)OC(C)=O ; "S1"]

| Reaction Time (hours) | HPLC Estimated Yield (%) |
|---|---|
| 20 | 32 |
| 28 | 51 |
| 45 | 63 |
| 60 | 72 |
| 72 | 89 |
| 96 | 97 |

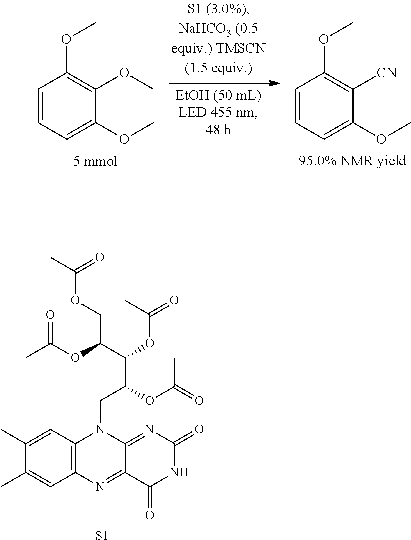 [SMILES: COc1cccc(OC)c1OC ; COc1cccc(OC)c1C#N ; CC(=O)OC[C@H](OC(C)=O)[C@H](OC(C)=O)[C@@H](Cn1c2nc(=O)[nH]c(=O)c-2nc2cc(C)c(C)cc21)OC(C)=O ; "S1"]

The reactions performed by following the general procedure 4 for photocatalyzed cyanation.

### Example 70: Attempted Unreactive Substrates for the Photocatalyzed Cyanation Reactions

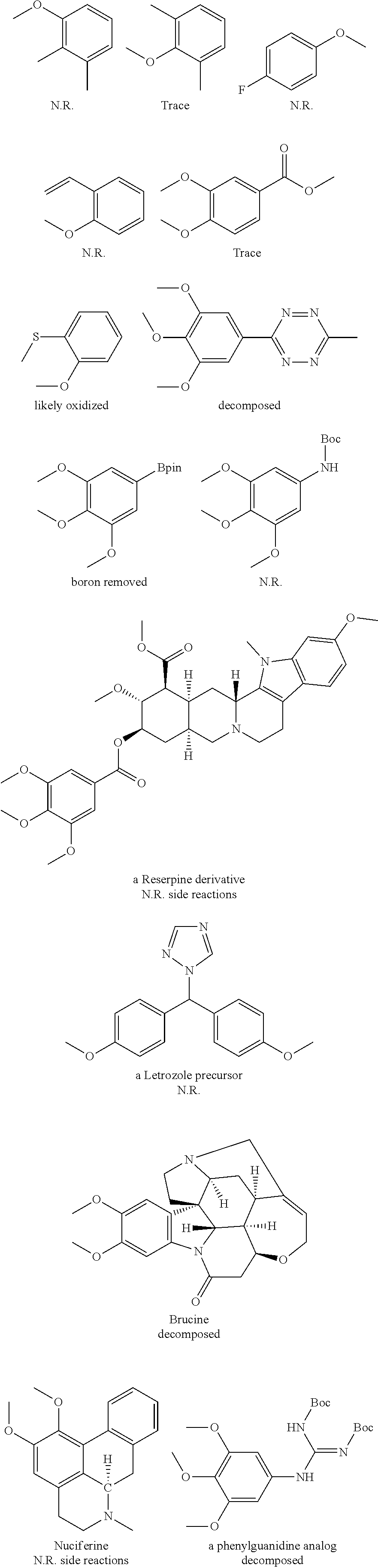

### Example 71: Reaction Inhabitation

The reactions performed by following the optimization procedure 1.

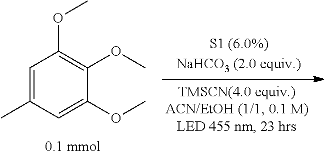

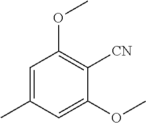

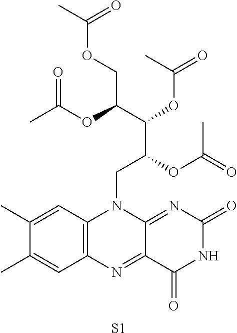 [SMILES: CC(=O)OC[C@H](OC(C)=O)[C@H](OC(C)=O)[C@@H](Cn1c2nc(=O)[nH]c(=O)c-2nc2cc(C)c(C)cc21)OC(C)=O ; "S1"]

| Entry | Condition variations | HPLC Estimated Yield (%) |
|---|---|---|
| 1 | None | 99.9 |
| 2 | Degassed for 10 min | 23.0 |
| | N₂ protected | |
| 3 | TEMPO (1.0 equiv.) | 99.0 (n = 2) |
| 4 | BHT (4 equiv.) | 52.1 |

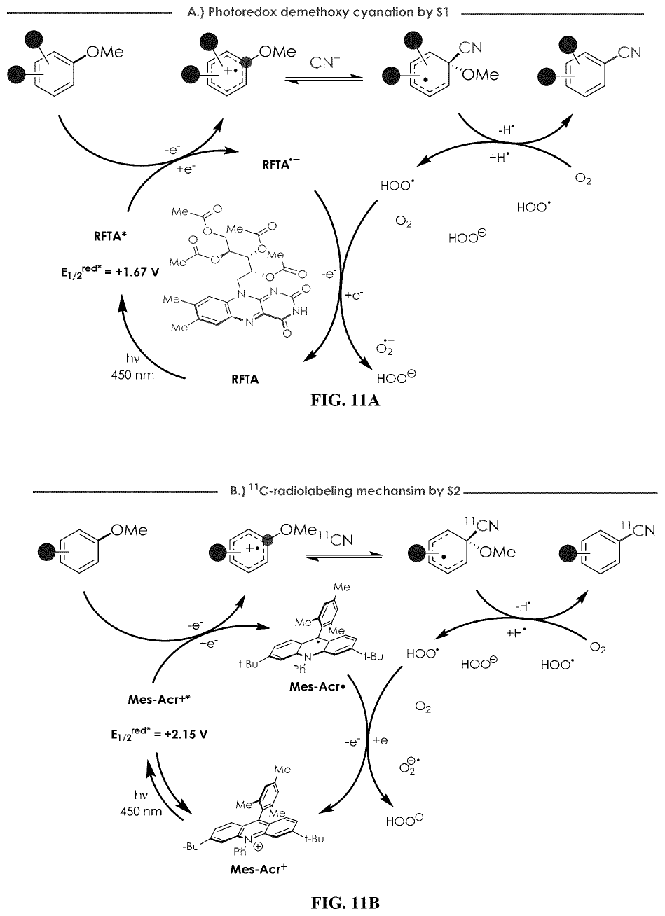

For proposed mechanism see FIGS. 11A and 11B.

### Example 72: Procedure and Data for [C]Nitrile Products Synthesis—General Procedure for Photocatalyzed ¹³C-Cyanation

With [¹³C]TMSCN. To a 4.32-dram (16 ml) vial that contained a Teflon-coated magnetic stir bar was added 0.02 mmol of S1 (10 mol %), NaHCO₃ (0.2 mmol, 1.0 equiv.) and arene (0.2 mmol, 1.0 equiv.). The reagent mixture was then attempted to be dissolved in EtOH (2.0 ml, 0.1 M). The vial was sealed with a PTFE-lined septum screw cap and stirred rapidly for approximately 5 min. [¹³C]TMSCN (0.4 mmol, 2.0 equiv.) was subsequently added. The vial was positioned on a stir plate approximately 5 cm from two blue LED lamps (Wolezek 36W LED, 450-460 nm) before stirring and irradiating for a designated time. The crude reaction mixture was then concentrated in vacuo and purified by normal-phase flash chromatography or reverse-phase flash liquid chromatography.

With K¹³CN. To a 4.32-dram (16 ml) vial that contained a Teflon-coated magnetic stir bar was added 0.02 mmol of S1 (10 mol %), NaHCO₃ (0.4 mmol, 2.0 equiv.) and arene (0.2 mmol, 1.0 equiv.). The reagent mixture was then attempted to be dissolved in EtOH/MeCN (1.0 ml/1.0 mL, 0.1 M). The vial was sealed with a PTFE-lined septum screw cap and stirred rapidly for approximately 5 minutes or K¹³CN (0.4-0.6 mmol, 2.0-3.0 equiv.) was subsequently added. The vial was positioned on a stir plate approximately 5 cm from two blue LED lamps (Wolezek 36W LED, 450-460 nm) before stirring and irradiating for a designated time. The crude reaction mixture was then concentrated in vacuo and purified by normal-phase flash chromatography.

### Characterization Data of Select Radiolabeled Compounds:

2,6-dimethoxy-4-methylbenzo-¹³C-nitrile ([¹³C]2b): HRMS (ESI) m/z, calculated for [M+H]⁺: 179.0902; found: 179.0898; ¹H NMR (400 MHz, Chloroform-d) δ 6.38-6.33 (m, 2H), 3.88 (s, 6H), 2.38 (s, 3H).

2,6-dimethoxybenzo-¹³C-nitrile ([¹³C]1b): HRMS (ESI) m/z, calculated for [M+H]⁺: 165.0745; found: 165.0742; ¹H NMR (400 Hz, Chloroform-d) δ 7.43 (t, 1H), 6.55 (dd, J=8.7 Hz, 2H), 3.91 (s, 6H).

Methyl 4-(cyano-¹³C)-3,5-dimethoxybenzoate ([¹³C]16b): HRMS (ESI) m/z, calculated for [M+H]⁺: 207.0851; found: 207.0846; ¹H NMR (400 Hz, Chloroform-d) of [¹³C]16b; δ 7.10 (s, 2H), 3.99 (s, 6H), 2.63 (s, 3H).

(S)—N-(2-(cyano-¹³C)-1,3,10-trimethoxy-9-oxo-5,6,7,9-tetrahydrobenzo[a]heptalen-7-yl)acetamide: HRMS (ESI) m/z, calculated for [M+H]⁺: 396.1641; found: 396.1639.

N,N′-(5-(4-(cyano-¹³C)-3,5-dimethoxybenzyl)pyrimidine-2,4-diyl)diacetamide ([¹³C]42b): HRMS (ESI) m/z, calculated for [M+Na]⁺: 393.1368; found: 393.1363. ¹H NMR (400 MHz, DMSO-d6): δ 10.36 (s, 2H), 8.33 (s, 1H), 6.64 (d, J=1.6 Hz, 2H), 5.75 (s, 1H), 3.92 (s, 2H), 3.83 (s, 6H), 2.16 (s, 3H), 2.11 (s, 3H).

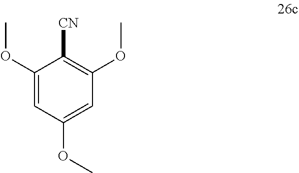

### Example 73: Photocatalytic Arene C—H Cyanation-Synthesis of 2,4,6-trimethoxybenzonitrile (26c)

The procedure and reaction set-up were the same as general procedure 1 for photocatalyzed cyanation. ¹H NMR (400 MHz, Chloroform-d) δ 7.26 (s, 1H), 6.06 (s, 7H), 3.87 (s, 3H), 3.85 (s, 1H).

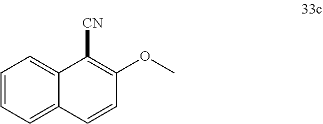

### Example 74: Synthesis of 2-methoxy-1-naphthonitrile (33c)

The procedure and reaction set-up were the same as general procedure 1 for photocatalyzed cyanation. ¹H NMR (500 MHz, Chloroform-d) δ 8.08 (d, J=8.4 Hz, 1H), 8.02 (d, J=9.2 Hz, 1H), 7.82 (d, J=8.2 Hz, 1H), 7.63 (t, 1H), 7.44 (t, 1H), 7.26 (d, J=9.2 Hz, 1H), 4.06 (s, 3H). ¹³C NMR (126 MHz, Chloroform-d) δ 161.75, 135.14, 133.69, 129.29, 128.58, 128.10, 125.19, 124.15, 115.81, 112.14, 95.34, 56.73. HRMS (ESI) m/z, calculated for [M+H]⁺: 184.0762; found: 184.0755.

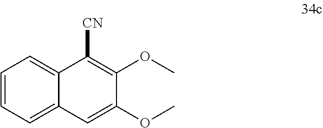

### Example 75: Synthesis of 2,3-dimethoxy-1-naphthonitrile (34c)

The procedure and reaction set-up were the same as general procedure 1 for photocatalyzed cyanation. 1H NMR (400 MHz, Chloroform-d) δ 8.07-8.03 (m, 1H), 7.77-7.72 (m, 1H), 7.55-7.46 (m, 2H), 7.36 (s, 1H), 4.17 (s, 3H), 4.00 (s, 3H). ¹³C NMR (101 MHz, Chloroform-d) δ 154.80, 151.18, 130.36, 127.96, 127.11, 126.71, 126.55, 124.51, 115.38, 112.44, 102.00, 62.19, 56.17. HRMS (ESI) m/z, calculated for [M+H]⁺: 214.0868; found: 214.0862.

### Example 76: Radiolabeling Experiments

### Reagents and Equipment Information

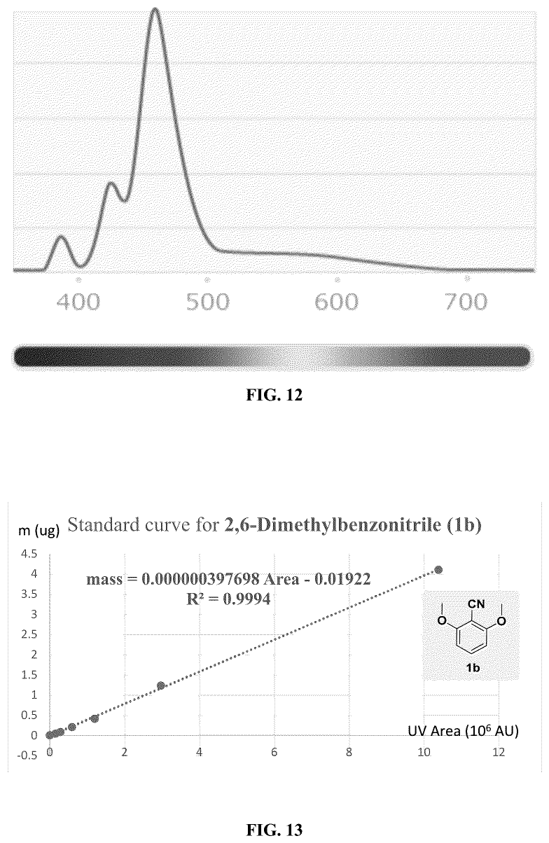

All chemicals are ACS reagent grade and used without further purification. Tetrabutylammonium hydroxide solution (54-56%, w/w) was purchased from sigma-Aldrich. Anhydrous ethanol was the Koptec's Pure Ethanol 200 Proof purchased from Fisher Scientific. TBAOH aqueous ethanol additive solution was prepared with 6.0 μL 54-56% TBAOH aqueous solution and 300 μL pure ethanol. The 450 nm blue diode laser (MDL-D-450, 450 nm, the power rating was set to 3.5W after fiber coupling) used for the labeling reaction was purchased from Changchun New Industries Optoelectronics Tech. Co., Ltd. The blue LED lamp (Kessil A160WE TUNA BLUE, 40W) was purchased from Kessil. The irradiation wavelengths observed are centered around 456 nm, 390 nm and 427 nm, with the major irradiation peak centered around 456 nm. ¹¹C activity was counted using a CRC-25 PET detector from Capintec. The reaction was timed with a stopwatch on cellphone. High-performance liquid chromatography (HPLC) was accomplished on a SHIMADZU chromatography system (Model CBM-20A) and analyzed using LabSolutions software. The λ absorbance detector and the model 2200 scaler ratemeter radiation detector were added to the HPLC system. The PET/CT imaging was acquired by Sedecal Super Argus 4R PET/CT instrument in Small Animal Imaging Facility of the Biomedical Research Imaging Center at the University of North Carolina at Chapel Hill. See FIG. 12.

### II. General Procedure for the Preparation of [¹¹C]TBACN

[¹¹C]CN—was firstly produced in form of [¹¹C]HCN after cyclotron bombardment followed by a two-step conversion.^{29,30} [¹¹C]TBACN(tetrabutylammonium cyanide)-TBAOH(tetrabutylammonium hydroxide) solution was obtained by delivering the gaseous [¹¹C]HCN into the TBAOH aqueous ethanol solution (0.65%, made with 6.0 μL 54-56% TBAOH aqueous solution and 500 μL pure ethanol) in a 5 mL V vial which sealed with an aluminum-rubber crimp cap and an outlet connected to the gaseous radio waste trap. The delivery gas flow usually lasted 10 minutes before the obtained [¹¹C]TBACN-TBAOH solution (typically 13 GBq to 18.5 GBq) which was finally aliquoted (15 μL-60 μL) into 5 mL V vials for the labeling reactions.

### III. General HPLC Conditions

General HPLC conditions for crude reaction analysis (Radiochemical conversion calculation and co-injection)

Column: Phenomenex, Kinetex® 5 m EVO C18 100 Å, 250×4.6 mm LC Column.

Condition A: Solvent A: 0.1% TFA water; Solvent B: 0.1% TFA acetonitrile. Isocratic elution at x % solvent B. Flow rate: 1 mL/minute. Injection volume: 1 mL (with 5% acetic acid solution).

Condition B: Solvent A: 10 mM COONH₄ water; Solvent B: acetonitrile. Isocratic elution at x % solvent B. Flow rate: 1 mL/minute. Injection volume: 1 mL (with water).

Condition C: Column: Phenomenex, Kinetex® 5 m F5 100 Å, 250×4.6 mm LC Column. Solvent A: 0.1% TFA water; Solvent B: 0.1% TFA acetonitrile; Isocratic elution at x % solvent B. Flow rate: 1 mL/minute. Injection volume: 1 mL (with 5% acetic acid solution).

### General HPLC Conditions for Quality Control Test and Co-Injection Test

Condition D: Column: Phenomenex, Kinetex® 5 m F5 100 Å, 250×4.6 mm LC Column. Solvent A: 0.1% TFA water; Solvent B: 0.1% TFA acetonitrile; Isocratic elution at x % solvent B. Flow rate: 1 mL/minute. Injection volume: 1 mL (with 5% acetic acid solution).

Condition E: Column: Phenomenex, Kinetex® 5 m EVO C18 100 Å, 250×4.6 mm LC Column. Solvent A: 0.1% TFA water; Solvent B: 0.1% TFA acetonitrile. Isocratic elution at x % solvent B. Flow rate: 1 mL/minute. Injection volume: 1 mL (with 5% acetic acid solution).

### Example 77: General Procedure for the Photocatalytic ¹¹C-Cyanation—General Procedure 1 by Catalyst S1 (RFTA) with a Laser or an LED Lamp

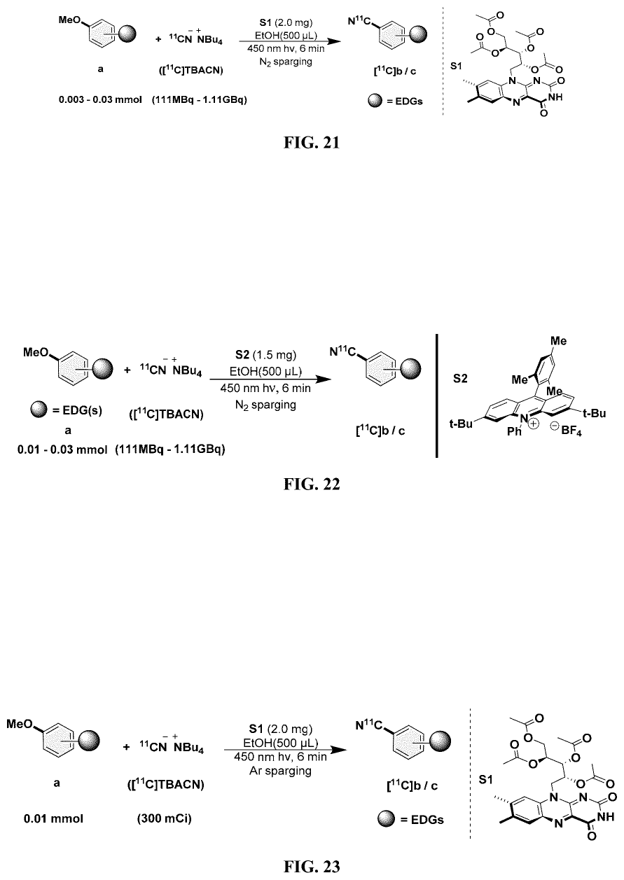

Follow a modified procedure from the photocatalytic fluorination reaction⁶⁸. The substrate (0.002-0.05 mmol) and photocatalyst (S1, 2.0 mg, FIG. 21; S2, 1.5 mg, FIG. 22) were weighed into a 1.5 mL Eppendorf Tube® and transferred with pure EtOH (500 μL) into a 5 mL V vial via pipette. TBAOH aqueous ethanol additive solution (1.3%, v/v, to compensate the volume of the [¹¹C]TBACN-TBAOH solution to 25 μL-30 μL) were then added to the V vial if needed, and 1 minute of ultra-sonication could help dissolving the substrate if necessary. Then, a 15-30 μL aliquot of [¹¹C]TBACN-TBAOH in EtOH (typically 111MBq-1.11GBq) was added to the reaction vial via pipette. The reaction V vial was then fixed on an aluminum block. A needle connected to an N₂ filled balloon was inserted to the bottom of the V vial and the reaction medium was continuously sparged throughout the entire reaction time. The reaction was then irradiated top-down with a laser (MDL-D-450, 450 nm, 3.5W after fiber coupling) or an A160WE Tuna Blue Kessil LED lamp for 6 minutes. The light source was turned off, the needle and balloon were removed and an aliquot of the reaction mixture (typically 7.4-37MBq) was taken for radio HPLC analysis.

### Example 78: General Procedure for the Photocatalytic ¹¹C-Cyanation—General Procedure 2 by Catalyst S2 (Mes-Acr-BF4⁺) with a Laser or an LED Lamp

The substrate (0.01-0.05 mmol) and photocatalyst (S2, 1.5 mg, FIG. 23) were weighed into a 1.5 mL Eppendorf Tube® and transferred with pure EtOH (500 μL) into a 5 mL V vial via pipette. TBAOH aqueous ethanol additive solution (1.3%, v/v, to compensate the volume of the [¹¹C]TBACN-TBAOH solution to 40-50 μL) were added to the V vial if needed, and 1 minute of ultra-sonication could help dissolving the substrate if necessary. Then, a 10-60 μl aliquot of [¹¹C]TBACN-TBAOH in EtOH (typically 111MBq-1.11GBq) was added to the reaction vial via pipette. The reaction V vial was then fixed on an aluminum block. A needle connected to an N₂ filled balloon was inserted to the bottom of the V vial and the reaction medium was continuously sparged throughout the entire reaction time. The reaction was then irradiated top-down with a laser (MDL-D-450, 450 nm, 3.5W after fiber coupling) or an A160WE Tuna Blue Kessil LED lamp for 6 minutes. The light source was turned off, the needle and balloon were removed and an aliquot of the reaction mixture (typically 7.4-37MBq) was taken for radio HPLC analysis.

### Example 79: General Procedure for the Photocatalytic ¹¹C-Cyanation—General Procedure 3 (High Radioactivity, >1.85GBq) Catalyst S1 (RFTA) with a Laser

The substrate (0.01 mmol) and photocatalyst (S1, 2.0 mg) were weighed into a 5 mL V vial A. 2.0 μL 54-56% TBAOH aqueous solution was then diluted with 300 pure ethanol in another 5 mL V vial B. In a hot cell, the V vial B, which was sealed with an aluminum-rubber crimp cap and an outlet connected to the gaseous radio waste trap, was then fixed on an aluminum block. The gaseous [¹¹C]HCN was delivered into the TBAOH aqueous ethanol solution. The delivery gas flow usually lasted 8 minutes before the obtained [¹¹C]TBACN-TBAOH solution (typically about 13GBq), which was then transferred into the V vial A with a syringe. 350 μL pure ethanol was then injected into V vial B to rinse the remaining ¹¹C-radioactivity before injecting them into the V vial A and the syringe was pulled up and down for several times to help mixing and dissolving the substrate and catalyst. Then V vial A was measured by a dose calibrator in the hot cell to determine the radioactivity (1.11GBq). Then the V vial A was then fixed on an aluminum block under a laser. A needle connected to an Argon stream line was inserted to the bottom of the V vial (uncapped) and the reaction medium was continuously sparged throughout the entire reaction time. The reaction was then irradiated top-down with a laser (MDL-D-450, 450 nm, 3.5W after fiber coupling) for 6 minutes. The laser was turned off, the needle was removed and an aliquot to all of the reaction mixture was taken for radio HPLC analysis or preparative separation.

### Example 80: General Procedure for the Photocatalytic ¹¹C-Cyanation—General Procedure 4 by Catalyst S1 (RFTA) with a Continuous Flow Device and an LED Lamp

The substrate (0.01-0.03 mmol) and photocatalyst (S1, 2.0 mg) were weighed into a 1.5 mL Eppendorf Tube® and transferred (with solvent when the substrate is liquid or oil) into a 5 mL V vial via pipette. Pure EtOH (500 μL) and TBAOH aqueous ethanol additive solution (1.3%, v/v, to compensate the volume of the [¹¹C]TBACN-TBAOH solution to 25 μL-30 μL) were sequentially added to the V vial, 1 minute of ultra-sonication could be applied to dissolve the substrate better. Then a 15-30 μL aliquot of [¹¹C]TBACN-TBAOH in EtOH (typically 111MBq-1.11GBq) was added to the reaction vial via pipette. The reaction V vial was then capped with an aluminum-rubber crimp cap. Then it was installed in between a syringe pump and the flow device (˜800 μL volume). The reaction solution was pushed to the starting point of the flow device with the syringe. The flow rate was set to be 0.13 mL/minute and the syringe pump was started with the syringe on it. An A160WE Tuna Blue Kessil LED lamp was turned on. After all the reaction solution had flowed through the flow device, the LED lamp was turned off and an aliquot of the reaction mixture (typically 7.4-29.6MBq) was taken for radio HPLC analysis. 4

### Example 81: General Procedure for the Photocatalytic ¹¹C-Cyanation—General Procedure 5 by Catalyst S2 (Mes-Acr⁻BF4⁺) with a Continuous Flow Device and an LED Lamp

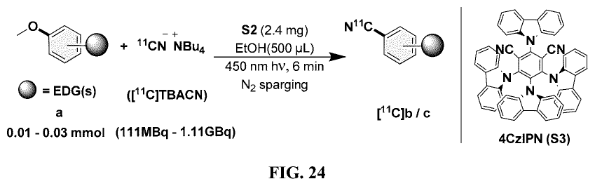

The substrate (0.01-0.03 mmol) and photocatalyst (S2, 1.5 mg, FIG. 24) were weighed into a 1.5 mL Eppendorf Tube® and transferred (with solvent when the substrate is liquid or oil) into a 5 mL V vial via pipette. Pure EtOH (500 μL) and TBAOH aqueous ethanol additive solution (1.3%, v/v, to compensate the volume of the [¹¹C]TBACN-TBAOH solution to 40-50 μL) were sequentially added to the V vial, 1 minute of ultra-sonication could be applied to dissolve the substrate better. Then a 15-60 μL aliquot of [¹¹C]TBACN-TBAOH in EtOH (typically 111MBq-1.11GBq) was added to the reaction V vial via pipette. The reaction V vial was then capped with an aluminum-rubber crimp cap. Then it was installed in between a syringe pump and the flow device (˜800 μL volume) with lines and needles. The reaction solution was pushed to the starting point of the flow device with the syringe. The flow rate was set to be 0.13 mL/minute and the syringe pump was started with the syringe on it. An A160WE Tuna Blue Kessil LED lamp was turned on. After all the reaction solution had flowed through the flow device, the LED lamp was turned off and an aliquot of the reaction mixture (typically 7.4-29.6MBq) was taken for radio HPLC analysis.

### Example 82: General Procedure for the Photocatalytic ¹¹C-Cyanation—General Procedure 6 by Catalyst S3 (4CzIPN) with a Laser

The substrate (0.01-0.05 mmol) and photocatalyst (S3, 2.5 mg) were weighed into a 1.5 mL Eppendorf Tube® and transferred with pure EtOH (500 μL) into a 5 mL V vial via pipette. TBAOH aqueous ethanol additive solution (1.3%, v/v, to compensate the volume of the [¹¹C]TBACN-TBAOH solution to 40-50 μL) were added to the V vial if needed, and 1 minute of ultra-sonication could help with dissolving the substrate if necessary. Then a 10-60 μL aliquot of [¹¹C]TBACN-TBAOH in EtOH (typically 111MBq-1.11GBq) was added to the reaction vial via pipette. The reaction V vial was then fixed on an aluminum block. A needle connected to an N₂ filled balloon was inserted to the bottom of the V vial and the reaction medium was continuously sparged throughout the entire reaction time. The reaction was then irradiated top-down with a laser (MDL-D-450, 450 nm, 3.5W after fiber coupling) for 6 min. The light source was turned off, the needle and balloon were removed and an aliquot of the reaction mixture (typically 7.4-37MBq) was taken for radio HPLC analysis.

### Example 83: General Procedure for the Photocatalytic ¹¹C-Cyanation—General Procedure of the Radio HPLC Analysis

The activity injected into HPLC was measured (this activity was denoted by a) and the time was recorded. The fraction corresponding to radiolabeled product was collected and the activity was measured (this activity was denoted by 3) and the time was recorded. The decay corrected p could be calculated from the recorded isolation time of each substrate. The radiochemical conversion (RCC) was obtained by dividing R by the decay corrected a. For most cases, the identity of the radiolabeled compound was confirmed by overlap the radio trace peak with the commercial or synthesized ¹³CN standard UV peak via HPLC.

Due to the limited time and radio activity scales when running the ¹¹C-labelling reactions, it's very difficult to run all the quality controls for every purified ¹¹C-labelled aryl nitrile product. For most of the labelling experiments, we managed to use the same C18 column on the same HPLC to conduct the isolation and analysis. Given the fact that the reaction works very well in normal ¹²C-cyanation, it would be very convincing that if the radio peaks of the ¹¹C-labelled aryl nitriles matched with the UV peaks of the authentic aryl [¹²C]nitrile product standards. Only in a few cases the products were isolated and confirmed with quality control. In a large portion of cases co-injection was carried out when doing the isolation and RCC analysis after reaction by mix the corresponding aryl [¹²C]nitrile product standard(s) into the aliquot from the crude reaction mixture. Consequently, we were able to confirm all the ¹¹C-labelled aryl nitrile products that, similarly as the [¹²C]nitriles, ranged from simple aromatics ([¹¹C]1b-[¹¹C]37b) to complex molecules ([¹¹C]38b-[¹¹C]42b) with the aryl nitrile core and potential bioactivities.

In some cases, co-injection of the reaction mixture above of the labeled compound with commercial or synthesized ¹³CN standard via HPLC was also used to further confirm the identity of the radiolabeled compound.

In a few cases, co-injection of the purified ¹¹CN-labeled compound with commercial or synthesized ¹³CN standard via HPLC was also used to further confirm the identity of the radiolabeled compound.

RCC (decay corrected or none decay corrected) was reported as average±SD % of triplicated reactions unless otherwise noted.

### Example 84: Radio Reaction Optimization-Reaction Time Study

The substrate (0.03 mmol) and photocatalyst (S1, 2.5 mg) were weighed into a 1.5 ml 5 Eppendorf Tube® and transferred with pure EtOH (500 μL) into a 5 mL V vial via pipette. TBAOH aqueous ethanol additive solution (1.3%, v/v, to compensate the volume of the [¹¹C]TBACN-TBAOH solution to 25 μL) were then added to the V vial if needed, and 1 minute of ultra-sonication could help dissolving the substrate if necessary. Then a 15-30 μL aliquot of [¹¹C]TBACN-TBAOH in EtOH (typically 5-15 mCi) was added to the reaction vial via pipette. The reaction V vial was then fixed on an aluminum block. A needle connected to an N₂ filled balloon was inserted to the bottom of the V vial and the reaction medium was continuously sparged throughout the entire reaction time. The reaction was then irradiated top-down with a laser (MDL-D-450, 450 nm, 3.5W after fiber coupling) for x minutes. The laser was turned off and an aliquot of the reaction mixture (typically 7.4-37MBq) was taken for radio HPLC analysis. The laser was then turned on again in order to continue the reaction for the next reaction time point study.

| 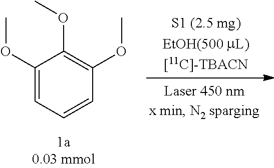 | | |
|---|---|---|
| 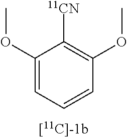 | | |
| 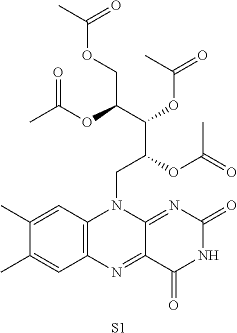 [SMILES: CC(=O)OC[C@H](OC(C)=O)[C@H](OC(C)=O)[C@@H](Cn1c2nc(=O)[nH]c(=O)c-2nc2cc(C)c(C)cc21)OC(C)=O ; "S1"] | | |
| Entry | x (min) | RCC (%) |
| 1 | 2 | 81.5 |
| 2 | 4 | 90.8 |
| 3 | 6 | 93.6 |
| 4 | 8 | 92.2 |

| 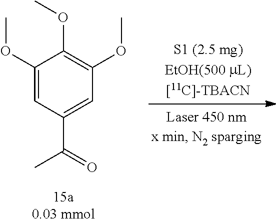 | | |
|---|---|---|
| 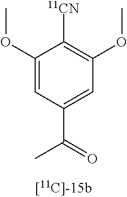 | | |
| 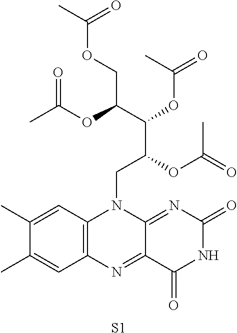 [SMILES: CC(=O)OC[C@H](OC(C)=O)[C@H](OC(C)=O)[C@@H](Cn1c2nc(=O)[nH]c(=O)c-2nc2cc(C)c(C)cc21)OC(C)=O ; "S1"] | | |
| Entry | x (min) | RCC (%) |
| 1 | 2 | 50.0 |
| 2 | 4 | 60.8 |
| 3 | 6 | 68.2 |
| 4 | 8 | 65.9 |

### Example 85: Radio Reaction Optimization-Catalyst Loading Amount Study

The substrate (0.03 mmol) and photocatalyst (S1, x mg) were weighed into a 1.5 ml Eppendorf Tube® and transferred with pure EtOH (500 μL) into a 5 mL V vial via pipette. TBAOH aqueous ethanol additive solution (1.3%, v/v, to compensate the volume of the [¹¹C]TBACN-TBAOH solution to 25 μL-30 μL) were then added to the V vial if needed, and 1 minute of ultra-sonication could help dissolving the substrate if necessary. Then a 15-30 μl aliquot of [¹¹C]TBACN-TBAOH in EtOH (typically 5-15 mCi) was added to the reaction vial via pipette. The reaction V vial was then fixed on an aluminum block. A needle connected to an N₂ filled balloon was inserted to the bottom of the V vial and the reaction medium was continuously sparged throughout the entire reaction time. The reaction was then irradiated top-down with a laser (MDL-D-450, 450 nm, 3.5W after fiber coupling) for 6 minutes.

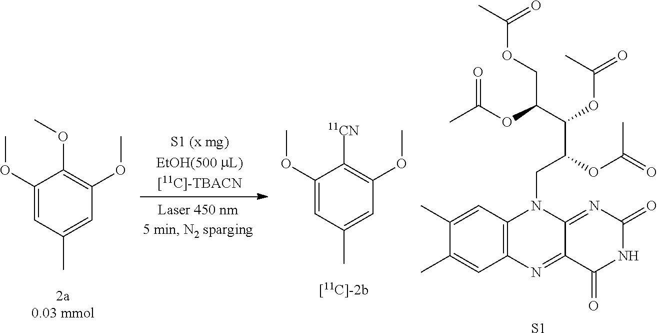

| Entry | RFTA (x mg) | RCC (%) |
|---|---|---|
| 1 | 1.0 | 84.0 |
| 2 | 1.5 | 84.4 |
| 3 | 2.0 | 93.9 |
| 4 | 2.5 | 88.0 |
| 5 | 3.0 | 80.1 |

### Example 86: Radio Reaction Optimization—Radio HPLC Analysis and Characterization of [¹¹C]Cyanide Labeled Arenes—Compound [¹¹C]1b

All ¹¹C-labelling reactions were performed according to general procedures at section 5.4 unless otherwise noted. For each labeling reaction, starting activity ([¹¹C]TBACN), injected and collected activities, isolation time, decay corrected injection activity, calculated radiochemical conversion (RCC), and calculated radiochemical yield (RCY) are summarized in a table for each substrate and procedure. All ¹¹C-labeled products were analyzed and characterized according to the general HPLC conditions listed in section 5.3. Representative crude radio-HPLC traces, HPLC traces of purification, and co-injection were listed if applicable.

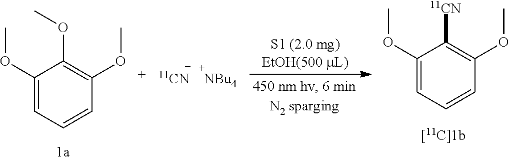

| | | | | | | | | Radio- | Radio- |
|---|---|---|---|---|---|---|---|---|---|
| | | | Activity | | | | Decay | chemical | chemical |
| | | Substrate | ([¹¹C] | Injected | Collected | Isolation | corrected | conversion | Isolation yield |
| Reaction | | Loading | TBACN) | dose | dose | Time | Injection | (RCC, | (RCY, |
| Entry | Substrate | (n, mmol) | (mCi) | (μCi) | (μCi) | (min) | Dose (μCi) | %, d.c.) | %, n.d.c) |
| 1 | 1a | 0.002 | 19.8 | 3093 | 1760 | 11 | 2128 | 85.6 | 56.9 |
| 2 | | 0.004 | 23 | 323 | 200 | 11 | 222 | 90.1 | 61.9 |
| 3 | | 0.004 | 14.8 | 727 | 436 | 12 | 483.5 | 90.2 | 60.0 |
| 4 | | 0.004 | 11.7 | 530 | 370 | 8 | 404 | 91.7 | — |
| 5 | | 0.004 | 23 | 605 | 43 | 8 | 46 | 93.6 | — |
| 6 | | 0.005 | 21.5 | 3335 | 1970 | 12 | 2218 | 88.8 | 59.1 |
| 7 | | 0.006 | 29.4 | 3860 | 2340 | 11 | 2656 | 88.1 | 60.6 |
| 8 | | 0.006 | 18.5 | 138 | 87 | 11 | 95 | 92.0 | 63.0 |
| 9 | | 0.006 | 14.5 | 662 | 487 | 8 | 504 | 96.5 | — |
| 10 | | 0.01 | 8.8 | 521 | 356 | 8 | 397 | 89.6 | — |
| 11 | | 0.03 | 22 | 876 | 600 | 8 | 667.5 | 90.0 | — |
| 12 | | 0.03 | 3.6 | 191 | 128 | 8 | 145.5 | 91.0 | — |
| 13 | | 0.03 | 13.5 | 582 | 415 | 8 | 443.5 | 93.6 | — |

Average RCC: 90.8 ± 2.8% (N = 12, n > = 0.004 mmol, decay corrected);
Average RCY: 60.3 ± 2.1% (N = 6, none decay corrected)

### Example 87: Radio Reaction Optimization—Radio HPLC Analysis and Characterization of [¹¹C]Cyanide Labeled Arenes—Compound [¹¹C]1d

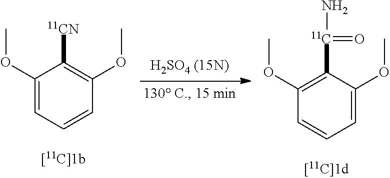

| | | | | | | Decay | |
|---|---|---|---|---|---|---|---|
| | | Activity | Injected | Collected | Isolation | corrected | Radiochemical |
| Reaction | | ([¹¹C]-1b) | dose | dose | time | injection | conversion |
| Entry | Substrate | (mCi) | (μCi) | (μCi) | (min) | dose (μCi) | (RCC, %, d.c.) |
| 1 | [¹¹C]1b | 2.56 | 222 | 119 | 6 | 181.0 | 65.7 |
| 2 | | 2.31 | 126 | 66 | 9 | 92.8 | 71.1 |
| 3 | | 1.97 | 125 | 94 | 5 | 105.4 | 89.2 |

Average RCC: 75.3 ± 12.3 (N = 3, decay corrected)

### HPLC-isolated RCCs of [¹¹C]1d

### Example 88: Radio Reaction Optimization—Radio HPLC Analysis and Characterization of [¹¹C]Cyanide Labeled Arenes—Compound [¹¹C]1d

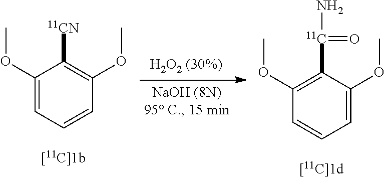

| | | Activity | | | | Decay | |
|---|---|---|---|---|---|---|---|
| | | ([¹¹C]- | Injected | Collected | Isolation | corrected | Radiochemical |
| Reaction | | 1b) | dose | dose | time | Injection | conversion |
| Entry | Substrate | (mCi) | (μCi) | (μCi) | (min) | dose (μCi) | (RCC, %, d.c.) |
| 1 | [¹¹C]1b | 1.5 | 180 | 106 | 6 | 146.8 | 72.2 |

RCC: 72.2% (decay corrected)

### HPLC-analysis RCCs of [¹¹C]1d

### Example 89: Radio Reaction Optimization—Radio HPLC Analysis and Characterization of [¹¹C]Cyanide Labeled Arenes—Compound [¹¹C]1d

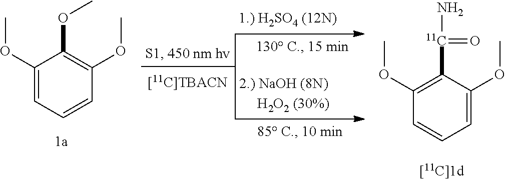

| | | | | | | | Decay | Radio- |
|---|---|---|---|---|---|---|---|---|
| | | | Activity | | | | corrected | chemical |
| | | Substrate | ([¹¹C] | Injected | Collected | Isolation | Injection | conversion |
| Reaction | | Loading | TBACN) | dose | dose | time | dose | (RCC, %, |
| Entry | Substrate | (n, mmol) | (mCi) | (μCi) | (μCi) | (min) | (μCi) | d.c.) |
| 1 | 1a | 0.007 | 22.0 | 196 | 132 | 6 | 159.8 | 82.6 |
| (acidic) | | | | | | | | |
| 2 | | 0.006 | 14.2 | 257 | 168 | 6 | 209.5 | 80.2 |
| (acidic) | | | | | | | | |
| 3 | | 0.01 | 23.2 | 124 | 41 | 7 | 97.7 | 42.0 |
| (basic) | | | | | | | | |

Average RCC (acidic): 81.4 ± 1.7% (N = 2, decay corrected);
RCC (basic): 42.0% (decay corrected)

### HPLC-isolated RCCs of [¹¹C]1d

### Example 90: Radio Reaction Optimization—Radio HPLC Analysis and Characterization of [¹¹C]Cyanide Labeled Arenes—Compound [¹¹C]1b

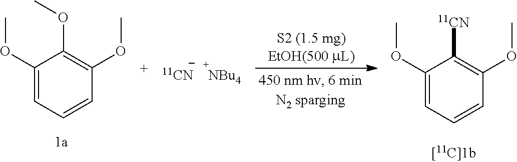

| | | | Activity | | | | Decay | |
|---|---|---|---|---|---|---|---|---|
| | | Substrate | ([¹¹C] | Injected | Collected | Isolation | corrected | Radiochemical |
| Reaction | | Loading | TBACN) | dose | dose | time | Injection | conversion |
| Entry | Substrate | (mmol) | (mCi) | (μCi) | (μCi) | (min) | dose (μCi) | (RCC, %, d.c.) |
| 1 | 1a | 0.03 | 2.7 | 167 | 60 | 9 | 123 | 49.0 |
| 2 | | 0.03 | 12.4 | 513 | 164 | 9 | 377.8 | 43.4 |
| 3 | | 0.03 | 5.5 | 355 | 132 | 8 | 270.5 | 48.8 |

Average RCC: 47.1 ± 3.2% (N = 3, decay corrected)

### HPLC-isolated RCCs of [¹¹C]1b by S2

### Example 91: Radio Reaction Optimization—Radio HPLC Analysis and Characterization of [¹¹C]Cyanide Labeled Arenes—Compound [¹¹C]1b

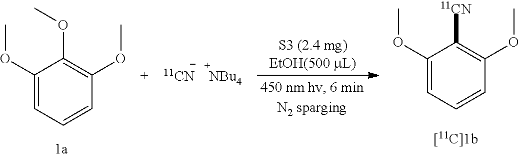

| | | | | | | | Decay | |
|---|---|---|---|---|---|---|---|---|
| | | | Activity | | | | corrected | |
| | | Substrate | ([¹¹C] | | | Isolation | Injection | Radiochemical |
| Reaction | | Loading | TBACN) | Injected | Collected | time | dose | conversion |
| Entry | Substrate | (mmol) | (mCi) | dose (μCi) | dose (μCi) | (min) | (μCi) | (RCC, %, d.c.) |
| 1 | 1a | 0.006 | 17.3 | 635 | 319 | 11 | 436.9 | 73.0 |

RCC: 73.0% (decay corrected)

### HPLC-isolated RCCs of [¹¹C]1b by S3

### Example 92: Radio Reaction Optimization—Radio HPLC Analysis and Characterization of [¹¹C]Cyanide Labeled Arenes—Compound [¹¹C]1b

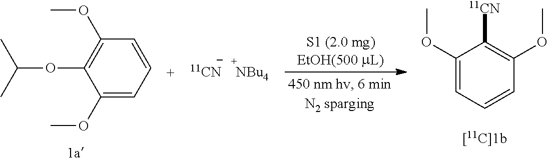

| | | | Activity | | | | Decay | |
|---|---|---|---|---|---|---|---|---|
| | | Substrate | ([¹¹C] | Injected | Collected | Isolation | corrected | Radiochemical |
| Reaction | | Loading | TBACN) | dose | dose | time | Injection | conversion |
| Entry | Substrate | (mmol) | (mCi) | (μCi) | (μCi) | (min) | dose (μCi) | (RCC, %, d.c.) |
| 1 | 1a′ | 0.01 | 10.4 | 569 | 395 | 7 | 448.5 | 88.0 |
| 2 | | 0.01 | 16.5 | 597 | 394 | 7 | 470.5 | 83.7 |
| 3 | | 0.01 | 19.4 | 2460 | 1505 | 11 | 1692.5 | 88.9 |

Average RCC: 86.9 ± 2.8% (N = 3, decay corrected)

HPLC-isolated RCCs of [¹¹C]1b from 1a′ by S1

### Example 93: Radio Reaction Optimization—Radio HPLC Analysis and Characterization of [¹¹C]Cyanide Labeled Arenes—Compound [¹¹C]2b

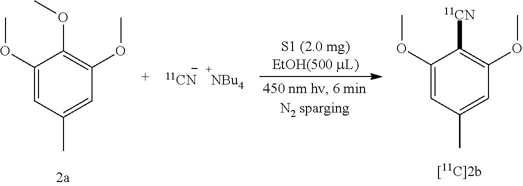

| | | | | | | | Decay | |
|---|---|---|---|---|---|---|---|---|
| | | | Activity | | | | corrected | Radio- |
| | | Substrate | ([¹¹C] | Injected | Collected | Isolation | Injection | chemical |
| Reaction | | Loading | TBACN) | dose | dose | time | dose | conversion |
| Entry | Substrate | (n, mmol) | (mCi) | (μCi) | (μCi) | (min) | (μCi) | (RCC, %) |
| 1 | 2a | 0.01 | 10.25 | 730 | 500 | 9 | 537.5 | 93.0 |
| | | | | | | | | (co-injection) |
| 2 | | 0.01 | 13.3 | 654 | 426 | 8 | 498 | 85.5 |
| 3 | | 0.01 | 25 | 1353 | 857 | 11 | 931 | 92.1 |
| 4 | | 0.03 | 26 | 720 | 496 | 9 | 530 | 93.6 |
| 5 | | 0.03 | 5.68 | 328 | 224 | 9 | 241.5 | 92.7 |
| 6 | | 0.03 | 20.5 | 603 | 391 | 9 | 444 | 88.1 |

Average RCC: 90.8 ± 3.3% (N = 6, decay corrected)

### HPLC-isolated RCCs of [¹¹C]2b by S1

### Example 94: Radio Reaction Optimization—Radio HPLC Analysis and Characterization of [¹¹C]Cyanide Labeled Arenes—Compound [¹¹C]2b

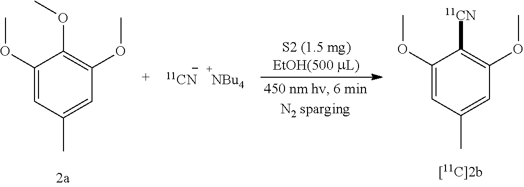

| | | | Activity | | | | Decay | |
|---|---|---|---|---|---|---|---|---|
| | | Substrate | ([¹¹C] | Injected | Collected | Isolation | corrected | Radiochemical |
| Reaction | | Loading | TBACN) | dose | dose | time | Injection | conversion |
| Entry | Substrate | (mmol) | (mCi) | (μCi) | (μCi) | (min) | dose (μCi) | (RCC, %, d.c.) |
| 1 | 2a | 0.01 | 3.3 | 269 | 82 | 10 | 191.5 | 42.8 |
| 2 | | 0.03 | 4.8 | 265 | 90 | 8 | 202 | 44.5 |

Average RCC: 43.7 ± 1.2% (N = 2, decay corrected)

### HPLC-isolated RCCs of [¹¹C]-2b by S2

### Example 95: Radio Reaction Optimization—Radio HPLC Analysis and Characterization of [¹¹C]Cyanide Labeled Arenes—Compound [¹¹C]2b

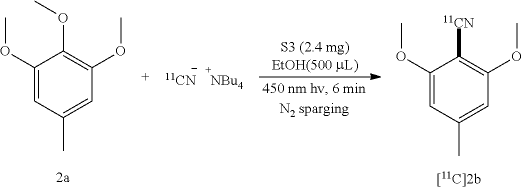

| | | | Activity | | | | Decay | Radio- |
|---|---|---|---|---|---|---|---|---|
| | | Substrate | ([¹¹C] | Injected | Collected | Isolation | corrected | chemical |
| Reaction | | Loading | TBACN) | dose | dose | time | Injection dose | conversion |
| Entry | Substrate | (mmol) | (mCi) | (μCi) | (μCi) | (min) | (μCi) | (RCC, %, d.c.) |
| 1 | 2a | 0.01 | 8.1 | 378 | 265 | 9 | 278.4 | 95.1 |

RCC: 95.1% (decay corrected)

### HPLC-isolated RCCs of [¹¹C]2b by S3

### Example 96: Radio Reaction Optimization—Radio HPLC Analysis and Characterization of [¹¹C]Cyanide Labeled Arenes—Compound [¹¹C]3b

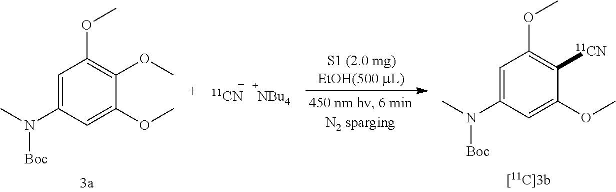

| | | | Activity | | | | Decay | |
|---|---|---|---|---|---|---|---|---|
| | | Substrate | ([¹¹C] | Injected | Collected | Isolation | corrected | Radiochemical |
| Reaction | | Loading | TBACN) | dose | dose | time | Injection dose | conversion |
| Entry | Substrate | (mmol) | (mCi) | (μCi) | (μCi) | (min) | (μCi) | (RCC, %, d.c.) |
| 1 | 3a | 0.03 | 4.5 | 181 | 47 | 8 | 137.9 | 34.2 |
| 2 | | 0.03 | 11.4 | 472 | 140 | 8 | 359.6 | 39.0 |
| 3 | | 0.03 | 12.5 | 652 | 168 | 10 | 464.1 | 36.2 |
| 4 | | 0.01 | 12.9 | 615 | 222 | 8 | 468.6 | 49.1 |

Average RCC: 36.5 ± 2.4% (N = 3, 0.03 mmol, decay corrected)

### HPLC-isolated RCCs of [¹¹C]3b by S1

### Example 97: Radio Reaction Optimization—Radio HPLC Analysis and Characterization of [¹¹C]Cyanide Labeled Arenes—Compound [¹¹C]4b

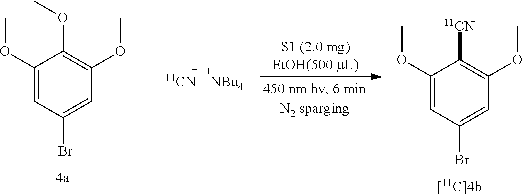

| | | | Activity | | | | Decay | |
|---|---|---|---|---|---|---|---|---|
| | | Substrate | ([¹¹C] | Injected | Collected | Isolation | corrected | Radiochemical |
| Reaction | | Loading | TBACN) | dose | dose | time | Injection dose | conversion |
| Entry | Substrate | (mmol) | (mCi) | (μCi) | (μCi) | (min) | (μCi) | (RCC, %, d.c.) |
| 1 | 4a | 0.01 | 18.9 | 637 | 158 | 7 | 502.1 | 31.5 |
| 2 | | 0.01 | 12.0 | 376 | 74 | 8 | 286.5 | 25.8 |
| 3 | | 0.01 | 12.5 | 544 | 87 | 8 | 414.5 | 21.1 |
| 4 | | 0.008 | 12.0 | 559 | 74 | 7 | 440.6 | 16.8 |
| 5 | | 0.03 | 20.0 | 478 | 4 | 7 | 377 | 1.0 |
| 6 | | 0.03 | 15.9 | 600 | 0 | — | — | 0 |
| 7 | | 0.03 | 7.1 | 263 | 0 | — | — | 0 |

Average RCC: 26.1 ± 5.2% (N = 3, 0.01 mmol, decay corrected)

### HPLC-isolated RCCs of [¹¹C]4b by S1

### Example 98: Radio Reaction Optimization—Radio HPLC Analysis and Characterization of [¹¹C]Cyanide Labeled Arenes—Compound [¹¹C]5b

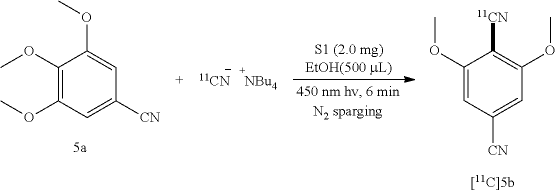

| | | | Activity | | | | Decay | |
|---|---|---|---|---|---|---|---|---|
| | | Substrate | ([¹¹C] | Injected | Collected | Isolation | corrected | Radiochemical |
| Reaction | | Loading | TBACN) | dose | dose | time | Injection dose | conversion |
| Entry | Substrate | (mmol) | (mCi) | (μCi) | (μCi) | (min) | (μCi) | (RCC, %, d.c.) |
| 1 | 5a | 0.03 | 9.7 | 619 | 21 | 7 | 471.6 | 4.5 |
| 2 | | 0.03 | 7.0 | 229 | 8 | 8 | 174.5 | 4.6 |
| 3 | | 0.03 | 12.0 | 360 | 14 | 11 | 248 | 5.6 |

Average RCC: 4.9 ± 0.6% (N = 3, decay corrected)

### HPLC-isolated RCCs of [¹¹C]5b by S1

### Example 99: Radio Reaction Optimization—Radio HPLC Analysis and Characterization of [¹¹C]Cyanide Labeled Arenes—Compound [¹¹C]5b

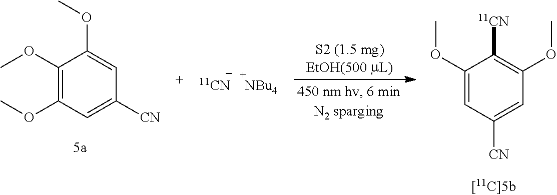

| | | | Activity | | | | Decay | |
|---|---|---|---|---|---|---|---|---|
| | | Substrate | ([¹¹C] | Injected | Collected | Isolation | corrected | Radiochemical |
| Reaction | | Loading | TBACN) | dose | dose | time | Injection dose | conversion |
| Entry | Substrate | (mmol) | (mCi) | (μCi) | (μCi) | (min) | (μCi) | (RCC, %, d.c.) |
| 1 | 5a | 0.03 | 4.7 | 317 | 63 | 8 | 241.5 | 26.2 |
| 2 | | 0.03 | 3.7 | 190 | 32 | 9 | 140 | 22.9 |
| 3 | | 0.03 | 4.1 | 211 | 38 | 9 | 155.4 | 24.6 |

Average RCC: 24.6 ± 1.7% (N = 3, decay corrected)

### HPLC-isolated RCCs of [¹¹C]5b by S2

### Example 100: Radio Reaction Optimization—Radio HPLC Analysis and Characterization of [¹¹C]Cyanide Labeled Arenes—Compound [¹¹C]6b

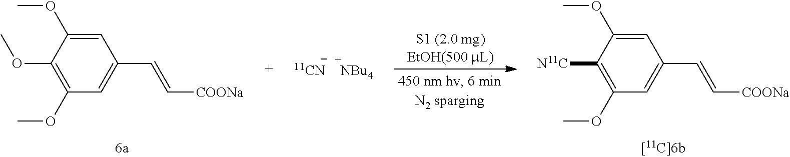

| | | | | | | | Decay | |
|---|---|---|---|---|---|---|---|---|
| | | Substrate | Activity | Injected | Collected | Isolation | corrected | Radiochemical |
| Reaction | | Loading | ([¹¹C]TBACN) | dose | dose | time | Injection dose | conversion |
| Entry | Substrate | (mmol) | (mCi) | (μCi) | (μCi) | (min) | (μCi) | (RCC, %, d.c.) |
| 1 | 6a | 0.01 | 24.6 | 702 | 412 | 8 | 534.5 | 77.1 |
| 2 | | 0.01 | 20.2 | 722 | 409 | 9 | 531 | 77.0 |
| 3 | | 0.01 | 16.8 | 223 | 91 | 9 | 164 | 55.5 |

Average RCC: 69.9 ± 12.4% (N = 3, decay corrected)

### HPLC-isolated RCCs of [¹¹C]6b by S1

### Example 101: Radio Reaction Optimization—Radio HPLC Analysis and Characterization of [¹¹C]Cyanide Labeled Arenes—Compound [¹¹C]7b

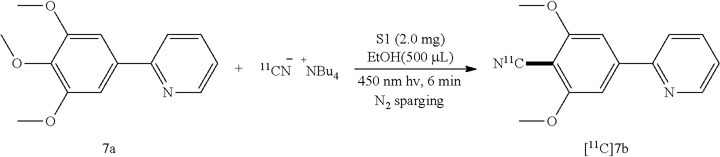

| | | | | | | | Decay | |
|---|---|---|---|---|---|---|---|---|
| | | Substrate | Activity | Injected | Collected | Isolation | corrected | Radiochemical |
| Reaction | | Loading | ([¹¹C]TBACN) | dose | dose | time | Injection dose | conversion |
| Entry | Substrate | (mmol) | (mCi) | (μCi) | (μCi) | (min) | (μCi) | (RCC, %, d.c.) |
| 1 | 7a | 0.01 | 3.7 | 302 | 188 | 9 | 222.4 | 84.5 |
| 2 | | 0.01 | 3.0 | 297 | 169 | 10 | 211.4 | 80.0 |
| 3 | | 0.01 | 3.3 | 362 | 210 | 9 | 266.6 | 78.8 |

Average RCC: 81.1 ± 3.0% (N = 3, decay corrected)

### HPLC-isolated RCCs of [¹¹C]7b by S1

### Example 102: Radio Reaction Optimization—Radio HPLC Analysis and Characterization of [¹¹C]Cyanide Labeled Arenes—Compound [¹¹C]8b

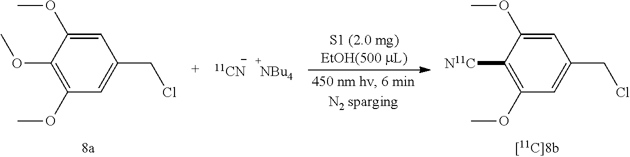

| | | | | | | | Decay | |
|---|---|---|---|---|---|---|---|---|
| | | Substrate | Activity | Injected | Collected | Isolation | corrected | Radiochemical |
| Reaction | | Loading | ([¹¹C]TBACN) | dose | dose | time | Injection dose | conversion |
| Entry | Substrate | (mmol) | (mCi) | (μCi) | (μCi) | (min) | (μCi) | (RCC, %, d.c.) |
| 1 | 8a | 0.01 | 3.15 | 322 | 136 | 11 | 221 | 61.5 |
| 2 | | 0.01 | 5.68 | 436 | 198 | 11 | 300 | 66.0 |
| | | | | | | | | (co-injection) |
| 3 | | 0.01 | 4.1 | 252 | 101 | 11 | 173 | 58.3 |

Average RCC: 61.9 ± 3.9% (N = 3, decay corrected)

### HPLC-isolated RCCs of [¹¹C]8b by S1

### Example 103: Radio Reaction Optimization—Radio HPLC Analysis and Characterization of [¹¹C]Cyanide Labeled Arenes—Compound [¹¹C]9a

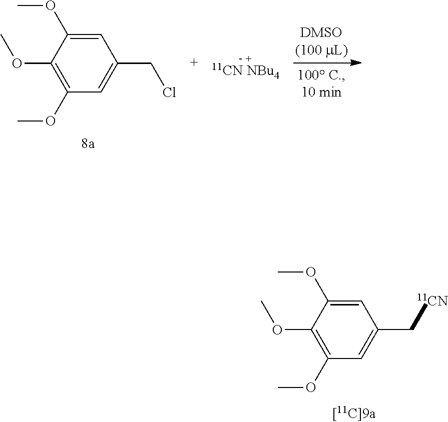

The S_{N}2 labeling reaction procedure. The substrate 8a (0.015 mmol) was firstly weighed into a 1.5 mL Eppendorf Tube® before being dissolved with anhydrous DMSO (100 μL). Next, a 10-60 μL aliquot of [¹¹C]TBACN-TBAOH in MeCN was added to a 5 mL V vial via pipette before the vial was sealed with an aluminum-rubber crimp cap with a venting needle and connected to argon stream line with another needle. Then in a hot cell, the vial was put onto a heating block and heated at 80° C. with the argon flowing on to completely remove the ethanol (2-3 minutes usually). Then the DMSO reactant solution was injected into the dried V vial containing the [¹¹C]TBACN before radio activity measurement confirmed no loss of the ¹¹CN. Then, the vial was put onto the heating block and heated at 100° C. for 10 minutes. Finally, after cooling down, 400 μL MeCN was added into the V vial to dilute the product mixture before an aliquot of the reaction mixture was taken for radio HPLC analysis.

| | | | Activity | | | | Decay | Radiochemical |
|---|---|---|---|---|---|---|---|---|
| | | Substrate | ([¹¹C] | Injected | Collected | Isolation | corrected | conversion |
| Reaction | | Loading | TBACN) | dose | dose | time | Injection dose | (RCC, |
| Entry | Substrate | (mmol) | (mCi) | (μCi) | (μCi) | (min) | (μCi) | %, d.c.) |
| 1 | 8a | 0.015 | 4.0 | 232 | 55 | 6 | 189.2 | 29.1 |

RCC: 29.1% (decay corrected)

### HPLC-isolated RCCs of [¹¹C]9a

### Example 104: Radio Reaction Optimization—Radio HPLC Analysis and Characterization of [¹¹C]Cyanide Labeled Arenes—Compound [¹¹C]9b

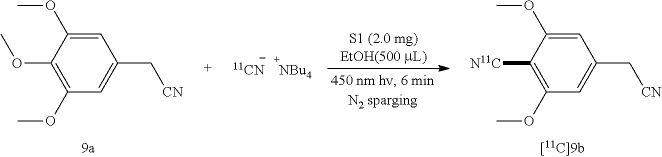

| | | | | | | | Decay | |
|---|---|---|---|---|---|---|---|---|
| | | Substrate | Activity | Injected | Collected | Isolation | corrected | Radiochemical |
| Reaction | | Loading | ([¹¹C]TBACN) | dose | dose | time | Injection dose | conversion |
| Entry | Substrate | (mmol) | (mCi) | (μCi) | (μCi) | (min) | (μCi) | (RCC, %, d.c.) |
| 1 | 9a | 0.008 | 15.6 | 680 | 298 | 8 | 518 | 57.5 |
| 2 | | 0.01 | 5.63 | 376 | 170 | 7 | 296.4 | 57.4 |
| 3 | | 0.005 | 21.6 | 584 | 230 | 9 | 430 | 53.5 |

Average RCC: 56.1 ± 2.3% (N = 3, decay corrected)

### HPLC-isolated RCCs of [¹¹C]9b by S1

### Example 105: Radio Reaction Optimization—Radio HPLC Analysis and Characterization of [¹¹C]Cyanide Labeled Arenes—Compound [¹¹C]10b

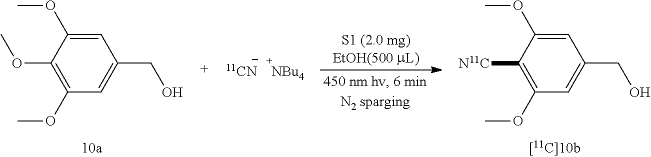

| | | | | | | | Decay | |
|---|---|---|---|---|---|---|---|---|
| | | Substrate | Activity | Injected | Collected | Isolation | corrected | Radiochemical |
| Reaction | | Loading | ([¹¹C]TBACN) | dose | dose | time | Injection dose | conversion |
| Entry | Substrate | (mmol) | (mCi) | (μCi) | (μCi) | (min) | (μCi) | (RCC, %, d.c.) |
| 1 | 10a | 0.01 | 11.9 | 545 | 343 | 8 | 415 | 82.6 |
| 2 | | 0.01 | 3.5 | 280 | 180 | 7 | 220.7 | 81.6 |
| 3 | | 0.01 | 8.9 | 527 | 350 | 8 | 401.5 | 87.2 |

Average RCC: 83.8 ± 3.0% (n = 3, decay corrected)

### HPLC-isolated RCCs of [¹¹C]10b by S1

### Example 106: Radio Reaction Optimization—Radio HPLC Analysis and Characterization of [¹¹C]Cyanide Labeled Arenes—Compound [¹¹C]10b

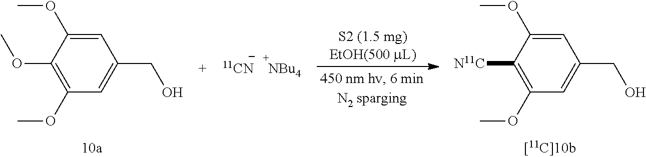

| | | | | | | | Decay | |
|---|---|---|---|---|---|---|---|---|
| | | Substrate | Activity | Injected | Collected | Isolation | corrected | Radiochemical |
| Reaction | | Loading | ([¹¹C]TBACN) | dose | dose | time | Injection dose | conversion |
| Entry | Substrate | (mmol) | (mCi) | (μCi) | (μCi) | (min) | (μCi) | (RCC, %, d.c.) |
| 1 | 10a | 0.01 | 3.3 | 236 | 70 | 8 | 179.8 | 38.9 |
| 2 | | 0.01 | 7.6 | 231 | 62 | 8 | 176 | 35.2 |

Average RCC: 37.1 ± 2.6% (N = 2, decay corrected)

### HPLC-isolated RCCs of [¹¹C]10b by S2

### Example 107: Radio Reaction Optimization—Radio HPLC Analysis and Characterization of [¹¹C]Cyanide Labeled Arenes—Compound [¹¹C]11b

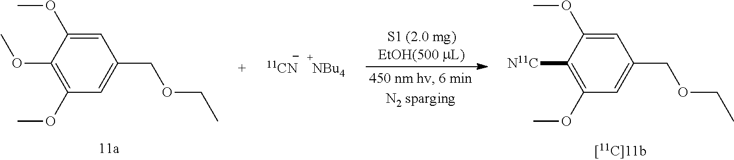

| | | | | | | | Decay | |
|---|---|---|---|---|---|---|---|---|
| | | Substrate | Activity | Injected | Collected | Isolation | corrected | Radiochemical |
| Reaction | | Loading | ([¹¹C]TBACN) | dose | dose | time | Injection dose | conversion |
| Entry | Substrate | (mmol) | (mCi) | (μCi) | (μCi) | (min) | (μCi) | (RCC, %, d.c.) |
| 1 | 11a | 0.01 | 11.6 | 457 | 283 | 9 | 336.5 | 84.1 |
| 2 | | 0.01 | 7.9 | 462 | 284 | 9 | 340.2 | 83.5 |
| 3 | | 0.01 | 12.7 | 577 | 356 | 8 | 439.6 | 81.0 |

Average RCC: 82.9 ± 1.6% (n = 3, decay corrected)

### HPLC-isolated RCCs of [¹¹C]11b by S1

### Example 108: Radio Reaction Optimization—Radio HPLC Analysis and Characterization of [¹¹C]Cyanide Labeled Arenes—Compound [¹¹C]11b

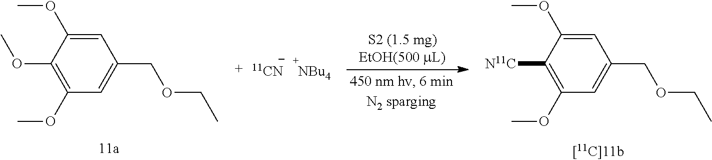

| | | | | | | | Decay | |
|---|---|---|---|---|---|---|---|---|
| | | Substrate | Activity | Injected | Collected | Isolation | corrected | Radiochemical |
| Reaction | | Loading | ([¹¹C]TBACN) | dose | dose | time | Injection dose | conversion |
| Entry | Substrate | (mmol) | (mCi) | (μCi) | (μCi) | (min) | (μCi) | (RCC, %, d.c.) |
| 1 | 11a | 0.01 | 5.95 | 331 | 116 | 9 | 244 | 47.5 |
| 2 | | 0.01 | 6.5 | 272 | 88 | 9 | 200.5 | 43.9 |

Average RCC: 45.7 ± 2.5% (N = 2, decay corrected)

### HPLC-isolated RCCs of [¹¹C]11b by S2

### Example 109: Radio Reaction Optimization—Radio HPLC Analysis and Characterization of [¹¹C]Cyanide Labeled Arenes—Compound [¹¹C]12b

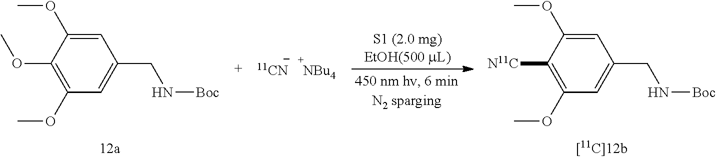

| | | | | | | | Decay | |
|---|---|---|---|---|---|---|---|---|
| | | Substrate | Activity | Injected | Collected | Isolation | corrected | Radiochemical |
| Reaction | | Loading | ([¹¹C]TBACN) | dose | dose | time | Injection dose | conversion |
| Entry | Substrate | (mmol) | (mCi) | (μCi) | (μCi) | (min) | (μCi) | (RCC, %, d.c.) |
| 1 | 12a | 0.01 | 12.7 | 566 | 329 | 11 | 389.4 | 84.5 |
| 2 | | 0.01 | 5.5 | 365 | 248 | 8 | 278 | 89.2 |
| 3 | | 0.01 | 20.6 | 730 | 502 | 9 | 537.6 | 93.4 |
| | | | | | | | | Co-injection |

Average RCC: 89.0 ± 4.5% (n = 3, decay corrected)

### HPLC-isolated RCCs of [¹¹C]12b by S1

### Example 110: Radio Reaction Optimization—Radio HPLC Analysis and Characterization of [¹¹C]Cyanide Labeled Arenes—Compound [¹¹C]12b

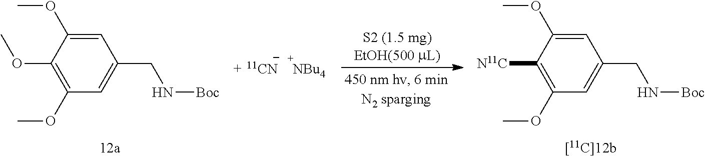

| | | | | | | | Decay | |
|---|---|---|---|---|---|---|---|---|
| | | Substrate | Activity | Injected | Collected | Isolation | corrected | Radiochemical |
| Reaction | | Loading | ([¹¹C]TBACN) | dose | dose | time | Injection dose | conversion |
| Entry | Substrate | (mmol) | (mCi) | (μCi) | (μCi) | (min) | (μCi) | (RCC, %, d.c.) |
| 1 | 12a | 0.02 | 2.15 | 216 | 81 | 11 | 148.6 | 54.5 |

RCC: 54.5% (decay corrected)

### HPLC-isolated RCCs of [¹¹C]12b by S2

### Example 111: Radio Reaction Optimization—Radio HPLC Analysis and Characterization of [¹¹C]Cyanide Labeled Arenes—Compound [¹¹C]13b

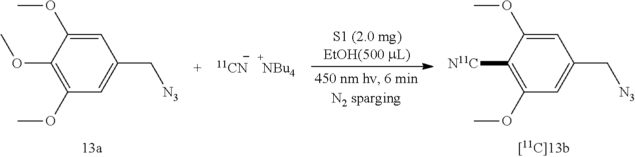

| | | | | | | | Decay | |
|---|---|---|---|---|---|---|---|---|
| | | Substrate | Activity | Injected | Collected | Isolation | corrected | Radiochemical |
| Reaction | | Loading | ([¹¹C]TBACN) | dose | dose | time | Injection dose | conversion |
| Entry | Substrate | (mmol) | (mCi) | (μCi) | (μCi) | (min) | (μCi) | (RCC, %, d.c.) |
| 1 | 13a | 0.01 | 7.65 | 287 | 131 | 10 | 204 | 64.1 |
| 2 | | 0.01 | 13.5 | 717 | 332 | 9 | 528 | 62.9 |
| 3 | | 0.01 | 8.2 | 378 | 158 | 10 | 269 | 58.7 |

Average RCC: 61.9 ± 2.8% (n = 3, decay corrected)

### HPLC-isolated RCCs of [¹¹C]13b by S1

### Example 112: Radio Reaction Optimization—Radio HPLC Analysis and Characterization of [¹¹C]Cyanide Labeled Arenes—Compound [¹¹C]13b

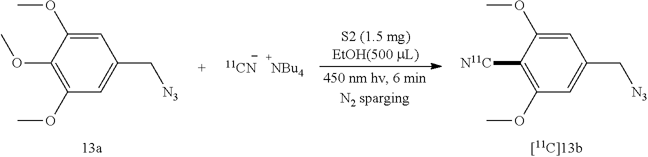

| | | | | | | | Decay | |
|---|---|---|---|---|---|---|---|---|
| | | Substrate | Activity | Injected | Collected | Isolation | corrected | Radiochemical |
| Reaction | | Loading | ([¹¹C]TBACN) | dose | dose | time | Injection dose | conversion |
| Entry | Substrate | (mmol) | (mCi) | (μCi) | (μCi) | (min) | (μCi) | (RCC, %, d.c.) |
| 1 | 12a | 0.01 | 4.75 | 233 | 40 | 10 | 165.9 | 24.1 |

RCC: 24.1% (decay corrected)

### HPLC-isolated RCCs of [¹¹C]13b by S2

### Example 113: Radio Reaction Optimization—Radio HPLC Analysis and Characterization of [¹¹C]Cyanide Labeled Arenes—Compound [¹¹C]14b

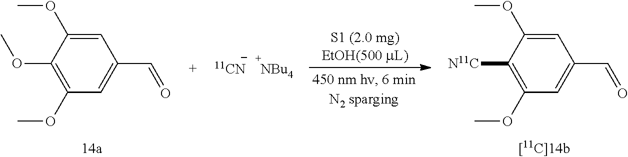

| | | | | | | | Decay | |
|---|---|---|---|---|---|---|---|---|
| | | Substrate | Activity | Injected | Collected | Isolation | corrected | Radiochemical |
| Reaction | | Loading | ([¹¹C]TBACN) | dose | dose | time | Injection dose | conversion |
| Entry | Substrate | (mmol) | (mCi) | (μCi) | (μCi) | (min) | (μCi) | (RCC, %, d.c.) |
| 1 | 14a | 0.03 | 13.3 | 484 | 221 | 7 | 381.5 | 57.9 |
| 2 | | 0.03 | 10.7 | 621 | 264 | 8 | 473 | 55.8 |
| 3 | | 0.03 | 17.5 | 648 | 288 | 9 | 477 | 60.4 |
| 4 | | 0.02 | 14.0 | 661 | 230 | 8 | 504 | 45.6 |
| 5 | | 0.02 | 19.6 | 530 | 184 | 8 | 404 | 45.5 |
| 6 | | 0.01 | 12.85 | 594 | 225 | 7 | 468 | 48.0 |
| | | | | | | | | Co-injection |

Average RCC: 58.0 ± 2.3% (n = 3, 0.03 mmol, decay corrected)

### HPLC-isolated RCCs of [¹¹C]14b by S1

### Example 114: Radio Reaction Optimization—Radio HPLC Analysis and Characterization of [¹¹C]Cyanide Labeled Arenes—Compound [¹¹C]14b

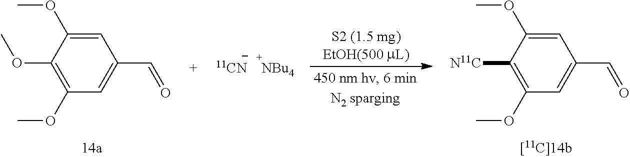

| | | | | | | | Decay | |
|---|---|---|---|---|---|---|---|---|
| | | Substrate | Activity | Injected | Collected | Isolation | corrected | Radiochemical |
| Reaction | | Loading | ([¹¹C]TBACN) | dose | dose | time | Injection dose | conversion |
| Entry | Substrate | (mmol) | (mCi) | (μCi) | (μCi) | (min) | (μCi) | (RCC, %, d.c.) |
| 1 | 14a | 0.03 | 7.6 | 322 | 67 | 8 | 245.3 | 27.3 |

RCC: 27.3% (decay corrected)

### HPLC-isolated RCCs of [¹¹C]14b by S2

### Example 115: Radio Reaction Optimization—Radio HPLC Analysis and Characterization of [¹¹C]Cyanide Labeled Arenes—Compound [¹¹C]15b

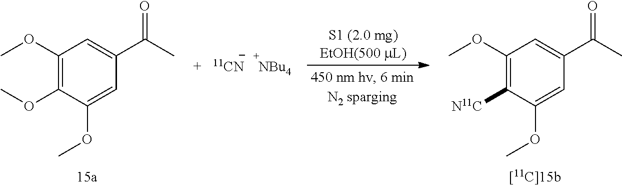

| | | | | | | | Decay | |
|---|---|---|---|---|---|---|---|---|
| | | Substrate | Activity | Injected | Collected | Isolation | corrected | Radiochemical |
| Reaction | | Loading | ([¹¹C]TBACN) | dose | dose | time | Injection dose | conversion |
| Entry | Substrate | (mmol) | (mCi) | (μCi) | (μCi) | (min) | (μCi) | (RCC, %, d.c.) |
| 1 | 15a | 0.03 | 8.7 | 524 | 257 | 9 | 385.9 | 68.1 |
| 2 | | 0.03 | 15.4 | 644 | 344 | 9 | 474.3 | 72.5 |
| 3 | | 0.03 | 59.0 | 680 | 353 | 8 | 518 | 68.2 |
| 4 | | 0.03 | 6.0 | 266 | 140 | 10 | 189.3 | 74.0 |
| 5 | | 0.01 | 2.92 | 240 | 82 | 8 | 183 | 45.0 |
| | | | | | | | | Co-injection |

Average RCC: 70.7 ± 3.0% (n = 4, 0.03 mmol, decay corrected)

### HPLC-isolated RCCs of [¹¹C]15b by S1

### Example 116: Radio Reaction Optimization—Radio HPLC Analysis and Characterization of [¹¹C]Cyanide Labeled Arenes—Compound [¹¹C]15b

| | |
|---|---|
| 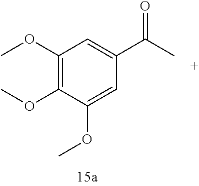 | 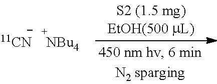 |

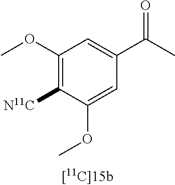

| | | Substrate | Activity | Injected | Collected | Isolation | corrected | Radiochemical |
|---|---|---|---|---|---|---|---|---|
| Reaction | | Loading | ([¹¹C]TBACN) | dose | dose | time | Injection dose | conversion |
| Entry | Substrate | (mmol) | (mCi) | (μCi) | (μCi) | (min) | (μCi) | (RCC, %, d.c.) |
| 1 | 15a | 0.03 | 3.7 | 262 | 71 | 10 | 186.5 | 38.1 |

RCC: 38.1% (decay corrected)

### HPLC-isolated RCCs of [¹¹C]15b by S2

### Example 117: Radio Reaction Optimization—Radio HPLC Analysis and Characterization of [¹¹C]Cyanide Labeled Arenes—Compound [¹¹C]16b

| | |
|---|---|
| 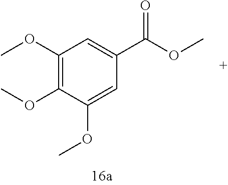 | 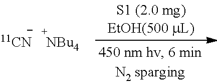 |

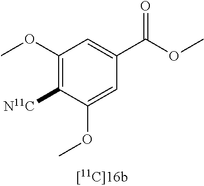

| | | | | | | | Decay | |
|---|---|---|---|---|---|---|---|---|
| | | Substrate | Activity | Injected | Collected | Isolation | corrected | Radiochemical |
| Reaction | | Loading | ([¹¹C]TBACN) | dose | dose | time | Injection dose | conversion |
| Entry | Substrate | (mmol) | (mCi) | (μCi) | (μCi) | (min) | (μCi) | (RCC, %, d.c.) |
| 1 | 16a | 0.03 | 11.5 | 443 | 227 | 11 | 304.8 | 74.5 |
| 2 | | 0.03 | 9.8 | 430 | 230 | 10 | 306 | 75.1 |
| 3 | | 0.03 | 19.9 | 519 | 256 | 10 | 369.4 | 69.4 |
| 4 | | 0.01 | 16.8 | 558 | 210 | 9 | 410.9 | 51.1 |
| | | | | | | | | Co-injection |

Average RCC: 73.0 ± 3.1% (n = 3, 0.03 mmol, decay corrected)

### HPLC-isolated RCCs of [¹¹C]16b by S1

### Example 118: Radio Reaction Optimization—Radio HPLC Analysis and Characterization of [¹¹C]Cyanide Labeled Arenes—Compound [¹¹C]16b

| | |
|---|---|
| 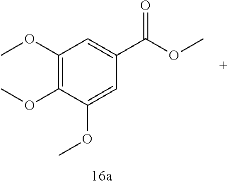 | 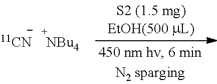 |

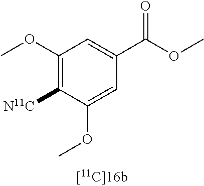

| | | | | | | | Decay | |
|---|---|---|---|---|---|---|---|---|
| | | Substrate | Activity | Injected | Collected | Isolation | corrected | Radiochemical |
| Reaction | | Loading | ([¹¹C]TBACN) | dose | dose | time | Injection dose | conversion |
| Entry | Substrate | (mmol) | (mCi) | (μCi) | (μCi) | (min) | (μCi) | (RCC, %, d.c.) |
| 1 | 16a | 0.03 | 3.6 | 144 | 22 | 13 | 92.5 | 23.8 |

RCC: 23.8% (decay corrected)

### HPLC-isolated RCCs of [¹¹C]16b by 2

### Example 119: Radio Reaction Optimization—Radio HPLC Analysis and Characterization of [¹¹C]Cyanide Labeled Arenes—Compound [¹¹C]17b

| | |
|---|---|
| 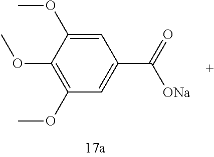 | 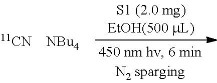 |

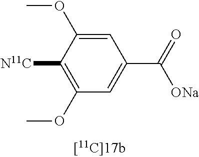

| | | | | | | | Decay | |
|---|---|---|---|---|---|---|---|---|
| | | Substrate | Activity | Injected | Collected | Isolation | corrected | Radiochemical |
| Reaction | | Loading | ([¹¹C]TBACN) | dose | dose | time | Injection dose | conversion |
| Entry | Substrate | (mmol) | (mCi) | (μCi) | (μCi) | (min) | (μCi) | (RCC, %, d.c.) |
| 1 | 17a | 0.01 | 11.3 | 560 | 303 | 7 | 441.5 | 68.7 |
| 2 | | 0.01 | 4.2 | 252 | 10 | 13 | 162 | 62.3 |
| 3 | | 0.01 | 7.5 | 398 | 199 | 8 | 303 | 65.6 |

Average RCC: 65.5 ± 3.2% (n = 3, decay corrected)

### HPLC-isolated RCCs of [¹¹C]17b by S1

### Example 120: Radio Reaction Optimization—Radio HPLC Analysis and Characterization of [¹¹C]Cyanide Labeled Arenes—Compound [¹¹C]18b

| | |
|---|---|
| 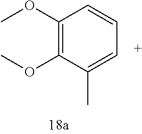 | 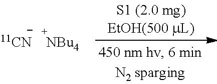 |

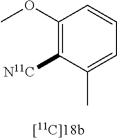

| | | | | | | | Decay | |
|---|---|---|---|---|---|---|---|---|
| | | Substrate | Activity | Injected | Collected | Isolation | corrected | Radiochemical |
| Reaction | | Loading | ([¹¹C]TBACN) | dose | dose | time | Injection dose | conversion |
| Entry | Substrate | (mmol) | (mCi) | (μCi) | (μCi) | (min) | (μCi) | (RCC, %, d.c.) |
| 1 | 18a | 0.03 | 22.0 | 592 | 371 | 9 | 436 | 85.1 |
| 2 | | 0.03 | 17.1 | 636 | 405 | 8 | 468.4 | 83.6 |
| 3 | | 0.03 | 16.3 | 740 | 470 | 9 | 545 | 86.3 |

Average RCC: 85.0 ± 1.4% (n = 3, decay corrected)

### HPLC-isolated RCCs of [¹¹C]18b by S1

### Example 121: Radio Reaction Optimization—Radio HPLC Analysis and Characterization of [¹¹C]Cyanide Labeled Arenes—Compound [¹¹C]18b

| | |
|---|---|
| 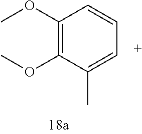 | 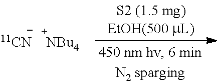 |

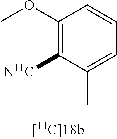

| | | | | | | | Decay | |
|---|---|---|---|---|---|---|---|---|
| | | Substrate | Activity | Injected | Collected | Isolation | corrected | Radiochemical |
| Reaction | | Loading | ([¹¹C]TBACN) | dose | dose | time | Injection dose | conversion |
| Entry | Substrate | (mmol) | (mCi) | (μCi) | (μCi) | (min) | (μCi) | (RCC, %, d.c.) |
| 1 | 18a | 0.03 | 6.9 | 380 | 104 | 9 | 280 | 37.1 |

RCC: 37.1% (decay corrected)

### HPLC-isolated RCCs of [¹¹C]18b by S1

### Example 122: Radio Reaction Optimization—Radio HPLC Analysis and Characterization of [¹¹C]Cyanide Labeled Arenes—Compound [¹¹C]19b

| | |
|---|---|
| 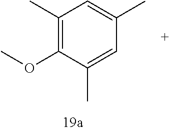 | 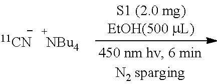 |

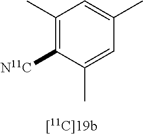

| | | | | | | | Decay | |
|---|---|---|---|---|---|---|---|---|
| | | Substrate | Activity | Injected | Collected | Isolation | corrected | Radiochemical |
| Reaction | | Loading | ([¹¹C]TBACN) | dose | dose | time | Injection dose | conversion |
| Entry | Substrate | (mmol) | (mCi) | (μCi) | (μCi) | (min) | (μCi) | (RCC, %, d.c.) |
| 1 | 19a | 0.03 | 5.85 | 248 | 126 | 9 | 182.6 | 69.0 |
| 2 | | 0.03 | 8.6 | 335 | 140 | 9 | 246.7 | 56.7 |
| 3 | | 0.03 | 7.3 | 279 | 105 | 9 | 205 | 51.2 |
| 4 | | 0.03 | 27.5 | 722 | 285 | 8 | 550 | 51.8 |
| 5 | | 0.01 | 18.0 | 616 | 177 | 8 | 469 | 37.7 |

Average RCC: 57.2 ± 8.3% (N = 4, n = 0.03 mmol, decay corrected)

### HPLC-isolated RCCs of [¹¹C]19b by S1

### Example 123: Radio Reaction Optimization—Radio HPLC Analysis and Characterization of [¹¹C]Cyanide Labeled Arenes—Compound [¹¹C]19b

| | |
|---|---|
| 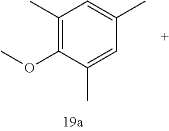 | 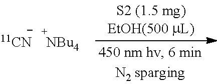 |

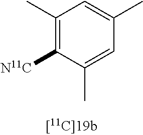

| | | | | | | | Decay | |
|---|---|---|---|---|---|---|---|---|
| | | Substrate | Activity | Injected | Collected | Isolation | corrected | Radiochemical |
| Reaction | | Loading | ([¹¹C]TBACN) | dose | dose | time | Injection dose | conversion |
| Entry | Substrate | (mmol) | (mCi) | (μCi) | (μCi) | (min) | (μCi) | (RCC, %, d.c.) |
| 1 | 19a | 0.03 | 4-7 | 420 | 70 | 9 | 309.3 | 22.6 |

RCC: 22.6% (decay corrected)

### HPLC-isolated RCCs of [¹¹C]19b by S2

### Example 124: Radio Reaction Optimization—Radio HPLC Analysis and Characterization of [¹¹C]Cyanide Labeled Arenes—Compound [¹¹C]20b

| | |
|---|---|
| 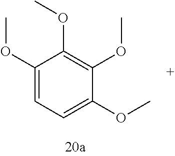 | 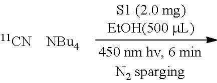 |

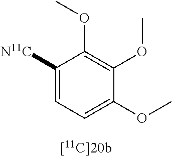

| | | | | | | | Decay | |
|---|---|---|---|---|---|---|---|---|
| | | Substrate | Activity | Injected | Collected | Isolation | corrected | Radiochemical |
| Reaction | | Loading | ([¹¹C]TBACN) | dose | dose | time | Injection dose | conversion |
| Entry | Substrate | (mmol) | (mCi) | (μCi) | (μCi) | (min) | (μCi) | (RCC, %, d.c.) |
| 1 | 20a | 0.03 | 12.6 | 523 | 119 | 8 | 398.5 | 30.0 |
| 2 | | 0.03 | 13.1 | 550 | 141 | 8 | 419 | 33.6 |
| 3 | | 0.03 | 8.9 | 289 | 79 | 8 | 220.2 | 36.0 |
| 4 | | 0.01 | 14.2 | 575 | 170 | 8 | 438 | 38.8 |

Average RCC: 33.2 ± 3.0% (n = 3, 0.03 mmol, decay corrected)

### HPLC-isolated RCCs of [¹¹C]20b by S1

### Example 125: Radio Reaction Optimization—Radio HPLC Analysis and Characterization of [¹¹C]Cyanide Labeled Arenes—Compounds [¹¹C]21b/21c

| 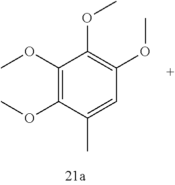 | | 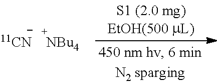 | | | | | | | | |
|---|---|---|---|---|---|---|---|---|---|---|
| 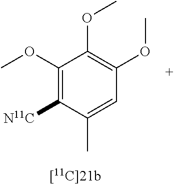 | | | | | | | | | | 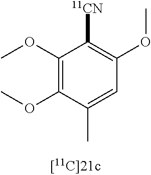 |
| | | | | | | | | Decay | | |
| | | | | | | | | corrected | Radiochemical | Radiochemical |
| | | Substrate | Activity | Injected | Collected | Collected | Isolation | Injection | conversion | conversion |
| Reaction | | Loading | ([¹¹C]TBACN) | dose | dose (b) | dose (c) | time | dose | (RCC, %, | (RCC, %, |
| Entry | Substrate | (mmol) | (mCi) | (μCi) | (μCi) | (μCi) | (min) | (μCi) | d.c.) of b | d.c.) of c |
| 1 | 21a | 0.03 | 16.0 | 600 | 175 | 47 | 11 | 412.8 | 42.4 | 11.4 |
| 2 | | 0.03 | 12.4 | 600 | 196 | 53 | 10 | 427 | 45.9 | 12.4 |
| 3 | | 0.03 | 15.3 | 665 | 200 | 54 | 10 | 473 | 42.3 | 11.4 |

Average RCC: 43.5 ± 2.1% (of b); Average RCC: 11.7 ± 0.6% (of c) (n = 3, decay corrected)

HPLC-isolated RCCs of [¹¹C]21b and [¹¹]C21c by S1

### Example 126: Radio Reaction Optimization—Radio HPLC Analysis and Characterization of [¹¹C]Cyanide Labeled Arenes—Compounds [¹¹C]22b/22c

| | |
|---|---|
| 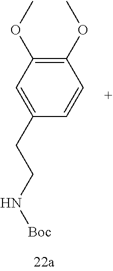 [SMILES: COc1ccc(CCNC(=O)OC(C)(C)C)cc1OC] | 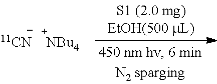 |

| | | |
|---|---|---|
| 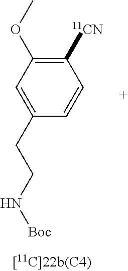 | 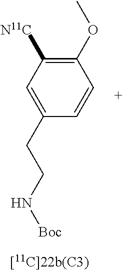 | 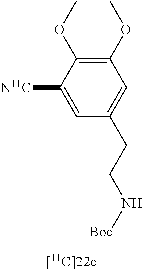 |

| | | | | | | | | Decay | | |
|---|---|---|---|---|---|---|---|---|---|---|
| | | | | | | | | corrected | Radiochemical | Radiochemical |
| | | Substrate | Activity | Injected | Collected | Collected | Isolation | Injection | conversion | conversion |
| Reaction | | Loading | ([¹¹C]TBACN) | dose | dose (b) | dose (c) | time | dose | (RCC, %, | (RCC, %, |
| Entry | Substrate | (mmol) | (mCi) | (μCi) | (μCi) | (μCi) | (min) | (μCi) | d.c.) of b | d.c.) of c |
| 1 | 22a | 0.03 | 16.1 | 712 | 309 | 55 | 11 | 489.9 | 63.1 | 11.2 |
| 2 | | 0.03 | 12.6 | 321 | 133 | 24 | 12 | 213.5 | 62.3 | 11.2 |
| 3 | | 0.03 | 21.4 | 742 | 275 | 61 | 12, 9 | 493, | 55.8 | 11.2 |
| | | | | | | | | 546.5 | | |

Average RCC: 60.4 ± 4.0% (of b); Average RCC: 11.2 ± 0.1% (of c) (n = 3, decay corrected)

HPLC-isolated RCCs of [¹¹C]22b and [¹¹]C22c by S1

### Example 127: Radio Reaction Optimization—Radio HPLC Analysis and Characterization of [¹¹C]Cyanide Labeled Arenes—Compound [¹¹C]23b/23c

| | |
|---|---|
| 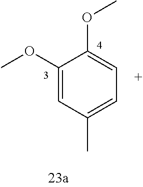 | 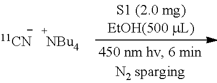 |

| | | |
|---|---|---|
| 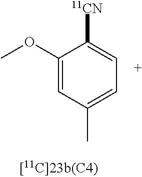 | 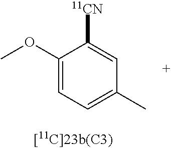 | 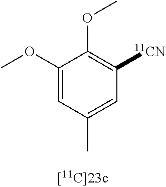 |

| | | | | | | | | Decay | | |
|---|---|---|---|---|---|---|---|---|---|---|
| | | | | | | | | corrected | Radiochemical | Radiochemical |
| | | Substrate | Activity | Injected | Collected | Collected | Isolation | Injection | conversion | conversion |
| Reaction | | Loading | ([¹¹C]TBACN) | dose | dose (b) | dose (c) | time | dose | (RCC, %, | (RCC, %, |
| Entry | Substrate | (mmol) | (mCi) | (μCi) | (μCi) | (μCi) | (min) | (μCi) | d.c.) of b | d.c.) of c |
| 1 | 23a | 0.03 | 14.5 | 625 | 192 | 58 | 9 | 460 | 41.8 | 12.6 |
| 2 | | 0.03 | 14.4 | 545 | 195 | 58 | 9 | 401.4 | 48.6 | 13.7 |
| 3 | | 0.03 | 13.4 | 729 | 250 | 71 | 10 | 519 | 48.2 | 13.7 |

Average RCC: 46.2 ± 3.8% (of b)(C4 + C3); Average RCC: 13.3 ± 0.6% of c (n = 3, decay corrected)

HPLC-isolated RCCs of [¹¹]C23b and [¹¹C]23e by S1

### Example 128: Radio Reaction Optimization—Radio HPLC Analysis and Characterization of [¹¹C]Cyanide Labeled Arenes—Compounds [¹¹C]24b/24c

| | |
|---|---|
| 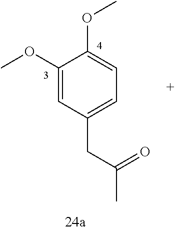 | 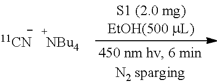 |

| | | |
|---|---|---|
| 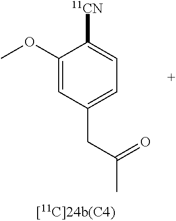 | 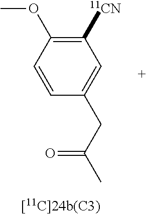 | 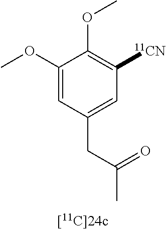 |

| | | | | | | | | Decay | | |
|---|---|---|---|---|---|---|---|---|---|---|
| | | | | | | | | corrected | Radiochemical | Radiochemical |
| | | Substrate | Activity | Injected | Collected | Collected | Isolation | Injection | conversion | conversion |
| Reaction | | Loading | ([¹¹C]TBACN) | dose | dose (b) | dose (c) | time | dose | (RCC, %, | (RCC, %, |
| Entry | Substrate | (mmol) | (mCi) | (μCi) | (μCi) | (μCi) | (min) | (μCi) | d.c.) of b | d.c.) of c |
| 1 | 24a | 0.03 | 14.4 | 422 | 193 | 32 | 8 | 321.5 | 60.0 | 10.0 |
| 2 | | 0.03 | 20.6 | 768 | 342 | 74 | 9 | 565.5 | 60.5 | 13.1 |
| 3 | | 0.03 | 9.1 | 388 | 193 | 38 | 9 | 285.7 | 67.5 | 13.3 |

Average RCC: 62.7 ± 4.2% (of b) (C4 + C3); Average RCC: 12.1 ± 1.9% of c (n = 3, decay corrected)

HPLC-isolated RCCs of [¹¹C]24b and [¹¹C]24c by S1

### Example 129: Radio Reaction Optimization—Radio HPLC Analysis and Characterization of [¹¹C]Cyanide Labeled Arenes—Compounds [¹¹C]25b

| | |
|---|---|
| 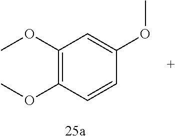 | 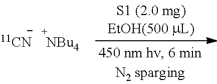 |

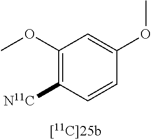

| | | | | | | | Decay | |
|---|---|---|---|---|---|---|---|---|
| | | Substrate | Activity | Injected | Collected | Isolation | corrected | Radiochemical |
| Reaction | | Loading | ([¹¹C]TBACN) | dose | dose | time | Injection dose | conversion |
| Entry | Substrate | (mmol) | (mCi) | (μCi) | (μCi) | (min) | (μCi) | (RCC, %, d.c.) |
| 1 | 25a | 0.05 | 16.0 | 293 | 17 | 8 | 223.2 | 7.6 |
| 2 | | 0.03 | 15.2 | 581 | 8 | 8 | 442.5 | 1.8 |
| 3 | | 0.01 | - | - | 0 | - | - | 0 |

RCC: 7.6% (n = 1, n = .05 mmol, decay corrected)

### HPLC-isolated RCCs of [¹¹C]25b by S1

### Example 130: Radio Reaction Optimization—Radio HPLC Analysis and Characterization of [¹¹C]Cyanide Labeled Arenes—Compounds [¹¹C]26c

| | |
|---|---|
| 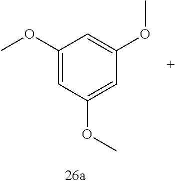 | 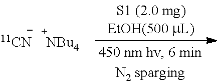 |

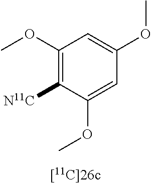

| | | | | | | | Decay | |
|---|---|---|---|---|---|---|---|---|
| | | Substrate | Activity | Injected | Collected | Isolation | corrected | Radiochemical |
| Reaction | | Loading | ([¹¹C]TBACN) | dose | dose | time | Injection dose | conversion |
| Entry | Substrate | (mmol) | (mCi) | (μCi) | (μCi) | (min) | (μCi) | (RCC, %, d.c.) |
| 1 | 26a | 0.02 | 8.1 | 471 | 330 | 8 | 359 | 91.9 |

RCC: 91.9% (decay corrected)

### HPLC-isolated RCCs of [¹¹C]26c by S1

### Example 131: Radio Reaction Optimization—Radio HPLC Analysis and Characterization of [¹¹C]Cyanide Labeled Arenes—Compounds [¹¹C]27b/27c

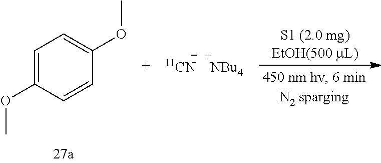

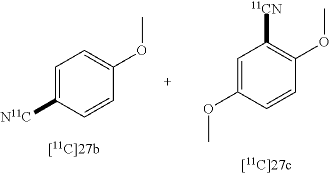

| | | | | | | | | Decay | | |
|---|---|---|---|---|---|---|---|---|---|---|
| | | | Activity | | | | | corrected | Radiochemical | Radiochemical |
| | | Substrate | ([¹¹C] | Injected | Collected | Collected | Isolation | Injection | conversion | conversion |
| Reaction | | Loading | TBACN) | dose | dose (b) | dose (c) | time | dose | (RCC, %, d.c.) | (RCC, %, d.c.) |
| Entry | Substrate | (mmol) | (mCi) | (μCi) | (μCi) | (μCi) | (min) | (μCi) | of b | of c |
| 1 | 27a | 0.04 | 13.1 | 501 | 139 | 29 | 10 | 356.6 | 39.0 | 8.1 |
| 2 | | 0.04 | 22.5 | 476 | 125 | 22 | 10 | 338.8 | 36.9 | 6.5 |
| 3 | | 0.04 | 22.0 | 910 | 242 | 41 | 11 | 626 | 38.7 | 6.5 |
| 4 | | 0.05 | 10.9 | 451 | 118 | 18 | 12 | 300 | 39.3 | 6.0 |
| 5 | | 0.03 | 18.5 | 709 | 143 | 29 | 11 | 487.8 | 29.3 | 2.3 |

Average RCC: 38.2 ± 1.1% (of b); Average RCC: 7.0 ± 0.9% (of c) (N = 3, n = 0.04 mmol, decay corrected)

HPLC-isolated RCCs of [¹¹C]27b and [¹¹C]27c by S1

### Example 132: Radio Reaction Optimization—Radio HPLC Analysis and Characterization of [¹¹C]Cyanide Labeled Arenes—Compounds [¹¹C]28b/28c

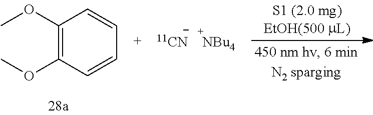

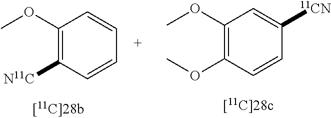

| | | | | | | | | Decay | | |
|---|---|---|---|---|---|---|---|---|---|---|
| | | | Activity | | | | | corrected | Radiochemical | Radiochemical |
| | | Substrate | ([¹¹C] | Injected | Collected | Collected | Isolation | Injection | conversion | conversion |
| Reaction | | Loading | TBACN) | dose | dose (b) | dose (c) | time | dose | (RCC, %, d.c.) | (RCC, %, d.c.) |
| Entry | Substrate | (mmol) | (mCi) | (μCi) | (μCi) | (μCi) | (min) | (μCi) | of b | of c |
| 1 | 28a | 0.03 | 13.3 | 459 | 260 | 22 | 9 | 338 | 76.9 | 6.5 |
| 2 | | 0.03 | 18.8 | 664 | 350 | 33 | 9 | 489 | 71.6 | 6.7 |
| 3 | | 0.03 | 14.8 | 577 | 287 | 27 | 10 | 410.7 | 69.9 | 6.6 |

Average RCC: 72.8 ± 3.71% (of b); Average RCC: 6.6 ± 0.1% (of c) (n = 3, decay corrected)

HPLC-isolated RCCs of [¹¹C]-28b and [¹¹C]-28c by S1

### Example 133: Radio Reaction Optimization—Radio HPLC Analysis and Characterization of [¹¹C]Cyanide Labeled Arenes—Compounds [¹¹C]28b/28c

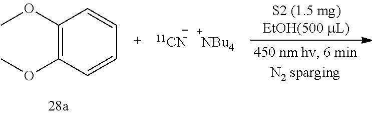

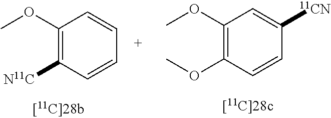

| | | | | | | | | Decay | | |
|---|---|---|---|---|---|---|---|---|---|---|
| | | | Activity | | | | | corrected | Radiochemical | Radiochemical |
| | | Substrate | ([¹¹C] | Injected | Collected | Collected | Isolation | Injection | conversion | conversion |
| Reaction | | Loading | TBACN) | dose | dose (b) | dose (c) | time | dose | (RCC, %, d.c.) | (RCC, %, d.c.) |
| Entry | Substrate | (mmol) | (mCi) | (μCi) | (μCi) | (μCi) | (min) | (μCi) | of b | of c |
| 1 | 28a | 0.03 | 4.6 | 251 | 60 | 5 | 10 | 178.7 | 33.6 | 2.8 |

RCC of b: 33.6%; RCC of c: 2.8% (decay corrected)

HPLC-isolated RCCs of [¹¹C]28b and [¹¹C]28c by S2

### Example 134: Radio Reaction Optimization—Radio HPLC Analysis and Characterization of [¹¹C]Cyanide Labeled Arenes—Compounds [¹¹C]29b/29c

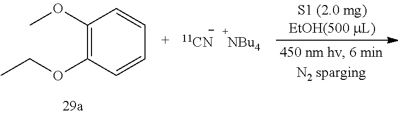

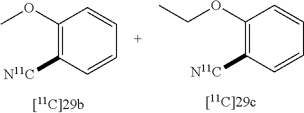

| | | | | | | | | Decay | | |
|---|---|---|---|---|---|---|---|---|---|---|
| | | | Activity | | | | | corrected | Radiochemical | Radiochemical |
| | | Substrate | ([¹¹C] | Injected | Collected | Collected | Isolation | Injection | conversion | conversion |
| Reaction | | Loading | TBACN) | dose | dose (b) | dose (c) | time | dose | (RCC, %, d.c.) | (RCC, %, d.c.) |
| Entry | Substrate | (mmol) | (mCi) | (μCi) | (μCi) | (μCi) | (min) | (μCi) | of b | of c |
| 1 | 29a | 0.03 | 14.1 | 530 | 112 | 125 | 11 | 364.6 | 30.7 | 34.3 |
| 2 | | 0.03 | 3.0 | 123 | 30 | 33 | 11 | 84.6 | 35.5 | 39.0 |
| 3 | | 0.03 | 14.4 | 690 | 173 | 200 | 11 | 474.7 | 36.4 | 42.1 |

Average RCC: 34.2 ± 3.1% (of b); Average RCC: 38.5 ± 3.9% (of c) (n = 3, decay corrected)

### Example 135: Radio Reaction Optimization—Radio HPLC Analysis and Characterization of [¹¹C]Cyanide Labeled Arenes—Compounds [¹¹C]29b/29c

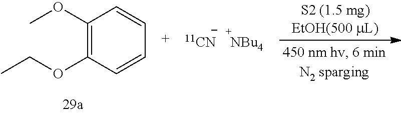

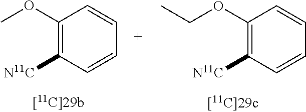

| | | | | | | | | Decay | | |
|---|---|---|---|---|---|---|---|---|---|---|
| | | | Activity | | | | | corrected | Radiochemical | Radiochemical |
| | | Substrate | ([¹¹C] | Injected | Collected | Collected | Isolation | Injection | conversion | conversion |
| Reaction | | Loading | TBACN) | dose | dose (b) | dose (c) | time | dose | (RCC, %, d.c.) | (RCC, %, d.c.) |
| Entry | Substrate | (mmol) | (mCi) | (μCi) | (μCi) | (μCi) | (min) | (μCi) | of b | of c |
| 1 | 29a | 0.02 | | 178 | 15 | 18 | 12 | 118.4 | 12.7 | 15.2 |

RCC of b: 12.7%; RCC of c: 15.2% (decay corrected)

HPLC-isolated RCCs of [¹¹C]29b and [¹¹C]29c by S2

### Example 136: Radio Reaction Optimization—Radio HPLC Analysis and Characterization of [¹¹C]Cyanide Labeled Arenes—Compounds [¹¹C]30b/30c

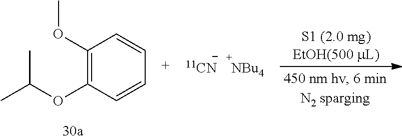

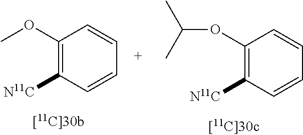

| | | | | | | | | Decay | | |
|---|---|---|---|---|---|---|---|---|---|---|
| | | | Activity | | | | | corrected | Radiochemical | Radiochemical |
| | | Substrate | ([¹¹C] | Injected | Collected | Collected | Isolation | Injection | conversion | conversion |
| Reaction | | Loading | TBACN) | dose | dose (b) | dose (c) | time | dose | (RCC, %, d.c.) | (RCC, %, d.c.) |
| Entry | Substrate | (mmol) | (mCi) | (μCi) | (μCi) | (μCi) | (min) | (μCi) | of b | of c |
| 1 | 30a | 0.02 | 5.2 | 186 | 22 | 70 | 6, 8 | 151.7, | 14.5 | 49.4 |
| | | | | | | | | 141.7 | | |
| 2 | | 0.02 | 3.8 | 247 | 27 | 80 | 7, 8 | 201.4, | 13.4 | 42.5 |
| | | | | | | | | 188.2 | | |
| 3 | | 0.02 | 28.0 | 698 | 91 | 253 | 6, 8 | 569.2, | 16.0 | 47.6 |
| | | | | | | | | 531.8 | | |

Average RCC: 14.6 ± 1.3% (of b); Average RCC: 46.5 ± 3.6% (of c) (N = 3, decay corrected)

HPLC-isolated RCCs of [¹¹C]30b and [¹¹C]30c by S1

### Example 137: Radio Reaction Optimization—Radio HPLC Analysis and Characterization of [¹¹C]Cyanide Labeled Arenes—Compounds [¹¹C]30b/30c

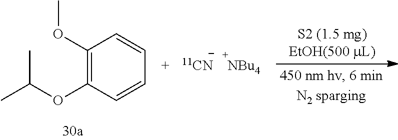

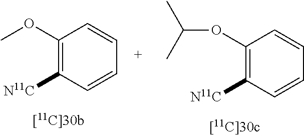

| | | | | | | | | Decay | | |
|---|---|---|---|---|---|---|---|---|---|---|
| | | | Activity | | | | | corrected | Radiochemical | Radiochemical |
| | | Substrate | ([¹¹C] | Injected | Collected | Collected | Isolation | Injection | conversion | conversion |
| Reaction | | Loading | TBACN) | dose | dose (b) | dose (c) | time | dose | (RCC, %, d.c.) | (RCC, %, d.c.) |
| Entry | Substrate | (mmol) | (mCi) | (μCi) | (μCi) | (μCi) | (min) | (μCi) | of b | of c |
| 1 | 30a | 0.02 | 4.3 | 252 | 5 | 23 | 8 | 192 | 2.6 | 12.0 |

RCC of b: 2.6%; RCC of c: 12.0% (decay corrected)

HPLC-isolated RCCs of [¹¹C]30b and [¹¹C]30c by S2

### Example 138: Radio Reaction Optimization—Radio HPLC Analysis and Characterization of [¹¹C]Cyanide Labeled Arenes—Compounds [¹¹C]31b/31c

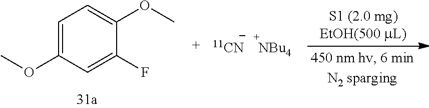

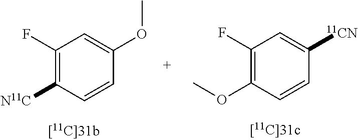

| | | | | | | | | Decay | | |
|---|---|---|---|---|---|---|---|---|---|---|
| | | | Activity | | | | | corrected | Radiochemical | Radiochemical |
| | | Substrate | ([¹¹C] | Injected | Collected | Collected | Isolation | Injection | conversion | conversion |
| Reaction | | Loading | TBACN) | dose | dose (b) | dose (c) | time | dose | (RCC, %, d.c.) | (RCC, %, d.c.) |
| Entry | Substrate | (mmol) | (mCi) | (μCi) | (μCi) | (μCi) | (min) | (μCi) | of b | of c |
| 1 | 31a | 0.03 | 15.9 | 646 | 232 | 50 | 9 | 475.7 | 48.7 | 10.5 |
| 2 | | 0.03 | 16.6 | 628 | 197 | 42 | 11 | 432 | 45.6 | 9.7 |
| 3 | | 0.03 | 9.7 | 487 | 172 | 38 | 11 | 335 | 51.3 | 11.3 |
| 4 | | 0.03 | 6.3 | 392 | 148 | 28 | 9 | 288.7 | 51.3 | 9.7 |

Average RCC: 48.5 ± 2.9% (of b); Average RCC: 10.5 ± 0.8% (of c) (N = 3, n = 0.03 mmol, decay corrected)

HPLC-isolated RCCs of [¹¹C]31b and [¹¹C]31c by S1

### Example 139: Radio Reaction Optimization—Radio HPLC Analysis and Characterization of [¹¹C]Cyanide Labeled Arenes—Compound [¹¹C]33b

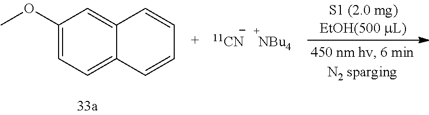

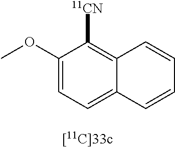

| | | | | | | | Decay | |
|---|---|---|---|---|---|---|---|---|
| | | | Activity | | | | corrected | |
| | | Substrate | ([¹¹C] | Injected | Collected | Isolation | Injection | Radiochemical |
| Reaction | | Loading | TBACN) | dose | dose | time | dose | conversion |
| Entry | Substrate | (mmol) | (mCi) | (μCi) | (μCi) | (min) | (μCi) | (RCC, %, d.c.) |
| 1 | 33a | 0.03 | 19.6 | 585 | 296 | 11 | 402.5 | 73.5 |
| 2 | | 0.03 | 3.1 | 122 | 61 | 9 | 89.8 | 68.0 |
| 3 | | 0.03 | 13.6 | 425 | 228 | 12 | 283 | 80.6 |

Average RCC: 74.0 ± 6.3% (N = 3, decay corrected)

### HPLC-isolated RCCs of [¹¹C]33c by S1

### Example 140: Radio Reaction Optimization—Radio HPLC Analysis and Characterization of [¹¹C]Cyanide Labeled Arenes—Compound [¹¹C]34c

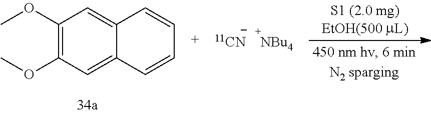

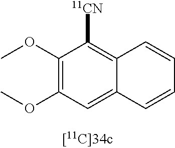

| | | | | | | | Decay | |
|---|---|---|---|---|---|---|---|---|
| | | | Activity | | | | corrected | |
| | | Substrate | ([¹¹C] | Injected | Collected | Isolation | Injection | Radiochemical |
| Reaction | | Loading | TBACN) | dose | dose | time | dose | conversion |
| Entry | Substrate | (mmol) | (mCi) | (μCi) | (μCi) | (min) | (μCi) | (RCC, %, d.c.) |
| 1 | 34a | 0.03 | 13.2 | 702 | 33 | 10 | 499.7 | 66.2 |
| 2 | | 0.03 | 19.5 | 629 | 235 | 12 | 418.3 | 56.2 |

Average RCC: 61.2 ± 7.1% (N = 2, decay corrected)

### HPLC-isolated RCCs of [¹¹C]34c by S1

### Example 141: Radio Reaction Optimization—Radio HPLC Analysis and Characterization of [¹¹C]Cyanide Labeled Arenes—Compound [¹¹C]34c

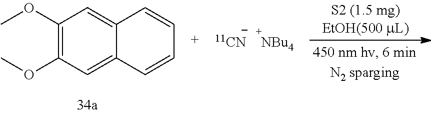

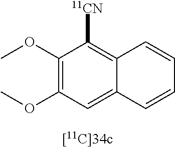

| | | | | | | | Decay | |
|---|---|---|---|---|---|---|---|---|
| | | | Activity | | | | corrected | |
| | | Substrate | ([¹¹C] | Injected | Collected | Isolation | Injection | Radiochemical |
| Reaction | | Loading | TBACN) | dose | dose | time | dose | conversion |
| Entry | Substrate | (mmol) | (mCi) | (μCi) | (μCi) | (min) | (μCi) | (RCC, %, d.c.) |
| 1 | 34a | 0.03 | | 174 | 39 | 12 | 115.7 | 33.7 |

Average RCC: 33.7% (decay corrected)

### HPLC-isolated RCCs of [¹¹C]34c by S1

### Example 142: Radio Reaction Optimization—Radio HPLC Analysis and Characterization of [¹¹C]Cyanide Labeled Arenes—Compounds [¹¹C]35b/35c/35d

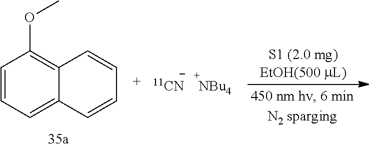

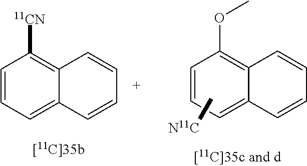

| | | | | | | | | Decay | | |
|---|---|---|---|---|---|---|---|---|---|---|
| | | | Activity | | | | | corrected | Radiochemical | Radiochemical |
| | | Substrate | ([¹¹C] | Injected | Collected | Collected | Isolation | Injection | conversion | conversion |
| Reaction | | Loading | TBACN) | dose | dose (b) | dose (c) | time | dose | (RCC, %, d.c.) | (RCC, %, d.c.) |
| Entry | Substrate | (mmol) | (mCi) | (μCi) | (μCi) | (μCi) | (min) | (μCi) | of b | of c |
| 1 | 35a | 0.03 | 8.9 | 400 | 79 | 67 + 31 | 10 | 284.7 | 27.7 | 23.5, 11.0 |
| 2 | | 0.03 | 11.9 | 435 | 68 | 75 + 32 | 12 | 289.3 | 23.5 | 25.9, 11.0 |

Average RCC: 25.1 ± 1.7% (of b); Average RCC: 24.8 ± 1.8% (of c) (N = 2, decay corrected)

### HPLC-isolated RCCs of [¹¹C]35 by S1

### Example 143: Radio Reaction Optimization—Radio HPLC Analysis and Characterization of [¹¹C]Cyanide Labeled Arenes—Compound [¹¹C]36b

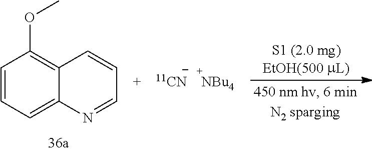

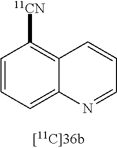

| | | | | | | | Decay | |
|---|---|---|---|---|---|---|---|---|
| | | | Activity | | | | corrected | |
| | | Substrate | ([¹¹C] | Injected | Collected | Isolation | Injection | Radiochemical |
| Reaction | | Loading | TBACN) | dose | dose | time | dose | conversion |
| Entry | Substrate | (mmol) | (mCi) | (μCi) | (μCi) | (min) | (μCi) | (RCC, %, d.c.) |
| 1 | 36a | 0.03 | 11.5 | 450 | 9 | 9 | 331 | 2.7 |
| 2 | | 0.01 | 17.0 | 611 | 5 | 7 | 481.5 | 1.0 |

RCC: 2.7% (N = 1, n = 0.03 mmol, decay corrected)

### HPLC-isolated RCCs of [¹¹C]36b by S1

### Example 144: Radio Reaction Optimization—Radio HPLC Analysis and Characterization of [¹¹C]Cyanide Labeled Arenes—Compound [¹¹C]36b

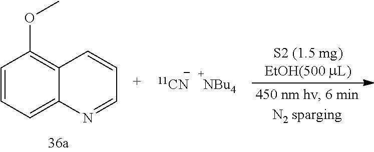

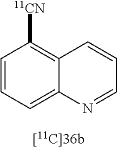

| | | | | | | | Decay | |
|---|---|---|---|---|---|---|---|---|
| | | | Activity | | | | corrected | |
| | | Substrate | ([¹¹C] | Injected | Collected | Isolation | Injection | Radiochemical |
| Reaction | | Loading | TBACN) | dose | dose | time | dose | conversion |
| Entry | Substrate | (mmol) | (mCi) | (μCi) | (μCi) | (min) | (μCi) | (RCC, %, d.c.) |
| 1 | 36a | 0.03 | 4.7 | 220 | 33 | 7 | 173.4 | 19.0 |
| 2 | | 0.01 | 8.4 | 461 | 60 | 9 | 339.5 | 17.7 |
| 3 | | 0.02 | 2.45 | 165 | 21 | 9 | 121.5 | 17.3 |

Average RCC: 18.0 ± 0.9% (N = 3, decay corrected)

### HPLC-isolated RCCs of [¹¹C]36b by S2

### Example 145: Radio Reaction Optimization—Radio HPLC Analysis and Characterization of [¹¹C]Cyanide Labeled Arenes—Compound [¹¹C]37c

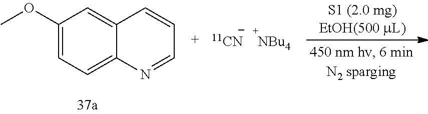

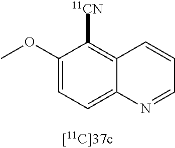

| | | | | | | | Decay | |
|---|---|---|---|---|---|---|---|---|
| | | | Activity | | | | corrected | |
| | | Substrate | ([¹¹C] | Injected | Collected | Isolation | Injection | Radiochemical |
| Reaction | | Loading | TBACN) | dose | dose | time | dose | conversion |
| Entry | Substrate | (mmol) | (mCi) | (μCi) | (μCi) | (min) | (μCi) | (RCC, %, d.c.) |
| 1 | 37a | 0.03 | 19.1 | 558 | 46 | 9 | 410.9 | 11.2 |
| 2 | | 0.03 | 20.5 | 575 | 41 | 9 | 423 | 9.7 |
| 3 | | 0.03 | 17.1 | 500 | 90 | 9 | 368 | 24.4 |

Average RCC: 15.1 ± 8.1% (N = 3, decay corrected)

### HPLC-isolated RCCs of [¹¹C]37c by S1

### Example 146: Radio Reaction Optimization—Radio HPLC Analysis and Characterization of [¹¹C]Cyanide Labeled Arenes—Compound [¹¹C]38b

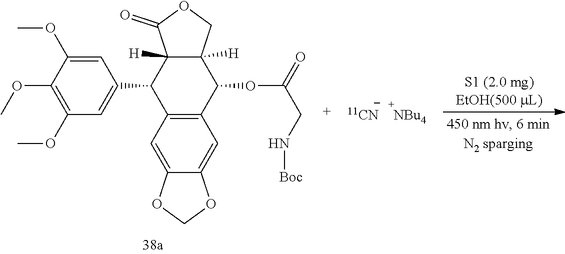

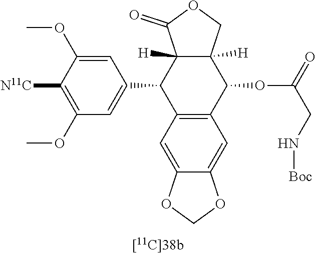

| | | | | | | | Decay | |
|---|---|---|---|---|---|---|---|---|
| | | Substrate | Activity | Injected | Collected | Isolation | corrected | Radiochemical |
| Reaction | | Loading | ([¹¹C]TBACN) | dose | dose | time | Injection dose | conversion |
| Entry | Substrate | (mmol) | (mCi) | (μCi) | (μCi) | (min) | (μCi) | (RCC, %, d.c.) |
| 1 | 38a | 0.01 | 25.3 | 700 | 182 | 10 | 498.2 | 36.5 |
| 2 | | 0.01 | 12.7 | 662 | 174 | 9 | 487.5 | 35.7 |
| 3 | | 0.01 | 22.5 | 585 | 155 | 10 | 416.4 | 37.2 |

Average RCC: 36.5 ± 0.8% (N = 3, decay corrected)

### HPLC-isolated RCCs of [¹¹C]38b by S1

### Example 147: Radio Reaction Optimization—Radio HPLC Analysis and Characterization

of [¹¹C]cyanide labeled arenes—Compound [¹¹C]39b

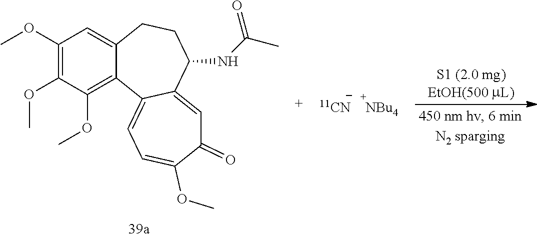 [SMILES: COc1cc2c(c(OC)c1OC)-c1ccc(OC)c(=O)cc1[C@@H](NC(C)=O)CC2 ; "11CN" ; "NBu4"]

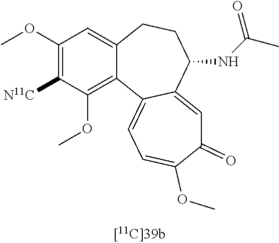

| | | | | | | | Decay | |
|---|---|---|---|---|---|---|---|---|
| | | Substrate | Activity | Injected | Collected | Isolation | corrected | Radiochemical |
| Reaction | | Loading | ([¹¹C]TBACN) | dose | dose | time | Injection dose | conversion |
| Entry | Substrate | (mmol) | (mCi) | (μCi) | (μCi) | (min) | (μCi) | (RCC, %, d.c.) |
| 1 | 39a | 0.03 | 15.0 | 597 | 262 | 7 | 470.5 | 55.7 |
| 2 | | 0.03 | 16.0 | 821 | 409 | 7 | 647 | 63.2 |
| 3 | | 0.03 | 4.6 | 158 | 84 | 7 | 124.5 | 67.5 |
| 4 | | 0.01 | 13.6 | 714 | 265 | 12 | 474.8 | 55.8 |

Average RCC: 62.1 ± 6.0% (N = 3, n = 0.03 mmol, decay corrected)

### HPLC-isolated RCCs of [¹¹C]39b by S1

### Example 148: Radio Reaction Optimization—Radio HPLC Analysis and Characterization of [¹¹C]Cyanide Labeled Arenes—Compound [¹¹C]39b

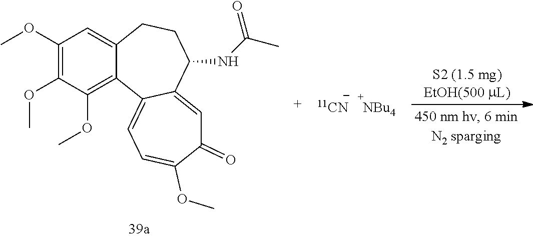 [SMILES: COc1cc2c(c(OC)c1OC)-c1ccc(OC)c(=O)cc1[C@@H](NC(C)=O)CC2 ; "11CN" ; "NBu4"]

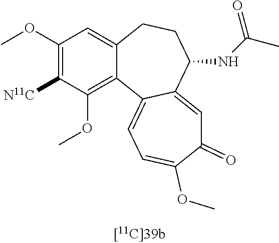

| | | | | | | | Decay | |
|---|---|---|---|---|---|---|---|---|
| | | Substrate | Activity | Injected | Collected | Isolation | corrected | Radiochemical |
| Reaction | | Loading | ([¹¹C]TBACN) | dose | dose | time | Injection dose | conversion |
| Entry | Substrate | (mmol) | (mCi) | (μCi) | (μCi) | (min) | (μCi) | (RCC, %, d.c.) |
| 1 | 39a | 0.03 | 3.52 | 158 | 45 | 9 | 116.5 | 38.6 |

RCC: 38.6% (decay corrected)

### HPLC-isolated RCCs of [¹¹C]39b by S2

### Example 149: Radio Reaction Optimization—Radio HPLC Analysis and Characterization of [¹¹C]Cyanide Labeled Arenes—Compound [¹¹C]40b

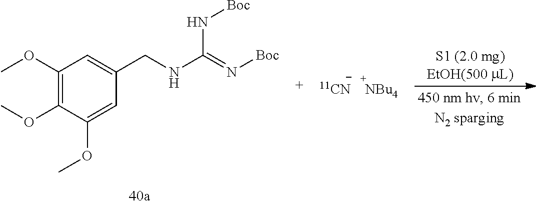 [SMILES: COc1cc(CN/C(=N/C(=O)OC(C)(C)C)NC(=O)OC(C)(C)C)cc(OC)c1OC ; "11CN" ; "NBu4"]

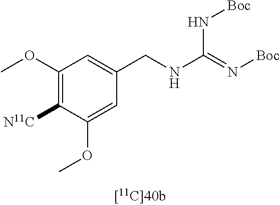

| | | | | | | | Decay | |
|---|---|---|---|---|---|---|---|---|
| | | Substrate | Activity | Injected | Collected | Isolation | corrected | Radiochemical |
| Reaction | | Loading | ([¹¹C]TBACN) | dose | dose | time | Injection dose | conversion |
| Entry | Substrate | (mmol) | (mCi) | (μCi) | (μCi) | (min) | (μCi) | (RCC, %, d.c.) |
| 1 | 40a | 0.01 | 6.2 | 393 | 85 | 8 | 299.4 | 28.4 |
| 2 | | 0.01 | 5.0 | 351 | 66 | 8 | 267.4 | 24.7 |
| 3 | | 0.01 | 11.5 | 638 | 140 | 9 | 469.8 | 29.8 |
| 4 | | 0.01 | 12.6 | 485 | 123 | 8 | 369.5 | 33.2 |

Average RCC: 29.0 ± 3.5% (N = 4, n = 0.01 mmol, decay corrected)

### HPLC-isolated RCCs of [¹¹C]40b by S1

### Example 150: Radio Reaction Optimization—Radio HPLC Analysis and Characterization of [¹¹C]Cyanide Labeled Arenes—Compound [¹¹C]40b

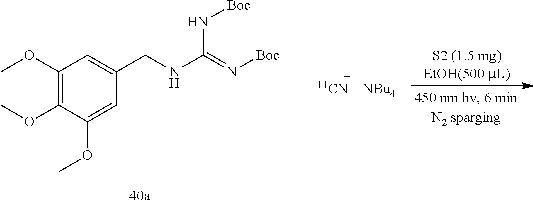 [SMILES: COc1cc(CN/C(=N/C(=O)OC(C)(C)C)NC(=O)OC(C)(C)C)cc(OC)c1OC ; "11CN" ; "NBu4"]

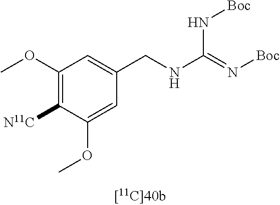

| | | | | | | | Decay | |
|---|---|---|---|---|---|---|---|---|
| | | Substrate | Activity | Injected | Collected | Isolation | corrected | Radiochemical |
| Reaction | | Loading | ([¹¹C]TBACN) | dose | dose | time | Injection dose | conversion |
| Entry | Substrate | (mmol) | (mCi) | (μCi) | (μCi) | (min) | (μCi) | (RCC, %, d.c.) |
| 1 | 40a | 0.012 | 11.5 | 260 | 5 | 8 | 198 | 2.6 |

RCC: 2.5% (decay corrected)

### HPLC-isolated RCCs of [¹¹C]-40b by S2

### Example 151: Radio Reaction Optimization—Radio HPLC Analysis and Characterization of [¹¹C]Cyanide Labeled Arenes—Compound [¹¹C]41b

`

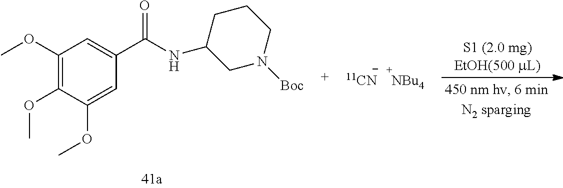 [SMILES: COc1cc(C(=O)NC2CCCN(C(=O)OC(C)(C)C)C2)cc(OC)c1OC ; "11CN" ; "NBu4"]

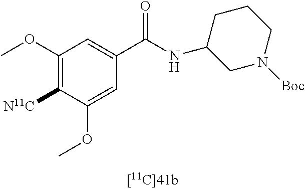

| | | | | | | | Decay | |
|---|---|---|---|---|---|---|---|---|
| | | Substrate | Activity | Injected | Collected | Isolation | corrected | Radiochemical |
| Reaction | | Loading | ([¹¹C]TBACN) | dose | dose | time | Injection dose | conversion |
| Entry | Substrate | (mmol) | (mCi) | (μCi) | (μCi) | (min) | (μCi) | (RCC, %, d.c.) |
| 1 | 41a | 0.02 | 7.9 | 173 | 33 | 8 | 131.8 | 25.0 |
| 2 | | 0.015 | 14.0 | 465 | 74 | 8 | 354.2 | 20.9 |
| 3 | | 0.025 | 8.35 | 220 | 27 | 8 | 167.6 | 16.1 |

Average RCC: 20.7 ± 4.5% (N = 3, decay corrected)

### HPLC-isolated RCCs of [AiC]41b by S n

### Example 152: Radio Reaction Optimization—Radio HPLC Analysis and Characterization of [¹¹C]Cyanide Labeled Arenes—Compound [(C]42b

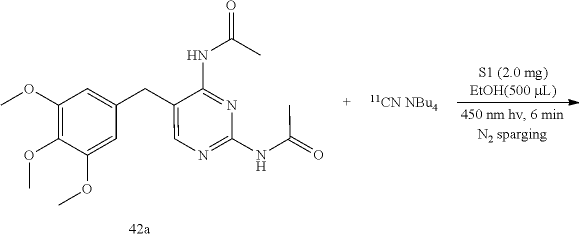 [SMILES: COc1cc(Cc2cnc(NC(C)=O)nc2NC(C)=O)cc(OC)c1OC ; "11CN" ; "NBu4"]

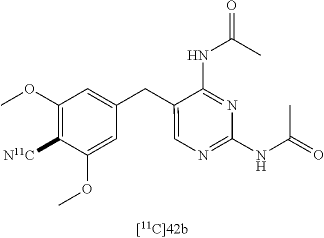

| | | | | | | | Decay | | |
|---|---|---|---|---|---|---|---|---|---|
| | | | | | | | corrected | | |
| | | Substrate | Activity | Injected | Collected | Isolation | Injection | Radiochemical | Radiochemical |
| Reaction | | Loading | ([¹¹C]TBACN) | dose | dose | time | dose | conversion | conversion |
| Entry | Substrate | (mmol) | (mCi) | (μCi) | (μCi) | (min) | (μCi) | (RCC, %, d.c.) | (RCY, %, n.d.c.) |
| 1 | 42a | 0.01 | 7.3 | 163 | 91 | 8 | 124.2 | 73.2 | — |
| 2 | | 0.01 | 8.7 | 408 | 222 | 9 | 300.5 | 73.9 | — |
| 3 | | 0.01 | 14.4 | 366 | 193 | 8 | 278.8 | 69.3 | — |
| 4 | | 0.01 | 7.3 | 545 | 255 | 14 | 338.6 | 75.3 | 46.8 |
| | | | | | | | | (qc-F5) | |
| 5 | | 0.0085 | 16.3 | 1885 | 850 | 13 | 1211.7 | 70.2 | 45.1 |
| 6 | | 0.006 | 31 | 4830 | 2300 | 13 | 2815.5 | 74.1 | 47.6 |
| 7 | | 0.005 | 29.9 | 5490 | 2440 | 15 | 3297 | 74.0 | 44.4 |

Average RCC: 72.9 ± 2.2% (N = 7, decay corrected); Average isolated RCY: 46.0 ± 1.5% (N = 4, none decay corrected)

### HPLC-isolated RCCs of [¹¹C]42b by S1

### Example 153: Radio Reaction Optimization—Radio HPLC Analysis and Characterization of [¹¹C]Cyanide Labeled Arenes—Compound [¹¹C]42d

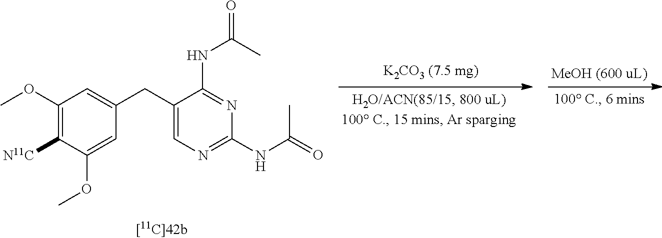

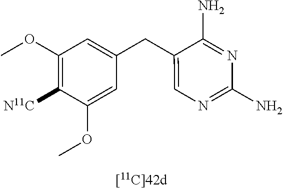

| | | | | | | Decay | |
|---|---|---|---|---|---|---|---|
| | | Activity | Injected | Collected | Isolation | corrected | Radiochemical |
| Reaction | | ([11C]-42b) | dose | dose | time | Injection dose | conversion |
| Entry | Substrate | (mCi) | (μCi) | (μCi) | (min) | (μCi) | (RCC, %, d.c.) |
| 1 | [¹¹C]42b | 1.35 | 417 | 292 | 9 | 307 | 95.1 |

RCC: 95.1% (decay corrected)

HPLC-isolated RCCs of [¹¹C]42d from [¹¹C]42b

### Example 154: Radio Reaction Optimization—Radio HPLC Analysis and Characterization of [¹¹C]Cyanide Labeled Arenes—Compound [¹¹C]42d

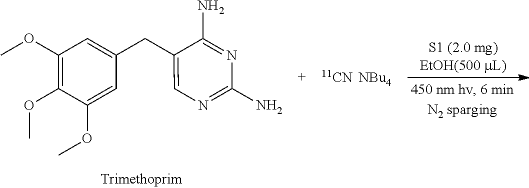 [SMILES: COc1cc(Cc2cnc(N)nc2N)cc(OC)c1OC ; "11CN" ; "NBu4"]

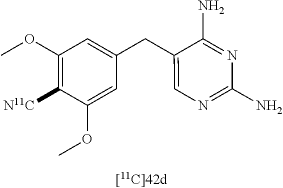

| | | | | | | Decay | |
|---|---|---|---|---|---|---|---|
| | | Activity | Injected | Collected | Isolation | corrected | Radiochemical |
| Reaction | | ([¹¹C]TBACN) | dose | dose | time | Injection dose | conversion |
| Entry | Substrate | (mCi) | (μCi) | (μCi) | (min) | (μCi) | (RCC, %, d.c.) |
| 1 | Trimethoprim | 7.15 | 402 | 25 | 13 | 258 | 9.7 |

RCC: 9.7% (decay corrected)

HPLC analysis RCCs of [¹¹C]42d from trimethoprim

### Example 155: Radio Reaction Optimization—Radio HPLC Analysis and Characterization of [¹¹C]Cyanide Labeled Arenes—Compound [¹¹C]43b

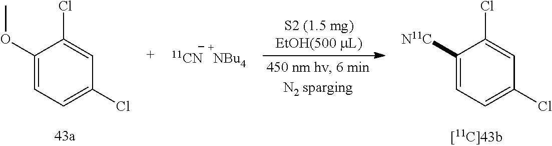

| | | | | | | | Decay | |
|---|---|---|---|---|---|---|---|---|
| | | Substrate | Activity | Injected | Collected | Isolation | corrected | Radiochemical |
| Reaction | | Loading | ([¹¹C]TBACN) | dose | dose b | time | Injection dose | conversion of b |
| Entry | Substrate | (mmol) | (mCi) | (μCi) | (μCi) | (min) | (μCi) | (RCC, %, d.c.) |
| 1 | 43a | 0.03 | 15.4 | 717 | 155 | 10 | 510 | 30.4 |
| 2 | | 0.03 | 3.0 | 241 | 75 | 9 | 177.5 | 42.3 |
| 3 | | 0.03 | 10.9 | 204 | 48 | 11 | 140 | 34.3 |
| 4 | Co- | 0.01 | 2.0 | 176 | 30 | 7 | 138.7 | 21.6 |
| | injection | | | | | | | |

Average RCC: 35.7 ± 6.1% (N = 3, decay corrected)

### HPLC-isolated RCCs of [¹¹C]43b by S2

### Example 156: Radio Reaction Optimization—Radio HPLC Analysis and Characterization of [¹¹C]Cyanide Labeled Arenes—Compound [¹¹C]44b

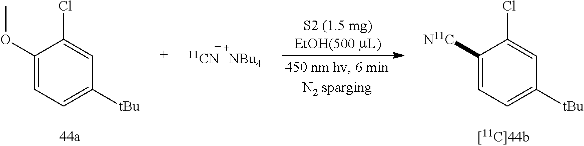

| | | | | | | | Decay | |
|---|---|---|---|---|---|---|---|---|
| | | Substrate | Activity | Injected | Collected | Isolation | corrected | Radiochemical |
| Reaction | | Loading | ([¹¹C]TBACN) | dose | dose b | time | Injection dose | conversion of b |
| Entry | Substrate | (mmol) | (mCi) | (μCi) | (μCi) | (min) | (μCi) | (RCC, %, d.c.) |
| 1 | 44a | 0.02 | 6.3 | 376 | 86 | 19 | 197 | 43.7 |
| 2 | | 0.02 | 5.0 | 172 | 53 | 10 | 122.4 | 43.3 |
| 3 | | 0.02 | 9.9 | 505 | 168 | 9 | 371.9 | 45.2 |

Average RCC: 44.1 ± 1.0% (N = 3, decay corrected)

### HPLC-isolated RCCs of [¹¹C]44b by S2

### Example 157: Radio Reaction Optimization—Radio HPLC Analysis and Characterization of [¹¹C]Cyanide Labeled Arenes—Compound [¹¹C]45b

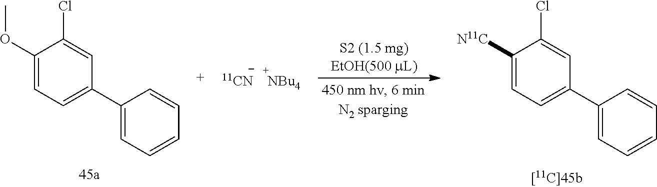

| | | | | | | | Decay | |
|---|---|---|---|---|---|---|---|---|
| | | Substrate | Activity | Injected | Collected | Isolation | corrected | Radiochemical |
| Reaction | | Loading | ([¹¹C]TBACN) | dose | dose b | time | Injection dose | conversion of b |
| Entry | Substrate | (mmol) | (mCi) | (μCi) | (μCi) | (min) | (μCi) | (RCC, %, d.c.) |
| 1 | 45a | 0.025 | 14.3 | 364 | 96 | 11 | 250.5 | 38.3 |
| 2 | | 0.02 | 4.7 | 194 | 38 | 13 | 124.7 | 30.4 |
| 3 | | 0.02 | 5.8 | 169 | 43 | 11 | 116.3 | 37 |

Average RCC: 35.2 ± 4.2% (N = 3, decay corrected)

### HPLC-isolated RCCs of [¹¹C]45b by S2

### Example 158: Radio Reaction Optimization—Radio HPLC Analysis and Characterization of [¹¹C]Cyanide Labeled Arenes—Compound [¹¹C]46b

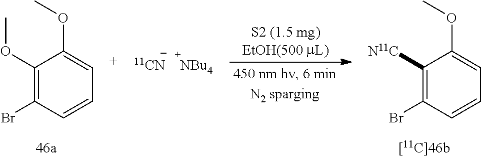

| | | | | | | | Decay | |
|---|---|---|---|---|---|---|---|---|
| | | Substrate | Activity | Injected | Collected | Isolation | corrected | Radiochemical |
| Reaction | | Loading | ([¹¹C]TBACN) | dose | dose b | time | Injection dose | conversion of b |
| Entry | Substrate | (mmol) | (mCi) | (μCi) | (μCi) | (min) | (μCi) | (RCC, %, d.c.) |
| 1 | 46a | 0.03 | 16.0 | 582 | 90 | 13 | 374 | 24.1 |
| 2 | | 0.03 | 3.7 | 205 | 37 | 11 | 141 | 26.2 |
| 3 | | 0.025 | 15.9 | 570 | 102 | 10 | 405.7 | 25.1 |

Average RCC: 25.1 ± 1.1% (N = 3, decay corrected)

### HPLC-isolated RCCs of [¹¹C]46b by S2

### Example 159: Radio Reaction Optimization—Radio HPLC Analysis and Characterization of [¹¹C]Cyanide Labeled Arenes—Compounds [¹¹C]47b (C4)/(C3)

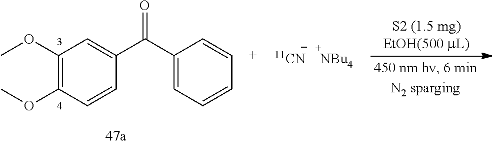

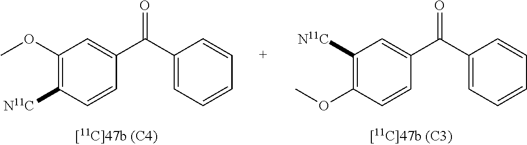

| | | | | | | | | Decay | Radiochemical | Radiochemical |
|---|---|---|---|---|---|---|---|---|---|---|
| | | Substrate | Activity | Injected | Collected | Collected | Isolation | corrected | conversion of | conversion of |
| Reaction | | Loading | ([¹¹C]TBACN) | dose | dose (b) | dose (c) | time | Injection dose | b | c |
| Entry | Substrate | (mmol) | (mCi) | (μCi) | (μCi) | (μCi) | (min) | (μCi) | (RCC, %, d.c.) | (RCC, %, d.c.) |
| 1 | 47a | 0.02 | 8.3 | 457 | 52 | 31 | 11 | 314.4 | 16.5 | 9.9 |
| 2 | | 0.02 | 1.2 | 78 | 8 | 5 | 10 | 55.5 | 14.4 | 9.0 |
| 3 | | 0.02 | 5.6 | 312 | 34 | 21 | 11 | 214.6 | 15.8 | 9.8 |
| 4 | | 0.02 | 7.4 | 334 | 42 | 27 | 10 | 237.7 | 17.7 | 11.4 |
| 5 | | 0.02 | 2.0 | 120 | 12 | 7 | 10 | 85.4 | 14.1 | 8.2 |

Average RCC of b: 15.7 ± 1.5%; Average RCC of c: 9.7 ± 1.2% (N = 5, decay corrected)

### HPLC-isolated RCCs of [¹¹C]47 by S2

### Example 160: Radio Reaction Optimization—Radio HPLC Analysis and Characterization of [¹¹C]Cyanide Labeled Arenes—Compounds [¹¹C]48b (C4)/(C3)

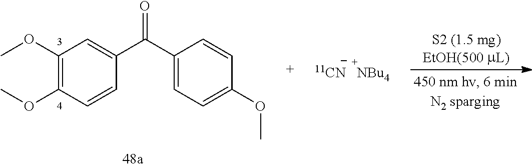

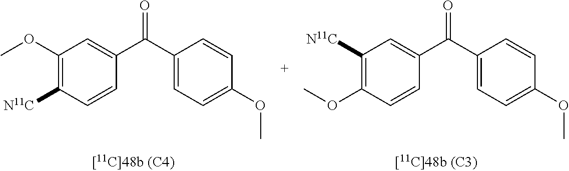

| | | | | | | | | Decay | Radiochemical | Radiochemical |
|---|---|---|---|---|---|---|---|---|---|---|
| | | | | | | | | corrected | conversion of | conversion of |
| | | Substrate | Activity | Injected | Collected | Collected | Isolation | Injection | b | c |
| Reaction | | Loading | ([¹¹C]TBACN) | dose | dose (b) | dose (c) | time | dose | (RCC, %, | (RCC, %, |
| Entry | Substrate | (mmol) | (mCi) | (μCi) | (μCi) | (μCi) | (min) | (μCi) | d.c.) | d.c.) |
| 1 | 48a | 0.03 | 3.6 | 188 | 29 | 24 | 9 | 138.4 | 21 | 17.3 |
| 2 | | 0.03 | 8.3 | 416 | 63 | 58 | 9 | 306.4 | 20.6 | 18.9 |
| 3 | | 0.03 | 12.1 | 498 | 86 | 68 | 9 | 366.7 | 23.5 | 18.5 |

Average RCC of b: 21.7 ± 1.6%; Average RCC of c: 18.2 ± 0.8% (N = 3, decay corrected)

### HPLC-isolated RCCs of [¹¹C]48 by S2

### Example 161: Radio Reaction Optimization—Radio HPLC Analysis and Characterization of [¹¹C]Cyanide Labeled Arenes—Compounds [¹¹C]49b (C4)/(C3)

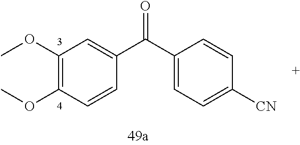 [SMILES: COc1ccc(C(=O)c2ccc(C#N)cc2)cc1OC]

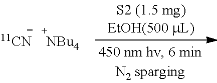

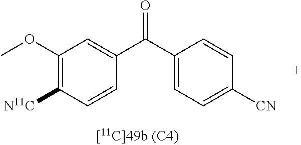

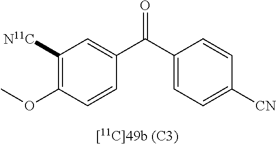

| | | | | | | | | Decay | Radio- | Radio- |
|---|---|---|---|---|---|---|---|---|---|---|
| | | | Activity | | | | | corrected | chemical | chemical |
| | | Substrate | ([¹¹C] | Injected | Collected | Collected | Isolation | Injection | conversion | conversion |
| Reaction | | Loading | TBACN) | dose | dose (b) | dose (c) | time | dose | of b (RCC, | of c (RCC, |
| Entry | Substrate | (mmol) | (mCi) | (μCi) | (μCi) | (μCi) | (min) | (μCi) | %, d.c.) | %, d.c.) |
| 1 | 49a | 0.025 | 6.3 | 215 | trace | trace | — | — | trace | trace |
| 2 | | 0.025 | 5.6 | 196 | 0 | 0 | — | — | 0 | 0 |
| 3 | | 0.04 | 7.2 | 210 | trace | trace | — | — | trace | trace |
| 4 | | 0.01 | 5.65 | 217 | 0 | 0 | — | — | 0 | 0 |

Trace. (N = 2)

### HPLC-isolated RCCs of [¹¹C]49 by S2

### Example 162: Radio Reaction Optimization—Radio HPLC Analysis and Characterization of [¹¹C]Cyanide Labeled Arenes—Compounds [¹¹C] 50b (C4)/(C3)

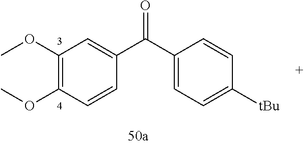 [SMILES: COc1ccc(C(=O)c2ccc(C(C)(C)C)cc2)cc1OC]

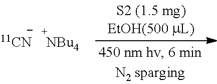

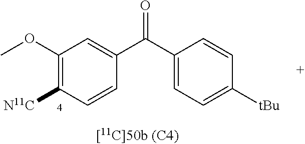

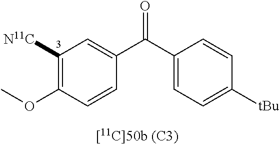

| | | | | | | | | Decay | Radio- | Radio- |
|---|---|---|---|---|---|---|---|---|---|---|
| | | | Activity | | | | | corrected | chemical | chemical |
| | | Substrate | ([¹¹C] | Injected | Collected | Collected | Isolation | Injection | conversion | conversion |
| Reaction | | Loading | TBACN) | dose | dose (b) | dose (c) | time | dose | of b (RCC, | of c (RCC, |
| Entry | Substrate | (mmol) | (mCi) | (μCi) | (μCi) | (μCi) | (min) | (μCi) | %, d.c.) | %, d.c.) |
| 1 | 50a | 0.025 | 4.0 | 188 | 31 | 28 | 15 | 113 | 27.4 | 24.8 |
| 2 | | 0.025 | 2.8 | 173 | 26 | 24 | 10 | 123 | 21.1 | 19.5 |
| 3 | | 0.025 | 17.5 | 655 | 56 | 55 | 13 | 421 | 13.3 | 13.1 |
| 4 | | 0.01 | 4.4 | 222 | 23 | 21 | 20 | 112.5 | 20.4 | 18.7 |
| 5 | | 0.015 | 3.7 | 296 | 32 | 24 | 13 | 190 | 16.8 | 12.6 |

Average RCC of b: 19.8 ± 5.3%; Average RCC of c: 17.7 ± 5.0% (N = 5, decay corrected)

### HPLC-isolated RCCs of [¹¹C]50 by S2

### Example 163: Radio Reaction Optimization—Radio HPLC Analysis and Characterization of [¹¹C]Cyanide Labeled Arenes—Compounds [¹¹C]51b (C4)/(C3)

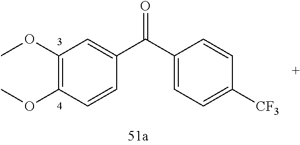 [SMILES: COc1ccc(C(=O)c2ccc(C(F)(F)F)cc2)cc1OC]

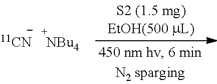

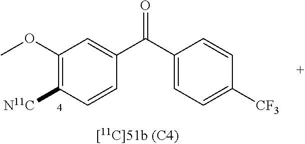

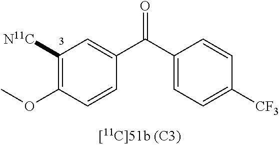

| | | | | | | | | Decay | Radio- | Radio- |
|---|---|---|---|---|---|---|---|---|---|---|
| | | | Activity | | | | | corrected | chemical | chemical |
| | | Substrate | ([¹¹C] | Injected | Collected | Collected | Isolation | Injection | conversion | conversion |
| Reaction | | Loading | TBACN) | dose | dose b | dose c | time | dose | of b (RCC, | of c (RCC, |
| Entry | Substrate | (mmol) | (mCi) | (μCi) | (μCi) | (μCi) | (min) | (μCi) | %, d.c.) | %, d.c.) |
| 1 | 51a | 0.02 | 22.5 | 528 | 40 | 38 | 13 | 339.4 | 11.8 | 11.2 |
| 2 | | 0.02 | 20.0 | 678 | 49 | 47 | 9 | 499 | 9.8 | 9.4 |
| 3 | | 0.02 | 3.7 | 148 | 15 | 12 | 12 | 98.4 | 15.2 | 12.2 |

Average RCC of b: 12.3 ± 2.7%; Average RCC of c: 10.9 ± 1.4% (N = 3, decay corrected)

### HPLC-isolated RCCs of [¹¹C]e51 by S2

### Example 164: Radio Reaction Optimization—Radio HPLC Analysis and Characterization of [¹¹C]Cyanide Labeled Arenes—Compounds [¹¹C]52b (C4)/(C3)

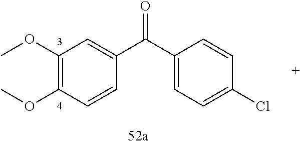 [SMILES: COc1ccc(C(=O)c2ccc(Cl)cc2)cc1OC]

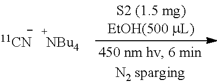

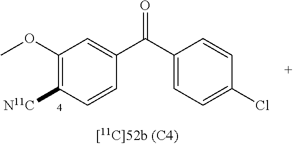

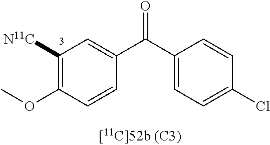

| | | | | | | | | Decay | Radio- | Radio- |
|---|---|---|---|---|---|---|---|---|---|---|
| | | | Activity | | | | | corrected | chemical | chemical |
| | | Substrate | ([¹¹C] | Injected | Collected | Collected | Isolation | Injection | conversion | conversion |
| Reaction | | Loading | TBACN) | dose | dose b | dose c | time | dose | of b (RCC, | of c (RCC, |
| Entry | Substrate | (mmol) | (mCi) | (μCi) | (μCi) | (μCi) | (min) | (μCi) | %, d.c.) | %, d.c.) |
| 1 | 52a | 0.02 | 8.0 | 321 | 41 | 40 | 10 | 228.5 | 17.9 | 17.5 |
| 2 | | 0.02 | 5.4 | 276 | 30 | 23 | 11 | 189.9 | 15.8 | 12.1 |
| 3 | | 0.02 | 7.7 | 280 | 25 | 18 | 11 | 192.6 | 13.0 | 9.3 |

Average RCC of b: 15.6 ± 2.5%; Average RCC of c: 13.0 ± 4.2% (N = 3, decay corrected)

### HPLC-isolated RCCs of [¹¹C]52 by S2

### Example 165: Radio Reaction Optimization—Radio HPLC Analysis and Characterization of [¹¹C]Cyanide Labeled Arenes—Compounds [¹¹C]53b (C4)/(C3)

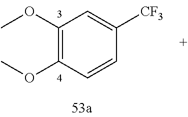

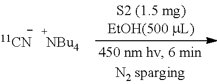

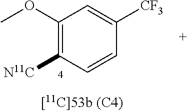

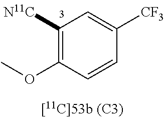

| | | | | | | | Decay | Radio- |
|---|---|---|---|---|---|---|---|---|
| | | | Activity | | | | corrected | chemical |
| | | Substrate | ([¹¹C] | Injected | Collected | Isolation | Injection | conversion |
| Reaction | | Loading | TBACN) | dose | dose b + c | time | dose | of b + c |
| Entry | Substrate | (mmol) | (mCi) | (μCi) | (μCi) | (min) | (μCi) | (RCC, %, d.c.) |
| 1 | 53a | 0.02 | 4.0 | 228 | 42 | 18 | 123.6 | 34.0 |
| 2 | | 0.02 | 2.7 | 356 | 93 | 8 | 271.2 | 34.3 |
| 3 | | 0.02 | 3.1 | 167 | 48 | 8 | 127.2 | 37.7 |

Average RCC of b + c: 35.3 ± 2.1% (N = 3, decay corrected)

### HPLC-isolated RCCs of [¹¹C]53 by S2

### Example 166: Radio Reaction Optimization—Radio HPLC Analysis and Characterization of [¹¹C]Cyanide Labeled Arenes—Compounds [¹¹C] 54b (C4)/(C3)

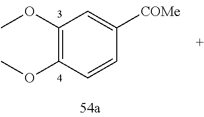

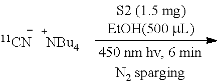

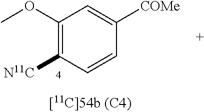

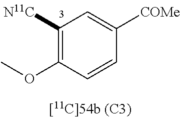

| | | | | | | | | Decay | Radio- | Radio- |
|---|---|---|---|---|---|---|---|---|---|---|
| | | | Activity | | | | | corrected | chemical | chemical |
| | | Substrate | ([¹¹C] | Injected | Collected | Collected | Isolation | Injection | conversion | conversion |
| Reaction | | Loading | TBACN) | dose | dose b | dose c | time | dose | of b (RCC, | of c (RCC, |
| Entry | Substrate | (mmol) | (mCi) | (μCi) | (μCi) | (μCi) | (min) | (μCi) | %, d.c.) | %, d.c.) |
| 1 | 54a | 0.03 | 18.0 | 552 | 84 | 59 | 7 | 435 | 19.3 | 13.6 |
| 2 | | 0.03 | 4.5 | 222 | 37 | 23 | 9 | 163.5 | 22.6 | 14.1 |
| 3 | | 0.03 | 8.4 | 310 | 65 | 37 | 8 | 236.2 | 27.5 | 15.7 |

Average RCC of b: 23.1 ± 4.1%; Average RCC of c: 14.5 ± 1.1% (N = 3, n = 0.03 mmol, decay corrected)

### HPLC-isolated RCCs of [¹¹C]54 by S2

### Example 167: Radio Reaction Optimization—Radio HPLC Analysis and Characterization of [¹¹C]Cyanide Labeled Arenes—Compounds [¹¹C]55b (C4)/(C3)

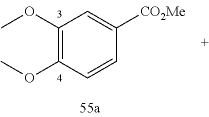

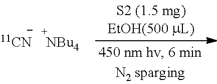

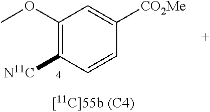

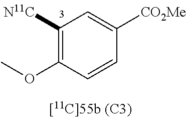

| | | | | | | | | Decay | Radio- | Radio- |
|---|---|---|---|---|---|---|---|---|---|---|
| | | | Activity | | | | | corrected | chemical | chemical |
| | | Substrate | ([¹¹C] | Injected | Collected | Collected | Isolation | Injection | conversion | conversion |
| Reaction | | Loading | TBACN) | dose | dose b | dose c | time | dose | of b (RCC, | of c (RCC, |
| Entry | Substrate | (mmol) | (mCi) | (μCi) | (μCi) | (μCi) | (min) | (μCi) | %, d.c.) | %, d.c.) |
| 1 | 55a | 0.03 | 4.8 | 196 | 28 | 23 | 8 | 149 | 18.8 | 15.4 |
| | | Co-inject | | | | | | | | |
| 2 | | 0.03 | 4.2 | 244 | 36 | 29 | 8 | 185.9 | 19.4 | 15.6 |
| 3 | | 0.03 | 5.45 | 337 | 65 | 56 | 8 | 256.8 | 25.3 | 21.8 |
| 4 | | 0.01 | 4.0 | 326 | 30 | 25 | 8 | 248.4 | 12.1 | 10.1 |

Average RCC of b: 21.2 ± 3.6%; Average RCC of c: 17.6 ± 3.6% (N = 3, n = 0.03 mmol, decay corrected)

### HPLC-isolated RCCs of [¹¹C]55 by S2

### Example 168: Radio Reaction Optimization—Radio HPLC Analysis and Characterization of [¹¹C]Cyanide Labeled Arenes—Compounds [¹¹C]56b (C4)/(C3)

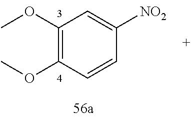

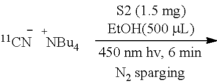

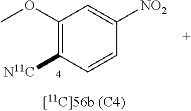

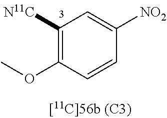

| | | | | | | | | Decay | Radio- | Radio- |
|---|---|---|---|---|---|---|---|---|---|---|
| | | | Activity | | | | | corrected | chemical | chemical |
| | | Substrate | ([¹¹C] | Injected | Collected | Collected | Isolation | Injection | conversion | conversion |
| Reaction | | Loading | TBACN) | dose | dose b | dose c | time | dose | of b (RCC, | of c (RCC, |
| Entry | Substrate | (mmol) | (mCi) | (μCi) | (μCi) | (μCi) | (min) | (μCi) | %, d.c.) | %, d.c.) |
| 1 | 56a | 0.03 | 23.0 | 575 | 80 | 45 | 10 | 409 | 19.6 | 11.0 |
| 2 | | 0.03 | 3.6 | 227 | 40 | 31 | 10 | 161.6 | 24.8 | 19.2 |
| 3 | | 0.03 | 3.9 | 140 | 16 | 12 | 10 | 99.5 | 16.1 | 12.1 |

Average RCC of b: 20.2 ± 4.4%; Average RCC of c: 14.1 ± 4.5% (N = 3, decay corrected)

### HPLC-isolated RCCs of [¹¹C]56 by S2

### Example 169: Radio Reaction Optimization—Radio HPLC Analysis and Characterization of [¹¹C]Cyanide Labeled Arenes—Compounds [¹¹C]57b (C4)/(C3)

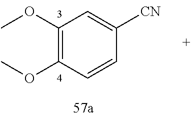

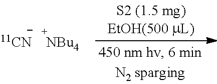

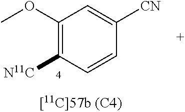

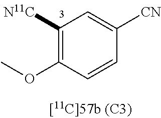

| | | | | | | | | Decay | Radio- | Radio- |
|---|---|---|---|---|---|---|---|---|---|---|
| | | | Activity | | | | | corrected | chemical | chemical |
| | | Substrate | ([¹¹C] | Injected | Collected | Collected | Isolation | Injection | conversion | conversion |
| Reaction | | Loading | TBACN) | dose | dose b | dose c | time | dose | of b (RCC, | of c (RCC, |
| Entry | Substrate | (mmol) | (mCi) | (μCi) | (μCi) | (μCi) | (min) | (μCi) | %, d.c.) | %, d.c.) |
| 1 | 57a | 0.03 | 14.4 | 598 | 138 | 75 | 9 | 440 | 31.4 | 17.0 |
| 2 | | 0.04 | 6.4 | 230 | 68 | 35 | 8 | 175.2 | 38.8 | 20.0 |
| 3 | | 0.02 | 5.85 | 303 | 76 | 36 | 8 | 230.8 | 32.9 | 15.6 |

Average RCC of b: 34.4 ± 3.9%; Average RCC of c: 17.5 ± 2.2% (N = 3, decay corrected)

### HPLC-isolated RCCs of [¹¹C]57 by S2

### Example 170: Radio Reaction Optimization—Radio HPLC Analysis and Characterization of [¹¹C]Cyanide Labeled Arenes—Compounds [¹¹C]58b (C4)/(C3)

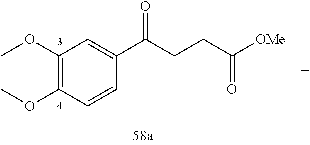 [SMILES: COC(=O)CCC(=O)c1ccc(OC)c(OC)c1]

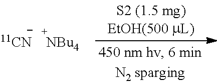

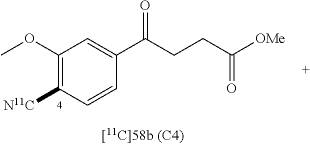

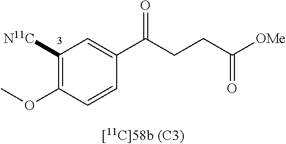

| | | | | | | | | Decay | Radio- | Radio- |
|---|---|---|---|---|---|---|---|---|---|---|
| | | | Activity | | | | | corrected | chemical | chemical |
| | | Substrate | ([¹¹C] | Injected | Collected | Collected | Isolation | Injection | conversion | conversion |
| Reaction | | Loading | TBACN) | dose | dose b | dose c | time | dose | of b (RCC, | of c (RCC, |
| Entry | Substrate | (mmol) | (mCi) | (μCi) | (μCi) | (μCi) | (min) | (μCi) | %, d.c.) | %, d.c.) |
| 1 | 58a | 0.02 | 12.3 | 467 | 50 | 26 | 12 | 310.5 | 16.1 | 8.4 |
| 2 | | 0.02 | 6.6 | 251 | 31 | 18 | 10 | 178.7 | 17.3 | 10.1 |
| 3 | | 0.02 | 4.7 | 228 | 21 | 15 | 11 | 156.8 | 13.4 | 9.6 |

Average RCC of b: 15.6 ± 2.0%; Average RCC of c: 9.4 ± 0.9% (N = 3, decay corrected)

### HPLC-isolated RCCs of [¹¹C]58 by S2

### Example 171: Radio Reaction Optimization—Radio HPLC Analysis and Characterization of [¹¹C]Cyanide Labeled Arenes—Compound [¹¹C]59b

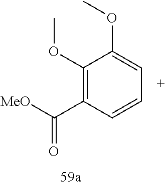

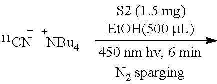

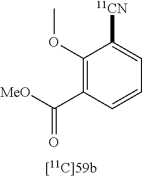

| | | | | | | | Decay | Radio- |
|---|---|---|---|---|---|---|---|---|
| | | | Activity | | | | corrected | chemical |
| | | Substrate | ([¹¹C] | Injected | Collected | Isolation | Injection | conversion |
| Reaction | | Loading | TBACN) | dose | dose b | time | dose | of b (RCC, |
| Entry | Substrate | (mmol) | (mCi) | (μCi) | (μCi) | (min) | (μCi) | %, d.c.) |
| 1 | 59a | 0.03 | 13.8 | 545 | 80 | 9 | 401.4 | 20 |
| 2 | | 0.03 | 5.1 | 250 | 24 | 10 | 177.9 | 13.5 |
| 3 | | 0.03 | 2.9 | 129 | 21 | 7 | 101.7 | 20.6 |

Average RCC of b: 18.0 ± 3.9% (N = 3, decay corrected)

### HPLC-isolated RCCs of [¹¹C]59b by S2

### Example 172: Radio Reaction Optimization—Radio HPLC Analysis and Characterization of [¹¹C]Cyanide Labeled Arenes—Compounds [¹¹C]60b/60c

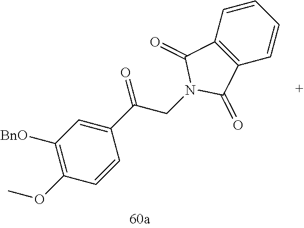 [SMILES: COc1ccc(C(=O)CN2C(=O)c3ccccc3C2=O)cc1OCc1ccccc1]

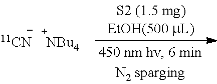

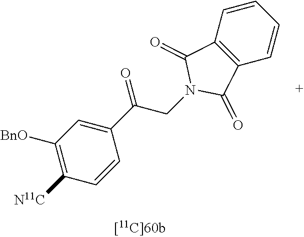

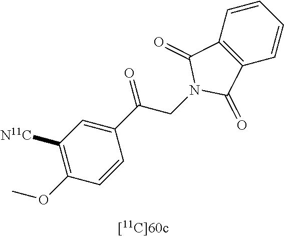

| | | | | | | | | Decay | Radio- | Radio- |
|---|---|---|---|---|---|---|---|---|---|---|
| | | | Activity | | | | | corrected | chemical | chemical |
| | | Substrate | ([¹¹C] | Injected | Collected | Collected | Isolation | Injection | conversion | conversion |
| Reaction | | Loading | TBACN) | dose | dose b | dose c | time | dose | of b (RCC, | of c (RCC, |
| Entry | Substrate | (mmol) | (mCi) | (μCi) | (μCi) | (μCi) | (min) | (μCi) | %, d.c.) | %, d.c.) |
| 1 | 60a | 0.02 | 19.7 | 655 | 0 | 0 | — | — | — | — |
| 2 | | 0.02 | 13.6 | 270 | 0 | 0 | — | — | — | — |
| 3 | | 0.02 | 6.1 | 145 | 0 | 0 | — | — | — | — |
| 4 | | 0.012 | 4.6 | 301 | 0 | 0 | — | — | — | — |
| 5 | | 0.01 | 12.5 | 286 | 0 | 0 | — | — | — | — |

N.R. (N = 5)

### HPLC-isolated RCCs of [¹¹C]60 by S2

### Example 173: Radio Reaction Optimization—Radio HPLC Analysis and Characterization of [¹¹C]Cyanide Labeled Arenes—Compounds [C]61b/61c

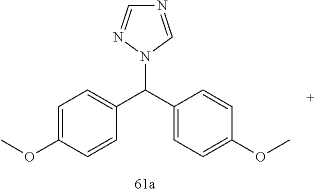

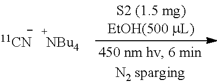

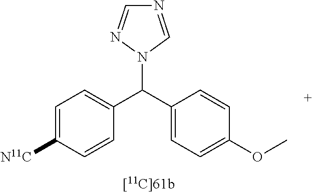

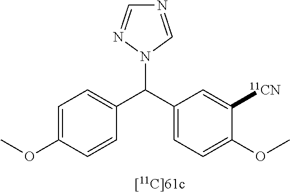

| | | | | | | | | Decay | Radio- | Radio- |
|---|---|---|---|---|---|---|---|---|---|---|
| | | | Activity | | | | | corrected | chemical | chemical |
| | | Substrate | ([¹¹C] | Injected | Collected | Collected | Isolation | Injection | conversion | conversion |
| Reaction | | Loading | TBACN) | dose | dose b | dose c | time | dose | of b (RCC, | of c (RCC, |
| Entry | Substrate | (mmol) | (mCi) | (μCi) | (μCi) | (μCi) | (min) | (μCi) | %, d.c.) | %, d.c.) |
| 1 | 61a | 0.02 | 6.7 | 232 | 24 | 32 | 11 | 159.6 | 15.0 | 20.1 |
| 2 | | 0.02 | 9.3 | 280 | 23 | 33 | 10 | 199 | 11.6 | 16.6 |
| 3 | | 0.02 | 3.0 | 144 | 12 | 18 | 10 | 102.5 | 11.7 | 17.6 |

Average RCC of b: 12.8 ± 1.9%; Average RCC of c: 18.1 ± 1.8% (N = 3, decay corrected)

HPLC-isolated RCCs of [¹¹C]61b and [¹¹C]61c by S2

### Example 174: Radio Reaction Optimization—Radio HPLC Analysis and Characterization of [¹¹C]Cyanide Labeled Arenes—Compound [¹¹C]62b

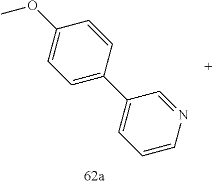

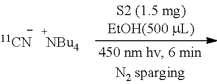

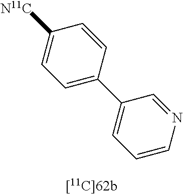

| | | | | | | | Decay | Radio- |
|---|---|---|---|---|---|---|---|---|
| | | | Activity | | | | corrected | chemical |
| | | Substrate | ([¹¹C] | Injected | Collected | Isolation | Injection | conversion |
| Reaction | | Loading | TBACN) | dose | dose b | time | dose | of b (RCC, |
| Entry | Substrate | (mmol) | (mCi) | (μCi) | (μCi) | (min) | (μCi) | %, d.c.) |
| 1 | 62a | 0.03 | 14.8 | 548 | 128 | 10 | 390 | 32.8 |
| 2 | | 0.03 | 15.9 | 496 | 122 | 8 | 377.9 | 32.3 |
| 3 | | 0.03 | 11.7 | 423 | 104 | 10 | 301 | 34.6 |
| 4 | | 0.03 | 3.9 | 185 | 50 | 10 | 131.7 | 38.0 |
| | | co-inject | | | | | | |
| 5 | N₂ pre- | 0.03 | 18.7 | 717 | 116 | 8 | 546 | 21.2 |
| | sparging | | | | | | | |

Average RCC: 34.4 ± 2.6% (N = 4, decay corrected)

### HPLC-isolated RCCs of [¹¹C]62b by S2

### Example 175: Radio Reaction Optimization—Radio HPLC Analysis and Characterization of [¹¹C]Cyanide Labeled Arenes—Compound [¹¹C]63b

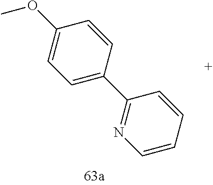

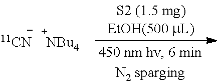

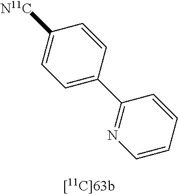

| | | | | | | | Decay | Radio- |
|---|---|---|---|---|---|---|---|---|
| | | | Activity | | | | corrected | chemical |
| | | Substrate | ([¹¹C] | Injected | Collected | Isolation | Injection | conversion |
| Reaction | | Loading | TBACN) | dose | dose b | time | dose | of b (RCC, |
| Entry | Substrate | (mmol) | (mCi) | (μCi) | (μCi) | (min) | (μCi) | %, d.c.) |
| 1 | 63a | 0.03 | 6.0 | 277 | 16 | 8 | 211 | 7.6 |
| 2 | | 0.03 | 10.3 | 453 | 27 | 9 | 333.6 | 8.1 |
| 3 | | 0.01 | 7.4 | 321 | 30 | 8 | 244.6 | 12.3 |
| | | co-inject | | | | | | |

Average RCC: 9.3 ± 2.6% (N = 3, decay corrected)

### HPLC-isolated RCCs of [¹¹C]63b by S2

### Example 176: Radio Reaction Optimization—Radio HPLC Analysis and Characterization of [¹¹C]Cyanide Labeled Arenes—Compound [¹¹C]64b

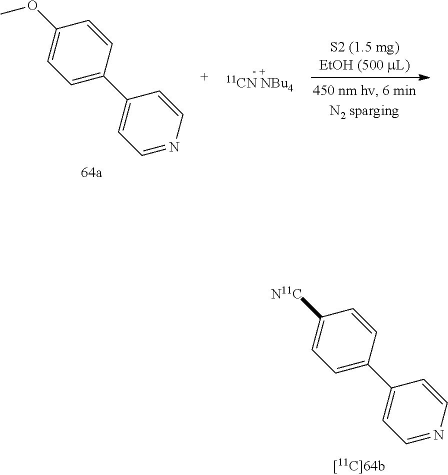

[¹¹C]Compound 64a (n=0.01 mmol) was isolated with HPLC chromatography (isocratic 35%, HPLC condition B) and compared to non-radioactive [¹²C]Compound 64a.

### Example 177: Radio Reaction Optimization—Radio HPLC Analysis and Characterization of [¹¹C]Cyanide Labeled Arenes—Compound [¹¹C]65b

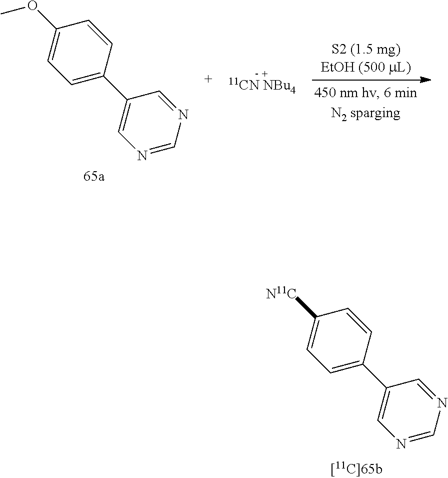

[¹¹C]Compound 65a (n=0.01 mmol) was isolated with HPLC chromatography (isocratic 35%, HPLC condition B) and compared to non-radioactive [¹²C]Compound 65a.

### Example 178: Molar Activity Calculation-Molar Activity Calculation for 2,6-Dimethoxybenzo-¹¹C-Nitrile ([¹¹C]1b) Obtained from ¹¹C-Radiocyanation of 1,2,3-Trimethoxylbenzene (1a)

Molar activity was calculated using a standard curve of the corresponding arene nitrile. A ¹²CN standard curve [X axis=UV area (254 nm), Y axis=mass (g)] was created from the HPLC trace from a standard solution of 2,6-dimethoxybenzonitrile (1b). The radiolabeled product from the labeling reaction was purified and collected via HPLC; the UV area (254 nm) overlapping with the radio peak was then recorded. The standard curve was used to calculate mass that was converted into the mole number. Dividing the product decay corrected activity by the mole number gives the molar activity in GBq/mol. In this example, the isolated product 2,6-dimethoxybenzo-¹¹C-nitrile ([¹¹C]1b) has a molar activity of 84.78±6.96 GBq/μmol, which is decay corrected to the end of bombardment (EOB). See Table 178.1 and 178.2 below for data of molar activity calculations carried out for a standard curve of 1b (and also see FIG. 13).

TABLE 178.1

| Area | m (μg) |
|---|---|
| 0 | 0 |
| 1.52E+05 | 0.041 |
| 2.86E+05 | 0.082 |
| 5.95E+05 | 0.205 |
| 1.20E+06 | 0.41 |
| 2.97E+06 | 1.23 |
| 1.04E+07 | 4.1 |

TABLE 178.2
Average molar activity: 84.78 ± 6.96 GBq/μmol

| | Decay corrected | UV Area | | | Molar activity | Molar activity |
|---|---|---|---|---|---|---|
| Entry | (EOB) Activity (μCi) | (AU) | m(μg) | n(μmol) | (Ci/μmol) | (GBq/μmol) |
| 1 | 1642 | 354901 | 0.1219 | 0.000747 | 2.197568526 | 81.31004 |
| 2 | 6530 | 1332566 | 0.5107 | 0.00313 | 2.086269006 | 77.19195 |
| 3 | 10680 | 1791571 | 0.6933 | 0.004249 | 2.513714322 | 93.00743 |
| 4 | 7093 | 1277663 | 0.4889 | 0.002996 | 2.367349407 | 87.59193 |

Average molar activity: 84.78 ± 6.96 GBq/μmol

### Example 179: PET Imaging Study

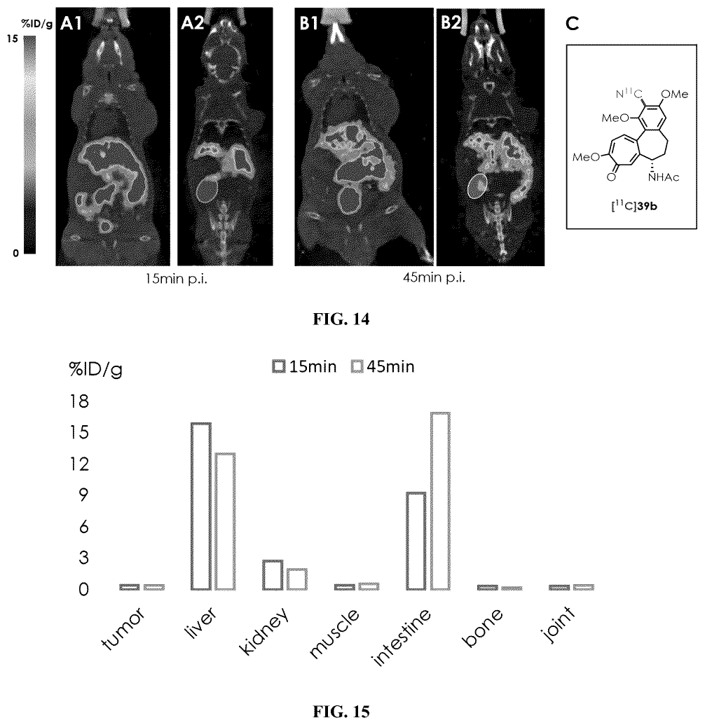

In drug discovery, radiolabeled lead compounds or their analogs can be further evaluated in vivo to explore their bioactivity, and to measure the pharmacodynamics and pharmacokinetics of the drug candidates. Using the developed method disclosed herein, ¹¹C-labeled aryl nitriles were prepared for distribution studies.¹⁵ Previously, colchicine (39a) was reported to be excreted primarily through the biliary system, intestines, and kidneys with certain toxicity.¹⁶ Herein, a nitrile analog of colchicine ([¹¹C]39b) was synthesized using the ¹¹C-cyanation disclosed herein catalyzed by S1. A small animal PET study was performed using [¹¹C]39b to evaluate its distribution profile in vivo. PET/CT image was acquired at 15 min (A1 and A2) and 45 min (B1 and B2) post intravenous tail vein injection of [¹¹C]39b. A1 and B1 show the liver coronal plane. A2 and B2 show the kidney coronal plane with the kidney circled. (C) Chemical structure of [¹¹C]39b. As shown in FIG. 14, [¹¹C]39b showed apparent uptake in liver, intestines, and kidneys, which was similar to the reported patter of 39a (see detailed PET imaging ROIs analysis in FIG. 15).

Specifically, BALB/c nude mice bearing PC3-PSMA tumors by subcutaneous injection of PC3-PSMA cells were prepared. The mice were used for imaging studies when the tumor size reached 500 mm. The cancer cell line was purchased from the Tissue Culture Facility, UNC Lineberger Comprehensive Cancer Center. All animal experiments and procedures complied with the protocols of the UNC Institutional Animal Care and Use Committee.

Approximately 3.7 MBq (˜0.12 MBq/g) of [¹¹C]39b was injected into the tail veins of the mouse. 15 min static PET and CT images at 15- and 45-minutes post-injection were acquired with animals under isoflurane anesthesia. The regions of interest (ROIs) were drawn in Amide software (amide.sourceforge.net) (see FIG. 15).³¹

### REFERENCES

1. Zhao, W., Lee, H. G., Buchwald, S. L., and Hooker, J. M. (2017). Direct (11)CN-Labeling of Unprotected Peptides via Palladium-Mediated Sequential Cross-Coupling Reactions. J Am Chem Soc 139, 7152-7155. 10.1021/jacs.7b02761.
2. Nordeman, P., Chow, S. Y., Odell, A. F., Antoni, G., and Odell, L. R. (2017). Palladium-mediated (11)C-carbonylations using aryl halides and cyanamide. Org Biomol Chem 15, 4875-4881. 10.1039/c7ob01064h.
3. Liu, L., Xu, Y., Shea, C., Fowler, J. S., Hooker, J. M., and Tonge, P. J. (2010). Radiosynthesis and bioimaging of the tuberculosis chemotherapeutics isoniazid, rifampicin and pyrazinamide in baboons. J Med Chem 53, 2882-2891. 10.1021/jm901858n.
4. Ponchant, M., Hinnen, F., Demphel, S., and Crouzel, C. (1997). [11C]copper(I) cyanide: a new radioactive precursor for 11C-cyanation and functionalization of haloarenes. Applied Radiation and Isotopes 48, 755-762. doi.org/10.1016/S0969-8043(96)00299-0.
5. Ma, L., Placzek, M. S., Hooker, J. M., Vasdev, N., and Liang, S. H. (2017). [(11)C]Cyanation of arylboronic acids in aqueous solutions. Chem Commun (Camb) 53, 6597-6600. 10.1039/c7cc02886e.
6. Yang, L., Brooks, A. F., Makaravage, K. J., Zhang, H., Sanford, M. S., Scott, P. J. H., and Shao, X. (2018). Radiosynthesis of [(11)C]LY2795050 for Preclinical and Clinical PET Imaging Using Cu(II)-Mediated Cyanation. ACS Med Chem Lett 9, 1274-1279. 10.1021/acsmedchemlett.8b00460.
7. Makaravage, K. J., Shao, X., Brooks, A. F., Yang, L., Sanford, M. S., and Scott, P. J. H. (2018). Copper(II)-Mediated [(11)C]Cyanation of Arylboronic Acids and Arylstannanes. Org Lett 20, 1530-1533. 10.1021/acs.orglett.8b00242.
8. M. Haziq Qureshi, R. W., and Christopher Marshall (2022). Top 200 Small Molecule Pharmaceuticals by Retail Sales in 2021. In njardarson.lab.arizona.edu/sites/njardarson.lab.arizona.edu/files/Top %20200%20Small %2 0Molecules %202021V3.pdf, ed.
9. Chen, W., Wang, H., Tay, N. E. S., Pistritto, V. A., Li, K. P., Zhang, T., Wu, Z., Nicewicz, D. A., and Li, Z. (2021). Arene radiofluorination enabled by photoredox-mediated halide interconversion. Nat Chem. 10.1038/s41557-021-00835-7.
10. Chen, W., Huang, Z., Tay, N. E. S., Giglio, B., Wang, M., Wang, H., Wu, Z., Nicewicz, D. A., and Li, Z. (2019). Direct arene C—H fluorination with (18)F(—) via organic photoredox catalysis. Science 364, 1170-1174. 10.1126/science.aav7019.
11. Holmberg-Douglas, N., and Nicewicz, D. A. (2019). Arene Cyanation via Cation-Radical Accelerated-Nucleophilic Aromatic Substitution. Org Lett 21, 7114-7118. 10.1021/acs.orglett.9b02678.
12. Popp, J. L., and Kirk, T. K. (1991). Oxidation of methoxybenzenes by manganese peroxidase and by Mn3+. Arch Biochem Biophys 288, 145-148. 10.1016/0003-9861(91)90176-j.
13. McManus, J. B., and Nicewicz, D. A. (2017). Direct C—H Cyanation of Arenes via Organic Photoredox Catalysis. J Am Chem Soc 139, 2880-2883. 10.1021/jacs.6bl2708.
14. Zhao, Y., Li, D., Wei, M., Du, R., and Yan, Z. (2021). The ester derivatives obtained by C-ring modification of podophyllotoxin induce apoptosis and inhibited proliferation in PC-3M cells via down-regulation of PI3K/Akt signaling pathway. Bioorg Med Chem Lett 46, 128174. 10.1016/j.bmcl.2021.128174.
15. Ben-Chetrit, E. (2018). Colchicine. Textbook of Autoinflammation, 729-749. 10.1007/978-3-319-98605-0_4
16. Zhang, W., Carpenter, K. L., and Lin, S. (2020). Electrochemistry Broadens the Scope of Flavin Photocatalysis: Photoelectrocatalytic Oxidation of Unactivated Alcohols. Angew Chem Int Ed Engl 59, 409-417. 10.1002/anie.201910300.
17. Lu, J., Pattengale, B., Liu, Q., Yang, S., Shi, W., Li, S., Huang, J., and Zhang, J. (2018). Donor-Acceptor Fluorophores for Energy-Transfer-Mediated Photocatalysis. J Am Chem Soc 140, 13719-13725. 10.1021/jacs.8b07271.
18. Garcia, L., Sendra, J., Miralles, N., Reyes, E., Carbo, J. J., Vicario, J. L., and Fernandez, E. (2018). Transition-Metal-Free Stereoselective Borylation of Allenamides. Chemistry 24, 14059-14063. 10.1002/chem.201803004.
19. Iwamoto, T., Okuzono, C., Adak, L., Jin, M., and Nakamura, M. (2019). Iron-catalysed enantioselective Suzuki-Miyaura coupling of racemic alkyl bromides. Chem Commun (Camb) 55, 1128-1131. 10.1039/c8cc09523j.
20. Joncour, A., Decor, A., Thoret, S., Chiaroni, A., and Baudoin, O. (2006). Biaryl axis as a stereochemical relay for the enantioselective synthesis of antimicrotubule agents. Angew Chem Int Ed Engl 45, 4149-4152. 10.1002/anie.200600451.
21. Hao-Jie Rong, C.-F. Y., Tao Chen, Yong-Qiang Wang, Bin-Ke Ning (2019). Synthesis of guanidines via the I2 mediated desulfurization of N,N′-di-Boc-thiourea. Tetrahedron Letters 60, 150970. 10.1016/j.tetlet.2019.150970.
22. Cochrane, E. J., Leonori, D., Hassall, L. A., and Coldham, I. (2014). Synthesis and kinetic resolution of N-Boc-2-arylpiperidines. Chem Commun (Camb) 50, 9910-9913. 10.1039/c4cc04576a.
23. Weiyuan Liu, L. C., Jing Pan, Ting Zhou, Kuaile Lin, Weicheng Zhou (2021). A novel and practical synthesis of Iclaprim. Synthetic Communications 51, 410-418. 10.1080/00397911.2020.1831023.
24. Rosenmund-von Braun Reaction. In Comprehensive Organic Name Reactions and Reagents, pp. 2425-2428. doi.org/10.1002/9780470638859.conrr547.
25. Zhao, C., Fang, W.-Y., Rakesh, K. P., and Qin, H.-L. (2018). Pd-Catalyzed one-pot dehydroxylative coupling of phenols with K4[Fe(CN)6] mediated by S02F2: a practical method for the direct conversion of phenols to aryl nitriles. Organic Chemistry Frontiers 5, 1835-1839. 10.1039/C8QO00295A.
26. D. Hayrapetyan, R. K. R., M. Kratz, K. Tschulik, L. J. Goo8en (2018). Electrochemical C—H Cyanation of Electron-Rich (Hetero)Arenes. Chemistry—A European Journal 24, 11288. 10.1002/chem.201802247.
27. Zhou, D. (2012) Preparation method of cinepazide maleate. China patent CN102887874B, patent application CN201210456321.3A.
28. Kim, J. Y., An, G.-i., Chun, K. S., and Rhee, H. (2005). A Short Synthesis of Trimebutine, 2-Dimethylamino-2-phenylbutyl 3,4,5-trimethylbenzoate. Bulletin of The Korean Chemical Society 26, 340-342.
29. Steinbach, J., Lebeda, O., and Walther, M. (2015). Proceedings of the 15th International Workshop on Targetry and Target Chemistry (Helmholtz-Zentrum Dresden-Rossendorf).
30. Iwata, R., Ido, T., Takahashi, T., Nakanishi, H., and Iida, S. (1987). Optimization of [11C]HCN production and no-carrier-added [1-11C]amino acid synthesis. Int J Rad Appl Instrum A 38, 97-102. 10.1016/0883-2889(87)90003-7.
31. Loening, A. M., and Gambhir, S. S. (2003). AMIDE: a free software tool for multimodality medical image analysis. Mol Imaging 2, 131-137. 10.1162/153535003322556877.
32. Yan, G., Zhang, Y., and Wang, J. (2017). Recent Advances in the Synthesis of Aryl Nitrile Compounds. Advanced Synthesis & Catalysis 359, 4068-4105. doi.org/10.1002/adsc.201700875.

1. A photoredox-catalyzed cyanation method comprising:
a) obtaining a reaction mixture comprising an arene or heteroarene substrate, a photocatalyst, a base additive, and a solvent;
b) contacting the reaction mixture with a cyanide source to afford a photocyanation reaction mixture; and
c) exposing the photocyanation reaction mixture to blue-violet light to form a cyano arene product or a cyano heteroarene product.

2. The method of claim 1, wherein the photocatalyst exhibits an excited state reduction potential ranging from about +2.5V vs. SCE to about +0.1V vs. SCE.

3. The method of claim 1, wherein the photocatalyst is selected from the group consisting of Mes-Acr-Ph+, 4CzIPN, RFTA, EOSIN, Ir-ll, and a combination thereof.

4. The method of claim 1, wherein the photocatalyst is present in an amount of from about 0.5 mol % to about 5 mol % with respect to the arene or heteroarene substrate.

5. The method of claim 1, wherein the cyanide source is selected from the group consisting of acetone cyanohydrin (ACH), tetrabutylammonium cyanide (TBACN or NBu₄CN), trimethylsilyl cyanide (TMSCN), sodium cyanide (NaCN), potassium cyanide (KCN), and combinations thereof.

6. The method of claim 1, wherein the cyanide source is non-radioactive and is present in an amount of from about 1.5 equiv. to about 4 equiv.

7. The method of claim 1, wherein the arene or heteroarene substrate is a biologically active molecule selected from the group consisting of a pharmacological agent and a pharmaceutical agent.

8. The method claim 1, wherein the arene or heteroarene substrate is monocyclic or multicyclic.

9. The method of claim 8, wherein the multicyclic aromatic ring is a naphthalene or a quinoline.

10. The method of claim 8, wherein the monocyclic aromatic ring or the multicyclic aromatic ring is substituted with at least one alkoxy group selected from the group consisting of —OCH₃, —OCH₂CH₃, —OCH(CH₃)₂, —OC(CH₃)₃ and —O(Ar).

11. The method of claim 10, wherein the cyano arene product or cyano heteroarene product is formed via a photoredox-catalyzed cation radical-accelerated nucleophilic aromatic substitution (CRA-S_{N}Ar), wherein the alkoxy group serves as a nucleofuge.

12. The method of claim 1, wherein the substrate comprises an alkoxy-substituted monocyclic aromatic ring according to Formula (II): 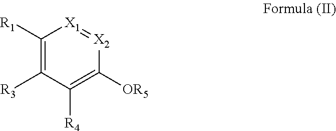
wherein X₁ and X₂ are each independently selected from the group consisting of —N and —CR₂;
R₁ and R₂ are each independently selected from the group consisting of —H, substituted or unsubstituted —(C₁-C₆) alkyl, substituted or unsubstituted —O(C₁-C₆) alkyl, substituted or unsubstituted —O(benzyl), substituted or unsubstituted —CH₂CO(C₁-C₆) alkyl, substituted or unsubstituted —(C₁-C₆) alkyl-NH(R₆), substituted or unsubstituted —N(C₁-C₆ alkyl)(R₆), —CH═CHCOOH, substituted or unsubstituted —CO(C₁-C₆) alkyl, substituted or unsubstituted —COO(C₁-C₆) alkyl, substituted or unsubstituted heteroaryl, and —COOH;
R₃ is selected from the group consisting of —H, substituted or unsubstituted —O(C₁-C₆) alkyl, substituted or unsubstituted —(C₁-C₆) alkyl, substituted or unsubstituted —CH₂CO(C₁-C₆) alkyl and substituted or unsubstituted —(C₁-C₆) alkyl-NH(R₆);
R₄ is selected from the group consisting of —H, substituted or unsubstituted —O(C₁-C₆) alkyl, substituted or unsubstituted —(C₁-C₆) alkyl, —Cl and —F;
R₅ is selected from the group consisting of substituted or unsubstituted (C₁-C₆) alkyl group, substituted or unsubstituted aryl group, and substituted or unsubstituted heteroaryl group;
R₆ is —H or an amine protecting group (PG); and
a pharmaceutically acceptable salt form thereof.

13. The method of claim 12, wherein X₁ and X₂ both are —CR₂.

14. The method of claim 12, wherein R₂ is selected from the group consisting of —H, —CH₃ and —OCH₃.

15. The method of claim 12, wherein R₃ is selected from the group consisting of —H, —OCH₃, —CH₃, —CH₂COCH₃, and —CH₂CH₂NH(R).

16. The method of claim 12, wherein R₄ is selected from the group consisting of —CH₃, —OCH₃, —OCH₂CH₃, —OCH(CH₃)₂, and —F.

17. The method of claim 12, wherein R₁ is selected from the group consisting of —CH₃, —OCH₃, —H, —C₁, -Ph, —C(CH₃)₃, —CH₂COCH₃, —CH₂CH₂NH(BOC), —N(CH₃)(BOC), —Br, —CN, —CH═CHCOOH; -pyridin-2-yl, —CH₂C₁, —CH₂CN, —CH₂OH, —CH₂OCH₂CH₃, —CH₂NH(BOC), —CH₂N₃, —COCH₂CH₂COOCH₃, —COH, —COCH₃, —COOCH₃, and —COOH.

18. The method of claim 12, wherein R₄ is —OCH₃ and X₂ is —CR₂, wherein R₂ is —H or —OCH₃.

19. The method of claim 1, wherein the arene substrate comprises a six membered aryl ring moiety such as

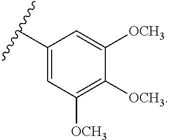 [SMILES: *c1cc(OC)c(OC)c(OC)c1]

20. The method of claim 19, wherein the arene substrate is selected from the group consisting of colchicine, benzyl guanidine, troxipide, trimethoprim, cinepazide, letrozole and trimebultine.

21. The method of claim 1, wherein the base additive is selected from the group consisting of Na₂CO₃, NaOAc, KHCO₃, NaHCO₃, DIPEA, KOAc, and a combination thereof.

22. The method of claim 1, wherein the cyanide source is a radioactive cyanide source comprising a radioisotope selected from the group consisting of ¹¹C and ¹³N.

23. The method of claim 22, wherein the cyanide source is selected from the group consisting of [¹³C]TMSCN, [¹³C]KCN, NBu₄[¹¹C]CN, TBA[¹¹C]CN, and a combination thereof.

24. The method of claim 22, wherein the radioactive cyanide source is NBu₄[¹¹C]CN.

25. The method of claim 22, wherein the radioactive cyanide source comprises an activity of from about 0.11 to about 1.1 GBq.

26. The method of claim 22, wherein the radiochemical yield (RCY) is at least 50%.
